(19) United States
(12) Patent Application Publication
(10) Pub. No.: US 2026/0176395 A1
(43) Pub. Date: Jun. 25, 2026

(54) RESIST COMPOSITION, METHOD FOR FORMING RESIST PATTERN, AND ACID DIFFUSION CONTROL AGENT

(71) Applicant: Tokyo Ohka Kogyo Co., Ltd., Kawasaki-shi (JP)

(72) Inventors: Tetsuya Matsushita, Kawasaki-shi (JP); Takatoshi Inari, Kawasaki-shi (JP)

(21) Appl. No.: 19/124,562

(22) PCT Filed: Oct. 25, 2023
(86) PCT No.: PCT/JP2023/038552

§ 371 Date: Apr. 25, 2025

(30) Foreign Application Priority Data
Oct. 31, 2022 (JP) 2022-174658
Oct. 31, 2022 (JP) 2022-174668
Oct. 31, 2022 (JP) 2022-174680
Oct. 31, 2022 (JP) 2022-174687

(51) Int. Cl. C08F 220/24

(52) U.S. Cl. CPC C08F 220/24; C07C 2601/14

(57) ABSTRACT

A resist composition which generates an acid upon light exposure and whose solubility in a developing solution is changed by an action of the acid, the resist composition including a resin component whose solubility in a developing solution is changed by the action of the acid, and a compound represented by General Formula (d0) in which Rd⁰¹ represents a monovalent organic group, Rd⁰² represents a monovalent organic group or a hydrogen atom, and Ld⁰¹ represents a single bond or a divalent linking group

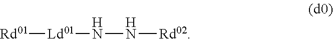

### TECHNICAL FIELD

The present invention relates to a resist composition, a method for forming a resist pattern, and an acid diffusion control agent.

Priority is claimed on Japanese Patent Application Nos. 2022-174658, 2022-174668, 2022-174687, and 2022-174680, filed Oct. 31, 2022, the contents of which are incorporated herein by reference.

### BACKGROUND ART

In recent years, in the production of semiconductor elements and liquid crystal display elements, advances in lithography technologies have led to a rapid progress in the field of pattern fining. Typically, these pattern fining technologies involve shortening the wavelength (increasing the energy) of the light source for exposure.

A resist material is required to have lithography characteristics such as resolution that enables reproduction of a fine-sized pattern, and sensitivity to these types of exposure light sources.

As a resist material that satisfies these requirements, a chemically amplified resist composition that contains a base material component whose solubility in a developing solution is changed by an action of an acid, and an acid generator component that generates an acid upon light exposure has been used in the related art.

In the resist pattern formation, the behavior of an acid generated from an acid generator component upon light exposure is considered as one factor that has a great influence on lithography characteristics. Meanwhile, a chemically amplified resist composition in which an acid generator component and an acid diffusion control agent that controls the diffusion of the acid generated from the acid generator component upon light exposure are used in combination has been suggested.

For example, Patent Document 1 describes a resist composition containing a photodecomposable base formed of a carboxylate as an acid diffusion control agent component.

### CITATION LIST

### Patent Document

Patent Document 1: Japanese Unexamined Patent Application, First Publication No. 2021-103230

### SUMMARY OF INVENTION

### Technical Problem

With further advances in lithography technologies, rapid progress in the field of pattern fining is being achieved together with the expansion of application fields. Along with this, a technology that enables formation of a fine pattern having a satisfactory shape is required in a case where a semiconductor element or the like is manufactured. Therefore, the resist composition is required to achieve higher sensitivity and to further improve lithography characteristics such as pattern dimension uniformity.

The present invention has been made in consideration of the above-described circumstances, and an object thereof is to provide a resist composition capable of achieving high sensitivity and having satisfactory lithography characteristics, a method for forming a resist pattern using the resist composition, and an acid diffusion control agent that can be used for producing the resist composition.

### Solution to Problem

In order to solve the above-described problems, the present invention has adopted the following configurations.

That is, according to a first aspect of the present invention, there is provided a resist composition which generates an acid upon light exposure and whose solubility in a developing solution is changed by an action of the acid, the resist composition including: a resin component (A1) whose solubility in a developing solution is changed by the action of the acid; and a compound (D0) represented by General Formula (d0).

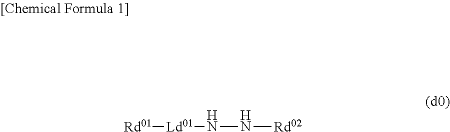

[In the formula, Rd⁰¹ represents a monovalent organic group, Rd⁰² represents a monovalent organic group or a hydrogen atom, and Ld⁰¹ represents a single bond or a divalent linking group.]

According to a second aspect of the present invention, there is provided a method for forming a resist pattern including: a step of forming a resist film on a support using the resist composition according to the first aspect, a step of exposing the resist film to light; and a step of developing the resist film exposed to light to form a resist pattern.

According to a third aspect of the present invention, there is provided a compound which is represented by General Formula (dc01).

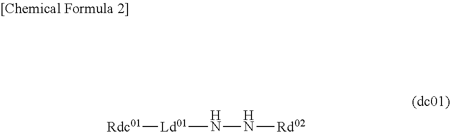

[In the formula, Rdc⁰¹ represents a monovalent organic group having at least two benzene rings which may have a substituent and having at least one heteroatom, Rd⁰² represents a monovalent organic group or a hydrogen atom, and Ld⁰¹ represents a single bond or a divalent linking group.]

According to a fourth aspect of the present invention, there is provided a compound which is represented by General Formula (d02-3).

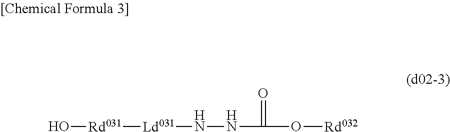

[In the formula, Rd⁰³¹ represents a divalent organic group, Rd⁰³² represents a monovalent organic group, and Ld⁰³¹ represents a single bond or a divalent linking group.]

According to a fifth aspect of the present invention, there is provided a compound which is represented by General Formula (d03-1-1).

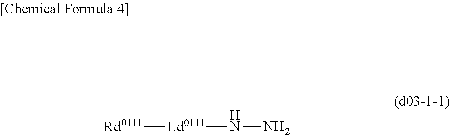

[In the formula, Rd⁰¹¹¹ represents a monovalent organic group having at least one iodine atom as a substituent, and Ld⁰¹¹¹ represents a divalent linking group.]

According to a sixth aspect of the present invention, there is provided a compound which is represented by General Formula (d03-2-1).

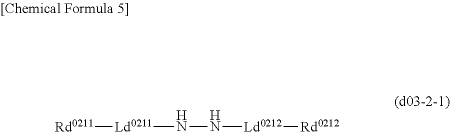

[In the formula, Rd⁰²¹¹ and Rd⁰²¹² each independently represent a monovalent organic group, and Ld⁰²¹¹ and Ld⁰²¹² each independently represent a divalent linking group. Here, at least one of Rd⁰²¹¹ or Rd⁰²¹² has at least one iodine atom as a substituent.]

According to a seventh aspect of the present invention, there is provided a compound which is represented by General Formula (d03-2-2).

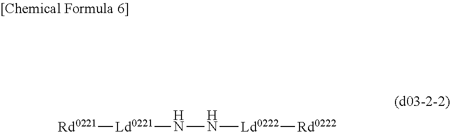

[In the formula, Rd⁰²²¹ represents a monovalent organic group, Rd⁰²²² represents a monovalent organic group having —(CH₂)ₚ—OH or a carboxy group as a substituent, p represents an integer of 1 to 3, and Ld⁰²²¹ and Ld⁰²²² each independently represent a single bond or a divalent linking group. Here, at least one of Rd⁰²²¹ or Rd⁰²²² has at least one iodine atom as a substituent.]

According to an eighth aspect of the present invention, there is provided an acid diffusion control agent including: a compound represented by General Formula (d0).

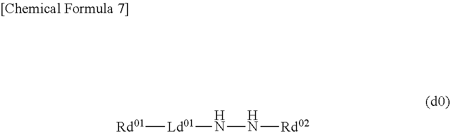

[In the formula, Rd⁰¹ represents a monovalent organic group, Rd⁰² represents a monovalent organic group or a hydrogen atom, and Ld⁰¹ represents a single bond or a divalent linking group.]

### Advantageous Effects of Invention

According to the present invention, it is possible to provide a resist composition capable of achieving high sensitivity and having satisfactory lithography characteristics, a method for forming a resist pattern using the resist composition, and an acid diffusion control agent that can be used for producing the resist composition.

### DESCRIPTION OF EMBODIMENTS

In the present specification and the scope of the present claims, the term “aliphatic” is a relative concept used with respect to “aromatic” and defines a group or compound that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “acid decomposable group” indicates a group having acid decomposability in which at least a part of a bond in the structure of the acid decomposable group can be cleaved by the action of an acid.

Examples of the acid decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved by the action of an acid and a group (ii) in which some bonds are cleaved by the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, typically non-polymers having a molecular weight of 500 or greater and less than 4000 (hereinafter, referred to as “low-molecular-weight compounds”) are used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, itaconic acid diester in which the substituent (R^{αx}) has been substituted with a substituent having an ester bond or α-hydroxyacryl ester in which the substituent (R^{αx}) has been substituted with a hydroxyalkyl group or a group obtained by modifying a hydroxyl group thereof can be described as acrylic acid ester. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the α-position may be substituted with a substituent. Further, the α-position denotes the first carbon atom adjacent to a functional group unless otherwise specified.

Examples of the substituent that substitutes the hydrogen atom at the α-position of hydroxystyrene include those for R^{αx}.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the present embodiment is a resist composition which generates an acid upon light exposure and whose solubility in a developing solution is changed by the action of the acid.

Such a resist composition contains a base material component (A) (hereinafter, also referred to as “component (A)”) whose solubility in a developing solution is changed by the action of the acid, and a compound (D0) (hereinafter, also referred to as “component (D0)”) represented by General Formula (d0).

The resist composition of the present embodiment has a function of generating an acid upon light exposure, the component (A) may generate an acid upon light exposure, and an additive component that is separately blended from the component (A) may generate an acid upon light exposure.

Specifically, the resist composition according to the present embodiment may be (1) a resist composition containing an acid generator component (B) (hereinafter referred to as “component (B)”) that generates an acid upon light exposure, may be (2) a resist composition in which the component (A) is a component that generates an acid upon light exposure, or may be (3) a resist composition in which the component (A) is a component that generates an acid upon light exposure and which further contains the component (B).

That is, in the cases of (2) and (3) described above, the component (A) is “base material component which generates an acid upon light exposure and whose solubility in a developing solution is changed by the action of the acid”. In a case where the component (A) is a base material component which generates an acid upon light exposure and whose solubility in a developing solution is changed due to the action of the acid, it is preferable that a component (A1) described below is a polymer compound which generates an acid upon light exposure and whose solubility in a developing solution is changed due to the action of the acid. As such a polymer compound, a resin having a constitutional unit that generates an acid upon light exposure can be used. As the constitutional unit that generates an acid upon light exposure, those which have been known can be used.

In a case where a resist film is formed of the resist composition according to the present embodiment and the resist film is selectively exposed, for example, since an acid is generated from the component (B) in an exposed portion of the resist film and the solubility of the component (A) in a developing solution is changed due to the action of the acid while the solubility of the component (A) in a developing solution is not changed in an unexposed portion of the resist film, a difference in solubility in the developing solution occurs between the exposed portion and the unexposed portion.

Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative tone.

The resist composition of the present embodiment may be a positive-tone resist composition or a negative-tone resist composition. Further, in the formation of a resist pattern, the resist composition according to the present embodiment may be applied to an alkali developing process using an alkali developing solution in the developing treatment, or a solvent developing process using a developing solution containing an organic solvent (organic developing solution) in the developing treatment.

### <Component (A)>

In the resist composition according to the present embodiment, the component (A) contains a resin component (A1) (hereinafter, also referred to as “component (A1)”) whose solubility in a developing solution is changed by the action of an acid.

Since the polarity of the base material component before and after the light exposure is changed by using the component (A1), an excellent development contrast can be obtained not only in an alkali developing process but also in a solvent developing process.

As the component (A), another polymer compound and/or a low-molecular-weight compound may be used in combination with the component (A1).

In the resist composition according to the present embodiment, the component (A) may be used alone or a combination of two or more kinds thereof may be used.

### ⋅In Regard to Component (A1)

The component (A1) is a resin component whose solubility in a developing solution is changed by the action of an acid.

As the component (A1), those having a constitutional unit (a1) containing an acid decomposable group whose polarity is increased by the action of an acid are preferable.

Further, the component (A1) may have other constitutional units as necessary in addition to the constitutional unit (a1).

<<Constitutional Unit (a1)>>

The constitutional unit (a1) is a constitutional unit that contains an acid decomposable group whose polarity is increased by the action of an acid.

Examples of the acid dissociable group are the same as those which have been suggested as the acid dissociable groups of the base resin for a chemically amplified resist composition.

Specific examples of the suggested acid dissociable group of the base resin for a chemically amplified resist composition include “acetal type acid dissociable group”, “tertiary alkyl ester type acid dissociable group”, “tertiary alkyloxycarbonyl acid dissociable group”, and “secondary alkyloxycarbonyl acid dissociable group” described below.

### Acetal Type Acid Dissociable Group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

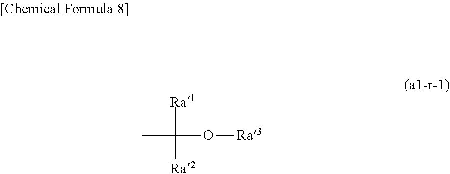

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ and Ra′² represents a hydrogen atom and more preferable that both Ra′¹ and Ra′² represent a hydrogen atom.

In a case where Ra′¹ or Ra′² represents an alkyl group, examples of the alkyl group include the same alkyl groups described as the substituent which may be bonded to the carbon atom at the α-position in the description on α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The number of carbon atoms in the monocycloalkane is preferably 3 to 6, and specifically, cyclopentane and cyclohexane are exemplary examples.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

In a case where the cyclic hydrocarbon group as Ra′³ becomes an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may include a substituent. Examples of this substituent include Ra^{x5} described above.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid Dissociable Group:

Examples of the acid dissociable group that protects a carboxy group among the polar groups include an acid dissociable group represented by General Formula (a1-r-2).

Among examples of the acid dissociable group represented by Formula (a1-r-2), a group formed of an alkyl group is referred to as “tertiary alkyl ester type acid dissociable group” for convenience.

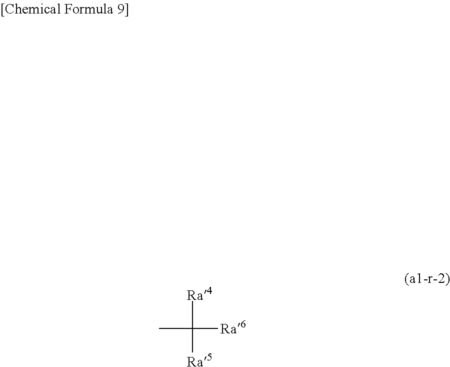

[In the formula, Ra′⁴ to Ra′⁶ each independently represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ represent an independent hydrocarbon group without being bonded to one another, suitable examples thereof include a group represented by General Formula (a1-r2-4).

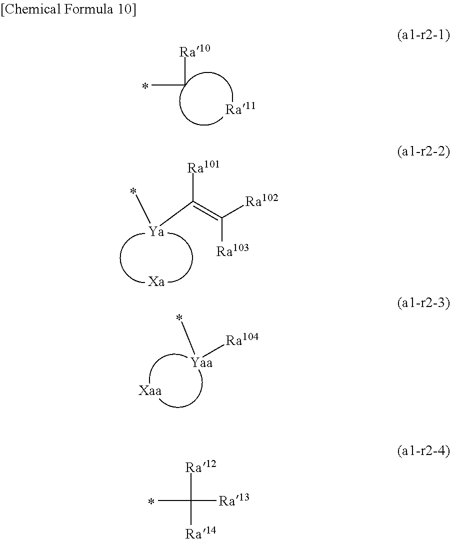

[In Formula (a1-r2-1), Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to one another to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a monovalent chain-like saturated hydrocarbon group having 1 to 10 carbon atoms. Some or all of hydrogen atoms contained in the chain-like saturated hydrocarbon group may be substituted. Ra′¹⁴ represents a hydrocarbon group which may have a substituent. * represents a bonding site (the same applies hereinafter).]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups described as the aliphatic hydrocarbon group (alicyclic hydrocarbon group) which is a monocyclic group or a polycyclic group as Ra′³ in Formula (a1-r-1). Among these, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group formed by further removing one or more hydrogen atoms from the cyclic monovalent hydrocarbon group (an aliphatic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0^{2,6}]decanyl group, a tricyclo[3.3.1.1^{3,7}]decanyl group, a tetracyclo[6.2.1.1^{3,6}.0^{2,7}]dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ are the same as those for Ra^{x5}.

Examples of the group having a carbon-carbon double bond generated by two or more of Ra¹⁰¹ to Ra¹⁰³ being bonded to one another to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), preferred examples of the aliphatic cyclic group that is formed by Xaa together with Yaa include the group described as the aliphatic hydrocarbon group which is a monocyclic group or a polycyclic group as Ra′³ in Formula (a1-r-1).

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ preferably represents a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent that Ra¹⁰⁴ in Formula (a1-r2-3) may have include a methyl group, an ethyl group, propyl group, a hydroxy group, a carboxy group, a halogen atom, an alkoxy group (a methoxy group, an ethoxy group, a propoxy group, a butoxy group, and the like), and an alkyloxycarbonyl group.

In Formula (a1-r2-4), Ra′¹² and Ra′¹³ each independently represent a monovalent chain-like saturated hydrocarbon group having 1 to 10 carbon atoms. Examples of the monovalent chain-like saturated hydrocarbon group having 1 to 10 carbon atoms as Ra′¹² and Ra′¹³ include the same one as the monovalent chain-like saturated hydrocarbon group having 1 to 10 carbon atoms as Ra¹⁰¹ to Ra¹⁰³ as described above. Some or all of hydrogen atoms contained in the chain-like saturated hydrocarbon group may be substituted.

Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent are those for Ra^{x5} described above.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The number of carbon atoms in the monocycloalkane is preferably 3 to 6, and specifically, cyclopentane and cyclohexane are exemplary examples.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

Examples of the aromatic hydrocarbon group as Ra′¹⁴ include the same groups as those for the aromatic hydrocarbon group as Ra¹⁰⁴. Among these, Ra′¹⁴ preferably represents a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be included in Ra′¹⁴ include the same groups as those for the substituent which may be included in Ra¹⁰⁴.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

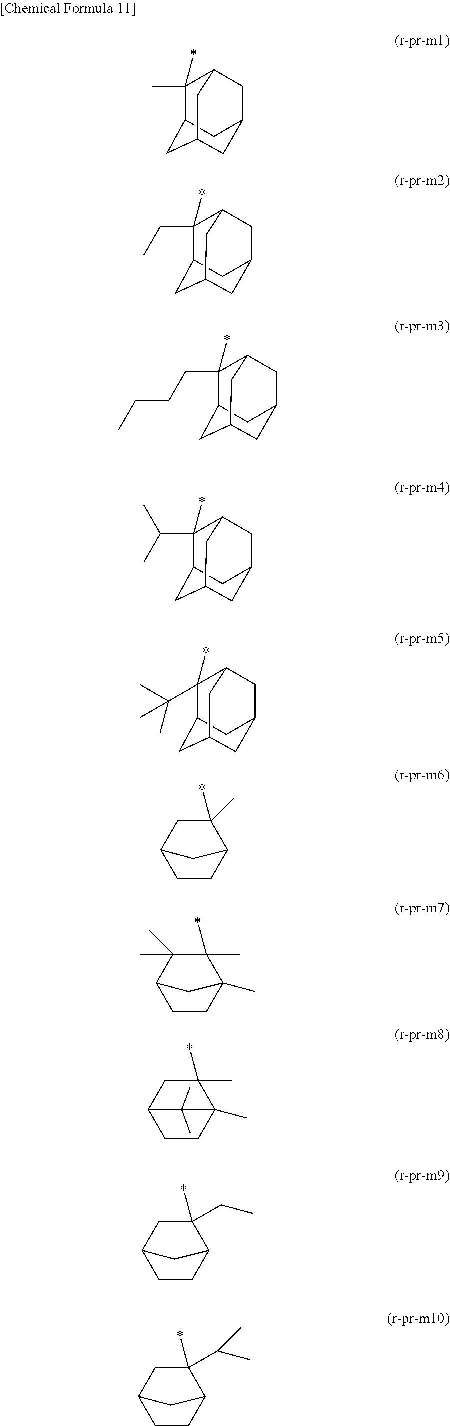

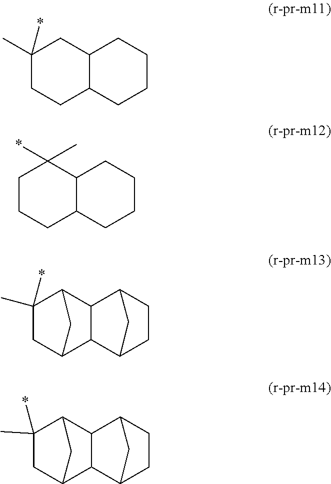

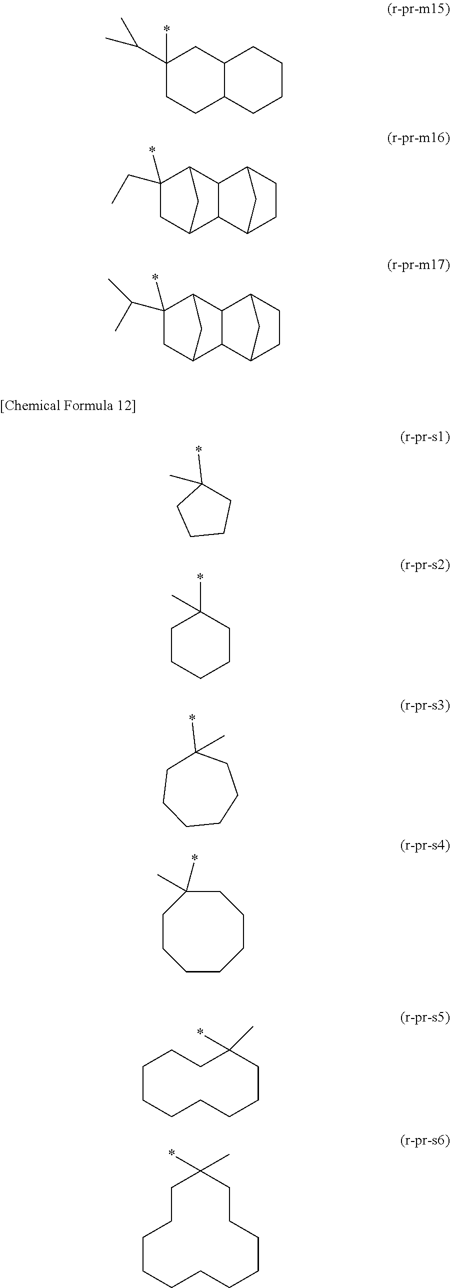

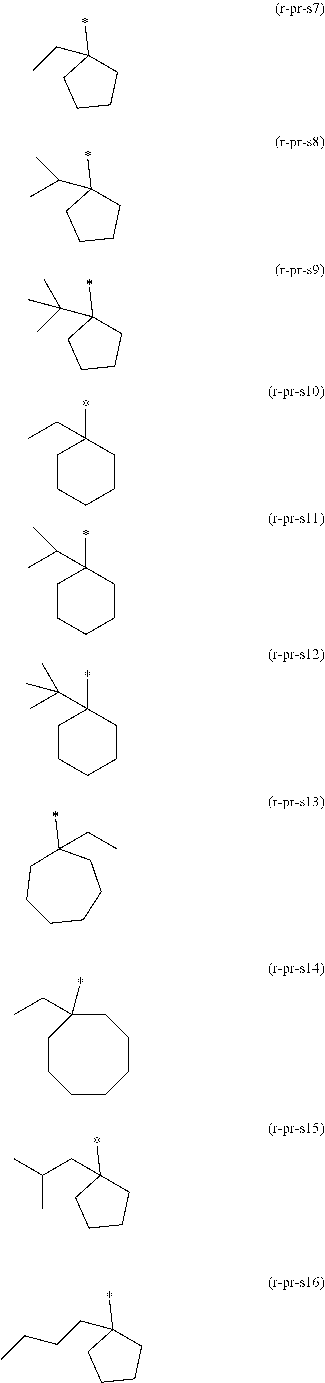

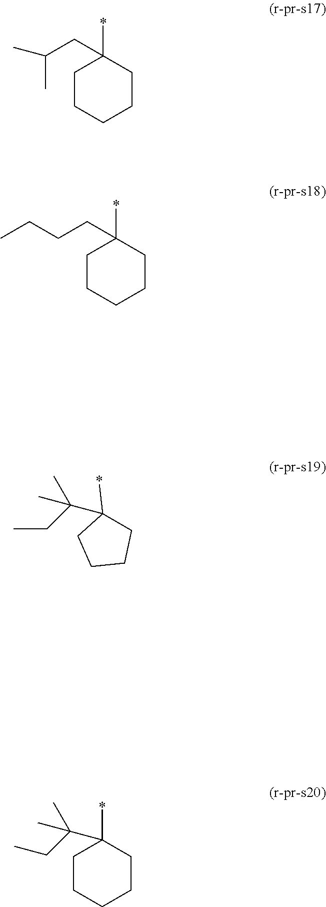 [SMILES: *C1(CC(C)C)CCCCC1 ; *C1(CCCC)CCCCC1 ; *C1(C(C)(C)CC)CCCC1 ; *C1(C(C)(C)CC)CCCCC1]

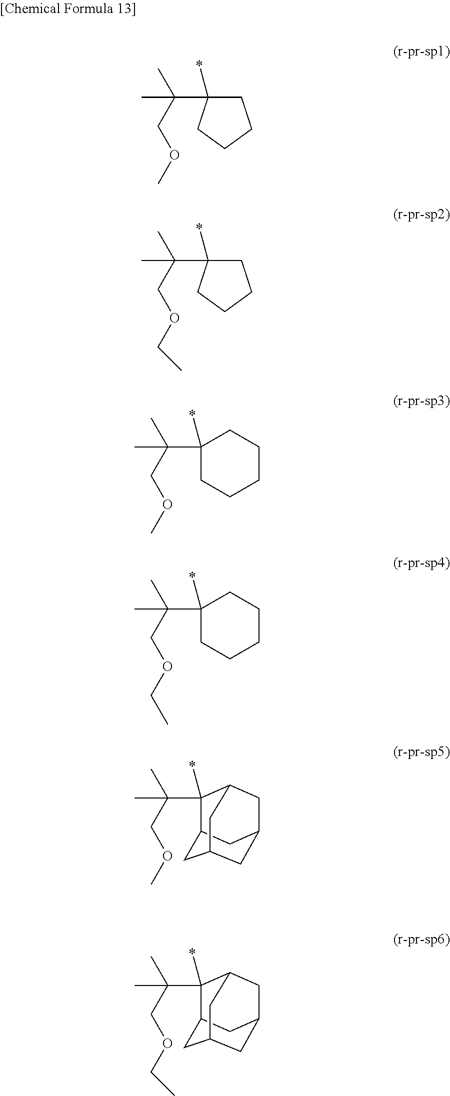

Specific examples of the group represented by Formula (a1-r2-2) are shown below.

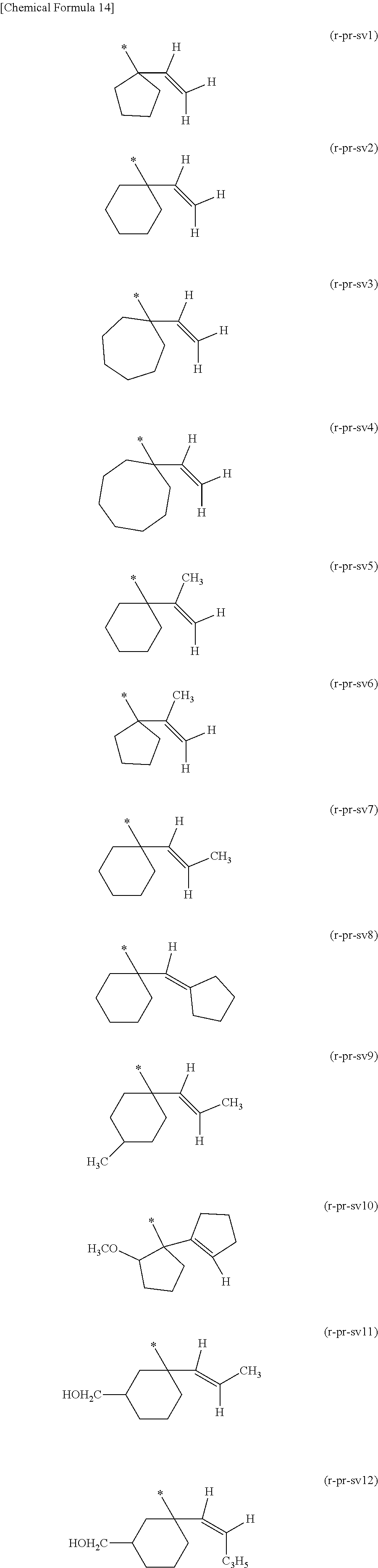

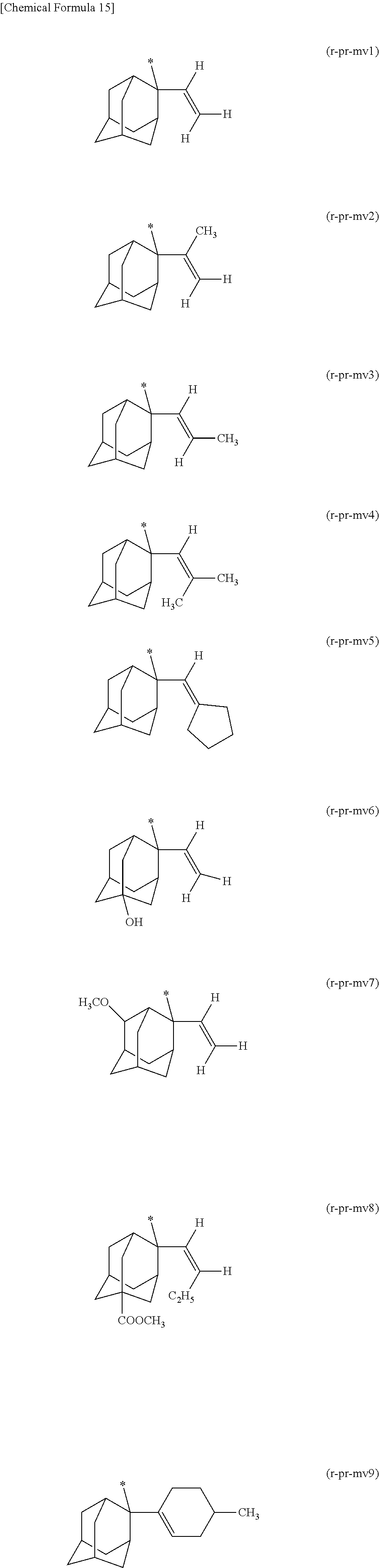

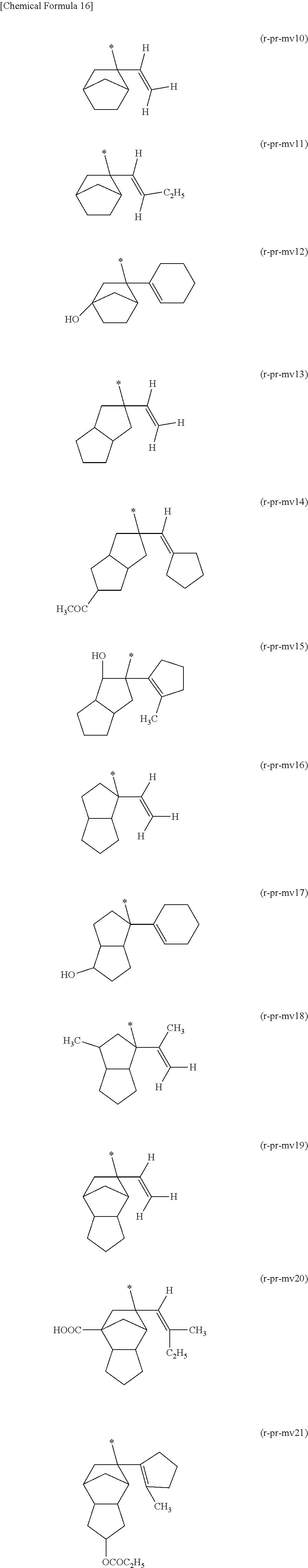

Specific examples of the group represented by Formula (a1-r2-3) are shown below.

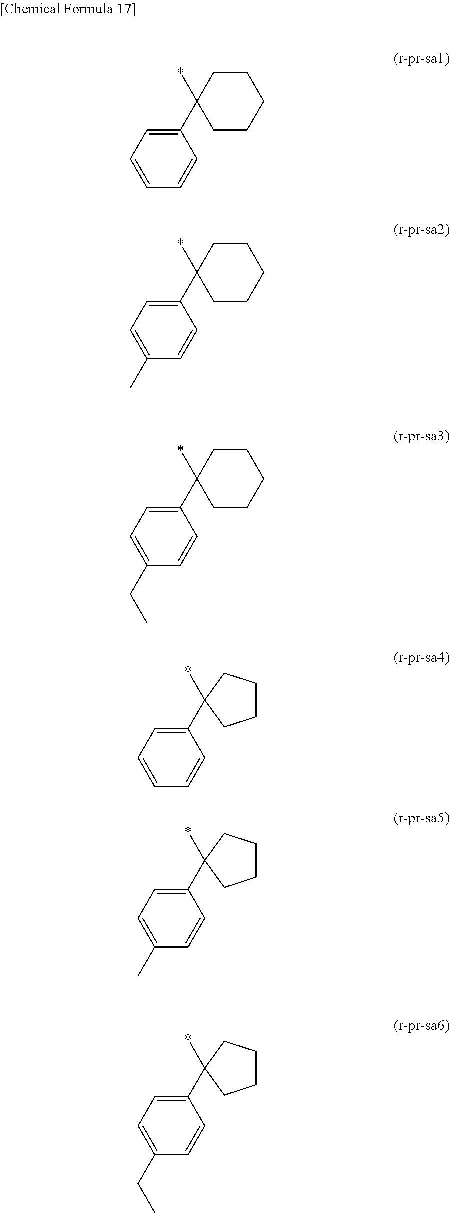

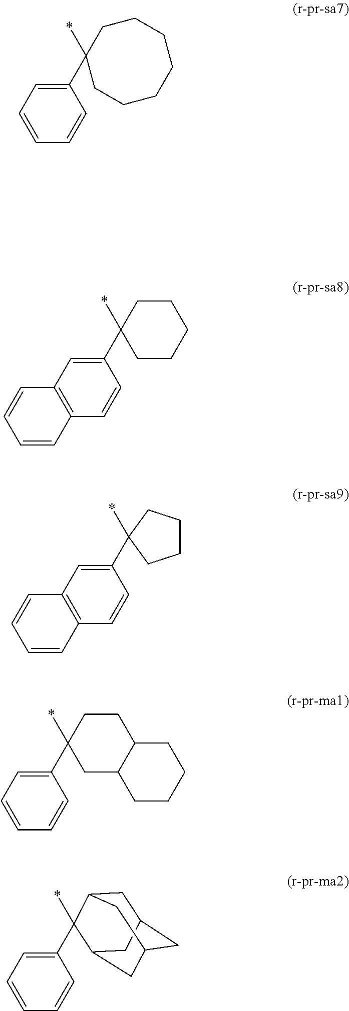 [SMILES: *C1(c2ccccc2)CCCCCCC1 ; *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

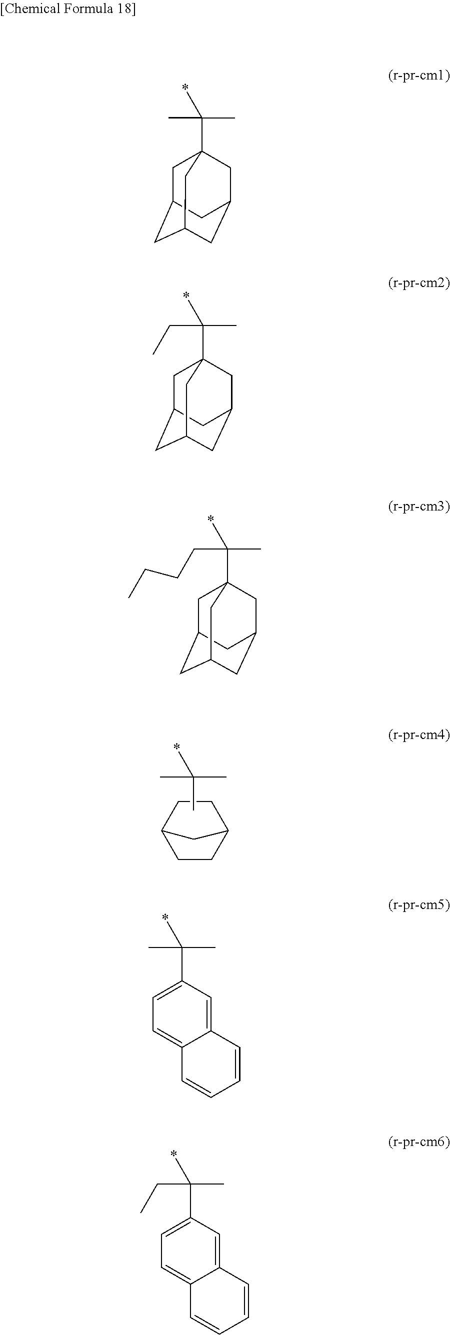

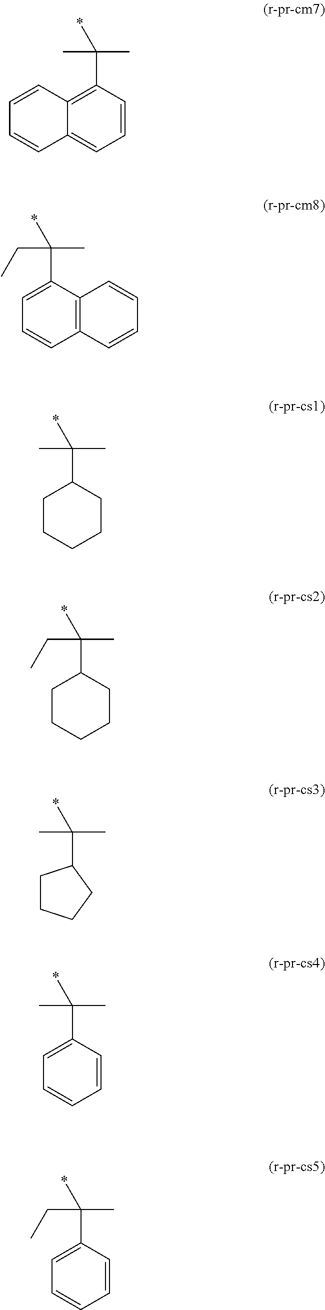

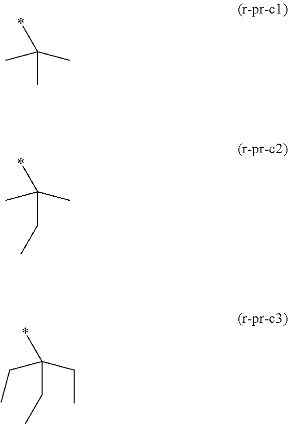

### Tertiary Alkyloxycarbonyl Acid Dissociable Group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

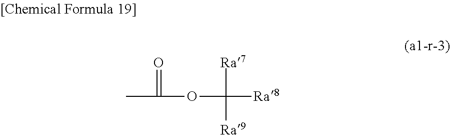

[In the formula, Ra′⁷ to Ra′⁹ each independently represent an alkyl group.]

In Formula (a1-r-3), Ra′⁷ to Ra′⁹ each represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

### Secondary Alkyl Ester Type Acid Dissociable Group:

Examples of the acid dissociable group that protects a carboxy group among the polar groups include an acid dissociable group represented by General Formula (a1-r-4).

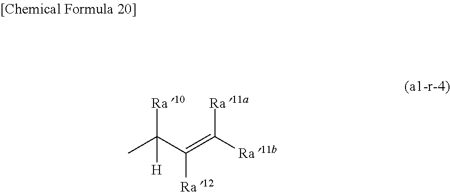

[In the formula, Ra′¹⁰ represents a hydrocarbon group. Ra′^{11a} and Ra′^{11b} each independently represent a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′¹⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} and Ra′^{11b} may have a substituent. Examples of this substituent include Ra^{x5} described above.

Ra′¹⁰ and Ra′^{11a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among the examples described above, as the ring formed by Ra′¹⁰ and Ra′^{11a} or Ra′^{11b} being bonded to each other, monocycloalkene, a ring in which some carbon atoms of monocycloalkene are substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a condensed ring. Specific examples of the condensed ring include indane.

The ring formed by Ra′¹⁰ and Ra′^{11a} or Ra′^{11b} being bonded to each other may have a substituent. Examples of this substituent include Ra^{x5} described above.

Ra′^{11a} or Ra′^{11b} and Ra′¹² may be bonded to each other to form a ring, and examples of the ring include the rings formed by Ra′¹⁰ and Ra′^{11a} or Ra′^{11b} being bonded to each other.

Specific examples of the group represented by Formula (a1-r-4) are shown below.

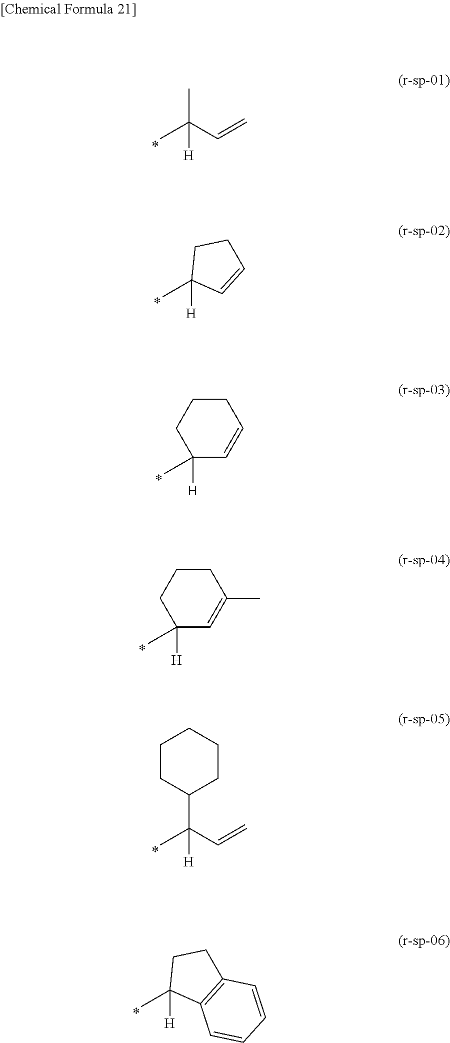

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include a constitutional unit represented by General Formula (a1-1), (a1-2), or (a1-3).

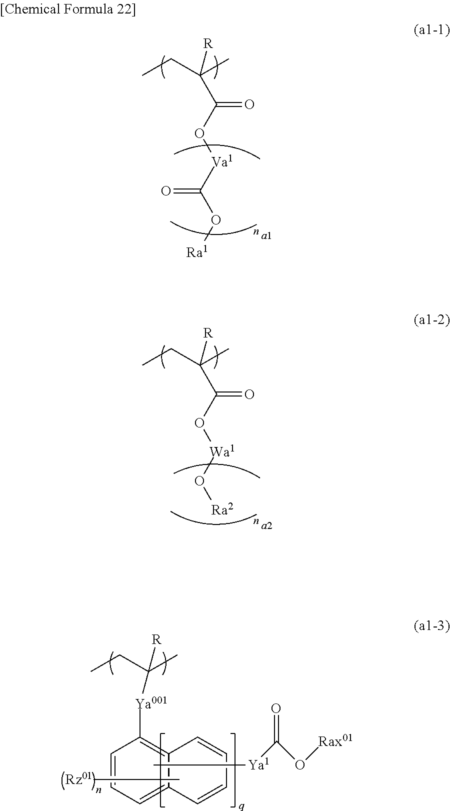

[In the formulae, R represents a hydrogen atom, an alkyl group having 1 to 5 carbon atoms, or a halogenated alkyl group having 1 to 5 carbon atoms. Va¹ represents a divalent hydrocarbon group which may have a substituent. nₐ₁ represents an integer of 0 to 2. Ra¹ represents an acid dissociable group represented by General Formula (a1-r-1), (a1-r-2), or (a1-r-4). Wa¹ represents a (nₐ₂+1)-valent hydrocarbon group, nₐ₂ represents an integer of 1 to 3, and Ra² represents an acid dissociable group represented by General Formula (a1-r-1) or (a1-r-3). Ya⁰⁰¹ represents a single bond or a divalent linking group. Ya⁰¹ represents a single bond or a divalent linking group. Rax⁰¹ represents an acid dissociable group represented by General Formula (a1-r-1), (a1-r-2), or (a1-r-4). Rz⁰¹ represents an alkyl group, a halogen atom, a halogenated alkyl group, a hydroxy group, or an alkoxy group. q represents an integer of 0 to 3. n represents an integer of 0 or greater. Here, n≤q×2+4 is satisfied.]

In Formulae (a1-1) to (a1-3), R represents a hydrogen atom, an alkyl group having 1 to 5 carbon atoms, or a halogenated alkyl group having 1 to 5 carbon atoms. The alkyl group having 1 to 5 carbon atoms as R is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R preferably represents a hydrogen atom, an alkyl group having 1 to 5 carbon atoms, or a fluorinated alkyl group having 1 to 5 carbon atoms, and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. The monocyclic alicyclic hydrocarbon group is preferably a group obtained by removing two hydrogen atoms from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. The number of carbon atoms in the polycycloalkane is preferably in a range of 7 to 12, and specific examples thereof include adamantane, norbornane, isobornane, tricyclo[5.2.1.0^{2,6}]decane, and tetracyclododecane.

In the alicyclic hydrocarbon group, some carbon atoms forming the ring structure may be substituted with substituents having a heteroatom. Examples of the substituents having a heteroatom include —O—, —C(═O)—O—, —S—, —S(═O)₂—, and —S(═O)₂—O—. Specific examples of the alicyclic hydrocarbon group substituted with a substituent having a heteroatom include a group obtained by removing one hydrogen atom from a lactone-containing cyclic group represented by any of General Formulae (a2-r-1) to (a2-r-7), and a group obtained by removing one hydrogen atom from a —SO₂— containing cyclic group represented by any of General Formulae (a5-r-1) to (a5-r-4).

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents an acid dissociable group represented by Formula (a1-r-1), (a1-r-2), or (a1-r-4).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent, trivalent, or tetravalent and more preferably divalent or trivalent.

In Formula (a1-2), Ra² represents an acid dissociable group represented by General Formula (a1-r-1) or (a1-r-3).

In Formula (a1-3), the divalent linking group as Ya⁰⁰¹ is not particularly limited, and suitable examples thereof include a divalent hydrocarbon group which may have a substituent and a divalent linking group having a heteroatom.

Among these, it is preferable that Ya⁰⁰¹ preferably represents an ester bond [—C(═O)—O— or —O—C(═O)—], an ether bond (—O—), a linear or branched alkylene group, an aromatic hydrocarbon group, a combination thereof, or a single bond. The alkylene group has preferably 1 to 10 carbon atoms, more preferably 1 to 6 carbon atoms, still more preferably 1 to 4 carbon atoms, and particularly preferably 1 to 3 carbon atoms.

Among these, Ya⁰⁰¹ represents more preferably a combination of an ester bond [—C(═O)—O— or —O—C(═O)—] and a linear alkylene group, or a single bond and still more preferably a single bond.

In Formula (a1-3), the divalent linking group as Ya⁰¹ is not particularly limited, and suitable examples thereof include a divalent hydrocarbon group which may have a substituent and a divalent linking group having a heteroatom.

Among these, it is preferable that Ya⁰¹ represents an ester bond [—C(═O)—O— or —O—C(═O)—], an ether bond (—O—), a linear or branched alkylene group, an aromatic hydrocarbon group, a combination thereof, or a single bond. Among these, Ya⁰¹ is more preferably a combination of an ester bond [—C(═O)—O— or —O—C(═O)—] and a linear alkylene group, or a single bond, and still more preferably a single bond.

In Formula (a1-3), Rax⁰¹ preferably represents an acid dissociable group represented by General Formula (a1-r-2) or (a1-r-4) and, among these, more preferably an acid dissociable group represented by General Formula (a1-r-2) and still more preferably a group represented by General Formula (a1-r2-1).

In Formula (a1-3), the alkyl group, the halogenated alkyl group, and the alkoxy group as Rz⁰¹ has preferably 1 to 10 carbon atoms, more preferably 1 to 5 carbon atoms, still more preferably 1 to 3 carbon atoms, and particularly preferably 1 or 2 carbon atoms. The alkyl group, the halogenated alkyl group, and the alkoxy group may be linear or branched.

As the halogen atom represented by Rz⁰¹, an iodine atom is preferable. As the halogen atom of the halogenated alkyl group represented by Rz⁰¹, a fluorine atom, an iodine atom, or a bromine atom is preferable, and a fluorine atom is more preferable. Rz⁰¹ preferably represents an alkoxy group or a hydroxy group and more preferably a hydroxy group.

In Formula (a1-3), q represents an integer of 0 to 3. A benzene structure is formed in a case where q represents 0, a naphthalene structure is formed in a case where q represents 1, an anthracene structure is formed in a case where q represents 2, and a tetracene structure is formed in a case where q represents 3.

In Formula (a1-3), n represents an integer of 0 or greater, preferably 0 to 5, more preferably 0 to 3, and still more preferably 1 or 2. In a case where n represents an integer of 2 or greater, two or more Rz⁰¹'s may be the same as or different from each other.

In Formula (a1-3), n≤q×2+4 is satisfied. For example, in a case where q represents 1 and thus a naphthalene structure is formed, all six hydrogen atoms of the naphthalene may be substituted with hydroxy groups. In addition, the substitution positions of Ya⁰⁰¹, the —Ya⁰¹-C(═O)—O—Ra⁰¹ group, and the hydroxy group in the naphthalene are not particularly limited.

Specific examples of the constitutional unit (a1) are shown below. In the formulae shown below, R^{α} represents a hydrogen atom, a methyl group, or a trifluoromethyl group.

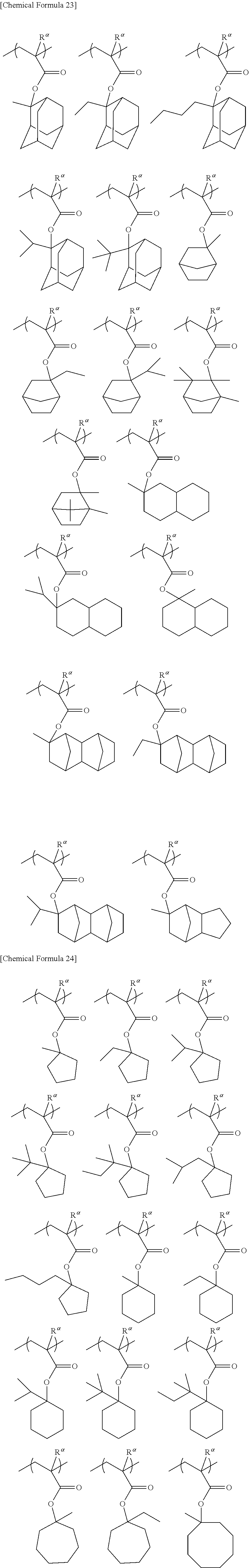

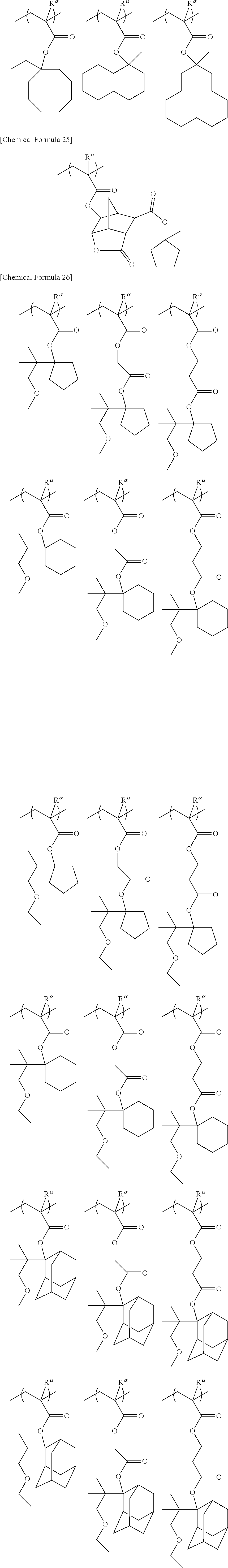

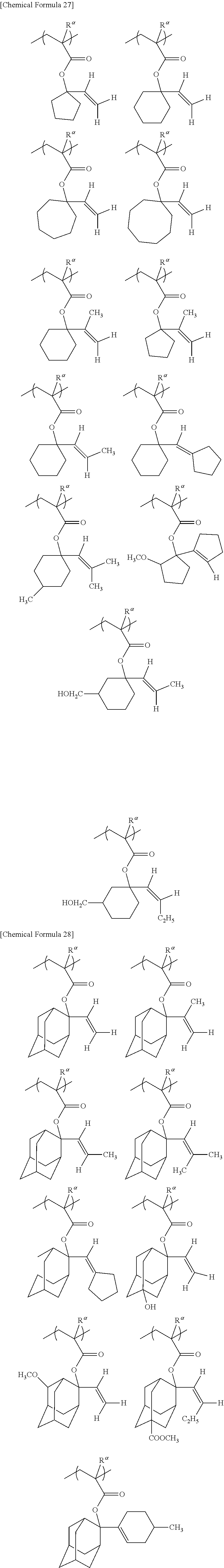

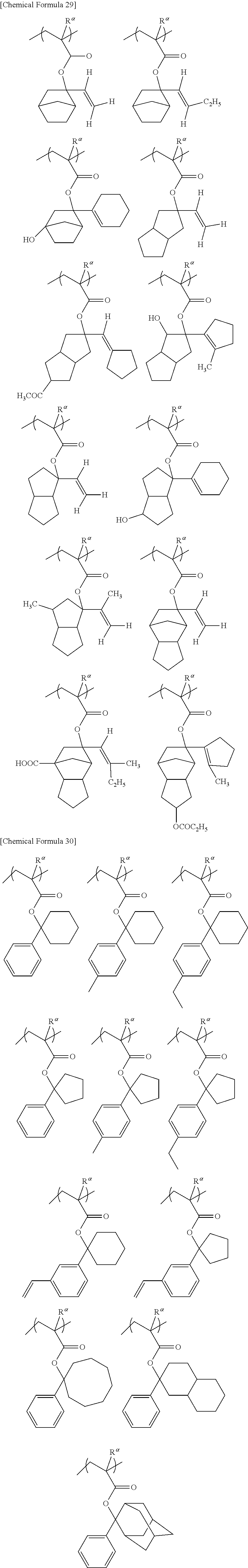

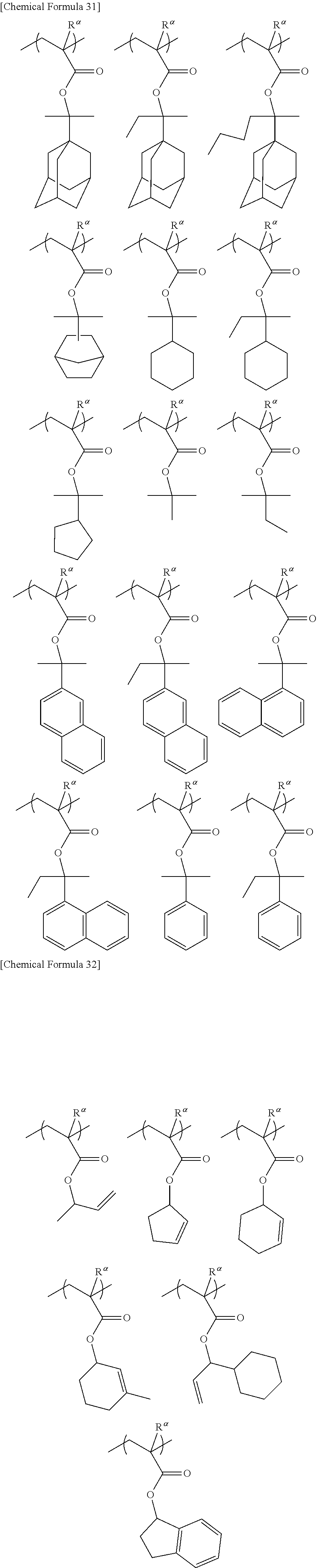

In the formulae shown below, R^{α} represents a hydrogen atom, a methyl group, or a trifluoromethyl group. Rz represents a hydrogen atom, an alkyl group, a halogen atom, a halogenated alkyl group, a hydroxy group, or an alkoxy group.

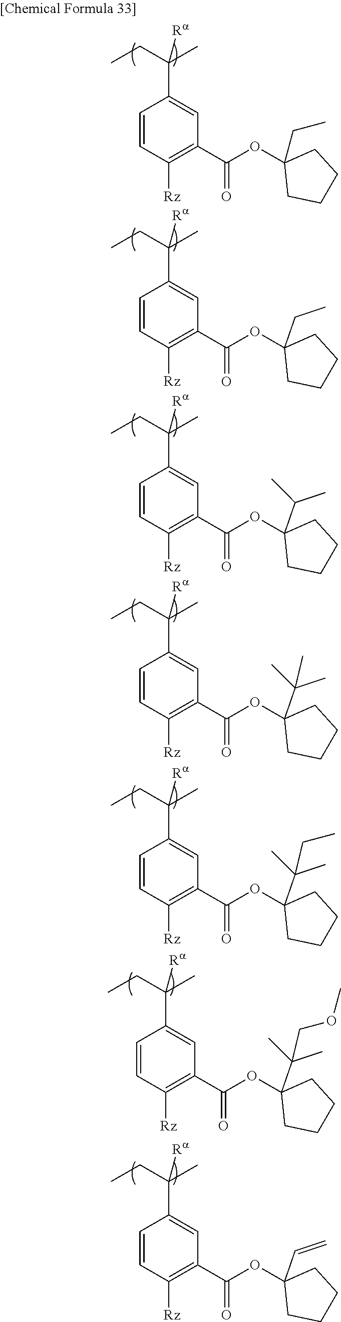

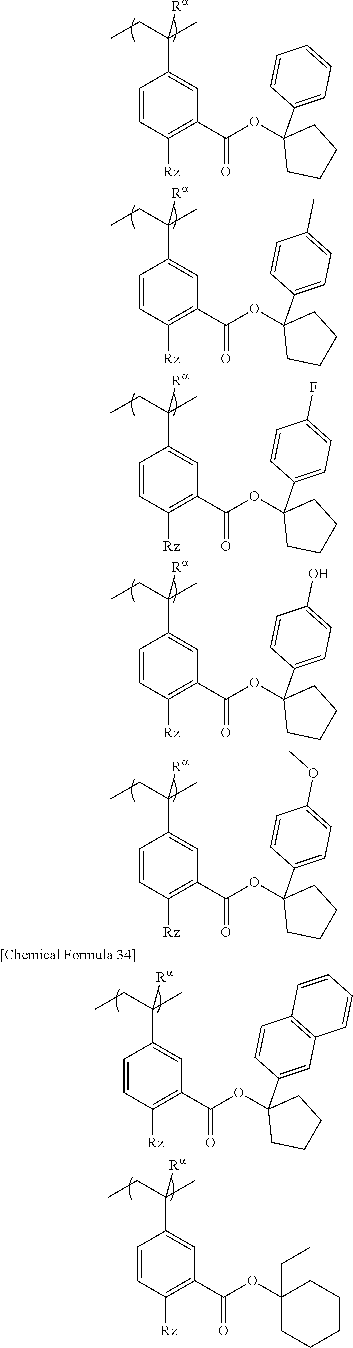

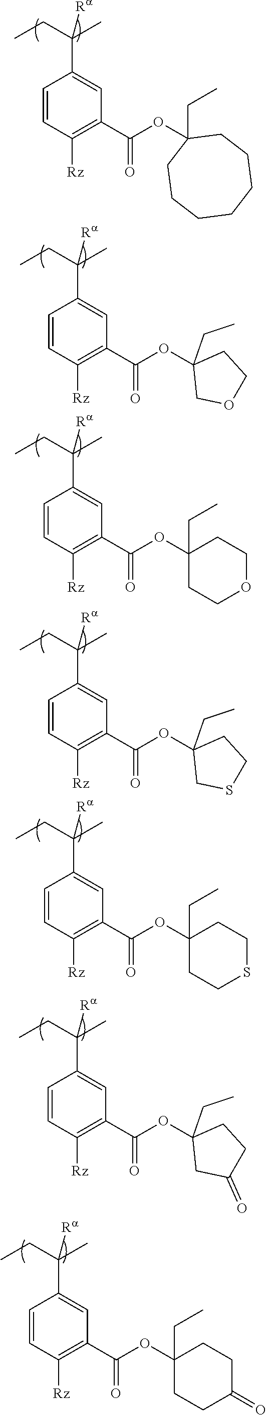

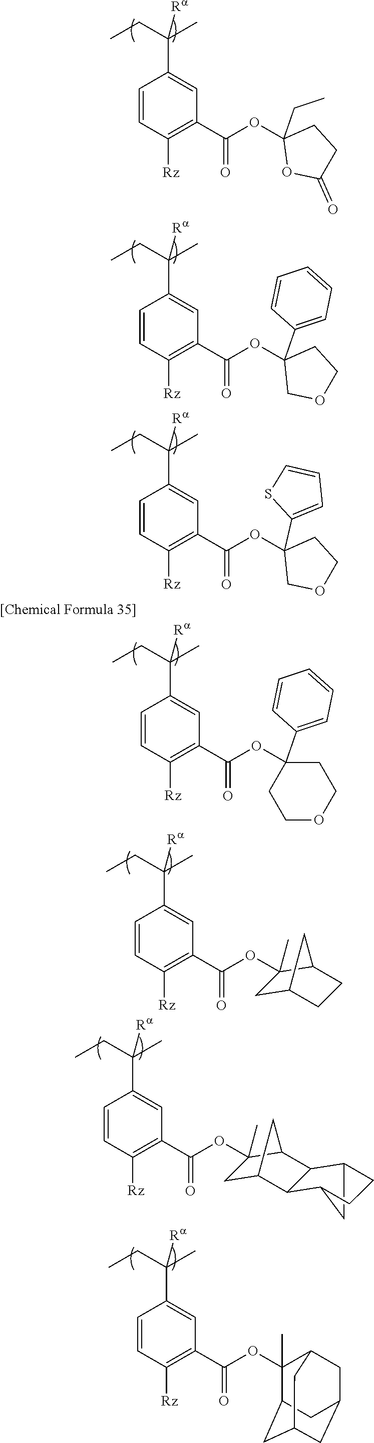

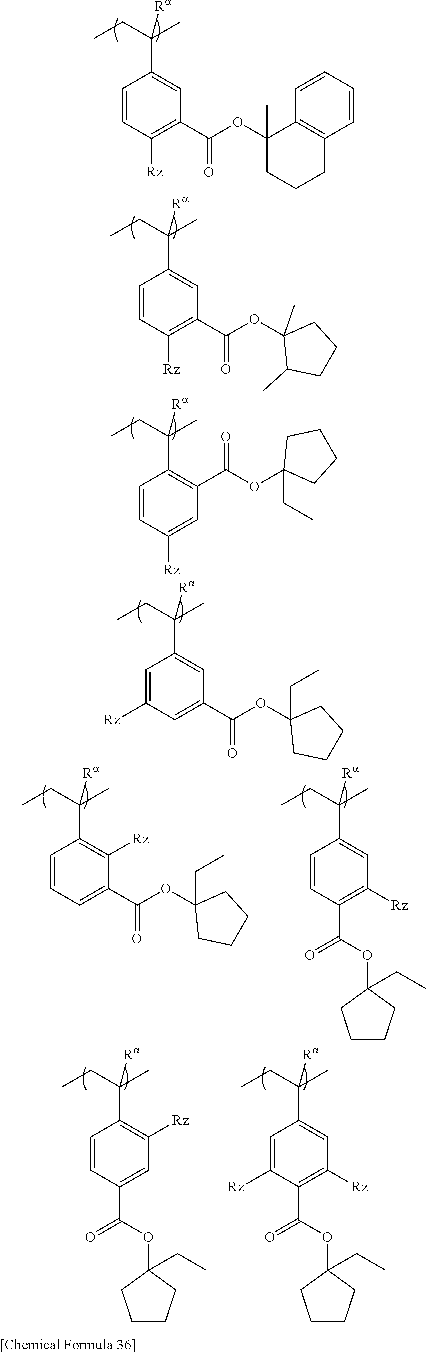

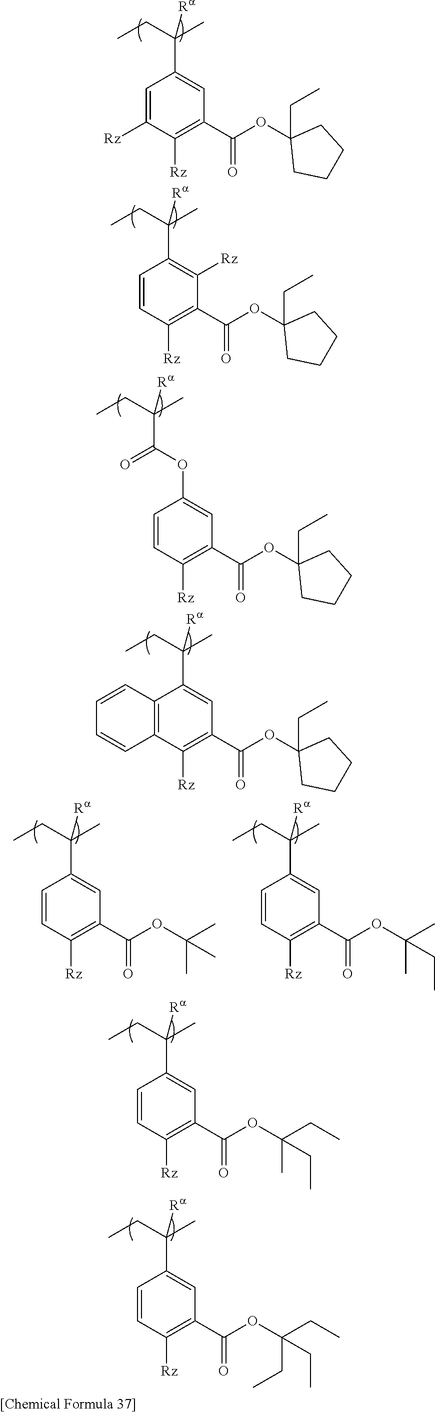

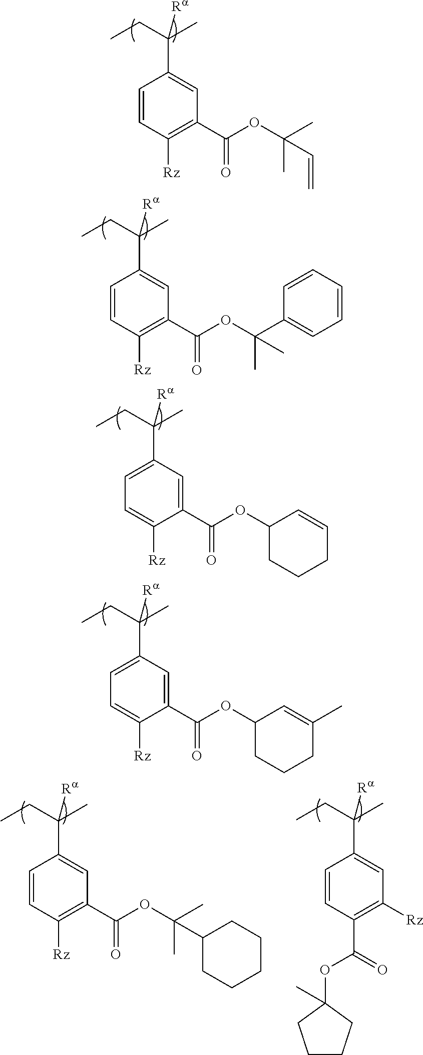

The constitutional unit (a1) included in the component (A1) may be used alone or two or more kinds thereof may be used.

Since the lithography characteristics (the sensitivity, the shape, and the like) are easily improved using electron beams or EUV, a constitutional unit represented by Formula (a1-1) is preferable as the constitutional unit (a1).

Among the examples, as the constitutional unit (a1), those having a constitutional unit represented by General Formula (a1-1-1) are particularly preferable.

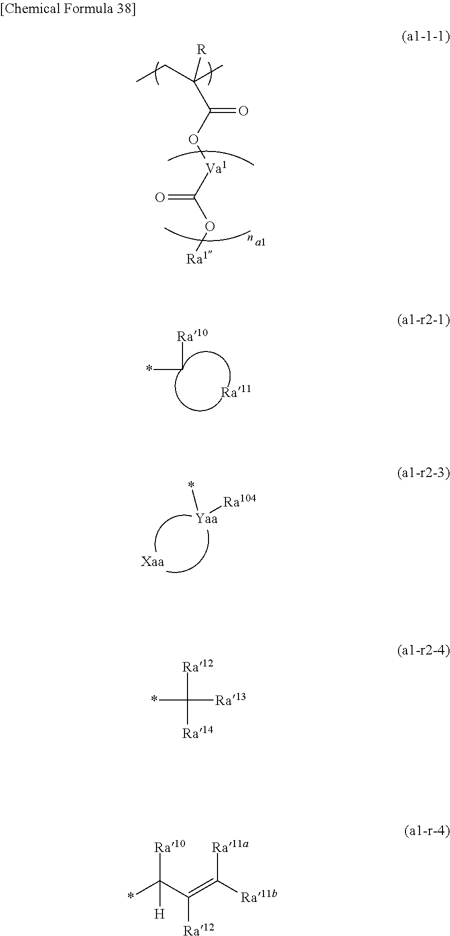

[In the formulae, Ra¹″ represents an acid dissociable group represented by General Formula (a1-r2-1), (a1-r2-3), (a1-r2-4), or (a1-r-4). * represents a bonding site.]

In Formula (a1-1-1), R, Va¹, and nₐ₁ each have the same definition as that for R, Va¹, and nₐ₁ in Formula (a1-1).

The acid dissociable group represented by General Formula (a1-12-1), (a1-r2-3), (a1-r2-4), or (a1-r-4) is as described above. Among these, it is preferable to select those in which the acid dissociable group is a cyclic group because the reactivity is enhanced for EB or EUV, which is preferable.

The proportion of the constitutional unit (a1) in the component (A1) is preferably in a range of 5% to 95% by mole, more preferably in a range of 10% to 90% by mole, still more preferably in a range of 30% to 70% by mole, and particularly preferably in a range of 40% to 60% by mole, with respect to the total amount (100% by mole) of all constitutional units constituting the component (A1).

In a case where the proportion of the constitutional unit (a1) is set to be greater than or equal to the lower limit of the above-described preferable ranges, lithography characteristics such as sensitivity, resolution, and roughness amelioration are improved. Further, in a case where the proportion of the constitutional unit (a1) is set to be less than or equal to the upper limits of the above-described preferable ranges, the constitutional unit (a1) and other constitutional units can be balanced, and the lithography characteristics are improved.

### <<Other Constitutional Units>>

The component (A1) may have other constitutional units as necessary in addition to the constitutional unit (a1) described above.

Examples of the other constitutional units include a constitutional unit represented by General Formula (a10-1); a constitutional unit (a5) that generates an acid upon light exposure; a constitutional unit (a6) having acid diffusion controllability; a constitutional unit (a2) containing a lactone-containing cyclic group, a —SO₂-containing cyclic group, or a carbonate-containing cyclic group; and a constitutional unit derived from a compound represented by General Formula (a8-1).

Constitutional Unit (a10):

The constitutional unit (a10) is a constitutional unit represented by General Formula (a10-1).

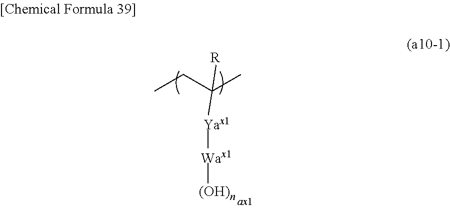

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In Formula (a10-1), R has the same definition as that for R in General Formula (a1-1). R preferably represents a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a10-1), Ya^{x1} represents a single bond or a divalent linking group. In the chemical formula, the divalent linking group as Ya^{x1} is not particularly limited, and suitable examples thereof include a divalent hydrocarbon group which may have a substituent and a divalent linking group having a heteroatom.

⋅Divalent Hydrocarbon Group which May have Substituent:

The divalent hydrocarbon group which may have a substituent may be an aliphatic hydrocarbon group or an aromatic hydrocarbon group.

### ⋅⋅Aliphatic Hydrocarbon Group

The aliphatic hydrocarbon group indicates a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### ⋅⋅Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### ⋅⋅Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. As the linear or branched aliphatic hydrocarbon group, the same groups as those described above are exemplary examples.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group obtained by removing two hydrogen atoms from a monocycloalkane. The number of carbon atoms in the monocycloalkane is preferably 3 to 6, and specifically, cyclopentane and cyclohexane are exemplary examples. As the polycyclic alicyclic hydrocarbon group, a group formed by removing two hydrogen atoms from a polycycloalkane is preferable. The number of carbon atoms in the polycycloalkane is preferably 7 to 12, and specifically, adamantane, norbornane, isobornane, tricyclo[5.2.1.0^{2,6}]decane, and tetracyclododecane are exemplary examples.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups are substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### ⋅⋅Aromatic Hydrocarbon Group

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (for example, biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### ⋅Divalent Linking Group Having Heteroatom:

Examples of the divalent linking group having a heteroatom include —O—, —C(═O)—O—, —O—C(═O)—, —C(═O)—, —O—C(═O)—O—, —C(═O)—NH—, —NH—, —NH—C(═NH)— (H may be substituted with a substituent such as an alkyl group or an acyl group), —S—, —S(═O)₂—, —S(═O)₂—O—, and a group represented by General Formula —Y²¹—O—Y²²—, —Y²¹—O—, —Y²¹—C(═O)—O—, —C(═O)—O—Y²¹—, —[Y²¹—C(═O)—O]_{m″}—Y²², —Y²¹—O—C(═O)—Y²², or —Y²¹—S(═O)₂—O—Y²²— [in the formulae, Y²¹ and Y²² each independently represent a divalent hydrocarbon group which may have a substituent, O represents an oxygen atom, and m″ represents an integer of 1 to 3].

In a case where the above-described divalent linking group containing a heteroatom is —C(═O)—NH—, —C(═O)—NH—C(═O)—, —NH—, or —NH—C(═NH)—, H may be substituted with a substituent such as an alkyl group and an acyl group. The substituent (alkyl group, acyl group, and the like) preferably has 1 to 10 carbon atoms, more preferably has 1 to 8 carbon atoms, and particularly preferably has 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²², Y²¹ and Y²² each independently represent a divalent hydrocarbon group which may have a substituent. Examples of the divalent hydrocarbon group include the same groups as described above.

As Y²¹, a linear aliphatic hydrocarbon group is preferable, a linear alkylene group is more preferable, a linear alkylene group having 1 to 5 carbon atoms is still more preferable, and a methylene group or an ethylene group is particularly preferable.

As Y²², a linear or branched aliphatic hydrocarbon group is preferable, and a methylene group, an ethylene group, or an alkylmethylene group is more preferable. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 1 to 3, preferably an integer of 1 or 2, more preferably 1, and particularly preferably 1. That is, a group represented by Formula —Y²¹—C(═O)—O—Y²²— is preferable as the group represented by Formula —[Y²¹—C(═O)—O]_{m″}—Y²²—. Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

Ya^{x1} preferably represents a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group obtained by removing (nₐₓ₁+1) hydrogen atoms from an aromatic ring which may have a substituent. The aromatic ring is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} also include a group obtained by removing (nₐₓ₁+1) hydrogen atoms from an aromatic compound including an aromatic ring (for example, biphenyl or fluorene) which may have two or more substituents.

Among these, Wa^{x1} preferably represents a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may have a substituent or may not have a substituent. Examples of the substituent include an alkyl group, an alkoxy group, a halogen atom, and a halogenated alkyl group. Examples of the alkyl group, the alkoxy group, the halogen atom, and the halogenated alkyl group, as the substituent, include the same groups as those for the above-described substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{α} represents a hydrogen atom, a methyl group, or a trifluoromethyl group.

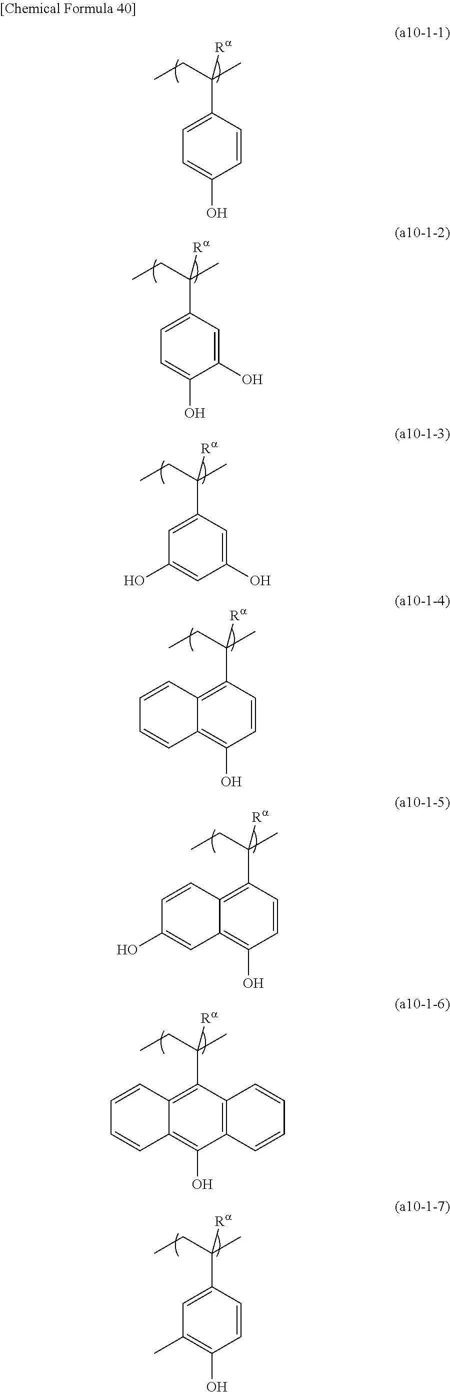

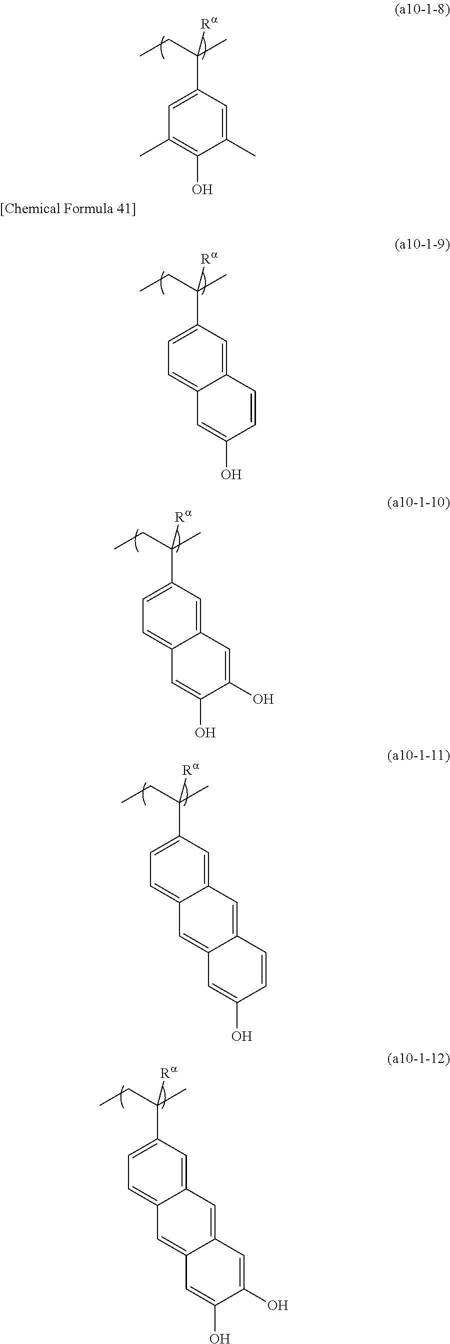

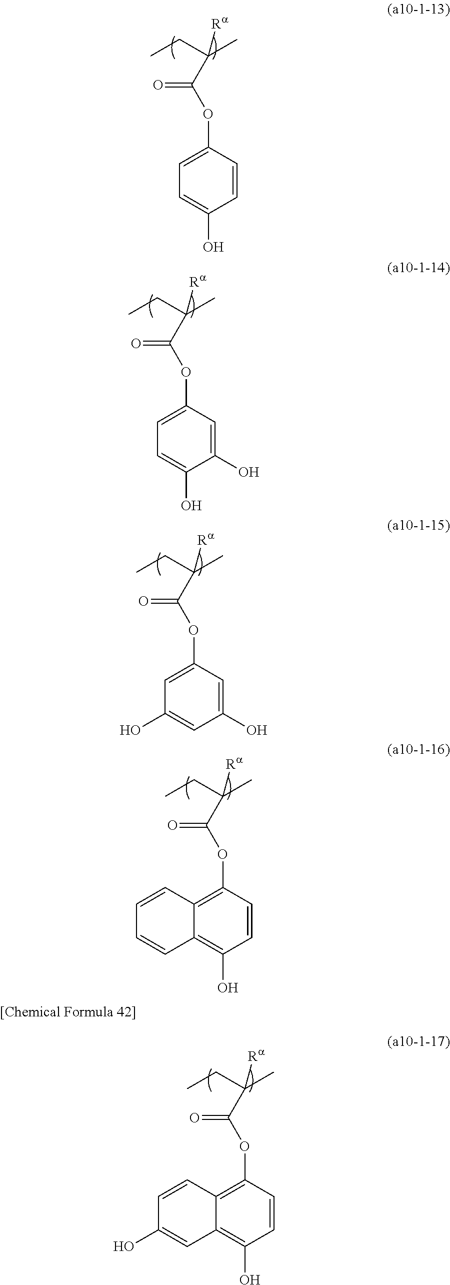

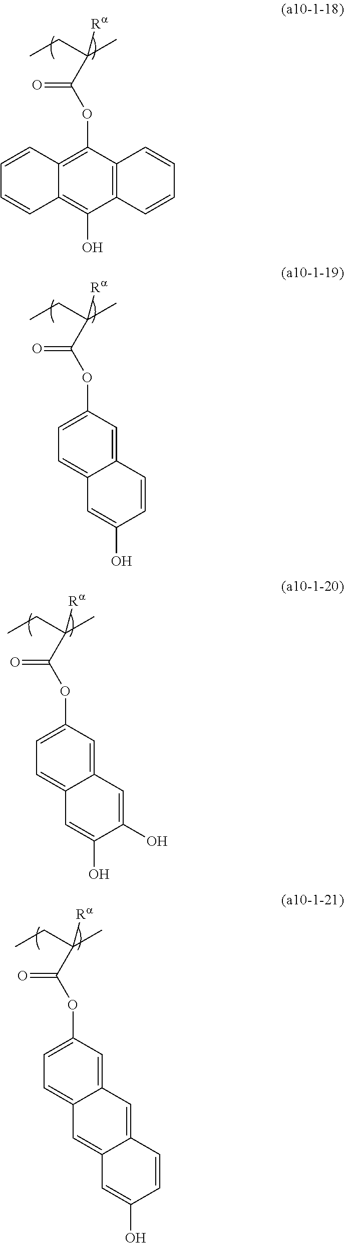

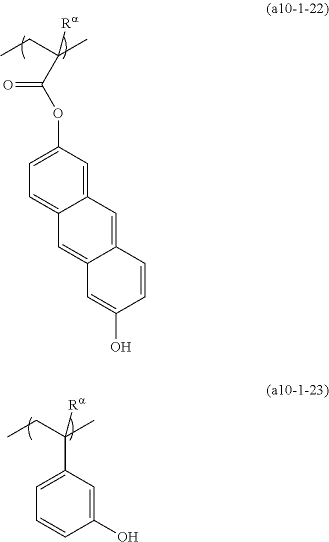

The constitutional unit (a10) included in the component (A1) may be used alone or two or more kinds thereof may be used. The component (A1) may or may not have the constitutional unit (a10), but it is preferable that the component (A1) has the constitutional unit (a10).

In a case where the component (A1) has the constitutional unit (a10), the proportion of the constitutional unit (a10) in the component (A1) is preferably in a range of 20% to 80% by mole, more preferably in a range of 25% to 70% by mole, and still more preferably in a range of 25% to 60% by mole with respect to the total amount (100% by mole) of all constitutional units constituting the component (A1).

In a case where the proportion of the constitutional unit (a10) is set to be greater than or equal to the above-described lower limits, the sensitivity is likely to be enhanced. Meanwhile, in a case where the proportion thereof is set to be less than or equal to the above-described upper limits, the constitutional unit (a10) and other constitutional units are likely to be balanced.

### Constitutional Unit (a5):

The constitutional unit (a5) is a constitutional unit that generates an acid upon light exposure. The constitutional unit (a5) is not particularly limited, and may have a structure that has been suggested so far as an acid generator for a chemically amplified resist composition. In a case where the component (A1) has the constitutional unit (a5), the acid generated upon light exposure is likely to be uniformly distributed in the resist film. In a case where the component (A1) has the constitutional unit (a5), the acid generated upon light exposure is likely to be uniformly distributed in the resist film. Examples of the constitutional unit (a5) include a constitutional unit having a structure described in the section of the component (B) below. Examples thereof include a constitutional unit having a structure represented by any of General Formulae (b-1) to (b-3).

Examples of the constitutional unit (a5) include a constitutional unit containing a group represented by General Formula (a5-0).

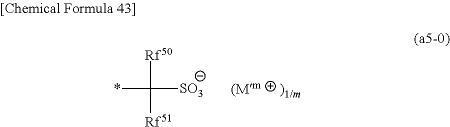

[In the formula, Rf⁵⁰ and Rf⁵¹ each independently represent a hydrogen atom, a fluorine atom, or a fluorinated alkyl group. m represents an integer of 1 or greater, and M′^{m+} represents an m-valent onium cation. * represents a bonding site.]

In General Formula (a5-an1), Rf⁵⁰ and Rf⁵¹ each independently represent a hydrogen atom, a fluorine atom, or a fluorinated alkyl group. From the viewpoint of acid strength, it is preferable that at least one of Rf⁵⁰ or Rf⁵¹ represents a fluorine atom and more preferable that both Rf⁵⁰ and Rf⁵¹ represent a fluorine atom.

Examples of the constitutional unit (a5) include a constitutional unit represented by General Formula (a5-1).

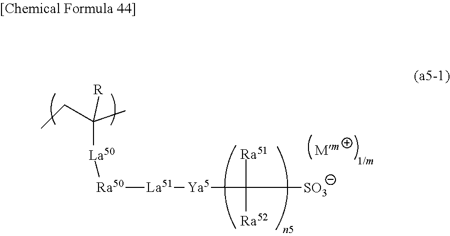

[In the formula, R represents an alkyl group having 1 to 5 carbon atoms, a halogenated alkyl group having 1 to 5 carbon atoms, a halogen atom, or a hydrogen atom. La⁵⁰ represents a divalent linking group or a single bond. Ra⁵⁰ represents a divalent hydrocarbon group which may have a substituent. La⁵¹ represents a divalent linking group. Ya⁵ represents a divalent linking group which may have a heteroatom or a single bond. Ra⁵¹ and Ra⁵² each independently represent a hydrogen atom, a fluorine atom, or a fluorinated alkyl group. n5 represents an integer of 1 to 4. m represents an integer of 1 or greater, and M′^{m+} represents an m-valent onium cation.]

### {Anion Moiety}

In Formula (a5-1), R has the same definition as that for R in General Formula (a1-1). R preferably represents a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a5-1), La⁵⁰ represents a divalent linking group or a single bond.

The divalent linking group as La⁵⁰ is not particularly limited, and examples thereof include a divalent hydrocarbon group which may have a substituent and a divalent linking group having a heteroatom. Examples of the divalent linking group as La⁵⁰ include the same groups as those for the divalent linking group as Ya^{x1} in General Formula (a10-1).

It is preferable that La⁵⁰ represents an ester bond [—C(═O)—O— or —O—C(═O)—], an ether bond (—O—), a linear or branched alkylene group, an aromatic hydrocarbon group, a combination thereof, or a single bond. Among these, La⁵⁰ represents more preferably an ester bond [—C(═O)—O— or —O—C(═O)—] or a single bond and still more preferably an ester bond [—C(═O)—O— or —O—C(═O)—].

In Formula (a5-1), Ra⁵⁰ represents a divalent hydrocarbon group which may have a substituent.

The divalent hydrocarbon group as Ra⁵⁰ may be an aliphatic hydrocarbon group or an aromatic hydrocarbon group. Examples of the divalent hydrocarbon group which may have a substituent as Ra⁵⁰ include the same groups as those for the divalent hydrocarbon group which may have a substituent, described in the section of Ya^{x1} in General Formula (a10-1).

Ra⁵⁰ preferably represents an aliphatic hydrocarbon group having a ring in the structure, a linear or branched alkylene group which may have a substituent, a linear or branched alkenylene group which may have a substituent, or an aromatic hydrocarbon group and more preferably an aliphatic hydrocarbon group having a ring in the structure. In the aliphatic hydrocarbon group having a ring in the structure, some carbon atoms constituting the ring structure may be substituted with substituents having a heteroatom. The cyclic aliphatic hydrocarbon group may be a polycyclic alicyclic group which may have a substituent or a monocyclic alicyclic group which may have a substituent.

It is preferable that Ra⁵⁰ represents a cyclic hydrocarbon group which may have a substituent. Specific examples of the cyclic hydrocarbon group include a group obtained by removing two or more hydrogen atoms from a benzene ring, a group obtained by removing two or more hydrogen atoms from a naphthalene ring, a group obtained by removing two or more hydrogen atoms from a polycycloalkane; a group obtained by removing one or more hydrogen atoms from a lactone-containing cyclic group; a lactone-containing cyclic group having a ring that contains —O—C(═O)— the ring skeleton; and a —SO₂-containing cyclic group having a ring that contains —SO₂— in the ring skeleton. Among these, it is preferable that Ra⁵⁰ represents an adamantanediyl group; a group obtained by removing one hydrogen atom from a lactone-containing cyclic group represented by any of General Formulae (a2-r-1) to (a2-r-7) or a group obtained by removing one hydrogen atom from a —SO₂-containing cyclic group represented by any of General Formulae (a5-r-1) to (a5-r-4).

In Formula (a5-1), La⁵¹ represents a divalent linking group.

Examples of the divalent linking group as La⁵¹ include the same groups as those for the divalent linking group as La⁵¹. Examples of the divalent linking group as La⁵¹ include a non-hydrocarbon oxygen atom-containing linking group such as an oxygen atom (ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination.

Examples of such a divalent linking group include linking groups each represented by General Formulae (L-a1-1) to (L-a1-8). Further, in General Formulae (L-a1-1) to (L-a1-8), V′¹⁰¹ in General Formulae (L-a1-1) to (L-a1-8) is bonded to Ra⁵⁰ in Formula (a5-1).

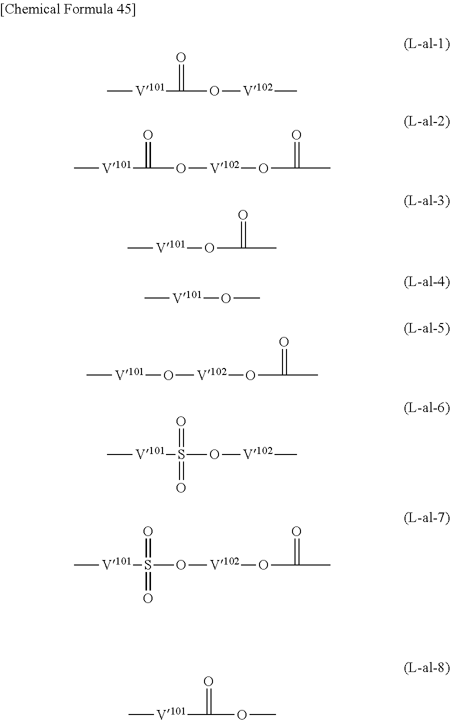

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

La⁵¹ preferably represents a divalent linking group having an ester bond or a divalent linking group having an ether bond, more preferably a linking group represented by any of Formulae (L-a1-1) to (L-a1-5) and (L-a1-8), and still more preferably a linking group represented by Formula (L-a1-3) or (L-a1-8).

In Formula (a5-1), Ya⁵ represents a divalent linking group which may have a heteroatom or a single bond.

The divalent linking group as Ya⁵ is not particularly limited, and suitable examples thereof include a divalent hydrocarbon group which may have a substituent and a divalent linking group having a heteroatom.

Examples of the divalent hydrocarbon group which may have a substituent and the divalent linking group having a heteroatom as Ya⁵ include the same groups as those for La⁵⁰ described above.

Among the examples, Ya⁵ preferably represents a linear or branched alkylene group or a single bond and more preferably a single bond.

In Formula (a5-1), Ra⁵¹ and Ra⁵² each independently represent a hydrogen atom, a fluorine atom, or a fluorinated alkyl group.

The fluorinated alkyl group is preferably a linear or branched fluorinated alkyl group having 1 to 5 carbon atoms and more preferably a trifluoromethyl group.

In Formula (a5-1), it is preferable that at least one of Ra⁵¹ and Ra⁰⁵² bonded to the carbon atom adjacent to SO₃⁻ represents a fluorine atom from the viewpoint of acid strength.

In Formula (a5-1), n5 represents an integer of 1 to 4 and preferably 1, 2, or 3.

### {Cation Moiety}

In Formula (a5-1), M′^{m+} represents an m-valent onium cation. It is preferable that M′^{m+} represents a sulfonium cation or an iodonium cation. m represents an integer of 1 or greater.

Preferred examples of the cation moiety ((M′^{m+})_{1/m}) include an organic cation represented by each of General Formulae (ca-1) to (ca-3).

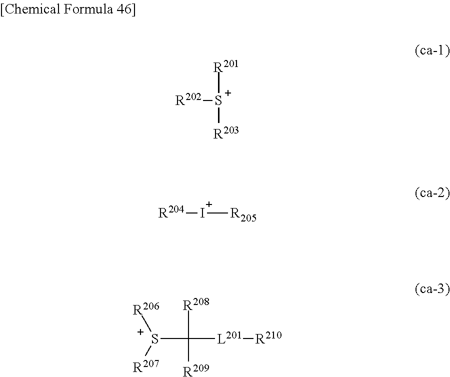

[In the formulae, R²⁰¹ to R²⁰⁷ each independently represent an aryl group, an alkyl group, or an alkenyl group, each of which may have a substituent. R²⁰¹ to R²⁰³ and R²⁰⁶ and R²⁰⁷ may be bonded to each other to form a ring together with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent.

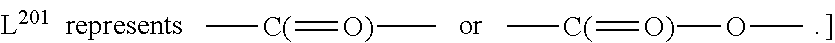

In General Formulae (ca-1) to (ca-3), examples of the aryl group as R²⁰¹ to R²⁰⁷ include an unsubstituted aryl group having 6 to 20 carbon atoms, where a phenyl group or a naphthyl group is preferable.

The alkyl group as R²⁰¹ to R²⁰⁷ is preferably a chain-like or cyclic alkyl group which has 1 to 30 carbon atoms.

The alkenyl group as R²⁰¹ to R²⁰⁷ preferably has 2 to 10 carbon atoms.

Examples of the substituent which may be contained in R²⁰¹ to R²⁰⁷ and R²¹⁰ include an alkyl group, a halogen atom, a halogenated alkyl group, a carbonyl group, a cyano group, an amino group, an aryl group, and groups each represented by General Formulae (ca-r-1) to (ca-r-7).

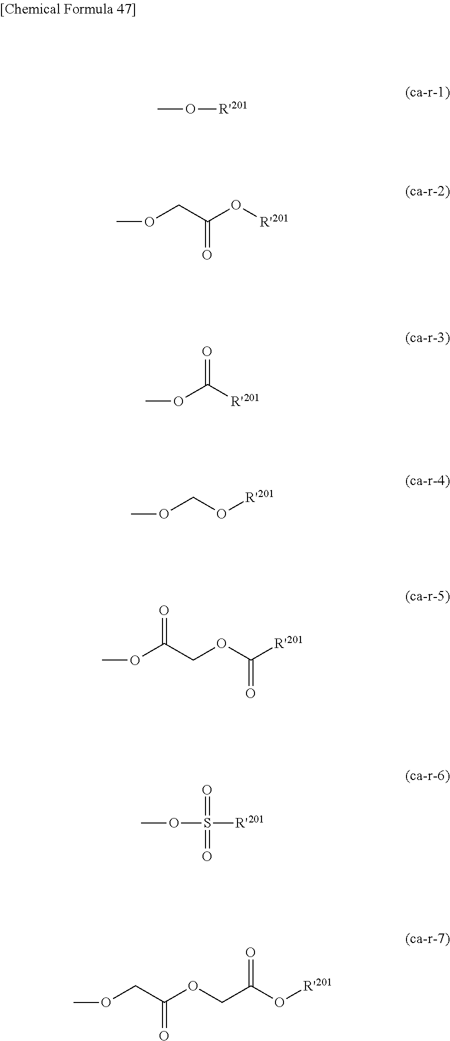

[In the formulae, R′²⁰¹'s each independently represents a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The number of carbon atoms in the monocycloalkane is preferably 3 to 6, and specifically, cyclopentane and cyclohexane are exemplary examples. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and heterocyclic groups each represented by Chemical Formulae (r-hr-1) to (r-hr-16).

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R′²⁰¹ may be linear or branched, and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include those for the acid dissociable group represented by Formula (a1-r-2) which are the exemplary examples of the cyclic group which may have a substituent and the chain-like alkyl group which may have a substituent, in addition to those described above.

Among the examples, R′²⁰¹ preferably represents a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (b5-r-1) to (b5-r-4).

In General Formulae (ca-1) to (ca-3), in a case where R²⁰¹ to R²⁰³ and R²⁰⁶ and R²⁰⁷ are bonded to each other to form a ring with a sulfur atom in the formula, these groups may be bonded to each other via a heteroatom such as a sulfur atom, an oxygen atom, or a nitrogen atom, or a functional group such as a carbonyl group, —SO—, —SO₂—, —SO₃—, —COO—, —CONH—, or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ each represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂-containing cyclic group which may have a substituent.

Examples of the aryl group as R²¹⁰ include an unsubstituted aryl group having 6 to 20 carbon atoms, and a phenyl group or a naphthyl group is preferable.

As the alkyl group represented by R²¹⁰, a chain-like or cyclic alkyl group having 1 to 30 carbon atoms is preferable.

The alkenyl group as R²¹⁰ preferably has 2 to 10 carbon atoms.

The —SO₂-containing cyclic group which may have a substituent as R²¹⁰ is preferably “—SO₂-containing polycyclic group” and more preferably a group represented by General Formula (b5-r-1).

Specific suitable examples of the cation represented by Formula (ca-1) include cations each represented by Chemical Formulae (ca-1-1) to (ca-1-75).

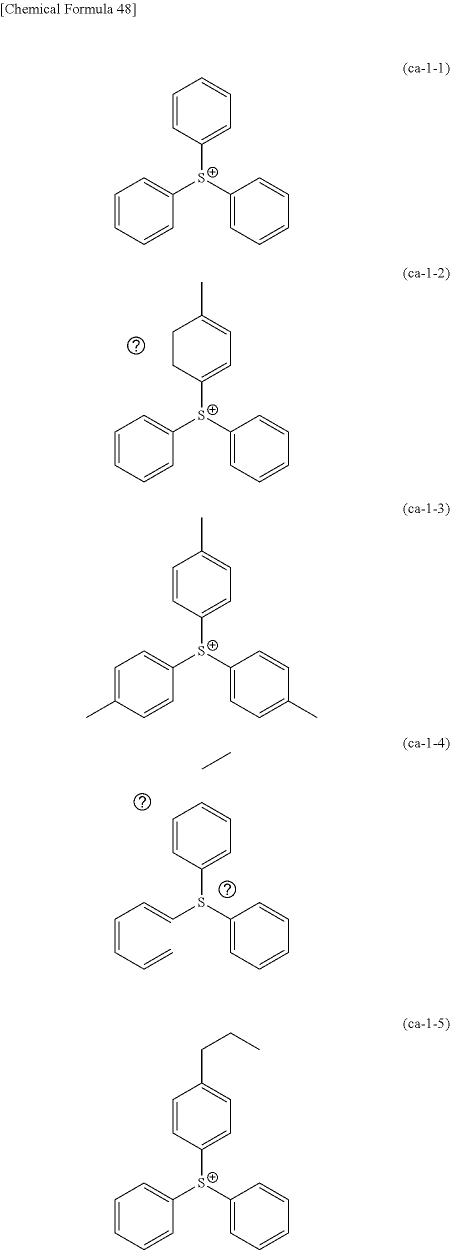

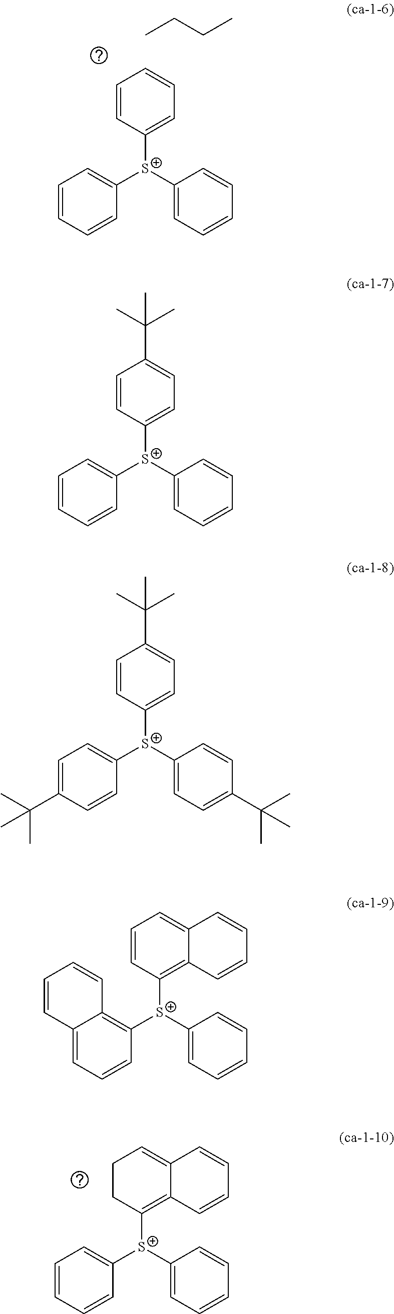

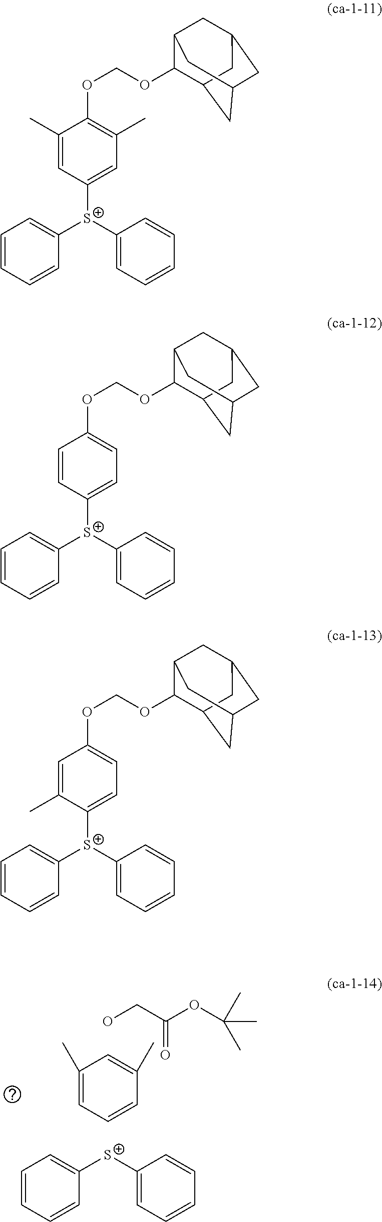

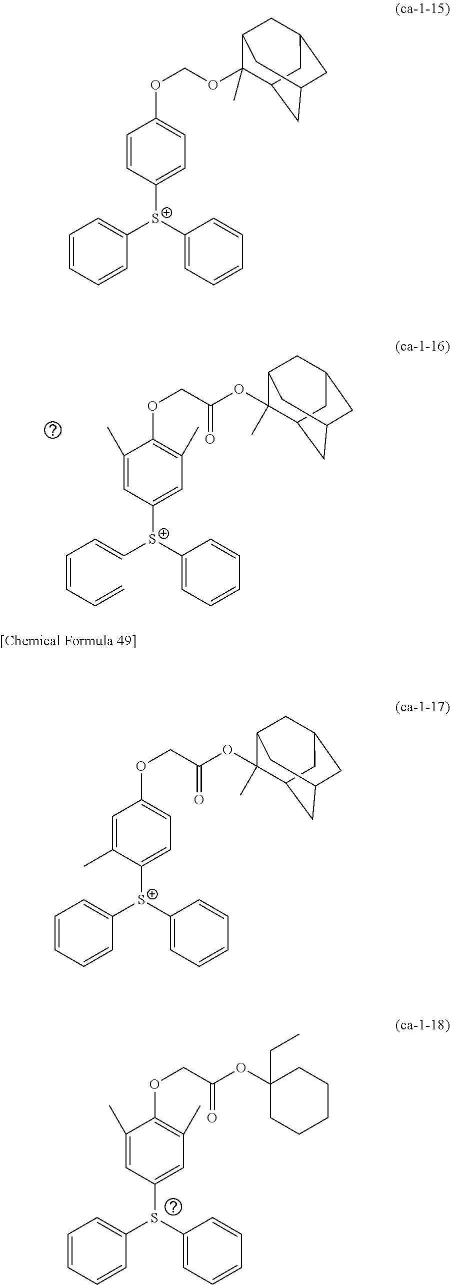

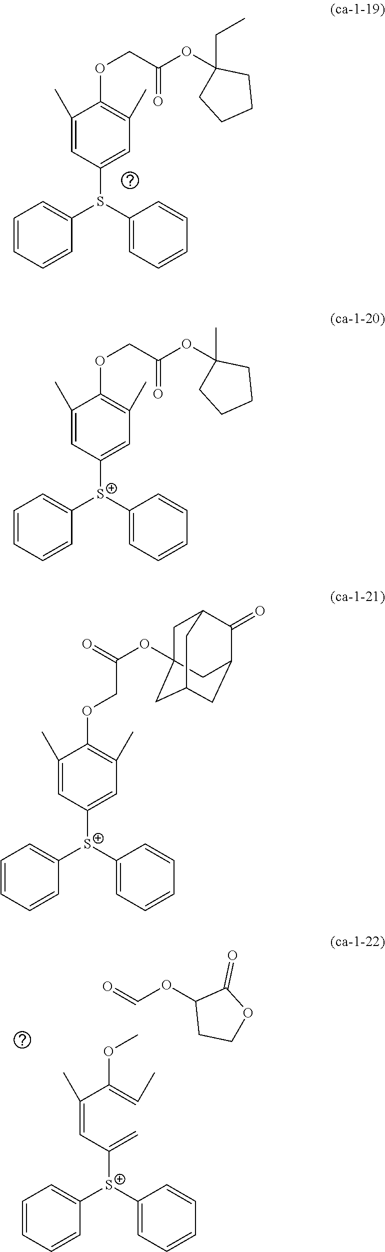

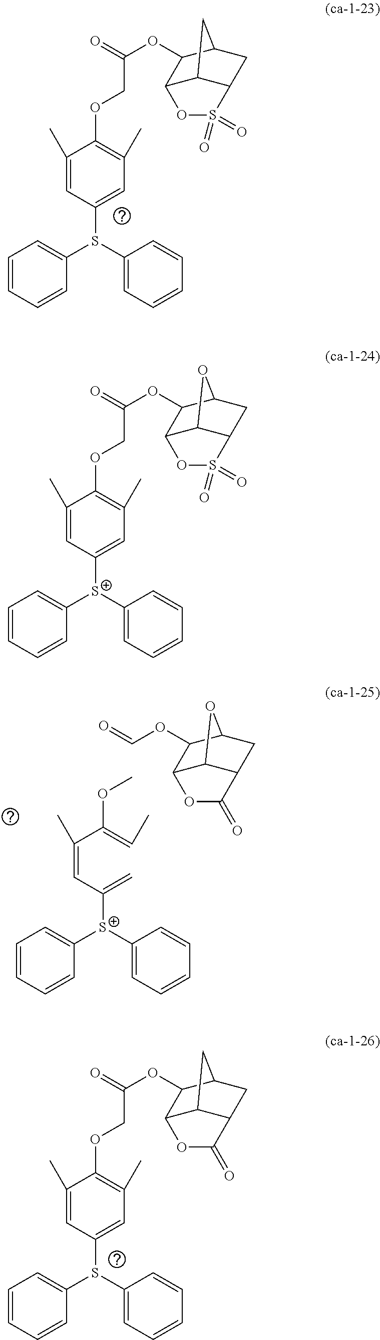

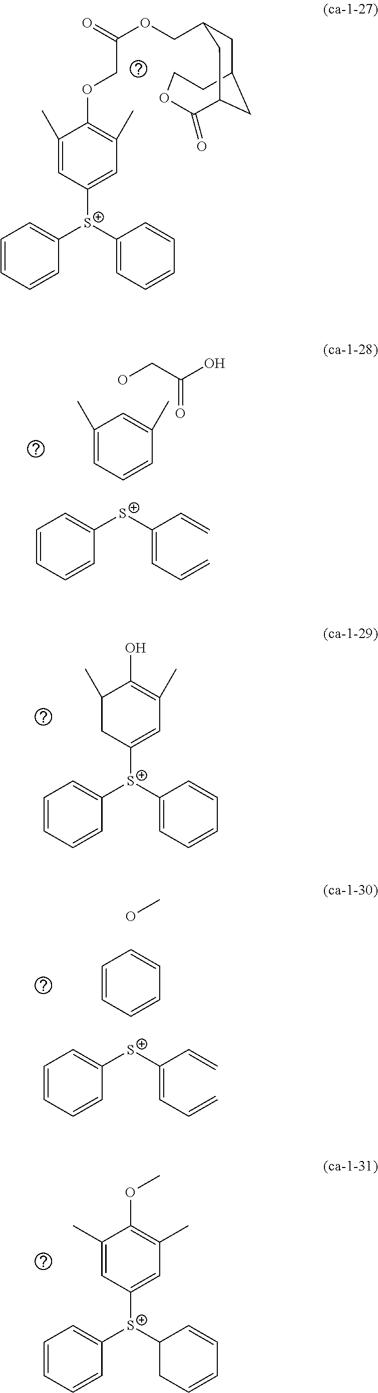

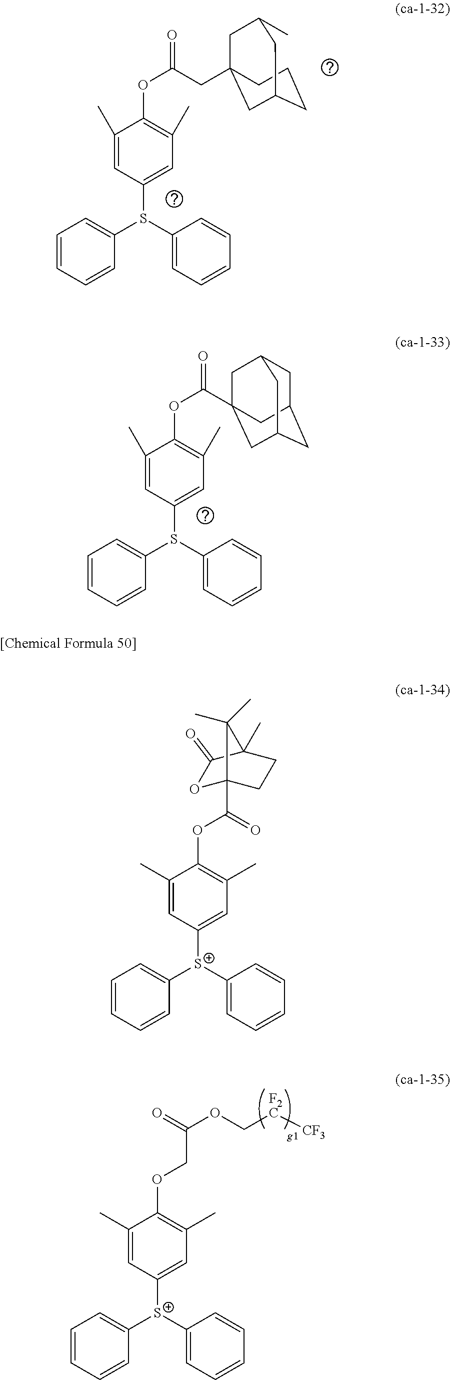

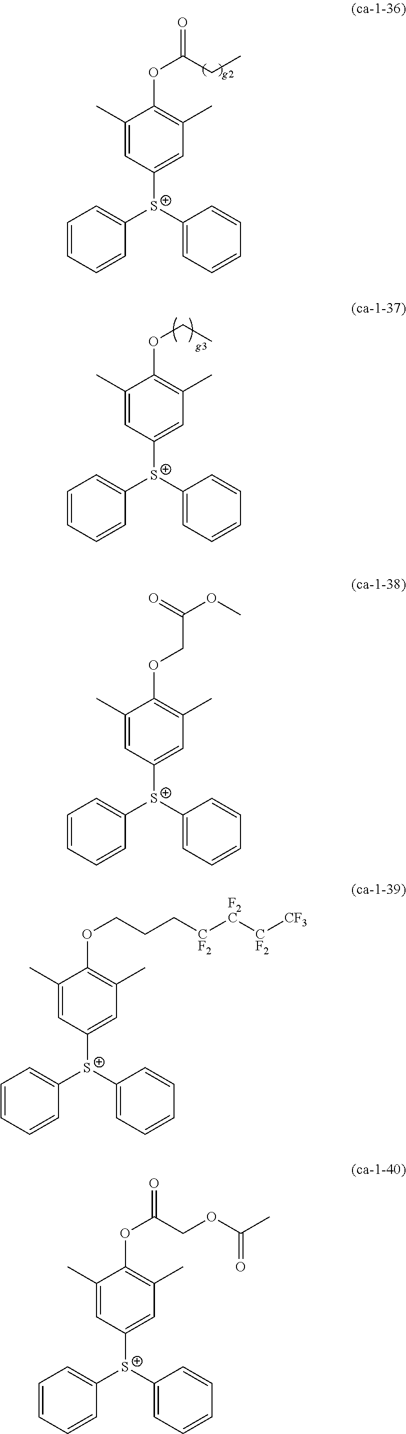

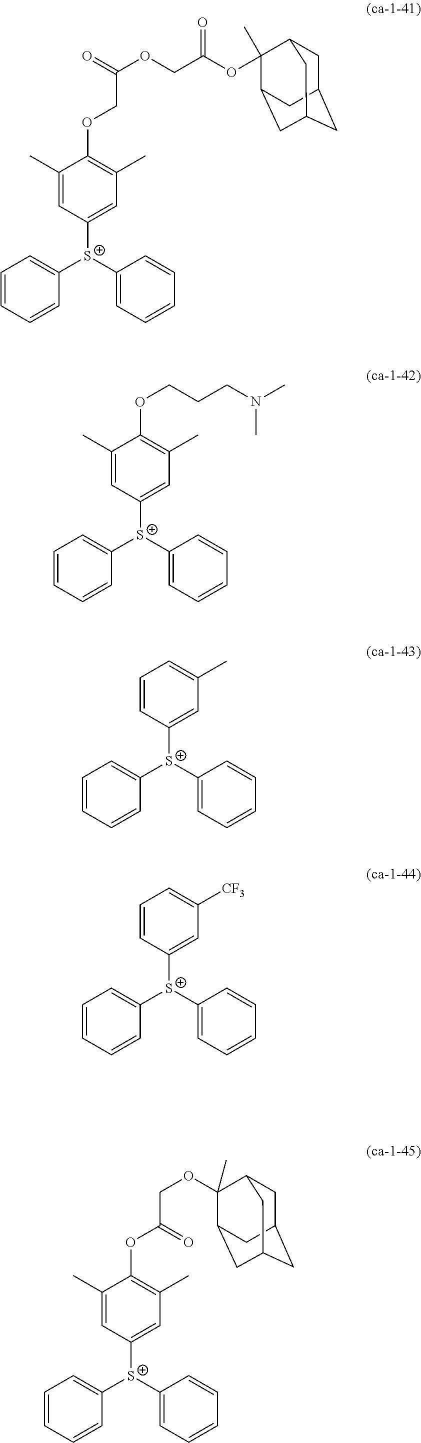 [SMILES: Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C ; Cc1cccc([S+](c2ccccc2)c2ccccc2)c1 ; FC(F)(F)c1cccc([S+](c2ccccc2)c2ccccc2)c1 ; Cc1cc([S+](c2ccccc2)c2ccccc2)cc(C)c1OC(=O)COC1(C)C2CC3CC(C2)CC1C3]

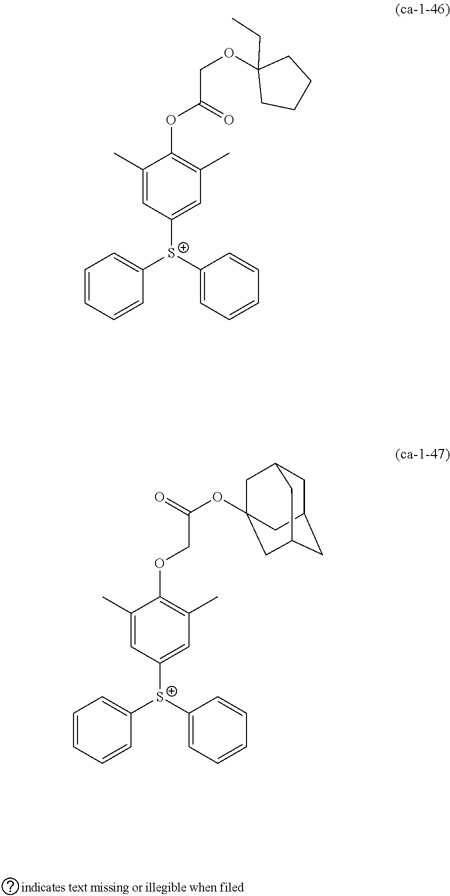 [SMILES: CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

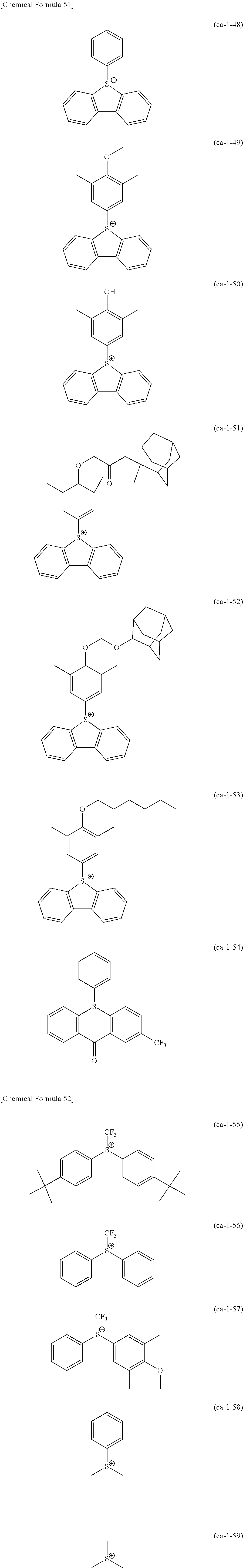

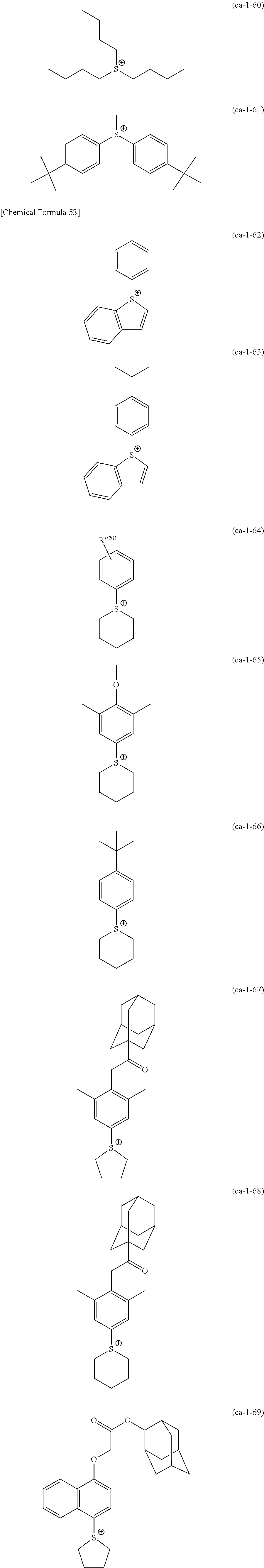

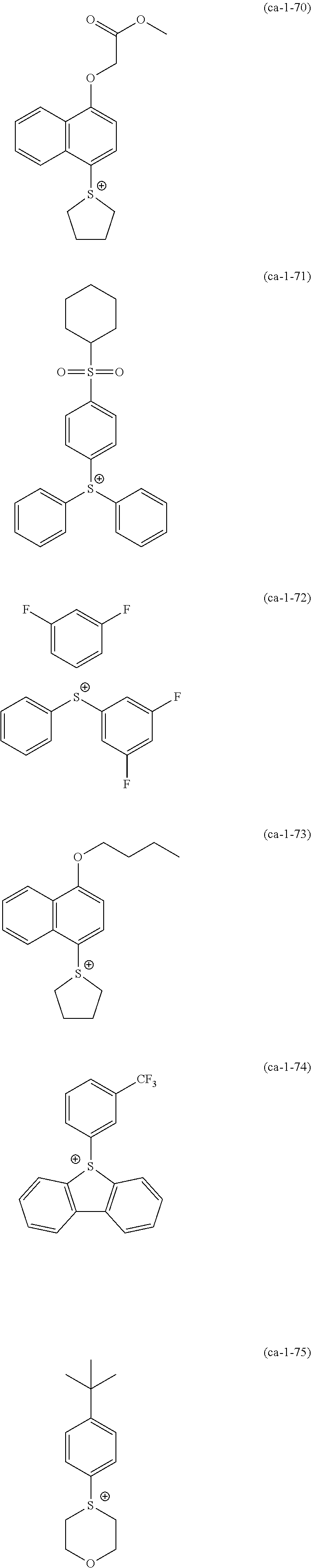

[In the formulae, R″²⁰¹ represents a hydrogen atom or a substituent, and examples of the substituent include the same groups as those for the substituents which may be included in R²⁰¹ to R²⁰⁷ and R²¹⁰ to R²¹².]

Specific examples of suitable cations represented by Formula (ca-2) include a diphenyliodonium cation and a bis(4-tert-butylphenyl) iodonium cation.

Specific examples of suitable cations represented by Formula (ca-3) include cations each represented by Formulae (ca-3-1) to (ca-3-6).

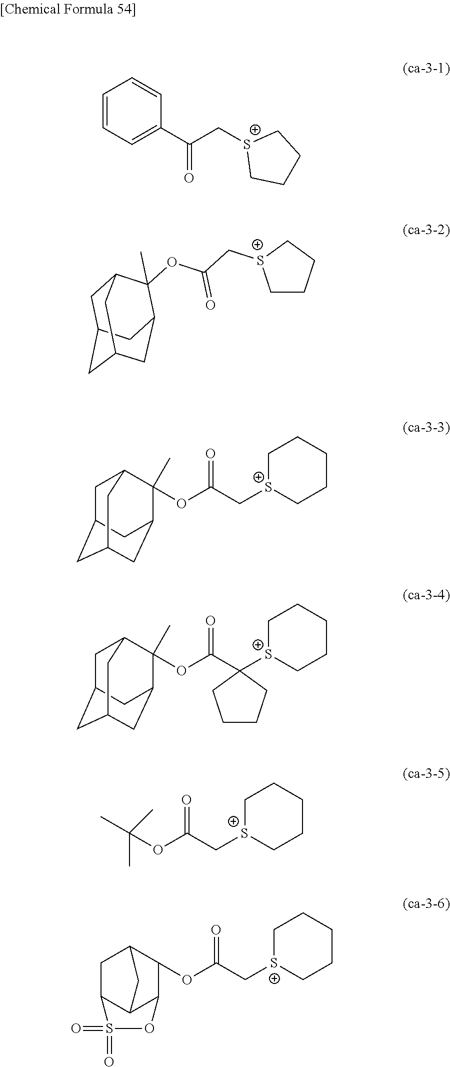 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

A cation represented by Formula (ca-1) is preferable as M′^{m+}. Further, an m-valent cation having a fluorine atom is preferable as M′^{m+}. A cation represented by Formula (ca-1f) is preferable as M′^{m+}.

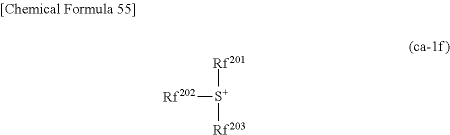

[In the formula, Rf²⁰¹ to Rf²⁰³ each independently represent an aryl group, an alkyl group, or an alkenyl group, which may have a substituent. Rf²⁰¹ to Rf²⁰³ may be bonded to each other to form a ring together with the sulfur atoms in the formula. Here, at least one of Rf²⁰¹ to Rf²⁰³ contains at least one fluorine atom.]

Rf²⁰¹ to Rf²⁰³ in Formula (ca-1f) each have the same definition as that for R²⁰¹ to R²⁰³ in Formula (ca-1). Here, at least one of Rf²⁰¹ to Rf²⁰³ has at least one fluorine atom. It is preferable that the cation represented by Formula (ca-1f) has three or more fluorine atoms. Any one of Rf²⁰¹ to Rf²⁰³ may have three or more fluorine atoms, or the total number of fluorine atoms in Rf²⁰¹ to Rf²⁰³ may be three or more.

Specific examples of the constitutional unit (a5) are shown below, but the present invention is not limited thereto.

In the formulae shown below, R^{α} represents a hydrogen atom, a methyl group, or a trifluoromethyl group. m and M′^{m+} each have the same definition as that for m and M′^{m+} in General Formula (a5-1).

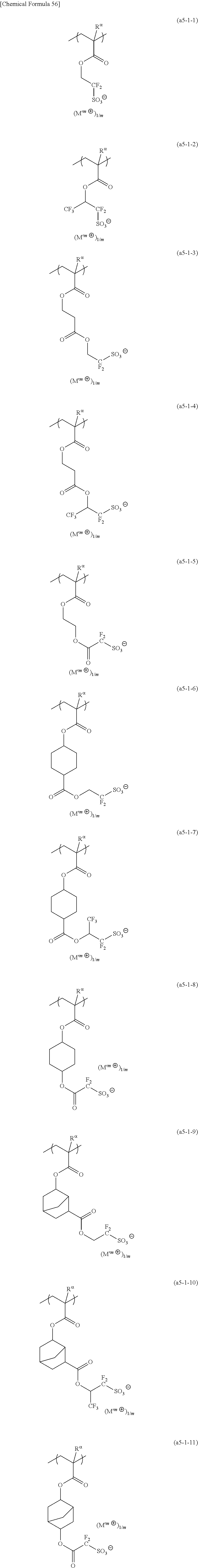

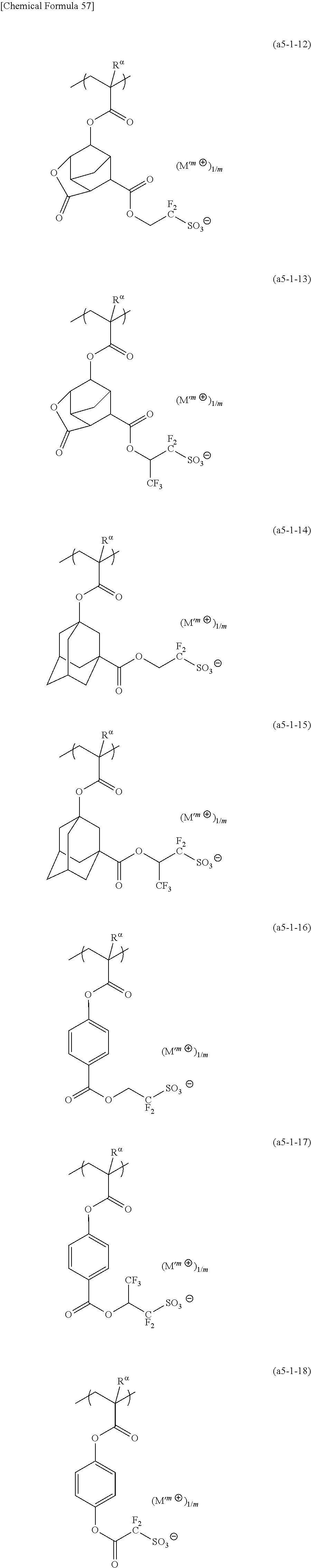

The constitutional unit (a5) of the component (A1) may be used alone or two or more kinds thereof may be used. The component (A1) may or may not have the constitutional unit (a5). In a case where the resist composition of the present embodiment does not contain a component (B) described below, it is preferable that the component (A1) has the constitutional unit (a5).

In a case where the component (A1) has the constitutional unit (a5), the proportion of the constitutional unit (a5) in the component (A1) is preferably in a range of 1% to 30% by mole, more preferably in a range of 2% to 20% by mole, and still more preferably in a range of 5% to 15% by mole with respect to the total amount (100% by mole) of all constitutional units constituting the component (A1).

In a case where the proportion of the constitutional unit (a5) is greater than or equal to the lower limits of the above-described preferable ranges, the sensitivity is likely to be further increased. Meanwhile, in a case where the proportion thereof is less than or equal to the upper limits of the above-described preferable ranges, the constitutional unit (a5) and other constitutional units are likely to be balanced.

### Constitutional Unit (a6):

The constitutional unit (a6) is a constitutional unit having acid diffusion controllability. The component (A1) may or may not have the constitutional unit (a6). As the constitutional unit (a6), a known constitutional unit can be used. Examples of the constitutional unit (a6) include a constitutional unit having a structure described in the section of the component (D1) and the component (D2) below. Examples thereof include a constitutional unit having a structure represented by any of General Formulae (d1-1) to (d1-3).

The constitutional unit (a6) contained in the component (A1) may be used alone or two or more kinds thereof may be used.

In a case where the component (A1) has the constitutional unit (a6), the proportion of the constitutional unit (a6) in the component (A1) is preferably in a range of 1% to 20% by mole, more preferably in a range of 2% to 15% by mole, and still more preferably in a range of 3% to 10% by mole with respect to the total amount (100% by mole) of all constitutional units constituting the component (A1).

In a case where the proportion of the constitutional unit (a6) is greater than or equal to the lower limits of the above-described preferable ranges, higher sensitivity is likely to be realized. Meanwhile, in a case where the proportion thereof is less than or equal to the upper limits of the above-described preferable ranges, the constitutional unit (a5) and other constitutional units are likely to be balanced.

### Constitutional Unit (a2):

The component (A1) may have a constitutional unit (a2) containing a lactone-containing cyclic group (here, constitutional units corresponding to the constitutional unit (a1) are excluded).

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

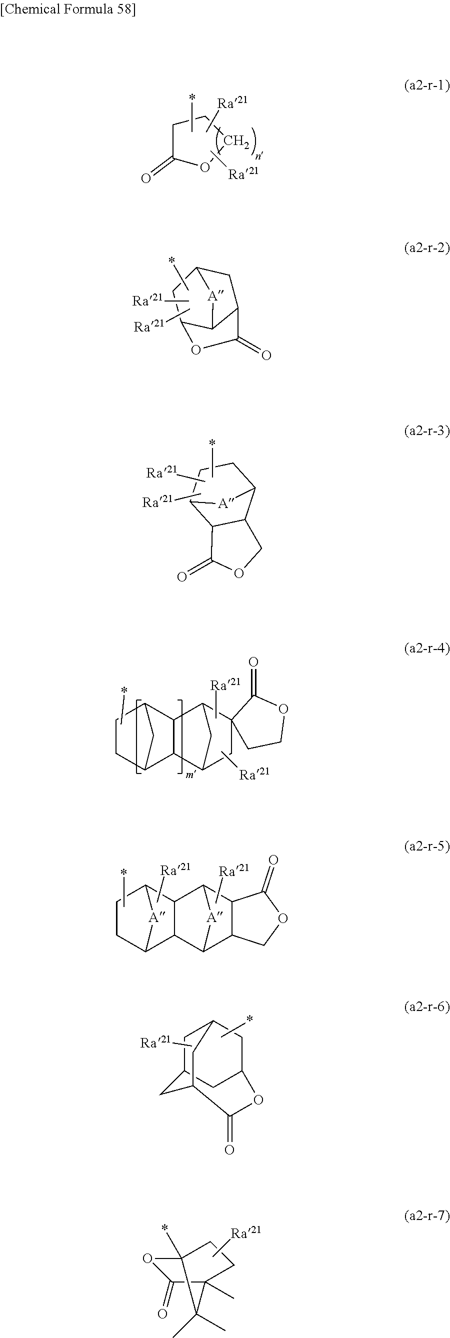

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site (the same applies hereinafter).]

In General Formulae (a2-r-1) to (a2-r-7), it is preferable that the alkyl group as Ra′²¹ is an alkyl group having 1 to 6 carbon atoms. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group described as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, or a lactone-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include a group in which one or more hydrogen atoms have been removed from a monocycloalkane such as cyclopentane or cyclohexane; and a group in which one or more hydrogen atoms have been removed from a polycycloalkane such as adamantane, norbornane, isobornane, tricyclo[5.2.1.0^{2,6}]decane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that each Ra′²¹ independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups that contain an oxygen atom or a sulfur atom include a group obtained by interposing —O— or —S— in the terminal of the alkylene group or between the carbon atoms of the alkylene group, and examples thereof include —O—CH₂—, —CH₂—O—CH₂—, —S—CH₂—, and —CH₂—S—CH₂—. A″ preferably represents an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

[chemical structure]

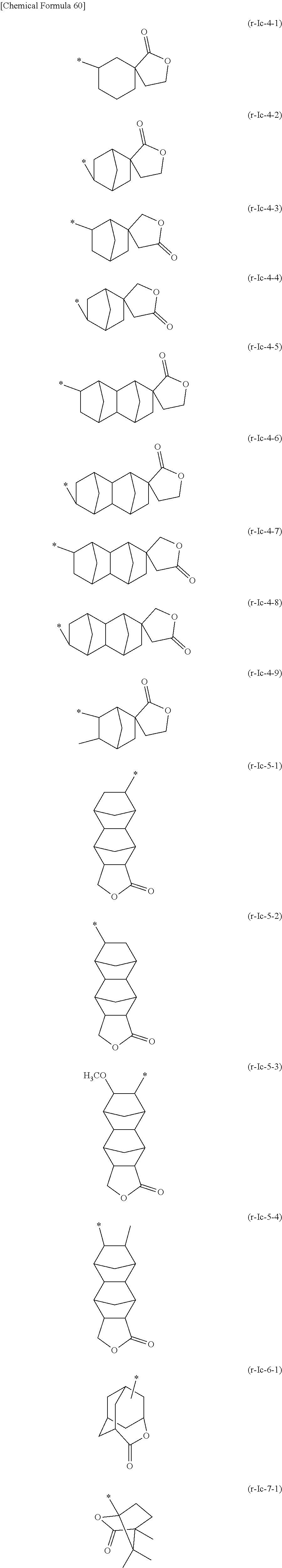

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

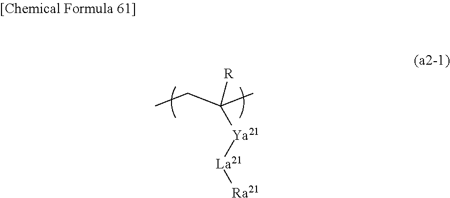

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group.]

In Formula (a2-1), R has the same definition as described above. R preferably represents a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a heteroatom. Examples of the divalent linking group as Ya²¹ include the same groups as those for the divalent linking group as Ya^{x1} in General Formula (a10-1).

It is preferable that Ya²¹ represents a single bond, an ester bond [—C(═O)—O—], an ether bond (—O—), a linear or branched alkylene group, or a combination thereof.

In Formula (a2-1), it is preferable that Ya²¹ represents a single bond and La²¹ represents —COO— or —OCO—.

In Formula (a2-1), Ra²¹ represents a lactone-containing cyclic group.

Suitable examples of the lactone-containing cyclic group as Ra²¹ include groups each represented by General Formulae (a2-r-1) to (a2-r-7).

The constitutional unit (a2) included in the component (A1) may be used alone or two or more kinds thereof may be used. The component (A1) may or may not have the constitutional unit (a2).

In a case where the component (A1) has the constitutional unit (a2), the proportion of the constitutional unit (a2) is preferably in a range of 1% to 20% by mole, more preferably in a range of 1% to 15% by mole, and still more preferably in a range of 1% to 10% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

### Constitutional Unit (a8):

The constitutional unit (a8) is a constitutional unit derived from a compound represented by General Formula (a8-1).

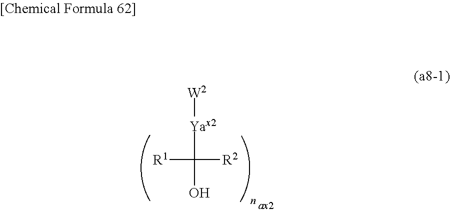

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms which may have a fluorine atom, or a hydrogen atom. R² and Ya^{x2} may be bonded to each other to form a ring structure. nₐₓ₂ represents an integer of 1 to 3.]

The term “polymerizable group” in the polymerizable group-containing group as W² is a group that enables a compound having the polymerizable group to be polymerized by radical polymerization or the like, and refers to a group containing multiple bonds between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group containing a heteroatom.

Suitable examples of the polymerizable group-containing group include a group represented by a chemical formula: C(R^{X11})(R^{X12})═C(R^{X13})—Ya^{x0}-.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}.

The condensed ring formed by Ya^{x2} and W² may have a substituent.

Specific examples of the constitutional unit (a8) are shown below.

In the following formulae, R^{α} represents a hydrogen atom, a methyl group, or a trifluoromethyl group.

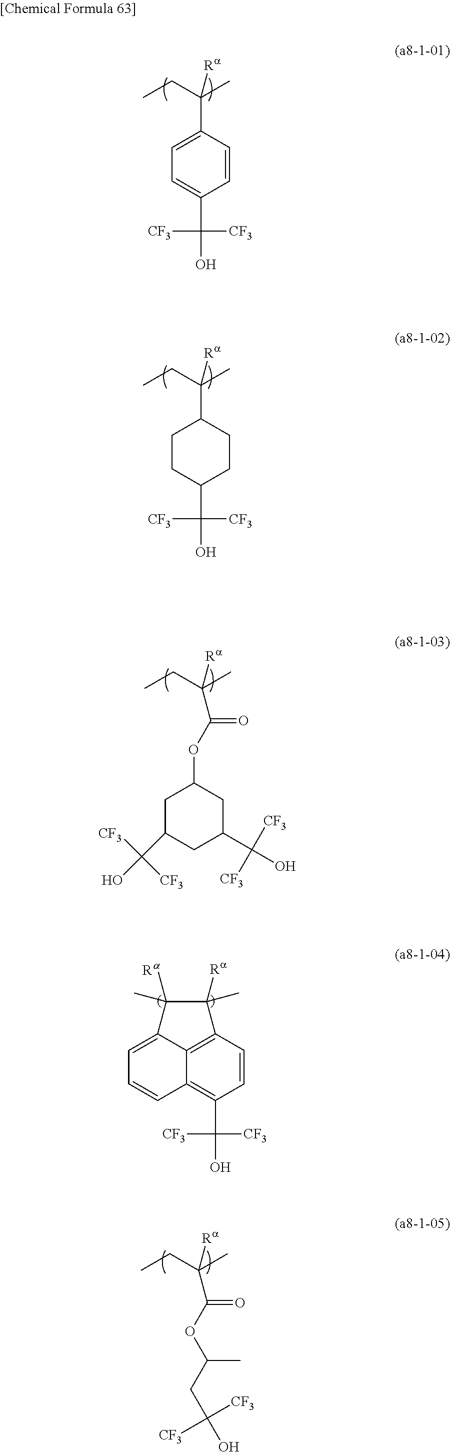

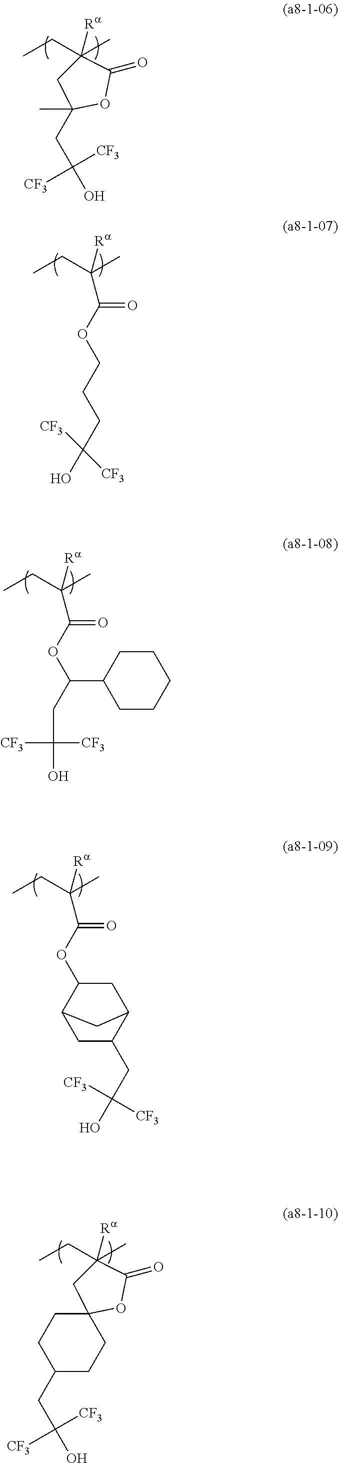

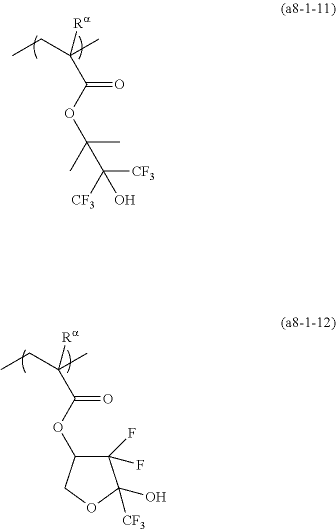

Among the examples, the constitutional unit (a8) is preferably at least one selected from the group consisting of constitutional units each represented by Chemical Formulae (a8-1-01) to (a8-1-04), (a8-1-06), (a8-1-08), (a8-1-09), and (a8-1-10) and more preferably at least one selected from the group consisting of constitutional units each represented by Chemical Formulae (a8-1-01) to (a8-1-04) and (a8-1-09).

The constitutional unit (a8) contained in the component (A1) may be used alone or two or more kinds thereof may be used. The component (A1) may or may not have the constitutional unit (a8).

The proportion of the constitutional unit (a8) in the component (A1) is preferably in a range of 0% to 50% by mole and more preferably in a range of 0% to 30% by mole with respect to the total amount (100% by mole) of all constitutional units constituting the component (A1).

The component (A1) contained in the resist composition may be used alone or a combination of two or more kinds thereof may be used.

Examples of the component (A1) include a polymer compound having a repeating structure of the constitutional unit (a1) and the constitutional unit (a10); and a polymer compound having a repeating structure of the constitutional unit (a1), the constitutional unit (a10), and the constitutional unit (a5).

Such a component (A1) can be produced by dissolving a monomer, from which each constitutional unit is derived, in a polymerization solvent and adding a radical polymerization initiator such as azobisisobutyronitrile (AIBN) or dimethyl azobisisobutyrate (for example, V-601) to the solution so that the polymerization is carried out.

Alternatively, such a component (A1) can be produced by dissolving, in a polymerization solvent, a monomer from which the constitutional unit (a1) is derived and a monomer from which an optional constitutional unit (for example, the constitutional unit (a10) or the constitutional unit (a5)) is derived, adding a radical polymerization initiator as described above thereto to carry out polymerization, and carrying out a deprotection reaction.

Further, a —C(CF₃)₂—OH group may be introduced into the terminal of the component (A1) during the polymerization using a chain transfer agent such as HS—CH₂—CH₂—CH₂—C(CF₃)₂—OH in combination. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 50000, more preferably in a range of 5000 to 40000, and still more preferably in a range of 5000 to 30000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern are excellent.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### ⋅In Regard to Component (A2)

In the resist composition of the present embodiment, a base material component (hereinafter, also referred to as “component (A2)”) which does not correspond to the component (A1) and whose solubility in a developing solution is changed by the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or a combination of two or more kinds thereof may be used.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having excellent various lithography characteristics such as high sensitivity, high resolution, and improved roughness is likely to be formed.

In the resist composition of the present embodiment, the amount of the component (A) may be adjusted according to the thickness of the resist film intended to be formed.

### <Base Component (D)>

The resist composition of the present embodiment contains a base component (hereinafter, also referred to as “component (D)”) that traps an acid generated upon light exposure (that is, controls diffusion of the acid), in addition to the component (A). The component (D) acts as a quencher (an acid diffusion control agent) which traps the acid generated in the resist composition upon light exposure.

### <<Component (D0)>>

The resist composition of the present embodiment contains a compound (D0) represented by General Formula (d0) (hereinafter, also referred to as “component (D0)”) as the component (D).

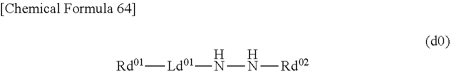

[In the formula, Rd⁰¹ represents a monovalent organic group, Rd⁰² represents a monovalent organic group or a hydrogen atom, and Ld⁰¹ represents a single bond or a divalent linking group.]

### First Embodiment

In General Formula (d0), Rd⁰¹ represents a monovalent organic group. Examples of the monovalent organic group as Rd⁰¹ include a hydrocarbon group which may have a substituent. The hydrocarbon group which may have a substituent may be an aliphatic hydrocarbon group which may have a substituent or an aromatic hydrocarbon group which may have a substituent. The aliphatic hydrocarbon group which may have a substituent may be saturated or unsaturated. Examples of the monovalent organic group as Rd⁰¹ include a cyclic group which may have a substituent, and a linear or branched aliphatic hydrocarbon group which may have a substituent.

Cyclic Group which May have Substituent:

The cyclic group which may have a substituent is preferably a cyclic hydrocarbon group which may have a substituent. The cyclic hydrocarbon group which may have a substituent may be an aromatic hydrocarbon group which may have a substituent or a cyclic aliphatic hydrocarbon group which may have a substituent.

In Formula (d0), the aromatic hydrocarbon group as Rd⁰¹ has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as Rd⁰¹ include benzene, fluorene, naphthalene, anthracene, phenanthrene, biphenyl, and an aromatic heterocyclic ring in which some carbon atoms constituting these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as Rd⁰¹ include a group in which one hydrogen atom has been removed from the aromatic ring (an aryl group such as a phenyl group or a naphthyl group) and a group in which one hydrogen atom of the aromatic ring is substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (d0), examples of the cyclic aliphatic hydrocarbon group as Rd⁰¹ include an aliphatic hydrocarbon group having a ring in the structure. As the aliphatic hydrocarbon group including a ring in the structure, an alicyclic hydrocarbon group (a group formed by removing one hydrogen atom from an aliphatic hydrocarbon ring), a group in which an alicyclic hydrocarbon group is bonded to the terminal of a linear or branched aliphatic hydrocarbon group, and a group in which an alicyclic hydrocarbon group is interposed in a linear or branched aliphatic hydrocarbon group are exemplary examples. The cyclic aliphatic hydrocarbon group may be saturated or may be unsaturated.

The alicyclic hydrocarbon group preferably has 3 to 20 carbon atoms and more preferably has 3 to 12 carbon atoms.

In Formula (d0), the alicyclic hydrocarbon group as Rd⁰¹ may be a polycyclic group or a monocyclic group. As the monocyclic alicyclic hydrocarbon group, a group in which one or more hydrogen atoms have been removed from a monocycloalkane is preferable. The monocycloalkane preferably has 3 to 6 carbon atoms. As specific examples of the monocycloalkane, cyclopentane, cyclohexane, and the like are exemplary examples. As the polycyclic alicyclic hydrocarbon group, a group formed by removing one or more hydrogen atoms from a polycycloalkane is preferable, and the polycycloalkane preferably has 7 to 30 carbon atoms. Specific examples of the polycycloalkane include a polycycloalkane having a crosslinked ring polycyclic skeleton such as adamantane, norbornane, isobornane, tricyclo[5.2.1.0^{2,6}]decane, and tetracyclododecane.

The linear or branched aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and particularly preferably has 1 to 3 carbon atoms. Examples of the linear or branched aliphatic hydrocarbon group include the same groups as those for R′²⁰¹ and the like in General Formulae (ca-r-1) to (ca-r-7).

In Formula (d0), the cyclic hydrocarbon group as Rd⁰¹ may have a heteroatom such as a heterocyclic ring. Specific examples thereof include lactone-containing cyclic groups each represented by General Formulae (a2-r-1) to (a2-r-7), —SO₂-containing cyclic group each represented by General Formulae (a5-r-1) to (a5-r-4), and heterocyclic groups each represented by Chemical Formulae (r-hr-1) to (r-hr-16).

In Formula (d0), the cyclic group as Rd⁰¹ may or may not have a substituent. Examples of the substituent in the cyclic group as Rd⁰¹ include an alkyl group, an alkoxy group, a halogen atom, a halogenated alkyl group, a hydroxyl group, a hydroxyalkyl group, a carboxy group, a carbonyl group, and a nitro group.

As the alkyl group as the substituent, an alkyl group having 1 to 5 carbon atoms is preferable, and a methyl group, an ethyl group, a propyl group, an n-butyl group, and a tert-butyl group, and the like are exemplary examples.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is preferable, and a methoxy group, an ethoxy group, and the like are exemplary examples.

As the halogen atom as the substituent, a fluorine atom and a chlorine atom are exemplary examples, and an iodine atom is preferable.

As the halogenated alkyl group as the substituent, a group in which some or all hydrogen atoms in the alkyl group having 1 to 5 carbon atoms are substituted with the halogen atoms (fluorine atom and the like) is an exemplary example.

The carbonyl group as the substituent is a group that substitutes a methylene group (—CH₂—) constituting the cyclic hydrocarbon group.

Examples of the hydroxyalkyl group as a substituent include a group in which some or all hydrogen atoms of an alkyl group having 1 to 5 carbon atoms are substituted with hydroxy groups.

The carbonyl group as the substituent is a group that substitutes a methylene group (—CH₂—) constituting the cyclic hydrocarbon group.

Among these, as the substituent in the cyclic group as Rd⁰¹ in Formula (d0), an alkyl group, an alkoxy group, a halogen atom, a halogenated alkyl group, a hydroxyl group, a hydroxyalkyl group, or a carboxy group is preferable, and a hydroxyl group, a hydroxyalkyl group, or a carboxy group is more preferable. It is preferable that the hydroxyalkyl group is a group represented by —(CH₂)ₙ—OH (n represents an integer of 1 to 6). n in the formula preferably represents an integer of 1 to 3 and more preferably 1 or 2.

Linear or Branched Aliphatic Hydrocarbon Group which May have Substituent:

The linear or branched aliphatic hydrocarbon group which may have a substituent as Rd⁰¹ may be a saturated aliphatic hydrocarbon group or an unsaturated aliphatic hydrocarbon group. The linear or branched aliphatic hydrocarbon group which may have a substituent preferably has 1 to 20 carbon atoms, more preferably has 1 to 15 carbon atoms, still more preferably has 1 to 10 carbon atoms, and particularly preferably has 1 to 6 carbon atoms. Here, the number of carbon atoms in a substituent is not included in the number of carbon atoms.

In Formula (d0), examples of the linear or branched aliphatic hydrocarbon group which may have a substituent as Rd⁰¹ include a linear or branched alkyl group which may have a substituent and a linear or branched alkenyl group which may have a substituent.

In Formula (d0), the linear alkyl group as Rd⁰¹ has preferably 1 to 20 carbon atoms, more preferably 1 to 15 carbon atoms, still more preferably 1 to 10 carbon atoms, and particularly preferably 1 to 6 carbon atoms.

The branched alkyl group preferably has 3 to 20 carbon atoms, more preferably has 3 to 15 carbon atoms, still more preferably has 3 to 10 carbon atoms, and particularly preferably has 3 to 6 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

In Formula (d0), the linear alkenyl group as Rd⁰¹ has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. The branched alkenyl group as Rd⁰¹ has preferably 3 to 10 carbon atoms, more preferably 3 to 5 carbon atoms, and still more preferably 3 or 4 carbon atoms. Examples of the branched alkenyl group include a 1-methylvinyl group, a 2-methylvinyl group, a 1-5 methylpropenyl group, and a 2-methylpropenyl group.

In Formula (d0), the linear or branched aliphatic hydrocarbon group as Rd⁰¹ may or may not have a substituent. Examples of the substituent in the linear or branched aliphatic hydrocarbon group as Rd⁰¹ include an alkoxy group, a halogen atom, a halogenated alkyl group, a hydroxyl group, a carboxy group, a carbonyl group, a nitro group, and an amino group.

In Formula (d0), Rd⁰¹ preferably represents a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent. As the substituent contained in the cyclic group as Rd⁰¹, an alkoxy group, a fluorine atom, a hydroxyl group, a hydroxyalkyl group, or a carboxy group is preferable, and a hydroxyl group, a hydroxyalkyl group, or a carboxy group is more preferable. It is preferable that the hydroxyalkyl group is a group represented by —(CH₂)ₙ—OH (n represents an integer of 1 to 6). n in the formula preferably represents an integer of 1 to 3 and more preferably 1 or 2.

In Formula (d0), Rd⁰² represents a monovalent organic group or a hydrogen atom. Examples of the monovalent organic group include the same groups as those for the monovalent organic group as Rd⁰¹.

The monovalent organic group as Rd⁰² may be linked to a divalent linking group having a heteroatom and the monovalent organic group described in the section of Rd⁰¹.

Examples of the divalent linking group having a heteroatom include the same groups as those for the divalent linking group having a heteroatom as Ya^{x1} in General Formula (a10-1). As the divalent linking group having a heteroatom, —C(═O)—O—, —O—C(═O)—, —O—, —C(═O)—, or a linear or branched aliphatic hydrocarbon group, or a combination of two or more thereof is preferable.

In Formula (d0), the monovalent organic group as Rd⁰² is preferably a cyclic group which may have a substituent, or a group in which a cyclic group which may have a substituent is bonded to a divalent linking group having —C(═O)—O—, —O—C(═O)—, —O—, or —C(═O)—. The cyclic group which may have a substituent is more preferably an aromatic hydrocarbon group which may have a substituent, still more preferably a phenyl group which may have a substituent or a naphthyl group which may have a substituent, and particularly preferably a phenyl group which may have a substituent. The substituent in the cyclic group is preferably an alkoxy group, a fluorine atom, a hydroxyl group, a hydroxyalkyl group, or a carboxy group and more preferably a hydroxyl group, a hydroxyalkyl group, or a carboxy group. It is preferable that the hydroxyalkyl group is a group represented by —(CH₂)ₙ—OH (n represents an integer of 1 to 6). n in the formula preferably represents an integer of 1 to 3 and more preferably 1 or 2.

In Formula (d0), it is preferable that at least one of Rd⁰¹ or Rd⁰² has an aromatic ring, and both Rd⁰¹ and Rd⁰² may have an aromatic ring. In a case where at least one of Rd⁰¹ or Rd⁰² has an aromatic ring, the sensitivity and lithography characteristics such as CDU are likely to be improved.

In Formula (d0), Rd⁰¹ and Rd⁰² may or may not contain an acid dissociable group. In a case where Rd⁰¹ and Rd⁰² contain an acid dissociable group, it is preferable that the total number of acid dissociable groups in Rd⁰¹ and Rd⁰² is not 2 or more. That is, in a case where one of Rd⁰¹ and Rd⁰² contains an acid dissociable group, it is preferable that the other does not contain an acid dissociable group. It is preferable that Rd⁰¹ and Rd⁰² each do not contain two or more acid dissociable groups. The compound (D0) is considered to release electrons and undergo oxidative decomposition upon light exposure. In a case where the compound (D0) contains an acid dissociable group, it is assumed that the compound (D0) is not decomposed unless the acid dissociable group is dissociated. Therefore, in a case where the compound (D0) contains two or more acid dissociable groups, the decomposition of the compound (D0) in the exposed portion is unlikely to proceed, and the sensitivity may be degraded.

In particular, a tert-butoxycarbonyl group (t-BOC) is relatively difficult to deprotect. Therefore, it is preferable that Rd⁰¹ and Rd⁰² do not contain a total of two or more t-BOCs. In a case where one of Rd⁰¹ and Rd⁰² contains t-BOC, it is preferable that the other does not contain t-BOC. In a case where one of Rd⁰¹ and Rd⁰² contains t-BOC, from the viewpoint of improving the sensitivity, the other has preferably an aromatic ring and more preferably an aromatic ring without containing t-BOC.

In Formula (d0), Ld⁰¹ represents a single bond or a divalent linking group. Examples of the divalent linking group include the same groups as those for the divalent linking group as Ya^{x1} in General Formula (a10-1).

In Formula (d0), as the divalent linking group represented by Ld⁰¹, —C(═O)—O—, —O—C(═O)—, —O—, —C(═O)—, or a linear or branched aliphatic hydrocarbon group, or a combination of two or more thereof is preferable. As the linear or branched aliphatic hydrocarbon group, a linear or branched alkylene group having 1 to 6 carbon atoms or a linear or branched alkenylene group having 2 to 6 carbon atoms is preferable. As the divalent linking group represented by Ld⁰¹, —C(═O)—O—, —O—C(═O)—, —O—, or —C(═O)— is more preferable.

It is preferable that the component (D0) is a compound represented by General Formula (d0-1) (hereinafter, also referred to as “component (D001)”) or a compound represented by General Formula (d0-2) (hereinafter, also referred to as “component (D002)”).

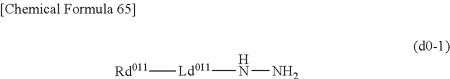

[In the formula, Rd⁰¹¹ represents a monovalent organic group, and Ld⁰¹¹ represents a single bond or a divalent linking group.]

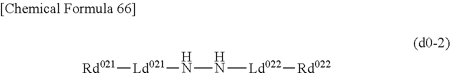

[In the formula, Rd⁰²¹ and Rd⁰²² each independently represent a monovalent organic group; and Ld⁰²¹ and Ld⁰²² each independently represent a single bond or a divalent linking group.]

⋅Compound represented by General Formula (d0-1) (component (D001))

In Formula (d0-1), Rd⁰¹¹ represents a monovalent organic group. Rd⁰¹¹ has the same definition as that for Rd⁰¹ in Formula (d0).

In Formula (d0-1), Rd⁰¹¹ preferably represents a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent. In a case where Rd⁰¹¹ has an aromatic ring, the sensitivity and lithography characteristics such as CDU are likely to be improved.

In Formula (d0-1), the substituent contained in the cyclic group as Rd⁰¹¹ is preferably an alkoxy group, a fluorine atom, a hydroxyl group, a hydroxyalkyl group, or a carboxy group and more preferably a hydroxyl group, a hydroxyalkyl group, or a carboxy group. It is preferable that the hydroxyalkyl group is a group represented by —(CH₂)ₙ—OH (n represents an integer of 1 to 6). n in the formula preferably represents an integer of 1 to 3 and more preferably 1 or 2.

In Formula (d0-1), Ld⁰¹¹ represents a single bond or a divalent linking group. Ld⁰¹¹ has the same definition as that for Ld⁰¹ in Formula (d0).

In Formula (d0-1), as the divalent linking group represented by Ld⁰¹¹, —C(═O)—O—, —O—C(═O)—, —O—, —C(═O)—, or a linear or branched aliphatic hydrocarbon group, or a combination of two or more thereof is preferable. As the linear or branched aliphatic hydrocarbon group, a linear or branched alkylene group having 1 to 6 carbon atoms or a linear or branched alkenylene group having 2 to 6 carbon atoms is preferable. As the divalent linking group represented by Ld⁰¹¹, —C(═O)—O—, —O—C(═O)—, —O—, or —C(═O)— is more preferable.

Specific examples of the component (D001) are shown below, but the present invention is not limited thereto.

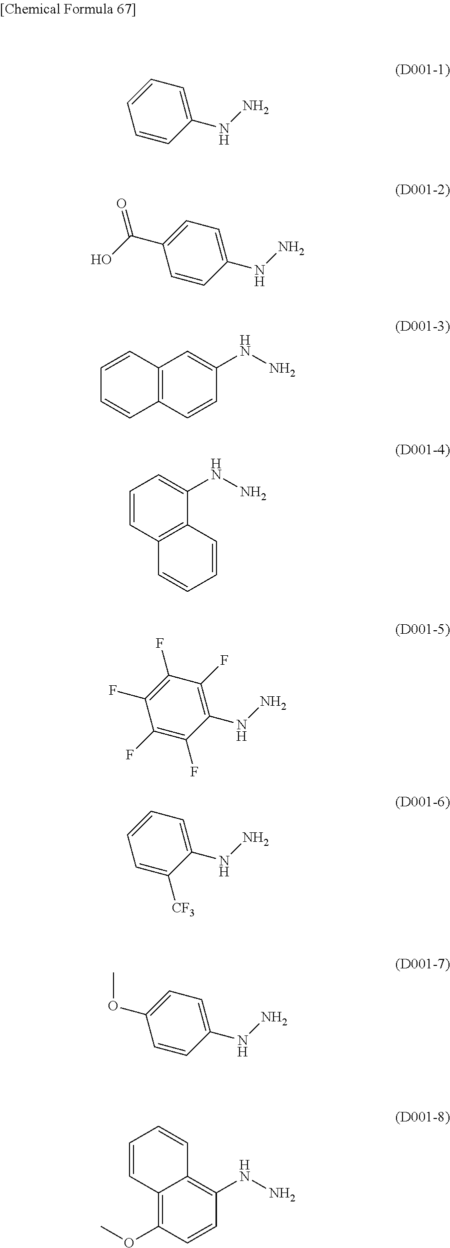

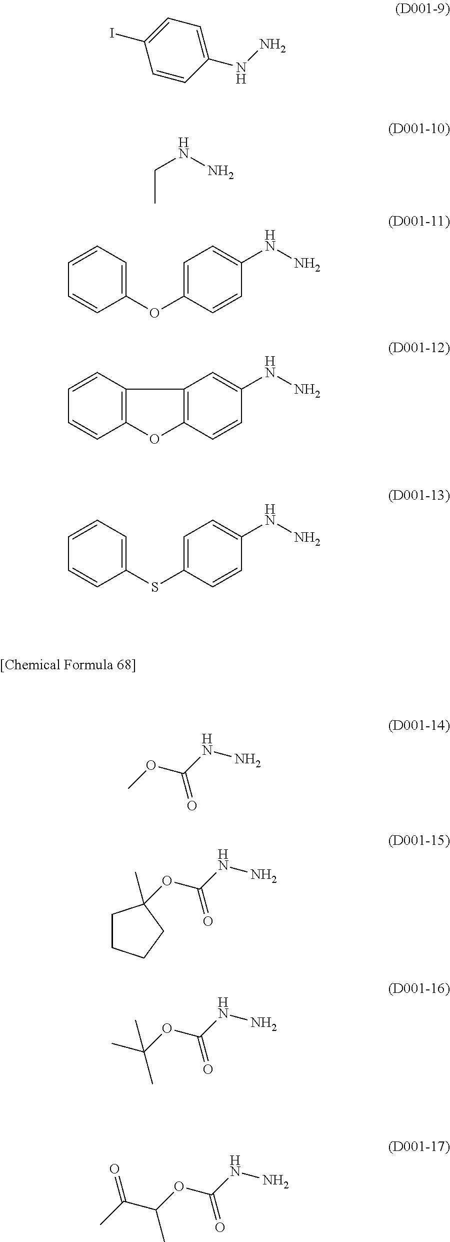

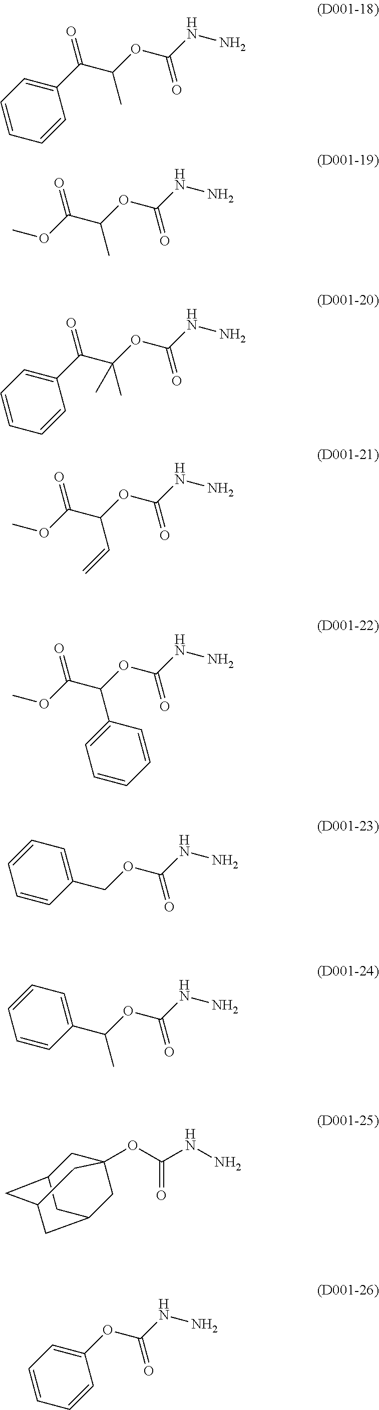

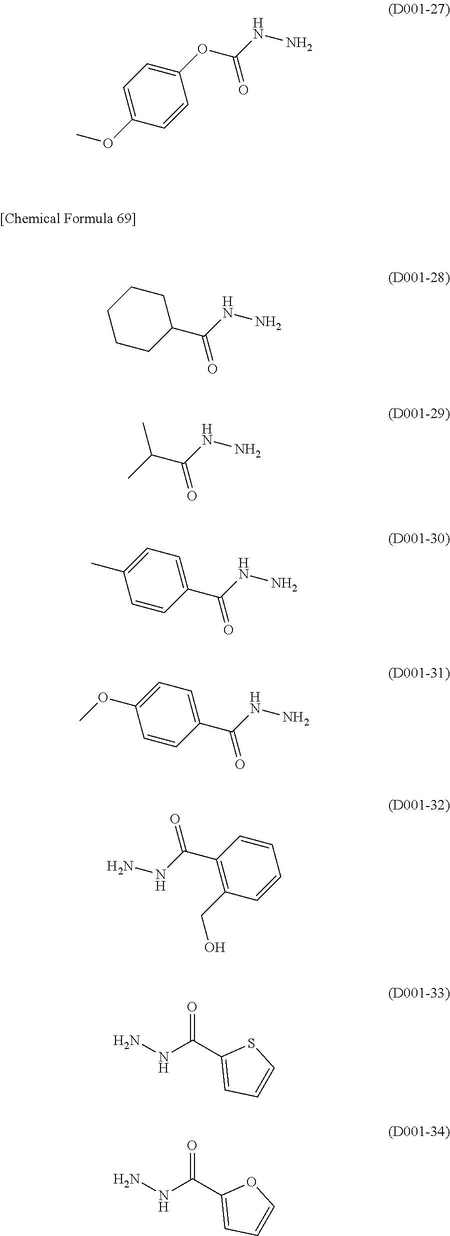

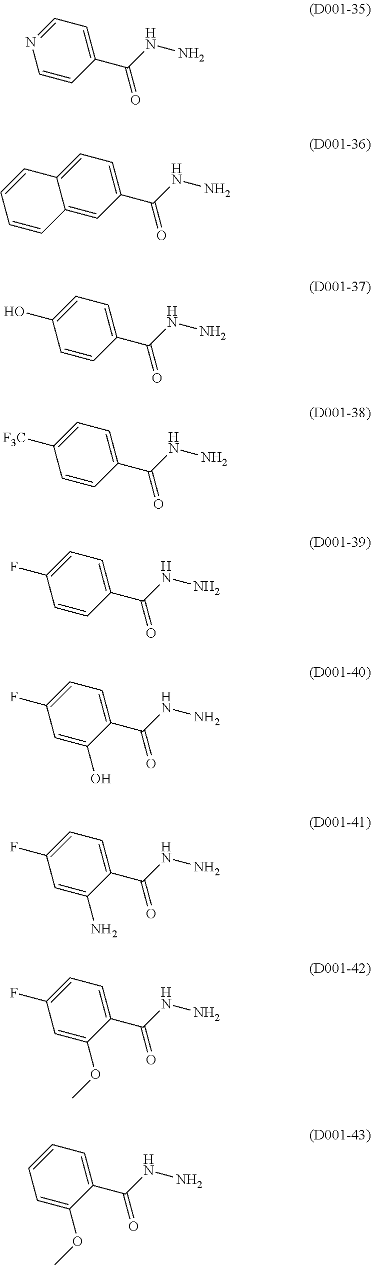

⋅Compound Represented by General Formula (d0-2) (Component (D002))

In Formula (d0-2), Rd⁰²¹ and Rd⁰²² each independently represent a monovalent organic group. Examples of Rd⁰²¹ include the same groups as those for Rd⁰¹ in Formula (d0). Examples of Rd⁰²² include the same groups as those for Rd⁰¹ in Formula (do).

In Formula (d0-2), Rd⁰²¹ and Rd⁰²² represent preferably a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent.

As the substituent contained in the cyclic group as Rd⁰²¹ and Rd⁰²², an alkoxy group, a fluorine atom, a hydroxyl group, a hydroxyalkyl group, or a carboxy group is preferable, and a hydroxyl group, a hydroxyalkyl group, or a carboxy group is more preferable. It is preferable that the hydroxyalkyl group is a group represented by —(CH₂)ₙ—OH (n represents an integer of 1 to 6). n in the formula preferably represents an integer of 1 to 3 and more preferably 1 or 2.

In Formula (d0-2), it is preferable that at least one of Rd⁰²¹ or Rd⁰²² has an aromatic ring, and both Rd⁰²¹ and Rd⁰²² may have an aromatic ring. In a case where at least one of Rd⁰²¹ or Rd⁰²² has an aromatic ring, the sensitivity and lithography characteristics such as CDU are likely to be improved.

In Formula (d0-2), Ld⁰²¹ and Ld⁰²² each independently represent a single bond or a divalent linking group. Examples of the divalent linking group as Ld⁰²¹ include the same groups as those for Ld⁰¹ in Formula (d0). Examples of the divalent linking group as Ld⁰²² include the same groups as those for Ld⁰¹ in Formula (d0).

In Formula (d0-2), as the divalent linking group represented by Ld⁰²¹ and Ld⁰²², —C(═O)—O—, —O—C(═O)—, —O—, —C(═O)—, or a linear or branched aliphatic hydrocarbon group, or a combination of two or more thereof is preferable. As the linear or branched aliphatic hydrocarbon group, a linear or branched alkylene group having 1 to 6 carbon atoms or a linear or branched alkenylene group having 2 to 6 carbon atoms is preferable. As the divalent linking group represented by Ld⁰²¹ and Ld⁰²², —C(═O)—O—, —O—C(═O)—, —O—, or —C(═O)— is more preferable.

In Formula (d0-2), examples of the combination of Ld⁰²¹ and Ld⁰²² include a combination in which both Ld⁰²¹ and Ld⁰²² represent a single bond; a combination in which Ld⁰²¹ represents a single bond and Ld⁰²² represents —C(═O)—O—, —O—C(═O)—, or —C(═O)—; a combination in which Ld⁰²¹ represents —C(═O)— and Ld⁰²² represents —C(═O)—O— or —O—C(═O)—; a combination in which both Ld⁰²¹ and Ld⁰²² represent —C(═O)—; and a combination in which both Ld⁰²¹ and Ld⁰²² represent —C(═O)—O— or —O—C(═O)—.

Specific examples of the component (D002) are shown below, but the present invention is not limited thereto.

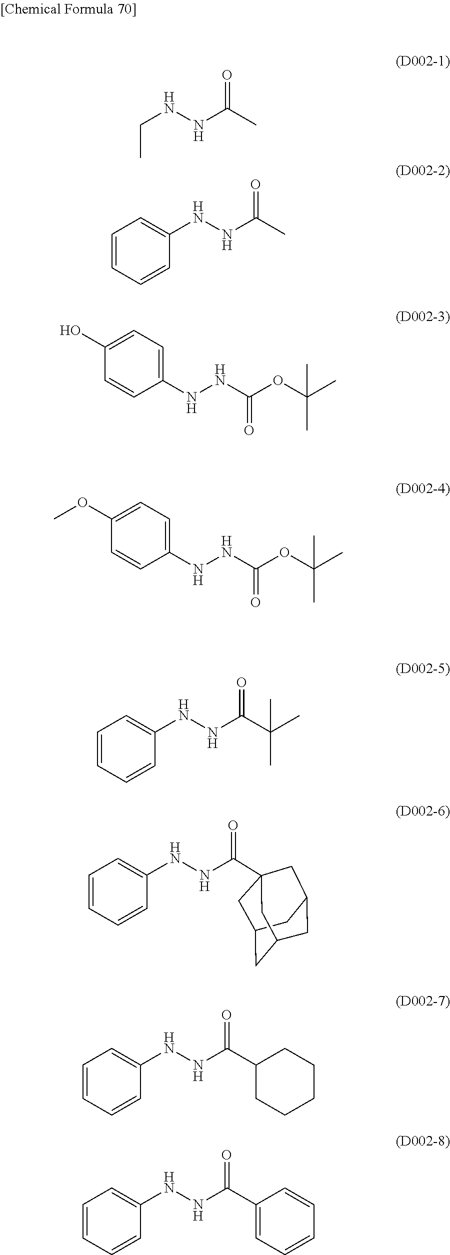

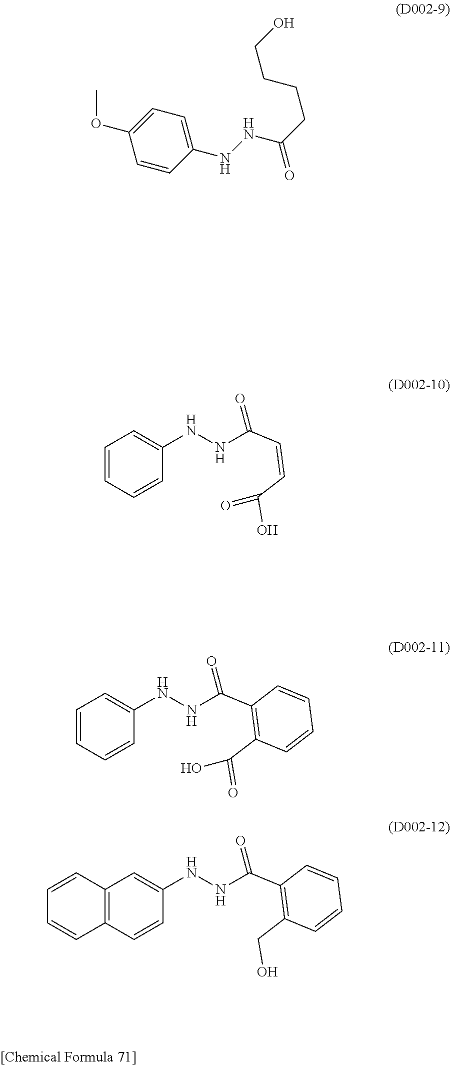 [SMILES: COc1ccc(NNC(=O)CCCCO)cc1 ; O=C(O)/C=C\C(=O)NNc1ccccc1 ; O=C(O)c1ccccc1C(=O)NNc1ccccc1 ; O=C(NNc1ccc2ccccc2c1)c1ccccc1CO]

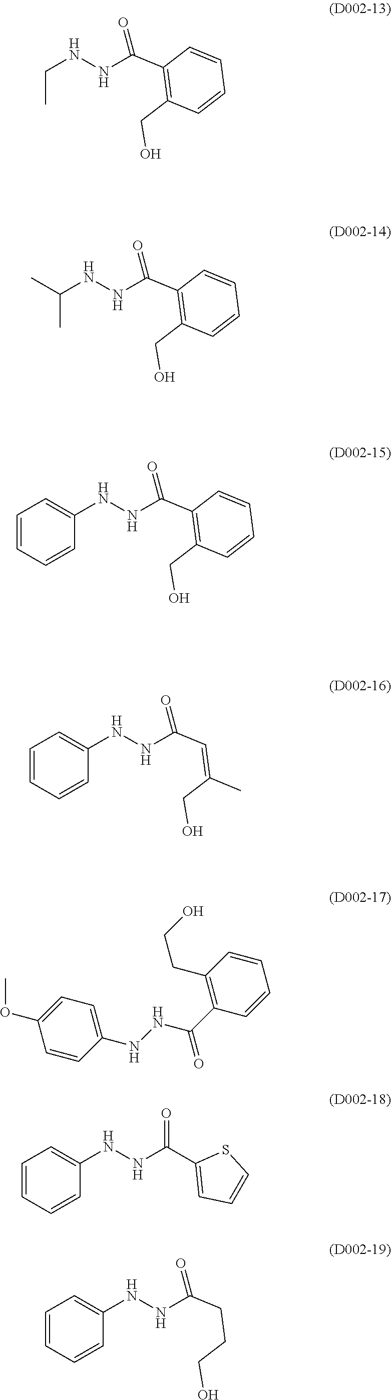

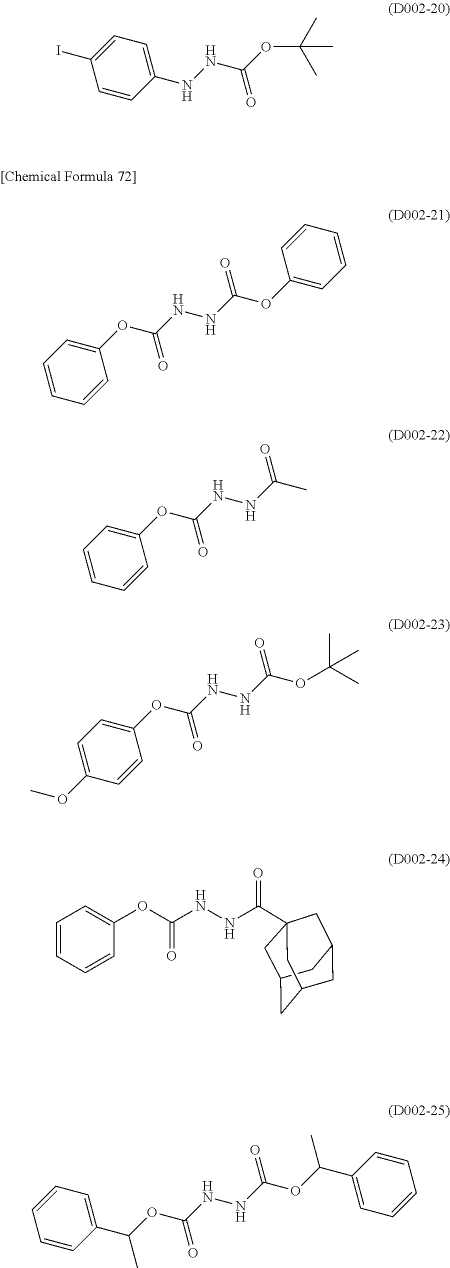

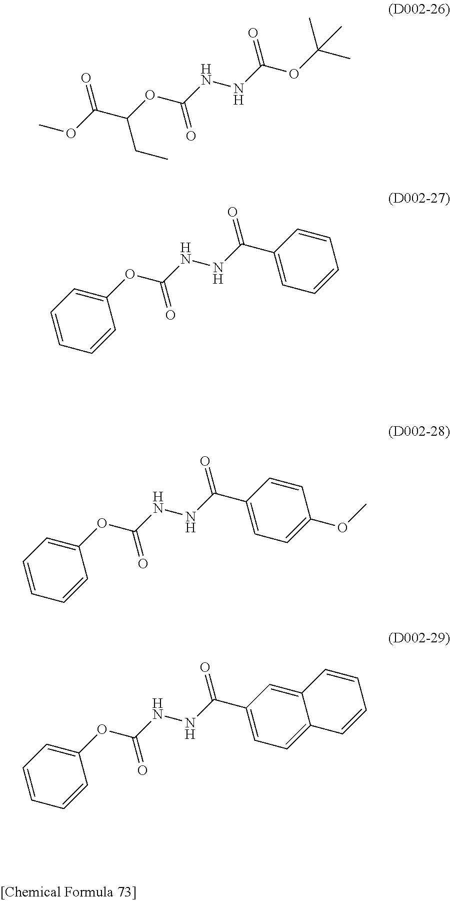 [SMILES: CCC(OC(=O)NNC(=O)OC(C)(C)C)C(=O)OC ; O=C(NNC(=O)c1ccccc1)Oc1ccccc1 ; COc1ccc(C(=O)NNC(=O)Oc2ccccc2)cc1 ; O=C(NNC(=O)c1ccc2ccccc2c1)Oc1ccccc1]

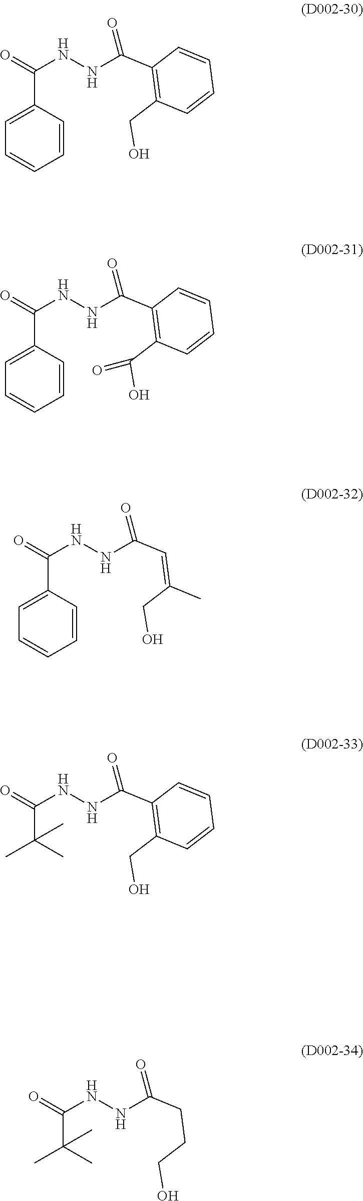 [SMILES: O=C(NNC(=O)c1ccccc1CO)c1ccccc1 ; O=C(NNC(=O)c1ccccc1C(=O)O)c1ccccc1 ; C/C(=C/C(=O)NNC(=O)c1ccccc1)CO ; CC(C)(C)C(=O)NNC(=O)c1ccccc1CO ; CC(C)(C)C(=O)NNC(=O)CCCO]

The component (P) may be used alone or a combination of two or more kinds thereof may be used. The component (D0) may be the component (D001), the component (D002), or a combination of the component (D001) and the component (D002).

### Second Embodiment

The resist composition of the present embodiment may contain a compound (D01) represented by General Formula (d01) (hereinafter, also referred to as “component (D01)”) as the component (D).

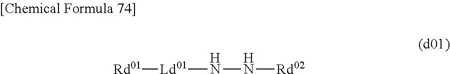

[In the formula, Rd⁰¹ represents a monovalent organic group having at least two benzene rings which may have a substituent; Rd⁰² represents a monovalent organic group or a hydrogen atom; and Ld⁰¹ represents a single bond or a divalent linking group.]

In General Formula (d01), Rd⁰¹ represents a monovalent organic group having at least two benzene rings which may have a substituent. Examples of the monovalent organic group having at least two benzene rings as Rd⁰¹ include a polycyclic aromatic hydrocarbon group, a group obtained by removing one hydrogen atom from a group in which two aromatic hydrocarbon groups are bonded to each other via a heteroatom such as —O— or —S—, and a group obtained by removing one hydrogen atom from a condensed ring in which two aromatic hydrocarbon groups are condensed with a cyclic group in which a heteroatom such as —O— or —S— forms a part of a ring skeleton.

In Formula (d01), the polycyclic aromatic hydrocarbon group as Rd⁰¹ has preferably 10 to 30 carbon atoms, more preferably 10 to 25 carbon atoms, and still more preferably 10 to 20 carbon atoms. Here, the number of carbon atoms in a substituent is not included in the number of carbon atoms.

In Formula (d01), examples of the aromatic ring contained in the polycyclic aromatic hydrocarbon group as Rd⁰¹ include fluorene, naphthalene, anthracene, phenanthrene, and biphenyl.

In Formula (d01), specific examples of the polycyclic aromatic hydrocarbon group as Rd⁰¹ include a group obtained by removing one hydrogen atom from the aromatic ring (an aryl group such as a naphthyl group), and a group in which one hydrogen atom of the aromatic ring is substituted with an alkylene group (for example, an arylalkyl group such as a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (d01), examples of the group as Rd⁰¹ obtained by removing one hydrogen atom from a group in which two aromatic hydrocarbon groups are bonded to each other via a heteroatom such as —O— or —S— include a group obtained by removing one hydrogen atom from diarylether, diarylthioether, or the like to which an aromatic ring such as benzene, fluorene, naphthalene, anthracene, phenanthrene, or biphenyl is bonded via a heteroatom such as —O— or —S—.

In Formula (d01), examples of the group as Rd⁰¹ obtained by removing one hydrogen atom from a condensed ring in which two aromatic hydrocarbon groups are condensed with a cyclic group in which a heteroatom such as —O— or —S— forms a part of a ring skeleton include a group obtained by removing one hydrogen atom from a dibenzothiophene ring, a group obtained by removing one hydrogen atom from a dibenzofuran ring, a group obtained by removing one hydrogen atom from a thioxanthone ring, a group obtained by removing one hydrogen atom from a xanthone ring, a group obtained by removing one hydrogen atom from a thianthrene ring, and a group obtained by removing one hydrogen atom from a phenoxathiin ring.

In Formula (d01), the monovalent organic group having at least two benzene rings as Rd⁰¹ may have a substituent. Examples of the substituent include an alkyl group having 1 to 5 carbon atoms, an alkoxy group having 1 to 5 carbon atoms, a fluorine atom, and a fluorinated alkyl group having 1 to 5 carbon atoms.

In General Formula (d01), it is preferable that Rd⁰¹ represents a group represented by General Formula (d01-r-1) or (d01-r-2).

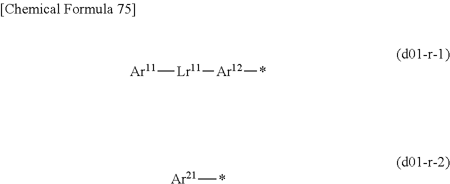

[In the formulae, Ar¹¹ represents a monovalent aromatic hydrocarbon group having at least one benzene ring which may have a substituent; Ar¹² represents a divalent aromatic hydrocarbon group having at least one benzene ring which may have a substituent; and Lr¹¹ represents —O— or —S—, where Ar¹¹ and Ar¹² may be bonded to each other to form a condensed ring together with Lr¹¹. Ar²¹ represents an aromatic hydrocarbon group having at least two benzene rings which may have a substituent. * represents a bonding site with respect to Ld⁰¹.]

In Formula (d01-r-1), Ar¹¹ represents a monovalent aromatic hydrocarbon group having at least one benzene ring which may have a substituent.

Examples of the monovalent aromatic hydrocarbon group having at least one benzene ring as Ar¹¹ include a group obtained by removing one hydrogen atom from an aromatic ring such as benzene, fluorene, naphthalene, anthracene, phenanthrene, or biphenyl. Among these, as the monovalent aromatic hydrocarbon group having at least one benzene ring as Ar¹¹, a phenyl group or a naphthyl group is preferable, and a phenyl group is more preferable.

In Formula (d01-r-1), examples of the substituent that may be contained in a benzene ring as Ar¹¹ include an alkyl group having 1 to 5 carbon atoms, an alkoxy group having 1 to 5 carbon atoms, a fluorine atom, and a fluorinated alkyl group having 1 to 5 carbon atoms.

In Formula (d01-r-1), Ar¹² represents a divalent aromatic hydrocarbon group having at least one benzene ring which may have a substituent.

Examples of the divalent aromatic hydrocarbon group having at least one benzene ring as Ar¹² include a group obtained by removing two hydrogen atoms from an aromatic ring such as benzene, fluorene, naphthalene, anthracene, phenanthrene, or biphenyl. Among these, as the divalent aromatic hydrocarbon group having at least one benzene ring as Ar¹², a phenylene group or a naphthylene group is preferable, and a phenylene group is more preferable.

In Formula (d01-r-1), examples of the substituent that may be contained in the benzene ring as Ar¹² include an alkyl group having 1 to 5 carbon atoms, an alkoxy group having 1 to 5 carbon atoms, a fluorine atom, and a fluorinated alkyl group having 1 to 5 carbon atoms.

In Formula (d01-r-1), Ar¹¹ and Ar¹² may be bonded to each other to form a condensed ring together with Lr¹¹.

In Formula (d01-r-1), in a case where Ar¹¹ and Ar¹² are bonded to each other to form a condensed ring together with Lr¹¹, examples of Ar¹¹-Lr¹¹-Ar¹²- include a group obtained by removing one hydrogen atom from a dibenzothiophene ring, a group obtained by removing one hydrogen atom from a dibenzofuran ring, a group obtained by removing one hydrogen atom from a thioxanthone ring, a group obtained by removing one hydrogen atom from a xanthone ring, a group obtained by removing one hydrogen atom from a thianthrene ring, and a group obtained by removing one hydrogen atom from a phenoxathiin ring. Among these, a group obtained by removing one hydrogen atom from a dibenzothiophene ring or a group obtained by removing one hydrogen atom from a dibenzofuran ring is preferable, and a group obtained by removing one hydrogen atom from a dibenzofuran ring is more preferable.

In Formula (d01-r-2), Ar²¹ represents an aromatic hydrocarbon group having at least two benzene rings which may have a substituent.

Examples of the aromatic hydrocarbon group having at least two benzene rings as Ar²¹ include a group obtained by removing one hydrogen atom from fluorene, naphthalene, anthracene, phenanthrene, or biphenyl. Among these, a naphthyl group is preferable.

In Formula (d01-r-2), examples of the substituent which may be contained in the benzene ring as Ar²¹ include an alkyl group having 1 to 5 carbon atoms, an alkoxy group having 1 to 5 carbon atoms, a fluorine atom, and a fluorinated alkyl group having 1 to 5 carbon atoms.

In Formula (d01), Rd⁰² represents a monovalent organic group or a hydrogen atom. Examples of the monovalent organic group as Rb⁰² include a hydrocarbon group which may have a substituent. The hydrocarbon group which may have a substituent may be an aliphatic hydrocarbon group which may have a substituent or an aromatic hydrocarbon group which may have a substituent. Examples of the monovalent organic group as Rd⁰² include a cyclic group which may have a substituent, and a linear or branched aliphatic hydrocarbon group which may have a substituent.

Cyclic Group which May have Substituent:

The cyclic group which may have a substituent is preferably a cyclic hydrocarbon group which may have a substituent. The cyclic hydrocarbon group which may have a substituent may be an aromatic hydrocarbon group which may have a substituent or an aliphatic hydrocarbon group which may have a substituent.

In Formula (d01), the aromatic hydrocarbon group as Rd⁰² has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

In Formula (d01), specific examples of the aromatic ring contained in the aromatic hydrocarbon group as Rd⁰² include benzene, fluorene, naphthalene, anthracene, phenanthrene, biphenyl, or an aromatic heterocyclic ring obtained by substituting some carbon atoms constituting these aromatic rings with heteroatoms. Examples of the heteroatom in the aromatic heterocyclic rings include an oxygen atom, a sulfur atom, and a nitrogen atom.

Specific examples of the aromatic hydrocarbon group as Rd⁰² in Formula (d01) include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (d01), examples of the cyclic aliphatic hydrocarbon group as Rd⁰² include an aliphatic hydrocarbon group having a ring in the structure. As the aliphatic hydrocarbon group including a ring in the structure, an alicyclic hydrocarbon group (a group formed by removing one hydrogen atom from an aliphatic hydrocarbon ring), a group in which an alicyclic hydrocarbon group is bonded to the terminal of a linear or branched aliphatic hydrocarbon group, and a group in which an alicyclic hydrocarbon group is interposed in a linear or branched aliphatic hydrocarbon group are exemplary examples.

The alicyclic hydrocarbon group preferably has 3 to 20 carbon atoms and more preferably has 3 to 12 carbon atoms.

In Formula (d01), the alicyclic hydrocarbon group as Rd⁰² may be a polycyclic group or a monocyclic group. As the monocyclic alicyclic hydrocarbon group, a group in which one or more hydrogen atoms have been removed from a monocycloalkane is preferable. The monocycloalkane preferably has 3 to 6 carbon atoms. As specific examples of the monocycloalkane, cyclopentane, cyclohexane, and the like are exemplary examples. As the polycyclic alicyclic hydrocarbon group, a group formed by removing one or more hydrogen atoms from a polycycloalkane is preferable, and the polycycloalkane preferably has 7 to 30 carbon atoms. Specific examples of the polycycloalkane include a polycycloalkane having a crosslinked ring polycyclic skeleton such as adamantane, norbornane, isobornane, tricyclo[5.2.1.0^{2,6}]decane, and tetracyclododecane.

The linear or branched aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and particularly preferably has 1 to 3 carbon atoms. Examples of the linear or branched aliphatic hydrocarbon group include the same groups as those for R′²⁰¹ and the like in General Formulae (ca-r-1) to (ca-r-7).

In Formula (d01), the cyclic hydrocarbon group as Rd⁰² may have a heteroatom, such as a heterocyclic ring. Specific examples thereof include lactone-containing cyclic groups each represented by General Formulae (a2-r-1) to (a2-r-7), —SO₂-containing cyclic group each represented by General Formulae (a5-r-1) to (a5-r-4), and heterocyclic groups each represented by Chemical Formulae (r-hr-1) to (r-hr-16).

In Formula (d01), the monovalent organic group as Rd⁰² is preferably a cyclic group which may have a substituent or a group in which a cyclic group which may have a substituent is bonded to a divalent linking group having —C(═O)—O—, —O—C(═O)—, —O—, or —C(═O)—. The cyclic group which may have a substituent is more preferably an aromatic hydrocarbon group which may have a substituent, still more preferably a phenyl group which may have a substituent or a naphthyl group which may have a substituent, and particularly preferably a phenyl group which may have a substituent. As the substituent contained in the cyclic group, a halogen atom, an alkoxy group, a hydroxyl group, an alkynyl group, —(CH₂)ₚ—OH, a carboxy group, or —C(═O)—O-Rd^{Al} is preferable, and —(CH₂)ₚ—OH, a carboxy group, or —C(═O)—O-Rd^{Al} is more preferable. p represents an integer of 1 to 3, preferably 1 or 2, and more preferably 1. Rd^{Al} represents a linear or branched aliphatic hydrocarbon group having 1 to 3 carbon atoms and preferably a methyl group.

In Formula (d01), Ld⁰¹ represents a single bond or a divalent linking group. Examples of the divalent linking group include the same groups as those for the divalent linking group as Ya^{x1} in General Formula (a10-1).

In Formula (d01), as the divalent linking group represented by Ld⁰¹, —C(═O)—O—, —O—C(═O)—, —O—, —C(═O)—, a linear or branched aliphatic hydrocarbon group, or a combination thereof is preferable. As the linear or branched aliphatic hydrocarbon group, a linear or branched alkylene group having 1 to 6 carbon atoms or a linear or branched alkenylene group having 2 to 6 carbon atoms is preferable. As the divalent linking group represented by Ld⁰¹, —C(═O)—O—, —O—C(═O)—, —O—, or —C(═O)— is more preferable.

From the viewpoint of dispersion in the resist film and the solubility of the developing solution, it is preferable that in Formula (d01), Rd⁰¹-Ld⁰¹- and -Rd⁰² bonded to the hydrazine skeleton (—NH—NH—) have different structures.

It is preferable that the component (D01) contains a compound represented by General Formula (d01-1) (hereinafter, also referred to as “component (D011)”) or a compound represented by General Formula (d01-2) (hereinafter, also referred to as “component (D012)”).

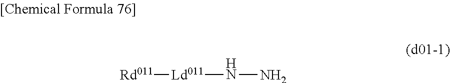

[In the formula, Rd⁰¹¹ represents a monovalent organic group having at least two benzene rings; and Ld⁰¹¹ represents a single bond or a divalent linking group.]

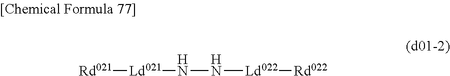

[In the formula, Rd⁰²¹ represents a monovalent organic group having at least two benzene rings; Rd⁰²² represents a monovalent organic group; and Ld⁰²¹ and Ld⁰²² each independently represent a single bond or a divalent linking group.]

⋅Compound Represented by General Formula (d01-1) (Component (D011))

In Formula (d01-1), the monovalent organic group having at least two benzene rings as Rd⁰¹¹ is the same as the monovalent organic group having at least two benzene rings which may have a substituent as Rd⁰¹ in Formula (d01).

In Formula (d01-1), it is preferable that Rd⁰¹¹ represents a group represented by Formula (d01-r-1) or (d01-r-2).

In Formula (d01-1), the divalent linking group as Ld⁰¹¹ is the same as the divalent linking group as Ld⁰¹ in Formula (d01).

In Formula (d01-1), as the divalent linking group represented by Ld⁰¹¹, —C(═O)—O—, —O—C(═O)—, —O—, —C(═O)—, a linear or branched aliphatic hydrocarbon group, or a combination thereof is preferable. As the linear or branched aliphatic hydrocarbon group, a linear or branched alkylene group having 1 to 6 carbon atoms or a linear or branched alkenylene group having 2 to 6 carbon atoms is preferable. The divalent linking group as Ld⁰¹¹ is more preferably —C(═O)—O—, —O—C(═O)—, —O—, or —C(═O)— and still more preferably —O—C(═O)—.

Specific examples of the component (D011) are shown below, but the present invention is not limited thereto.

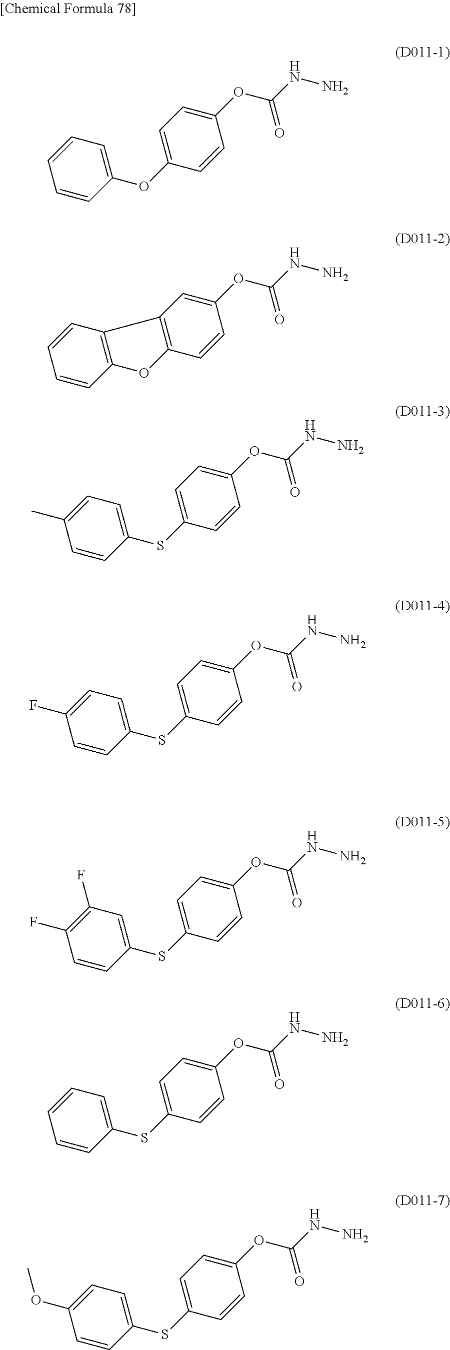

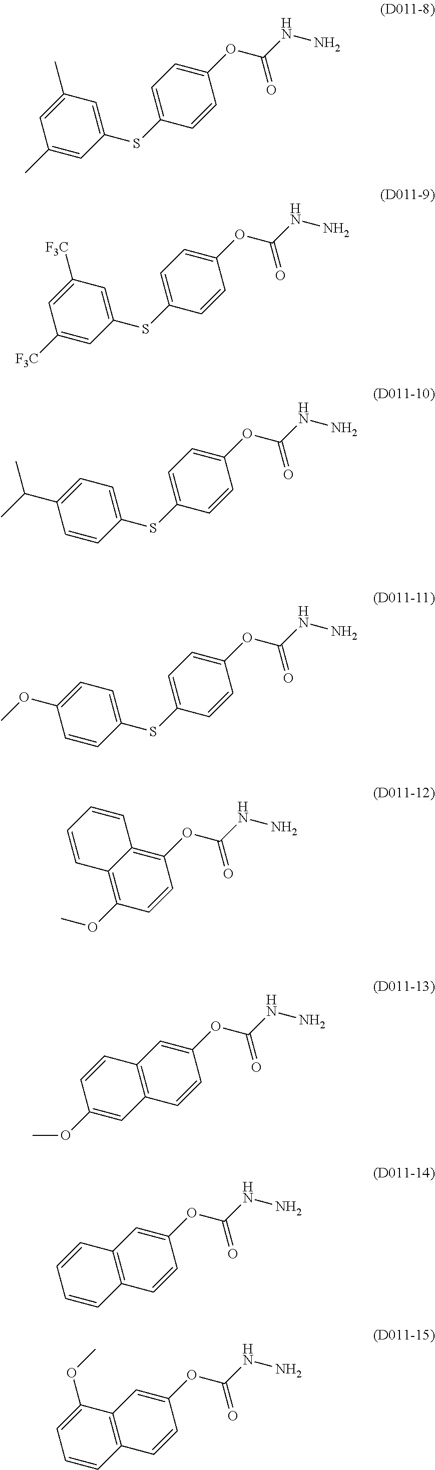

⋅Compound Represented by General Formula (d01-2) (Component (D012))

In Formula (d01-2), the monovalent organic group having at least two benzene rings as Rd⁰²¹ is the same as the monovalent organic group having at least two benzene rings which may have a substituent as Rd⁰¹ in Formula (d01).

It is preferable that Rd⁰²¹ represents a group represented by Formula (d01-r-1) or (d01-r-2).

In Formula (d01-2), the monovalent organic group as Rd⁰²² is the same as the monovalent organic group in Formula (d01).

In Formula (d01-2), it is preferable that Rd⁰²² represents a cyclic group which may have a substituent or a group in which a cyclic group which may have a substituent is bonded to a divalent linking group having —C(═O)—O—, —O—C(═O)—, —O—, or —C(═O)—. The cyclic group which may have a substituent is more preferably an aromatic hydrocarbon group which may have a substituent, still more preferably a phenyl group which may have a substituent or a naphthyl group which may have a substituent, and particularly preferably a phenyl group which may have a substituent. As the substituent contained in the cyclic group, an iodine atom, an alkoxy group, a fluorine atom, a hydroxyl group, —(CH₂)ₚ—OH, a carboxy group, or —C(═O)—O-Rd^{Al} is preferable, and an iodine atom, —(CH₂)ₚ—OH, a carboxy group, or —C(═O)—O-Rd^{Al} is more preferable. p represents an integer of 1 to 3, preferably 1 or 2, and more preferably 1. Rd^{Al} represents a linear or branched aliphatic hydrocarbon group having 1 to 3 carbon atoms and preferably a methyl group.

In Formula (d01-2), the divalent linking group as Ld⁰²¹ and Ld⁰²² is the same as the divalent linking group as Ld⁰¹ in Formula (d01).

In Formula (d01-2), as the divalent linking group represented by Ld⁰²¹ and Ld⁰²², —C(═O)—O—, —O—C(═O)—, —O—, —C(═O)—, a linear or branched aliphatic hydrocarbon group, or a combination thereof is preferable. As the linear or branched aliphatic hydrocarbon group, a linear or branched alkylene group having 1 to 6 carbon atoms or a linear or branched alkenylene group having 2 to 6 carbon atoms is preferable. As the divalent linking group represented by Ld⁰²¹ and Ld⁰²², —C(═O)—O—, —O—C(═O)—, —O—, or —C(═O)— is more preferable, and —O—C(═O)— is still more preferable.

Specific examples of the component (D012) are shown below, but the present invention is not limited thereto.

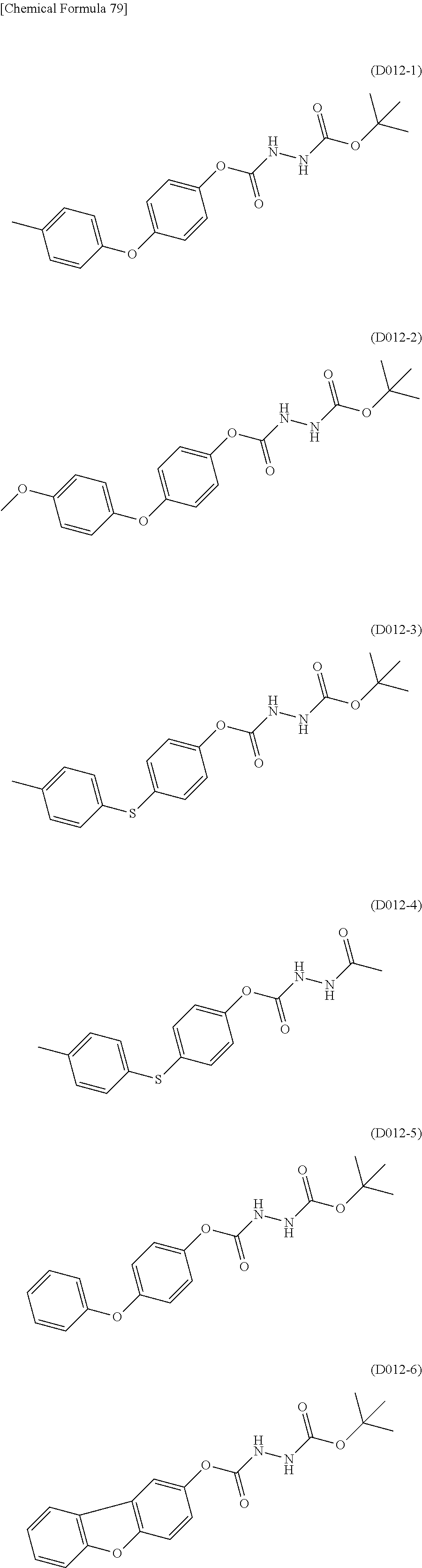

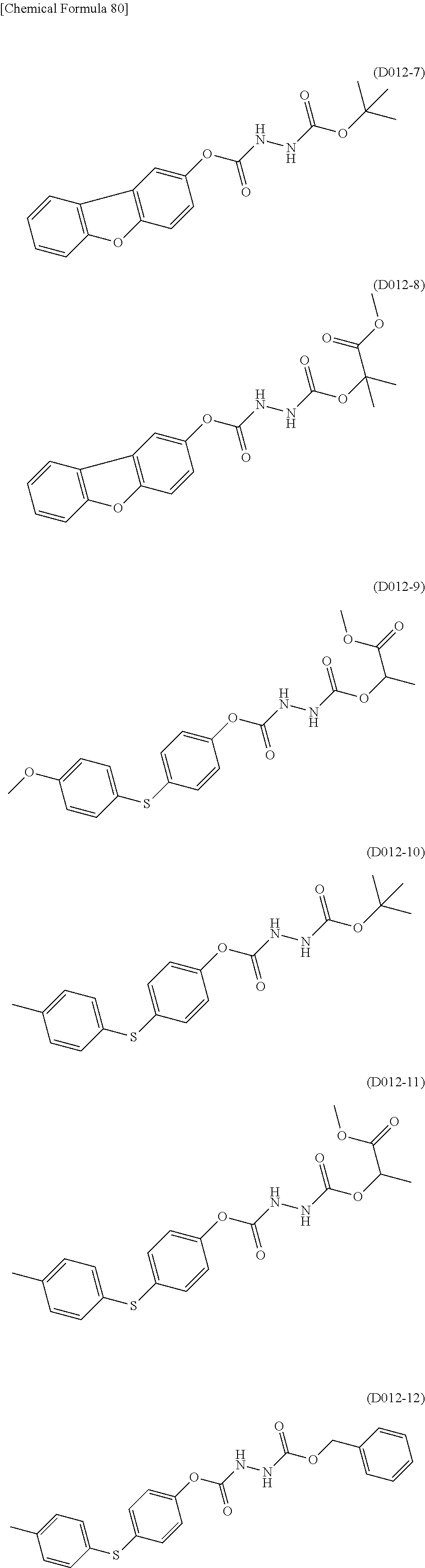

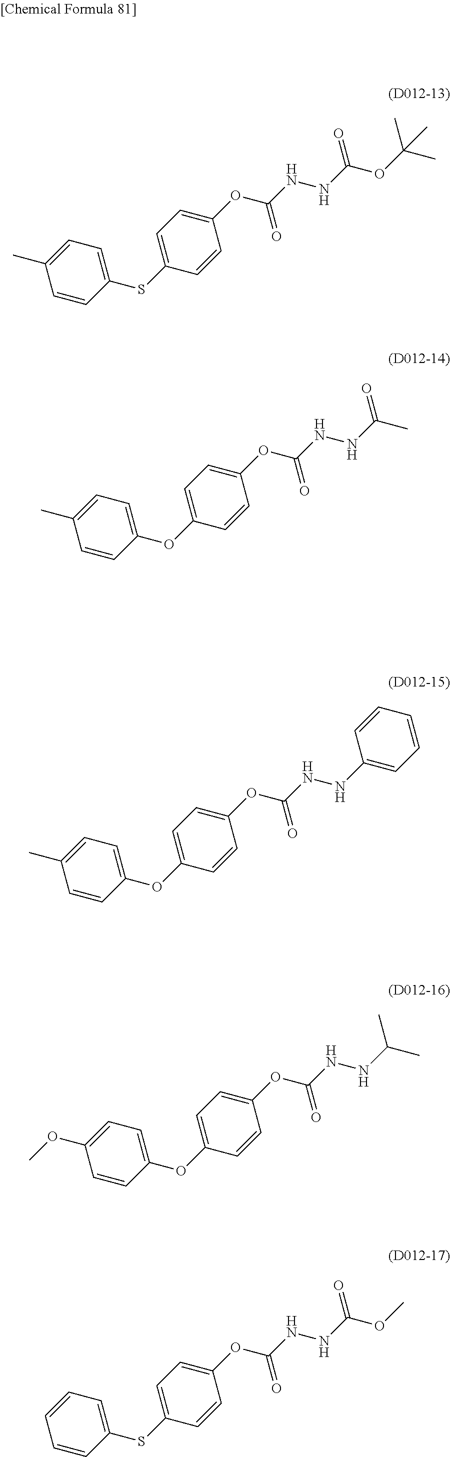

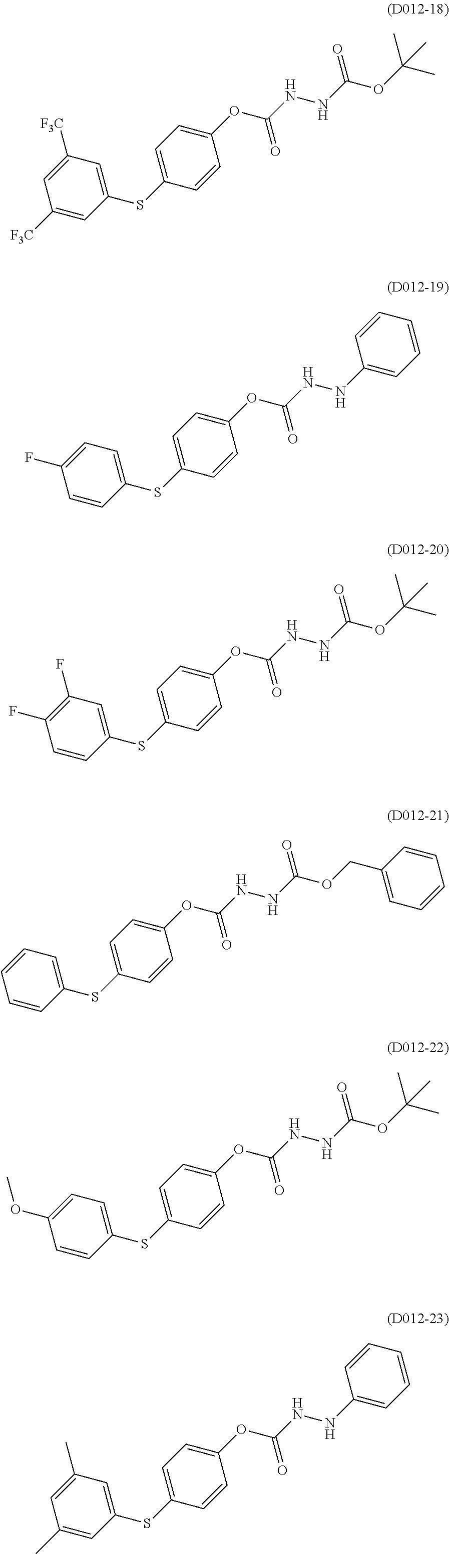

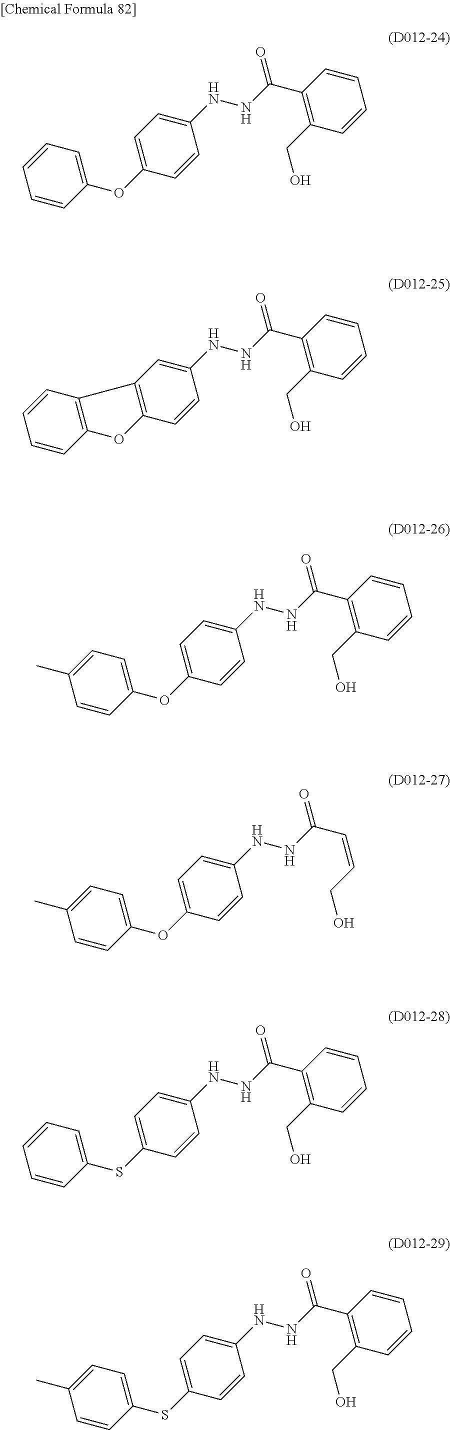 [SMILES: O=C(NNc1ccc(Oc2ccccc2)cc1)c1ccccc1CO ; O=C(NNc1ccc2oc3ccccc3c2c1)c1ccccc1CO ; Cc1ccc(Oc2ccc(NNC(=O)c3ccccc3CO)cc2)cc1 ; Cc1ccc(Oc2ccc(NNC(=O)/C=C\CO)cc2)cc1 ; O=C(NNc1ccc(Sc2ccccc2)cc1)c1ccccc1CO ; Cc1ccc(Sc2ccc(NNC(=O)c3ccccc3CO)cc2)cc1]

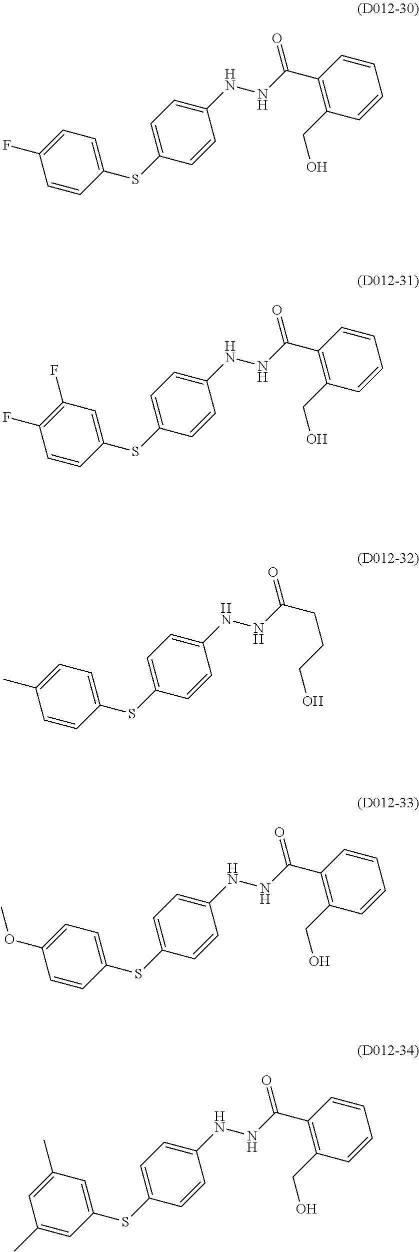 [SMILES: O=C(NNc1ccc(Sc2ccc(F)cc2)cc1)c1ccccc1CO ; O=C(NNc1ccc(Sc2ccc(F)c(F)c2)cc1)c1ccccc1CO ; Cc1ccc(Sc2ccc(NNC(=O)CCCO)cc2)cc1 ; COc1ccc(Sc2ccc(NNC(=O)c3ccccc3CO)cc2)cc1 ; Cc1cc(C)cc(Sc2ccc(NNC(=O)c3ccccc3CO)cc2)c1]

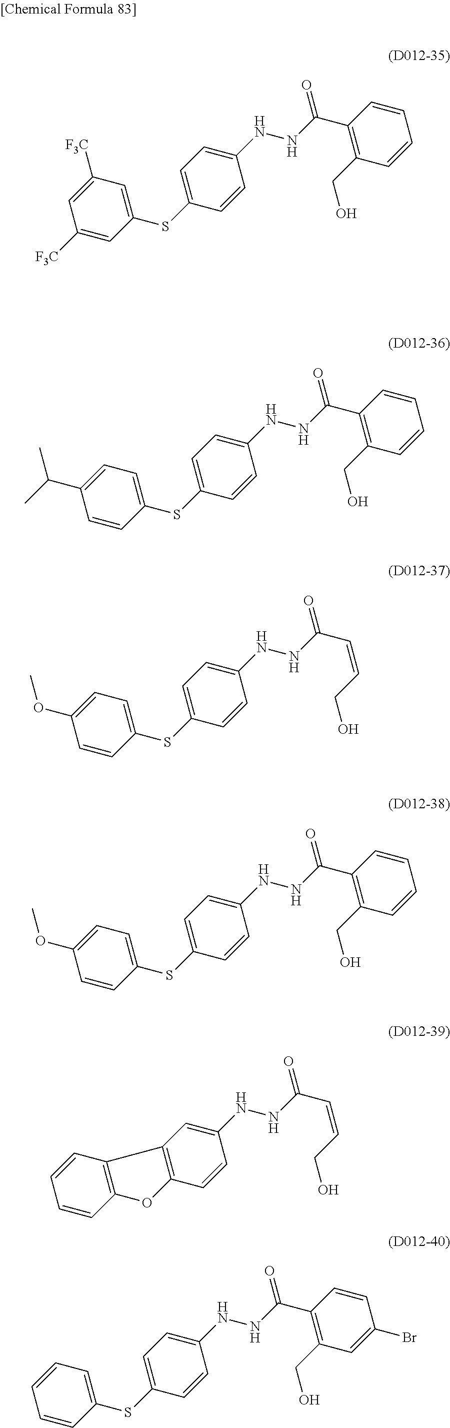 [SMILES: O=C(NNc1ccc(Sc2cc(C(F)(F)F)cc(C(F)(F)F)c2)cc1)c1ccccc1CO ; CC(C)c1ccc(Sc2ccc(NNC(=O)c3ccccc3CO)cc2)cc1 ; COc1ccc(Sc2ccc(NNC(=O)/C=C\CO)cc2)cc1 ; COc1ccc(Sc2ccc(NNC(=O)c3ccccc3CO)cc2)cc1 ; O=C(/C=C\CO)NNc1ccc2oc3ccccc3c2c1 ; O=C(NNc1ccc(Sc2ccccc2)cc1)c1ccc(Br)cc1CO]

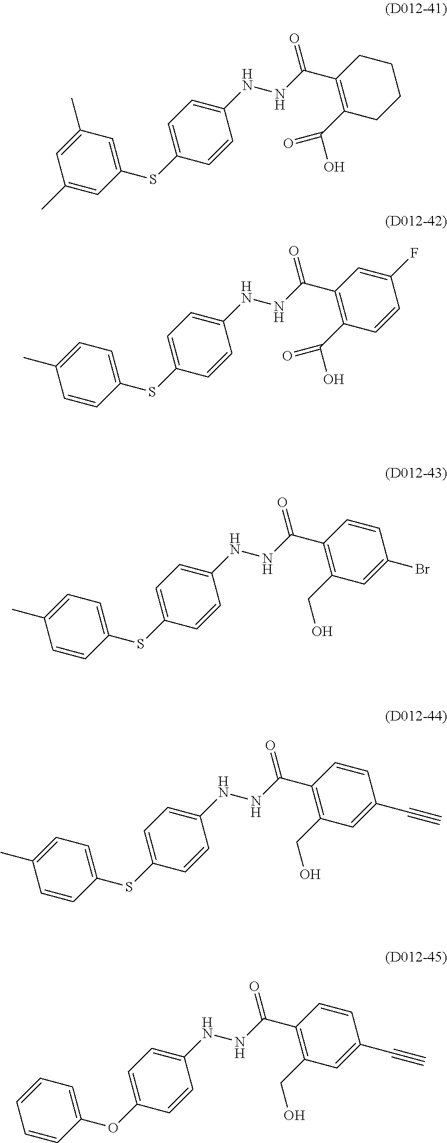 [SMILES: Cc1cc(C)cc(Sc2ccc(NNC(=O)C3=C(C(=O)O)CCCC3)cc2)c1 ; Cc1ccc(Sc2ccc(NNC(=O)c3cc(F)ccc3C(=O)O)cc2)cc1 ; Cc1ccc(Sc2ccc(NNC(=O)c3ccc(Br)cc3CO)cc2)cc1 ; C#Cc1ccc(C(=O)NNc2ccc(Sc3ccc(C)cc3)cc2)c(CO)c1 ; C#Cc1ccc(C(=O)NNc2ccc(Oc3ccccc3)cc2)c(CO)c1]

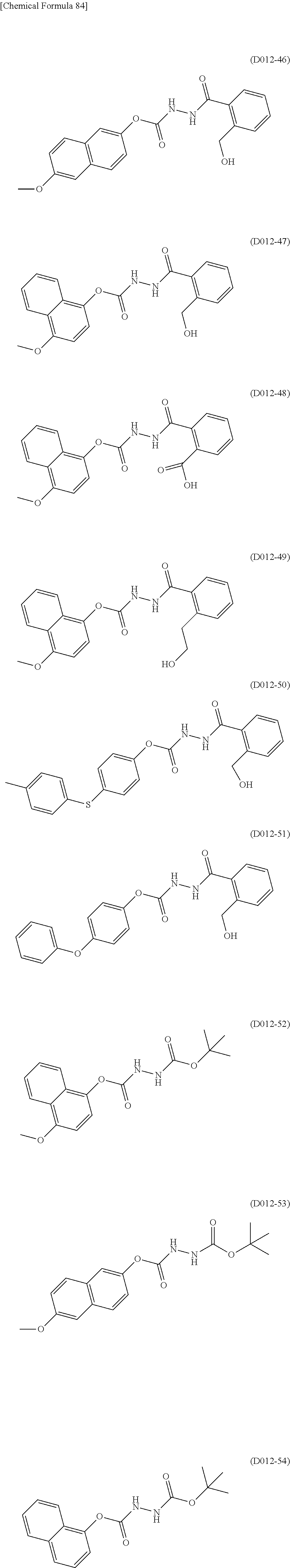

The component (D01) may be used alone or a combination of two or more kinds thereof may be used. The component (D01) may be the component (D011), the component (D012), or a combination of the component (D011) and the component (D012).

### Third Embodiment

### <<Component (D02)>>

The resist composition of the present embodiment may contain a compound (D02) represented by General Formula (d02) (hereinafter, also referred to as “component (D02)”) as the component (D).

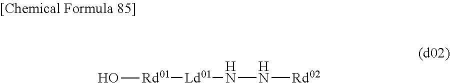

[In the formula, Rd⁰¹ represents a divalent organic group; Rd⁰² represents a monovalent organic group or a hydrogen atom; and Ld⁰¹ represents a single bond or a divalent linking group.]

In General Formula (d02), Rd⁰¹ represents a divalent organic group. Examples of the divalent organic group as Rd⁰¹ include a divalent hydrocarbon group which may have a substituent. Examples of the divalent hydrocarbon group which may have a substituent as Rd⁰¹ include the same groups as those for the divalent hydrocarbon group which may have a substituent as Va¹ in General Formula (a1-1). The divalent hydrocarbon group which may have a substituent as Rd⁰¹ may be an aliphatic hydrocarbon group which may have a substituent or an aromatic hydrocarbon group which may have a substituent. The aliphatic hydrocarbon group which may have a substituent may be saturated or unsaturated. Examples of the divalent organic group as Rd⁰¹ include a cyclic group which may have a substituent and a linear or branched aliphatic hydrocarbon group which may have a substituent.

Cyclic Group which May have Substituent:

The cyclic group which may have a substituent is preferably a cyclic hydrocarbon group which may have a substituent. The cyclic hydrocarbon group which may have a substituent may be an aromatic hydrocarbon group which may have a substituent or a cyclic aliphatic hydrocarbon group which may have a substituent.

In Formula (d02), the aromatic hydrocarbon group as Rd⁰¹ has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as Rd⁰¹ in Formula (d02) include benzene, fluorene, naphthalene, anthracene, phenanthrene, biphenyl, and an aromatic heterocyclic ring in which some carbon atoms constituting these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as Rd⁰¹ in Formula (d02) include a group obtained by removing two hydrogen atoms from the aromatic ring (an arylene group such as a phenylene group or a naphthylene group); and a group in which one hydrogen atom of a group (aryl group) obtained by removing one hydrogen atoms from the aromatic hydrocarbon ring is substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group (alkyl chain in the arylalkyl group) is preferably in a range of 1 to 4, more preferably 1 or 2, and particularly preferably 1.

In Formula (d02), examples of the cyclic aliphatic hydrocarbon group as Rd⁰¹ include an aliphatic hydrocarbon group having a ring in the structure. Examples of the aliphatic hydrocarbon group having a ring in the structure include an alicyclic hydrocarbon group (a group obtained by removing two hydrogen atoms from an aliphatic hydrocarbon ring), a group in which an alicyclic hydrocarbon group is bonded to the terminal of a linear or branched aliphatic hydrocarbon group, and a group in which an alicyclic hydrocarbon group is interposed in the middle of a linear or branched aliphatic hydrocarbon group. The cyclic aliphatic hydrocarbon group may be saturated or may be unsaturated.

The alicyclic hydrocarbon group preferably has 3 to 20 carbon atoms and more preferably has 3 to 12 carbon atoms.

In Formula (d02), the alicyclic hydrocarbon group as Rd⁰¹ may be a polycyclic group or a monocyclic group. The monocyclic alicyclic hydrocarbon group is preferably a group obtained by removing two hydrogen atoms from a monocycloalkane. The monocycloalkane preferably has 3 to 6 carbon atoms. As specific examples of the monocycloalkane, cyclopentane, cyclohexane, and the like are exemplary examples. As the polycyclic alicyclic hydrocarbon group, a group obtained by removing two hydrogen atoms from a polycycloalkane is preferable, and the polycycloalkane preferably has 7 to 30 carbon atoms. Specific examples of the polycycloalkane include a polycycloalkane having a crosslinked ring polycyclic skeleton such as adamantane, norbornane, isobornane, tricyclo[5.2.1.0^{2,6}]decane, and tetracyclododecane.

The linear or branched aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and particularly preferably has 1 to 3 carbon atoms. Examples of the linear or branched aliphatic hydrocarbon group include the same groups as those for Va¹ and the like in General Formula (a1-1).

In Formula (d02), the cyclic hydrocarbon group as Rd⁰¹ may have a heteroatom, such as a heterocyclic ring.b Specific examples thereof include a group obtained by removing one hydrogen atom from a lactone-containing cyclic group represented by any of General Formulae (a2-r-1) to (a2-r-7), a —SO₂-containing cyclic group represented by any of General Formulae (a5-r-1) to (a5-r-4), and a heterocyclic group represented by any of Chemical Formulae (r-hr-1) to (r-hr-16).

In Formula (d02), the cyclic group as Rd⁰¹ may or may not have a substituent. Examples of the substituent in the cyclic group as Rd⁰¹ include an alkyl group, an alkoxy group, a halogen atom, a halogenated alkyl group, a hydroxyl group, a carboxy group, a carbonyl group, and a nitro group.

As the alkyl group as the substituent, an alkyl group having 1 to 5 carbon atoms is preferable, and a methyl group, an ethyl group, a propyl group, an n-butyl group, and a tert-butyl group, and the like are exemplary examples.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is preferable, and a methoxy group, an ethoxy group, and the like are exemplary examples.

As the halogen atom as the substituent, a fluorine atom and a chlorine atom are exemplary examples, and an iodine atom is preferable.

As the halogenated alkyl group as the substituent, a group in which some or all hydrogen atoms in the alkyl group having 1 to 5 carbon atoms are substituted with the halogen atoms (fluorine atom and the like) is an exemplary example.

The carbonyl group as the substituent is a group that substitutes a methylene group (—CH₂—) constituting the cyclic hydrocarbon group.

Linear or Branched Aliphatic Hydrocarbon Group which May have Substituent:

In Formula (d02), the linear or branched aliphatic hydrocarbon group which may have a substituent as Rd⁰¹ may be a saturated aliphatic hydrocarbon group or an unsaturated aliphatic hydrocarbon group. The linear or branched aliphatic hydrocarbon group which may have a substituent preferably has 1 to 20 carbon atoms, more preferably has 1 to 15 carbon atoms, still more preferably has 1 to 10 carbon atoms, and particularly preferably has 1 to 6 carbon atoms. Here, the number of carbon atoms in a substituent is not included in the number of carbon atoms.

In Formula (d02), examples of the linear or branched aliphatic hydrocarbon group which may have a substituent as Rd⁰¹ include a linear or branched alkylene group which may have a substituent and a linear or branched alkenylene group which may have a substituent.

In Formula (d02), the linear alkylene group as Rd⁰¹ has preferably 1 to 20 carbon atoms, more preferably 1 to 15 carbon atoms, still more preferably 1 to 10 carbon atoms, and particularly preferably 1 to 6 carbon atoms.

The branched alkylene group has preferably 2 to 20 carbon atoms, more preferably 2 to 15 carbon atoms, still more preferably 2 to 10 carbon atoms, and particularly preferably 2 to 6 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

In Formula (d02), the linear alkenylene group as Rd⁰¹ has preferably 3 to 10 carbon atoms, more preferably 3 to 5 carbon atoms, still more preferably 3 or 4 carbon atoms, and particularly preferably 3 carbon atoms. Examples of the linear alkenylene group include —CH═CH—(CH₂)ₙ—, —(CH₂)ₘ—CH═CH—(CH₂)ₙ— (m and n each independently represent an integer of 1 to 5, preferably an integer of 1 to 3, and more preferably 1 or 2). The branched alkenylene group as Rd⁰¹ has preferably 3 to 10 carbon atoms, more preferably 3 to 5 carbon atoms, and still more preferably 3 or 4 carbon atoms. Examples of the branched alkenyl group include —CH(CH═CH₂)—, and —CH((CH₂)ₙCH═CH₂)— (n represents an integer of 1 to 5, preferably an integer of 1 to 3, and more preferably 1 or 2).

In Formula (d02), the linear or branched aliphatic hydrocarbon group as Rd⁰¹ may or may not have a substituent. Examples of the substituent in the linear or branched aliphatic hydrocarbon group as Rd⁰¹ include an alkoxy group, a halogen atom, a halogenated alkyl group, a hydroxyl group, a carboxy group, a carbonyl group, a nitro group, and an amino group.

In Formula (d02), Rd⁰¹ preferably represents a cyclic group which may have a substituent, a linear alkylene group which may have a substituent, or a linear alkenylene group which may have a substituent and more preferably an aromatic hydrocarbon group which may have a substituent. As the aromatic ring contained in the aromatic hydrocarbon group, a benzene ring is preferable, and a benzene ring is more preferable.

In Formula (d02), examples of the group represented by -Rd⁰¹-OH include a group represented by General Formula (Rd2-1).

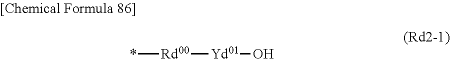

[In the formula, Rd⁰⁰ represents a divalent hydrocarbon group which may have a substituent; and Yd⁰¹ represents an alkylene group having 1 to 3 carbon atoms or a carbonyl group. * represents a bonding site that is bonded to Ld⁰¹ in Formula (d02).]

In Formula (Rd2-1), Rd⁰⁰ represents a divalent hydrocarbon group which may have a substituent. Examples of the divalent hydrocarbon group which may have a substituent as Rd⁰⁰ include the same groups as those for Rd⁰¹ in Formula (d02). Rd⁰⁰ has preferably 1 to 20 carbon atoms, more preferably 1 to 10 carbon atoms, and still more preferably 1 to 6 carbon atoms. Rd⁰⁰ preferably represents a cyclic group which may have a substituent, an alkylene group which may have a substituent, or an alkenylene group which may have a substituent, more preferably an aromatic hydrocarbon group which may have a substituent, an alkylene group which may have a substituent, or an alkenylene group which may have a substituent, still more preferably a phenylene group, a naphthylene group, a methylene group, an ethylene group, or an ethenylene group, and particularly preferably a phenylene group. The hydrocarbon group as Rd⁰⁰ may or may not have a substituent, but it is preferable that the hydrocarbon group as Rd⁰⁰ does not have a substituent.

In Formula (Rd2-1), Yd⁰¹ represents an alkylene group having 1 to 3 carbon atoms or a carbonyl group (—C(═O)—). Yd⁰¹ preferably represents an alkylene group having 1 to 3 carbon atoms, preferably an ethylene group or a methylene group, and more preferably a methylene group.

Specific examples of the group represented by Formula (Rd2-1) are shown below, but the present invention is not limited thereto. In the following formulae, * represents a bonding site that is bonded to Ld⁰¹ in Formula (d02).

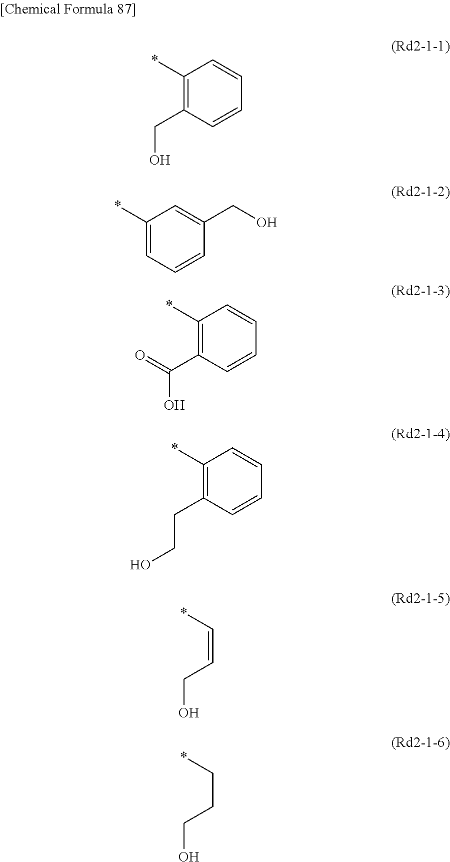

In Formula (d02), Rd⁰² represents a monovalent organic group or a hydrogen atom. Examples of the monovalent organic group as Rd⁰² include a monovalent hydrocarbon group which may have a substituent. The monovalent hydrocarbon group which may have a substituent may be an aliphatic hydrocarbon group which may have a substituent or an aromatic hydrocarbon group which may have a substituent. The aliphatic hydrocarbon group which may have a substituent may be saturated or unsaturated. Examples of the monovalent organic group as Rd⁰² include a cyclic group which may have a substituent, and a linear or branched aliphatic hydrocarbon group which may have a substituent.

Cyclic Group which May have Substituent:

The cyclic group which may have a substituent is preferably a cyclic hydrocarbon group which may have a substituent. The cyclic hydrocarbon group which may have a substituent may be an aromatic hydrocarbon group which may have a substituent or a cyclic aliphatic hydrocarbon group which may have a substituent.

In Formula (d02), the aromatic hydrocarbon group as Rd⁰² has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as Rd⁰² in Formula (d02) include benzene, fluorene, naphthalene, anthracene, phenanthrene, biphenyl, and an aromatic heterocyclic ring obtained by substituting some carbon atoms constituting these aromatic rings with heteroatoms. Examples of the heteroatom in the aromatic heterocyclic rings include an oxygen atom, a sulfur atom, and a nitrogen atom.

Specific examples of the aromatic hydrocarbon group as Rd⁰² include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (d02), the aromatic hydrocarbon group as Rd⁰² may be a group obtained by removing one hydrogen atom from a group in which two aromatic hydrocarbon groups are bonded to each other via a heteroatom such as —O— or —S—. Examples of such an aromatic hydrocarbon group include a group obtained by removing one hydrogen atom from diarylether, diarylthioether, or the like to which an aromatic ring such as benzene, fluorene, naphthalene, anthracene, phenanthrene, or biphenyl is bonded via a heteroatom such as —O— or —S—.

Examples of the group obtained by removing one hydrogen atom from a condensed ring in which two aromatic hydrocarbon groups are condensed with a cyclic group in which a heteroatom such as —O— or —S— forms a part of a ring skeleton include a group obtained by removing one hydrogen atom from a dibenzothiophene ring, a group obtained by removing one hydrogen atom from a dibenzofuran ring, a group obtained by removing one hydrogen atom from a thioxanthone ring, a group obtained by removing one hydrogen atom from a xanthone ring, a group obtained by removing one hydrogen atom from a thianthrene ring, and a group obtained by removing one hydrogen atom from a phenoxathiin ring.

In Formula (d02), examples of the cyclic aliphatic hydrocarbon group as Rd⁰² include an aliphatic hydrocarbon group having a ring in the structure. As the aliphatic hydrocarbon group including a ring in the structure, an alicyclic hydrocarbon group (a group formed by removing one hydrogen atom from an aliphatic hydrocarbon ring), a group in which an alicyclic hydrocarbon group is bonded to the terminal of a linear or branched aliphatic hydrocarbon group, and a group in which an alicyclic hydrocarbon group is interposed in a linear or branched aliphatic hydrocarbon group are exemplary examples. The cyclic aliphatic hydrocarbon group may be saturated or may be unsaturated.

The alicyclic hydrocarbon group preferably has 3 to 20 carbon atoms and more preferably has 3 to 12 carbon atoms.

In Formula (d02), the alicyclic hydrocarbon group as Rd⁰² may be a polycyclic group or a monocyclic group. As the monocyclic alicyclic hydrocarbon group, a group in which one or more hydrogen atoms have been removed from a monocycloalkane is preferable. The monocycloalkane preferably has 3 to 6 carbon atoms. As specific examples of the monocycloalkane, cyclopentane, cyclohexane, and the like are exemplary examples. As the polycyclic alicyclic hydrocarbon group, a group formed by removing one or more hydrogen atoms from a polycycloalkane is preferable, and the polycycloalkane preferably has 7 to 30 carbon atoms. Specific examples of the polycycloalkane include a polycycloalkane having a crosslinked ring polycyclic skeleton such as adamantane, norbornane, isobornane, tricyclo[5.2.1.0^{2,6}]decane, and tetracyclododecane.

The linear or branched aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and particularly preferably has 1 to 3 carbon atoms. Examples of the linear or branched aliphatic hydrocarbon group include the same groups as those for R′201 and the like in General Formulae (ca-r-1) to (ca-r-7).

In Formula (d02), the cyclic hydrocarbon group as Rd⁰² may have a heteroatom, such as a heterocyclic ring. Specific examples thereof include lactone-containing cyclic groups each represented by General Formulae (a2-r-1) to (a2-r-7), —SO₂-containing cyclic group each represented by General Formulae (a5-r-1) to (a5-r-4), and heterocyclic groups each represented by Chemical Formulae (r-hr-1) to (r-hr-16).

In Formula (d02), the cyclic group as Rd⁰² may or may not have a substituent. Examples of the substituent in the cyclic group as Rd⁰² include an alkyl group, an alkoxy group, a halogen atom, a halogenated alkyl group, a carboxy group, a carbonyl group, and a nitro group.

As the alkyl group as the substituent, an alkyl group having 1 to 5 carbon atoms is preferable, and a methyl group, an ethyl group, a propyl group, an n-butyl group, and a tert-butyl group, and the like are exemplary examples.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is preferable, and a methoxy group, an ethoxy group, and the like are exemplary examples.

As the halogenated alkyl group as the substituent, a group in which some or all hydrogen atoms in the alkyl group having 1 to 5 carbon atoms are substituted with the halogen atoms (fluorine atom and the like) is an exemplary example.

The carbonyl group as the substituent is a group that substitutes a methylene group (—CH₂—) constituting the cyclic hydrocarbon group.

Linear or Branched Aliphatic Hydrocarbon Group which May have Substituent:

In Formula (d02), the linear or branched aliphatic hydrocarbon group which may have a substituent as Rd⁰² may be a saturated aliphatic hydrocarbon group or an unsaturated aliphatic hydrocarbon group. The linear or branched aliphatic hydrocarbon group which may have a substituent preferably has 1 to 20 carbon atoms, more preferably has 1 to 15 carbon atoms, still more preferably has 1 to 10 carbon atoms, and particularly preferably has 1 to 6 carbon atoms. Here, the number of carbon atoms in a substituent is not included in the number of carbon atoms.

In Formula (d02), examples of the linear or branched aliphatic hydrocarbon group which may have a substituent as Rd⁰² include a linear or branched alkyl group which may have a substituent and a linear or branched alkenyl group which may have a substituent.

In Formula (d02), the linear alkyl group as Rd⁰² has preferably 1 to 20 carbon atoms, more preferably 1 to 15 carbon atoms, still more preferably 1 to 10 carbon atoms, even still more preferably 1 to 6 carbon atoms, particularly preferably 1 to 3 carbon atoms, and most preferably 1 or 2 carbon atoms.

The branched alkyl group preferably has 3 to 20 carbon atoms, more preferably has 3 to 15 carbon atoms, still more preferably has 3 to 10 carbon atoms, and particularly preferably has 3 to 6 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, a 4-methylpentyl group, and a tert-butyl group.

In Formula (d02), the linear alkenyl group as Rd⁰² has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. The branched alkenyl group as Rd⁰² has preferably 3 to 10 carbon atoms, more preferably 3 to 5 carbon atoms, and still more preferably 3 or 4 carbon atoms. Examples of the branched alkenyl group include a 1-methylvinyl group, a 2-methylvinyl group, a 1-methylpropenyl group, and a 2-methylpropenyl group.

In Formula (d02), the linear or branched aliphatic hydrocarbon group as Rd⁰² may or may not have a substituent. Examples of the substituent in the linear or branched aliphatic hydrocarbon group as Rd⁰² include an alkoxy group, a halogen atom, a halogenated alkyl group, a hydroxyl group, a carboxy group, a carbonyl group, a nitro group, and an amino group.

In Formula (d02), Rd⁰² preferably represents a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent.

In Formula (d02), the monovalent organic group as Rd⁰² may be formed such that a divalent linking group having a heteroatom and a hydrocarbon group which may have a substituent described above are linked to each other.

Examples of the divalent linking group having a heteroatom include the same groups as those for the divalent linking group having a heteroatom as Ya^{x1} in General Formula (a10-1). As the divalent linking group having a heteroatom, —C(═O)—O—, —O—C(═O)—, —O—, —C(═O)—, a linear or branched aliphatic hydrocarbon group, or a combination of two or more thereof is preferable, and a divalent linking group having —C(═O)—O—, —O—C(═O)—, —O—, —C(═O)—, —C(═O)—O-Ak-C(═O)—O—, or —O—C(═O)-Ak-O—C(═O)— (Ak represents a linear or branched alkylene group or a linear or branched alkenylene group) is more preferable.

In Formula (d02), as the monovalent organic group represented by Rd⁰², a cyclic group which may have a substituent; a linear or branched aliphatic hydrocarbon group which may have a substituent; a group in which a cyclic group which may have a substituent is bonded to a divalent linking group having —C(═O)—O—, —O—C(═O)—, —O—, or —C(═O)—; or a group in which a linear or branched aliphatic hydrocarbon group which may have a substituent is bonded to a divalent linking group having —C(═O)—O—, —O—C(═O)—, —O—, or —C(═O)— is preferable.

The cyclic group which may have a substituent is more preferably an aromatic hydrocarbon group which may have a substituent, still more preferably a phenyl group which may have a substituent, a naphthyl group which may have a substituent, a group obtained by removing one hydrogen atom from a diphenyl sulfide ring which may have a substituent, or a group obtained by removing one hydrogen atom from a diphenyl ether ring which may have a substituent, and particularly preferably a naphthyl group which may have a substituent. Examples of the substituent contained in the cyclic group include an alkoxy group, an alkyl group, and a fluorine atom.

The linear or branched aliphatic hydrocarbon group which may have a substituent is preferably a linear or branched alkyl group which may have a substituent, more preferably a linear or branched alkyl group having 1 to 6 carbon atoms which may have a substituent, and still more preferably a linear or branched alkyl group having 1 to 4 carbon atoms which may have a substituent. Examples of the substituent contained in the linear or branched hydrocarbon group include a fluorine atom.

In Formula (d02), at least one of Rd⁰¹ or Rd⁰² has preferably an aromatic ring, and both Rd⁰¹ and Rd⁰² may have an aromatic ring. In a case where at least one of Rd⁰¹ or Rd⁰² has an aromatic ring, the sensitivity and lithography characteristics such as CDU are likely to be improved.

In Formula (d02), Ld⁰¹ represents a single bond or a divalent linking group. Examples of the divalent linking group include the same groups as those for the divalent linking group as Ya^{x1} in General Formula (a10-1).

In Formula (d02), as the divalent linking group represented by Ld⁰¹, —C(═O)—O—, —O—C(═O)—, —O—, —C(═O)—, a linear or branched aliphatic hydrocarbon group, or a combination of two or more thereof is preferable. As the linear or branched aliphatic hydrocarbon group, a linear or branched alkylene group having 1 to 6 carbon atoms or a linear or branched alkenylene group having 2 to 6 carbon atoms is preferable. As the divalent linking group represented by Ld⁰¹, —C(═O)—O—, —O—C(═O)—, —O—, or —C(═O)— is more preferable, and —C(═O)— is still more preferable.

It is preferable that the component (D02) is a compound represented by General Formula (d02-1) (hereinafter, also referred to as “component (D021)”) or a compound represented by General Formula (d02-2) (hereinafter, also referred to as “component (D022)”).

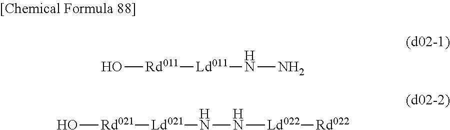

[In Formula (d02-1), Rd⁰¹¹ represents a divalent organic group, and Ld⁰¹¹ represents a single bond or a divalent linking group.

In Formula (d02-2), Rd⁰²¹ represents a divalent organic group, Rd⁰²¹ represents a monovalent organic group, and Ld⁰²¹ and Ld⁰²² each independently represent a single bond or a divalent linking group.]

⋅Compound Represented by General Formula (d02-1) (Component (D021))

In Formula (d02-1), Rd⁰¹¹ represents a divalent organic group. Rd⁰¹¹ has the same definition as that for Rd⁰¹ in Formula (d02).

In Formula (d02-1), Rd⁰¹¹ preferably represents a cyclic group which may have a substituent, a linear alkylene group which may have a substituent, or a linear alkenylene group which may have a substituent and more preferably an aromatic hydrocarbon group which may have a substituent.

In Formula (d02-1), in a case where Rd⁰¹¹ has an aromatic ring, the sensitivity and lithography characteristics such as CDU are likely to be improved. The aromatic ring contained in Rd⁰¹¹ is preferably a benzene ring or a naphthalene ring and more preferably a benzene ring.

In Formula (d02-1), a group represented by Formula (Rd2-1) is preferable as the group represented by -Rd⁰¹¹-OH.

In Formula (d02-1), Ld⁰¹¹ represents a single bond or a divalent linking group. Ld⁰¹¹ has the same definition as that for Ld⁰¹ in Formula (d02).

In Formula (d02-1), as the divalent linking group represented by Ld⁰¹¹, —C(═O)—O—, —O—C(═O)—, —O—, —C(═O)—, a linear or branched aliphatic hydrocarbon group, or a combination of two or more thereof is preferable. As the linear or branched aliphatic hydrocarbon group, a linear or branched alkylene group having 1 to 6 carbon atoms or a linear or branched alkenylene group having 2 to 6 carbon atoms is preferable. As the divalent linking group represented by Ld⁰¹¹, —C(═O)—O—, —O—C(═O)—, —O—, or —C(═O)— is more preferable, and —C(═O)— is still more preferable.

Specific examples of the component (D021) are shown below, but the present invention is not limited thereto.

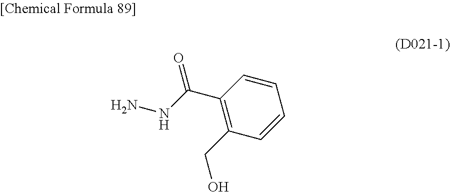 [SMILES: NNC(=O)c1ccccc1CO]

⋅Compound Represented by General Formula (d02-2) (Component (D022))

In Formula (d02-2), Rd⁰²¹ represents a divalent organic group. Rd⁰²¹ has the same definition as that for Rd⁰¹ in Formula (d02).

In Formula (d02-2), Rd⁰²¹ preferably represents a cyclic group which may have a substituent, a linear alkylene group which may have a substituent, or a linear alkenylene group which may have a substituent and more preferably an aromatic hydrocarbon group which may have a substituent.

In Formula (d02-2), in a case where Rd⁰²¹ has an aromatic ring, the sensitivity and the lithography characteristics such as CDU are likely to be improved. The aromatic ring contained in Rd⁰²¹ is preferably a benzene ring or a naphthalene ring and more preferably a benzene ring.

In Formula (d02-2), a group represented by Formula (Rd-1) is preferable as the group represented by -Rd⁰²¹-OH.

In Formula (d02-2), Rd⁰²² represents a monovalent organic group. Examples of Rd⁰²² include a monovalent hydrocarbon group which may have a substituent.

Examples of the monovalent hydrocarbon group which may have a substituent as Rd⁰²² include the same groups as those for the monovalent hydrocarbon group which may have a substituent as Rd⁰² in Formula (d02).

In Formula (d02-2), Rd⁰²² preferably represents a cyclic group which may have a substituent or a linear or branched aliphatic hydrocarbon group which may have a substituent and more preferably an aromatic hydrocarbon group which may have a substituent or a linear or branched alkyl group which may have a substituent.

The cyclic group which may have a substituent is more preferably an aromatic hydrocarbon group which may have a substituent, still more preferably a phenyl group which may have a substituent, a naphthyl group which may have a substituent, a group obtained by removing one hydrogen atom from a diphenyl sulfide ring which may have a substituent, or a group obtained by removing one hydrogen atom from a diphenyl ether ring which may have a substituent, and particularly preferably a naphthyl group which may have a substituent. Examples of the substituent contained in the cyclic group include an alkoxy group, an alkyl group, and a fluorine atom.

The linear or branched aliphatic hydrocarbon group which may have a substituent is preferably a linear or branched alkyl group which may have a substituent, more preferably a linear or branched alkyl group having 1 to 6 carbon atoms which may have a substituent, and still more preferably a linear or branched alkyl group having 1 to 4 carbon atoms which may have a substituent. Examples of the substituent contained in the linear or branched hydrocarbon group include a fluorine atom.

In Formula (d02-2), at least one of Rd⁰²¹ or Rd⁰²² preferably has an aromatic ring, and both Rd⁰²¹ and Rd⁰²² may have an aromatic ring. In a case where at least one of Rd⁰²¹ or Rd⁰²² has an aromatic ring, the sensitivity and lithography characteristics such as CDU are likely to be improved.

In Formula (d02-2), Ld⁰²¹ and Ld⁰²² each independently represent a single bond or a divalent linking group. Examples of the divalent linking group as Ld⁰²¹ include the same groups as those for Ld⁰¹ in Formula (d02). Examples of the divalent linking group as Ld⁰²² include the same groups as those for Ld⁰¹ in Formula (d02).

In Formula (d02-2), as the divalent linking group represented by Ld⁰²¹ and Ld⁰²², —C(═O)—O—, —O—C(═O)—, —O—, —C(═O)—, a linear or branched aliphatic hydrocarbon group, or a combination of two or more thereof is preferable. As the linear or branched aliphatic hydrocarbon group, a linear or branched alkylene group having 1 to 6 carbon atoms or a linear or branched alkenylene group having 2 to 6 carbon atoms is preferable.

In Formula (d02-2), as the divalent linking group represented by Ld⁰²¹, —C(═O)—O—, —O—C(═O)—, —O—, or —C(═O)— is preferable, and —C(═O)— is more preferable.

In Formula (d02-2), as the divalent linking group represented by Ld⁰²², a divalent linking group having —C(═O)—O—, —O—C(═O)—, —O—, —C(═O)—, —C(═O)—O-Ak-C(═O)—O— (Ak represents a linear or branched alkylene group or a linear or branched alkenylene group), or —O—C(═O)-Ak-O—C(═O)— (Ak represents a linear or branched alkylene group or a linear or branched alkenylene group) is preferable. As the group represented by -Ld⁰²²-Rd⁰²², —C(═O)—O-Rd⁰²², —O—C(═O)-Rd⁰²², —O-Rd⁰²², —C(═O)-Rd⁰²², —C(═O)—O-Ak-C(═O)—O-Rd⁹²², or —O—C(═O)-Ak-O—C(═O)-Rd⁰²² (Ak represents a linear or branched alkylene group or a linear or branched alkenylene group) is preferable, and —C(═O)—O-Rd⁰²² or —C(═O)—O-Ak-C(═O)—O-Rd⁰²² is more preferable.

It is preferable that the component (D022) is a compound represented by General Formula (d02-3).

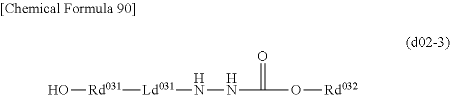

[In the formula, Rd⁰³¹ represents a divalent organic group, Rd⁰³² represents a monovalent organic group, and Ld⁰³¹ represents a single bond or a divalent linking group.]

In Formula (d02-3), Rd⁰³¹ represents a divalent organic group. Rd⁰³¹ has the same definition as that for Rd⁰²¹ in Formula (d02-2).

In Formula (d02-3), Rd⁰³² represents a monovalent organic group. Examples of the monovalent organic group as Rd⁰³² include a monovalent hydrocarbon group which may have a substituent. Examples of the monovalent hydrocarbon group which may have a substituent as Rd⁰³² include the same groups as those for the monovalent hydrocarbon group which may have a substituent as Rd⁰² in Formula (d02).

The component (D022) may be a compound represented by General Formula (d02-3-1).

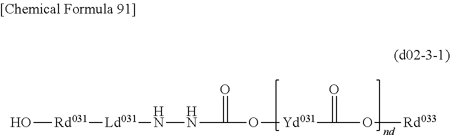

[In the formula, Rd⁰³¹ represents a divalent organic group, Rd⁰³³ represents a monovalent organic group, Ld⁰³¹ represents a single bond or a divalent linking group, Yd⁰³¹ represents a linear or branched alkylene group or a linear or branched alkenylene group, and nd represents 0 or 1.]

In Formula (d02-3-1), Rd⁰³¹ and Ld⁰³¹ each have the same definition as that for Rd⁰³¹ and Ld⁰³¹ in Formula (d02-3).

In Formula (d02-3-1), Rd⁰³¹ represents a monovalent organic group. Examples of the monovalent organic group include a monovalent hydrocarbon group which may have a substituent. Examples of the monovalent hydrocarbon group which may have a substituent as Rd⁰³³ include the same groups as those for the monovalent hydrocarbon group which may have a substituent as Rd⁰² in Formula (d02).

In Formula (d02-3-1), Yd⁰³¹ represents a linear or branched alkylene group or a linear or branched alkenylene group.

In Formula (d02-3-1), the linear alkylene group as Yd⁰³¹ has preferably 1 to 6 carbon atoms, more preferably 1 to 3 carbon atoms, still more preferably 1 or 2 carbon atoms, and particularly preferably 1 carbon atom. The branched alkylene group as Yd⁰³¹ has preferably 2 to 6 carbon atoms, more preferably 2 or 3 carbon atoms, and still more preferably 2 carbon atoms.

In Formula (d02-3-1), the linear alkenylene group as Yd⁰³¹ has preferably 3 to 6 carbon atoms, more preferably 3 to 5 carbon atoms, still more preferably 3 or 4 carbon atoms, and particularly preferably 3 carbon atoms. The branched alkenylene group as Yd⁰³¹ has preferably 3 to 6 carbon atoms, more preferably 3 to 5 carbon atoms, still more preferably 3 or 4 carbon atoms, and particularly preferably 3 carbon atoms.

In Formula (d02-3-1), nd represents 0 or 1 and preferably 0.

Specific examples of the component (D022) are shown below, but the present invention is not limited thereto.

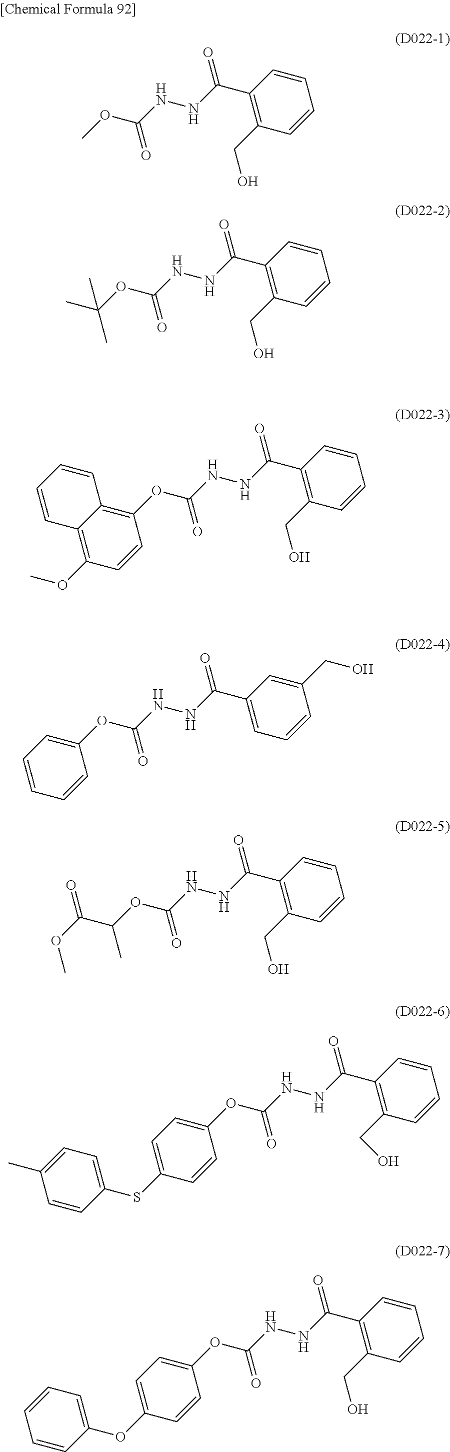

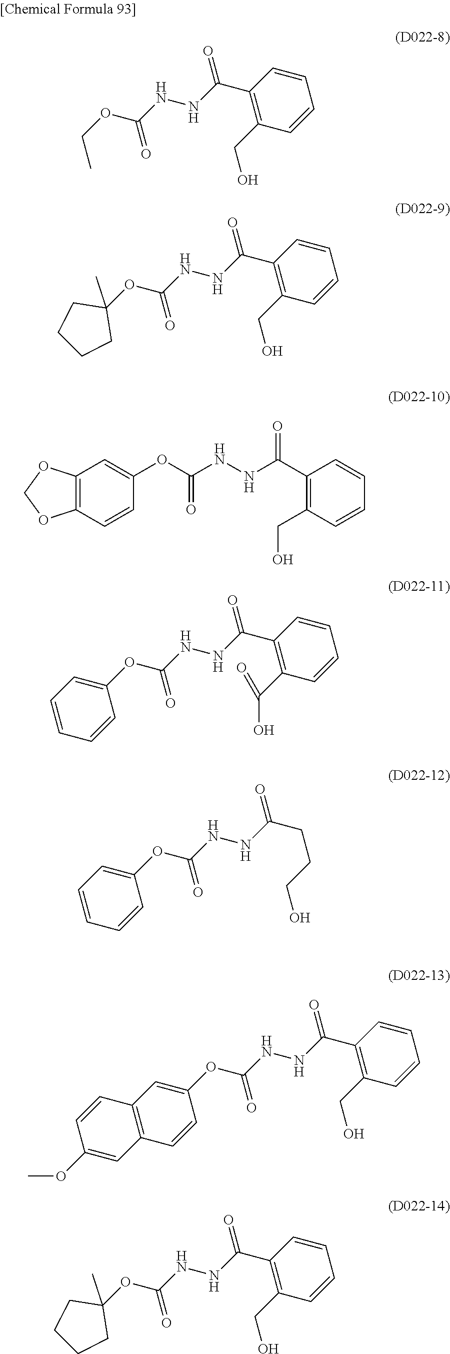

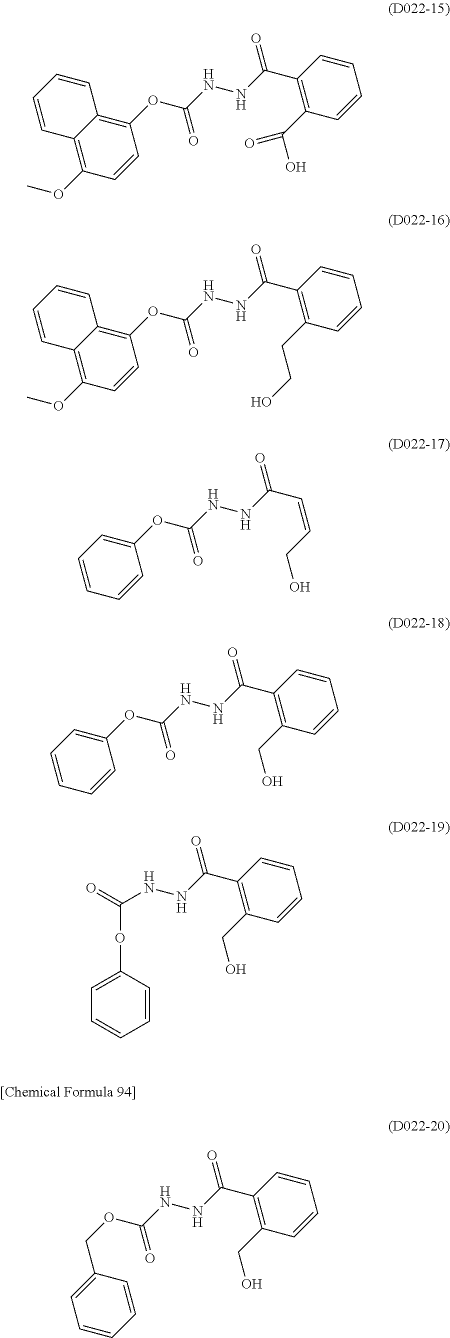 [SMILES: COc1ccc(OC(=O)NNC(=O)c2ccccc2C(=O)O)c2ccccc12 ; COc1ccc(OC(=O)NNC(=O)c2ccccc2CCO)c2ccccc12 ; O=C(/C=C\CO)NNC(=O)Oc1ccccc1 ; O=C(NNC(=O)c1ccccc1CO)Oc1ccccc1 ; O=C(NNC(=O)c1ccccc1CO)Oc1ccccc1 ; O=C(NNC(=O)c1ccccc1CO)OCc1ccccc1]

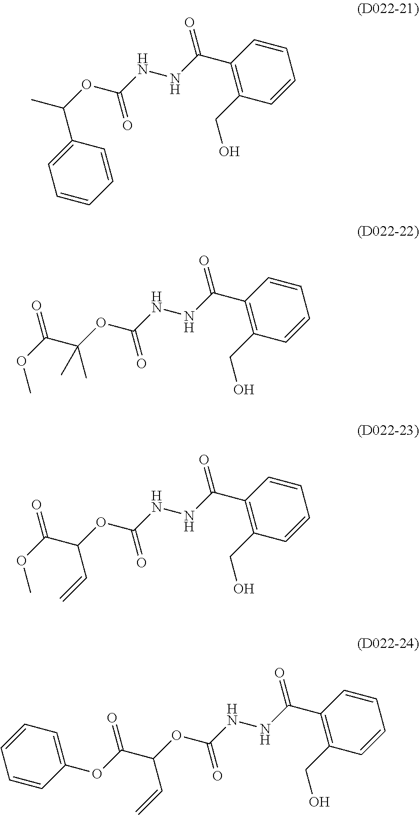 [SMILES: CC(OC(=O)NNC(=O)c1ccccc1CO)c1ccccc1 ; COC(=O)C(C)(C)OC(=O)NNC(=O)c1ccccc1CO ; C=CC(OC(=O)NNC(=O)c1ccccc1CO)C(=O)OC ; C=CC(OC(=O)NNC(=O)c1ccccc1CO)C(=O)Oc1ccccc1]

The component (D02) may be used alone or a combination of two or more kinds thereof may be used. The component (D02) may be the component (D021), the component (D022), or a combination of the component (D021) and the component (D022).

### Fourth Embodiment

### <<Component (D03)>>

The resist composition of the present embodiment may contain a compound (D03) represented by General Formula (d03) (hereinafter, also referred to as “component (D03)”) as the component (D).

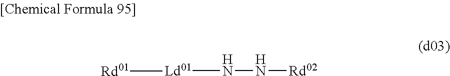

[In the formula, Rd⁰¹ represents a monovalent organic group, Rd⁰² represents a monovalent organic group or a hydrogen atom, and Ld⁰¹ represents a single bond or a divalent linking group. Here, at least one of Rd⁰¹ or Rd⁰² has at least one iodine atom as a substituent.]

In General Formula (d03), Rd⁰¹ represents a monovalent organic group. Examples of the monovalent organic group as Rd⁰¹ include a hydrocarbon group which may have a substituent. The hydrocarbon group which may have a substituent may be an aliphatic hydrocarbon group which may have a substituent or an aromatic hydrocarbon group which may have a substituent. Examples of the monovalent organic group as Rd⁰¹ include a cyclic group which may have a substituent, and a linear or branched aliphatic hydrocarbon group which may have a substituent.

Cyclic Group which May have Substituent:

The cyclic group which may have a substituent is preferably a cyclic hydrocarbon group which may have a substituent. The cyclic hydrocarbon group which may have a substituent may be an aromatic hydrocarbon group which may have a substituent or an aliphatic hydrocarbon group which may have a substituent.

In Formula (d03), the aromatic hydrocarbon group as Rd⁰¹ has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

In Formula (d03), specific examples of the aromatic ring contained in the aromatic hydrocarbon group as Rd⁰¹ include benzene, fluorene, naphthalene, anthracene, phenanthrene, biphenyl, or an aromatic heterocyclic ring obtained by substituting some carbon atoms constituting these aromatic rings with heteroatoms. Examples of the heteroatom in the aromatic heterocyclic rings include an oxygen atom, a sulfur atom, and a nitrogen atom.

Specific examples of the aromatic hydrocarbon group as Rd⁰¹ in Formula (d03) include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (d03), examples of the cyclic aliphatic hydrocarbon group as Rd⁰¹ include an aliphatic hydrocarbon group having a ring in the structure. As the aliphatic hydrocarbon group including a ring in the structure, an alicyclic hydrocarbon group (a group formed by removing one hydrogen atom from an aliphatic hydrocarbon ring), a group in which an alicyclic hydrocarbon group is bonded to the terminal of a linear or branched aliphatic hydrocarbon group, and a group in which an alicyclic hydrocarbon group is interposed in a linear or branched aliphatic hydrocarbon group are exemplary examples.

The alicyclic hydrocarbon group preferably has 3 to 20 carbon atoms and more preferably has 3 to 12 carbon atoms.

In Formula (d03), the alicyclic hydrocarbon group as Rd⁰¹ may be a polycyclic group or a monocyclic group. As the monocyclic alicyclic hydrocarbon group, a group in which one or more hydrogen atoms have been removed from a monocycloalkane is preferable. The monocycloalkane preferably has 3 to 6 carbon atoms. As specific examples of the monocycloalkane, cyclopentane, cyclohexane, and the like are exemplary examples. As the polycyclic alicyclic hydrocarbon group, a group formed by removing one or more hydrogen atoms from a polycycloalkane is preferable, and the polycycloalkane preferably has 7 to 30 carbon atoms. Specific examples of the polycycloalkane include a polycycloalkane having a crosslinked ring polycyclic skeleton such as adamantane, norbornane, isobornane, tricyclo[5.2.1.0^{2,6}]decane, and tetracyclododecane.

The linear or branched aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and particularly preferably has 1 to 3 carbon atoms. Examples of the linear or branched aliphatic hydrocarbon group include the same groups as those for R′201 and the like in General Formulae (ca-r-1) to (ca-r-7).

In Formula (d03), the cyclic hydrocarbon group as Rd⁰¹ may have a heteroatom, such as a heterocyclic ring. Specific examples thereof include lactone-containing cyclic groups each represented by General Formulae (a2-r-1) to (a2-r-7), —SO₂-containing cyclic group each represented by General Formulae (a5-r-1) to (a5-r-4), and heterocyclic groups each represented by Chemical Formulae (r-hr-1) to (r-hr-16).

In Formula (d03), the cyclic group as Rd⁰¹ may or may not have a substituent. Examples of the substituent in the cyclic group as Rd⁰¹ include an alkyl group, an alkoxy group, a halogen atom, a halogenated alkyl group, a hydroxyl group, a carboxy group, a carbonyl group, and a nitro group.

As the alkyl group as the substituent, an alkyl group having 1 to 5 carbon atoms is preferable, and a methyl group, an ethyl group, a propyl group, an n-butyl group, and a tert-butyl group, and the like are exemplary examples.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is preferable, and a methoxy group, an ethoxy group, and the like are exemplary examples.

As the halogen atom as the substituent, a fluorine atom and a chlorine atom are exemplary examples, and an iodine atom is preferable.

As the halogenated alkyl group as the substituent, a group in which some or all hydrogen atoms in the alkyl group having 1 to 5 carbon atoms are substituted with the halogen atoms (fluorine atom and the like) is an exemplary example.

The carbonyl group as the substituent is a group that substitutes a methylene group (—CH₂—) constituting the cyclic hydrocarbon group.

Among these, in Formula (d03), the substituent in the cyclic group as Rd⁰¹ is preferably an alkyl group, an alkoxy group, a halogen atom, a halogenated alkyl group, a hydroxyl group, or a carboxy group and more preferably a hydroxyl group or a carboxy group.

Linear or Branched Aliphatic Hydrocarbon Group which May have Substituent:

In Formula (d03), the linear or branched aliphatic hydrocarbon group which may have a substituent as Rd⁰¹ may be a saturated aliphatic hydrocarbon group or an unsaturated aliphatic hydrocarbon group. The linear or branched aliphatic hydrocarbon group which may have a substituent preferably has 1 to 20 carbon atoms, more preferably has 1 to 15 carbon atoms, still more preferably has 1 to 10 carbon atoms, and particularly preferably has 1 to 6 carbon atoms. Here, the number of carbon atoms in a substituent is not included in the number of carbon atoms.

In Formula (d03), examples of the linear or branched aliphatic hydrocarbon group which may have a substituent as Rd⁰¹ include a linear or branched alkyl group which may have a substituent and a linear or branched alkenyl group which may have a substituent.

In Formula (d03), the linear alkyl group as Rd⁰¹ has preferably 1 to 20 carbon atoms, more preferably 1 to 15 carbon atoms, still more preferably 1 to 10 carbon atoms, and particularly preferably 1 to 6 carbon atoms.

The branched alkyl group preferably has 3 to 20 carbon atoms, more preferably has 3 to 15 carbon atoms, still more preferably has 3 to 10 carbon atoms, and particularly preferably has 3 to 6 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

In Formula (d03), the linear alkenyl group as Rd⁰¹ has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. The branched alkenyl group as Rd⁰¹ has preferably 3 to 10 carbon atoms, more preferably 3 to 5 carbon atoms, and still more preferably 3 or 4 carbon atoms. Examples of the branched alkenyl group include a 1-methylvinyl group, a 2-methylvinyl group, a 1-methylpropenyl group, and a 2-methylpropenyl group.

In Formula (d03), the linear or branched aliphatic hydrocarbon group as Rd⁰¹ may or may not have a substituent. Examples of the substituent in the linear or branched aliphatic hydrocarbon group as Rd⁰¹ include an alkoxy group, a halogen atom, a halogenated alkyl group, a hydroxyl group, a carboxy group, a carbonyl group, a nitro group, and an amino group.

In Formula (d03), Rd⁰¹ preferably represents a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent. As the substituent contained in the cyclic group as Rd⁰¹, an iodine atom, an alkoxy group, a fluorine atom, a hydroxyl group, —(CH₂)ₚ—OH, a carboxy group, or Rd^{Ar}-S— is preferable, and an iodine atom, —(CH₂)ₚ—OH, or Rd^{Ar}-S— is more preferable. p represents an integer of 1 to 3, preferably 1 or 2, and more preferably 1. Rd^{Ar} represents an aromatic hydrocarbon group which may have a substituent, which is the same as the aromatic hydrocarbon group which may have a substituent as Rd⁰¹. The substituent which may be contained in the aromatic hydrocarbon group as Rd^{Ar} is the same as the substituent contained in the cyclic group as Rd⁰¹. Among the examples, an iodine atom is preferable.

In Formula (d03), Rd⁰² represents a monovalent organic group or a hydrogen atom. Examples of the monovalent organic group include the same groups as those for the monovalent organic group as Rd⁰¹.

The monovalent organic group as Rd⁰² may be linked to a divalent linking group having a heteroatom and the monovalent organic group described in the section of Rd⁰¹.

Examples of the divalent linking group having a heteroatom include the same groups as those for the divalent linking group having a heteroatom as Ya^{x1} in General Formula (a10-1). As the divalent linking group having a heteroatom, —C(═O)—O—, —O—C(═O)—, —O—, —C(═O)—, a linear or branched aliphatic hydrocarbon group, or a combination thereof is preferable.

In Formula (d03), the monovalent organic group as Rd⁰² is preferably a cyclic group which may have a substituent or a group in which a cyclic group which may have a substituent is bonded to a divalent linking group having —C(═O)—O—, —O—C(═O)—, —O—, or —C(═O)—. The cyclic group which may have a substituent is more preferably an aromatic hydrocarbon group which may have a substituent, still more preferably a phenyl group which may have a substituent or a naphthyl group which may have a substituent, and particularly preferably a phenyl group which may have a substituent. As the substituent contained in the cyclic group, an iodine atom, an alkoxy group, a fluorine atom, a hydroxyl group, —(CH₂)ₚ—OH, a carboxy group, or —C(═O)—O-Rd^{Al} is preferable, and an iodine atom, —(CH₂)ₚ—OH, a carboxy group, or —C(═O)—O-Rd^{Al} is more preferable. p represents an integer of 1 to 3, preferably 1 or 2, and more preferably 1. Rd^{Al} represents a linear or branched aliphatic hydrocarbon group having 1 to 3 carbon atoms and preferably a methyl group.

In Formula (d03), at least one of Rd⁰¹ or Rd⁰² has at least one iodine atom as a substituent. The number of iodine atoms contained in the component (D03) is not particularly limited, but is preferably in a range of 1 to 6, more preferably in a range of 1 to 3, and still more preferably 1 or 2 from the viewpoints of the sensitivity and the solubility.

In Formula (d03), it is preferable that at least one of Rd⁰¹ or Rd⁰² has an aromatic ring, and both Rd⁰¹ and Rd⁰² may have an aromatic ring. In a case where at least one of Rd⁰¹ or Rd⁰² has an aromatic ring, the sensitivity and lithography characteristics such as CDU are likely to be improved.

In Formula (d03), Rd⁰¹ and Rd⁰² may or may not contain an acid dissociable group. In a case where Rd⁰¹ and Rd⁰² contains an acid dissociable group, it is preferable that the total number of acid dissociable groups in Rd⁰¹ and Rd⁰² is not 2 or more. That is, in a case where one of Rd⁰¹ and Rd⁰² contains an acid dissociable group, it is preferable that the other does not contain an acid dissociable group. It is preferable that Rd⁰¹ and Rd⁰² each do not contain two or more acid dissociable groups. The compound (D03) is considered to release electrons and undergo oxidative decomposition upon light exposure. In a case where the compound (D03) contains an acid dissociable group, it is assumed that the compound (D03) is not decomposed unless the acid dissociable group is dissociated. Therefore, in a case where the compound (D03) contains two or more acid dissociable groups, the decomposition of the compound (D03) in the exposed portion is unlikely to proceed, and the sensitivity may be degraded.

In particular, a tert-butoxycarbonyl group (t-BOC) is relatively difficult to deprotect. Therefore, it is preferable that Rd⁰¹ and Rd⁰² do not contain a total of two or more t-BOCs. In a case where one of Rd⁰¹ and Rd⁰² contains t-BOC, it is preferable that the other does not contain t-BOC. In a case where one of Rd⁰¹ and Rd⁰² contains t-BOC, from the viewpoint of improving the sensitivity, the other has preferably an aromatic ring and more preferably an aromatic ring without containing t-BOC.

In Formula (d03), Ld⁰¹ represents a single bond or a divalent linking group.

Examples of the divalent linking group include the same groups as those for the divalent linking group as Ya^{x1} in General Formula (a10-1).

In Formula (d03), as the divalent linking group represented by Ld⁰¹, —C(═O)—O—, —O—C(═O)—, —O—, —C(═O)—, a linear or branched aliphatic hydrocarbon group, or a combination thereof is preferable. As the linear or branched aliphatic hydrocarbon group, a linear or branched alkylene group having 1 to 6 carbon atoms or a linear or branched alkenylene group having 2 to 6 carbon atoms is preferable. As the divalent linking group represented by Ld⁰¹, —C(═O)—O—, —O—C(═O)—, —O—, or —C(═O)— is more preferable.

From the viewpoints of the solubility and the dispersibility in a film, it is preferable that in Formula (d03), Rd⁰¹-Ld⁰¹- and -Rd⁰² bonded to a hydrazine skeleton (—NH—NH—) have different structures.

It is preferable that the component (D03) is a compound represented by General Formula (d03-1) (hereinafter, also referred to as “component (D031)”) or a compound represented by General Formula (d03-2) (hereinafter, also referred to as “component (D032)”).

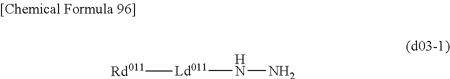

[In the formula, Rd⁰¹¹ represents a monovalent organic group having at least one iodine atom as a substituent, and Ld⁰¹¹ represents a single bond or a divalent linking group.]

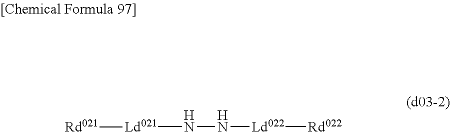

[In the formula, Rd⁰²¹ and Rd⁰²² each independently represent a monovalent organic group, and Ld⁰²¹ and Ld⁰²² each independently represent a single bond or a divalent linking group. Here, at least one of Rd⁰²¹ or Rd⁰²² has at least one iodine atom as a substituent.]

⋅Compound Represented by General Formula (d03-1) (Component (D031))

In Formula (d03-1), Rd⁰¹¹ represents a monovalent organic group. Rd⁰¹¹ has the same definition as that for Rd⁰¹ in Formula (d03).

In Formula (d03-1), Rd⁰¹¹ preferably represents a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent. In a case where Rd⁰¹¹ has an aromatic ring, the sensitivity and lithography characteristics such as CDU are likely to be improved.

In Formula (d03-1), the substituent contained in the cyclic group as Rd⁰¹¹ is preferably an iodine atom, an alkoxy group, a fluorine atom, a hydroxyl group, —(CH₂)ₚ—OH, a carboxy group, or Rd^{Ar}-S— and more preferably an iodine atom or Rd^{Ar}-S—. p represents an integer of 1 to 3, preferably 1 or 2, and more preferably 1.

In Formula (d03-1), Ld⁰¹¹ represents a single bond or a divalent linking group, Ld⁰¹¹ has the same definition as that for Ld⁰¹ in Formula (d03).

In Formula (d03-1), as the divalent linking group represented by Ld⁰¹¹, —C(═O)—O—, —O—C(═O)—, —O—, —C(═O)—, a linear or branched aliphatic hydrocarbon group, or a combination thereof is preferable. As the linear or branched aliphatic hydrocarbon group, a linear or branched alkylene group having 1 to 6 carbon atoms or a linear or branched alkenylene group having 2 to 6 carbon atoms is preferable. The divalent linking group as Ld⁰¹¹ is more preferably —C(═O)—O—, —O—C(═O)—, —O—, or —C(═O)— and still more preferably —O—C(═O)—.

A compound represented by General Formula (d03-1-1) (hereinafter, also referred to as “component (D0311)”) is preferable as the component (D031).

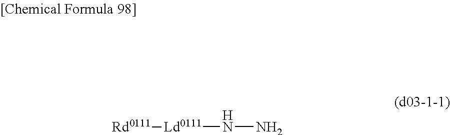

[In the formula, Rd⁰¹¹¹ represents a monovalent organic group having at least one iodine atom as a substituent, and Ld⁰¹¹¹ represents a divalent linking group.]

In Formula (d03-1-1), Rd⁰¹¹¹ has the same definition as that for Rd⁰¹ in Formula (d03). Here, Rd⁰¹¹¹ has at least one iodine atom as a substituent.

In Formula (d03-1-1), Rd⁰¹¹¹ preferably represents a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent. In a case where Rd⁰¹¹¹ has an aromatic ring, the sensitivity and lithography characteristics such as CDU are likely to be improved.

In Formula (d03-1-1), as the substituent contained in the cyclic group as Rd⁰¹¹¹, an iodine atom, an alkoxy group, a fluorine atom, a hydroxyl group, —(CH₂)ₚ—OH, a carboxy group, or Rd^{Ar}-S— is preferable, and an iodine atom or Rd^{Ar}-S— is more preferable. p represents an integer of 1 to 3, preferably 1 or 2, and more preferably 1.

In Formula (d03-1-1), Ld⁰¹¹¹ represents a single bond or a divalent linking group. Ld⁰¹¹¹ has the same definition as that for Ld⁰¹ in Formula (d03).

In Formula (d03-1-1), as the divalent linking group represented by Ld⁰¹¹¹, —C(═O)—O—, —O—C(═O)—, —O—, or —C(═O)— is more preferable, and —O—C(═O)— is still more preferable.

Specific examples of the component (D031) are shown below, but the present invention is not limited thereto.

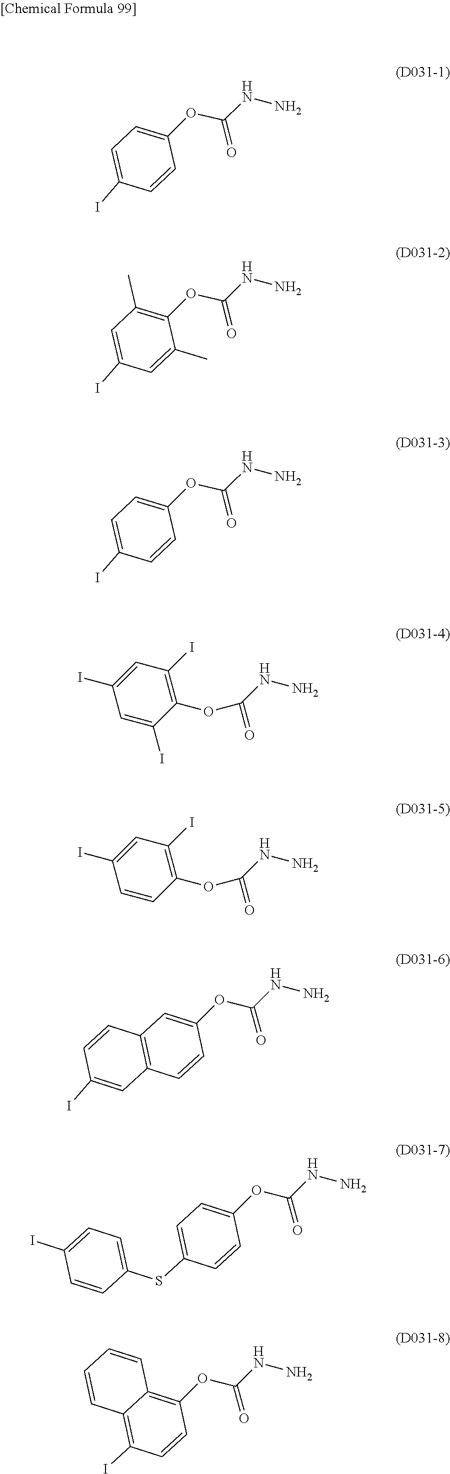

⋅Compound Represented by General Formula (d03-2) (Component (D032))

In Formula (d03-2), Rd⁰²¹ and Rd⁰²² each independently represent a monovalent organic group. Examples of Rd⁰²¹ include the same groups as those for Rd⁰¹ in Formula (d03). Examples of Rd⁰²² include the same groups as those for Rd⁰¹ in Formula (d03).

In Formula (d03-2), Rd⁰²¹ preferably represents a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent.

In Formula (d03-2), the substituent contained in the cyclic group as Rd⁰²¹ is preferably an iodine atom, an alkoxy group, a fluorine atom, a hydroxyl group, —(CH₂)ₚ—OH, a carboxy group, Rd^{Ar}-S—, or —C(═O)—O-Rd^{Al} and more preferably an iodine atom or —(CH₂)ₚ—OH. p represents an integer of 1 to 3, preferably 1 or 2, and more preferably 1. Rd^{Al} represents a linear or branched aliphatic hydrocarbon group having 1 to 3 carbon atoms and preferably a methyl group.

In Formula (d03-2), Rd⁰²² preferably represents a cyclic group which may have a substituent or a linear or branched aliphatic hydrocarbon group, more preferably an aromatic hydrocarbon group which may have a substituent or a linear or branched aliphatic hydrocarbon group, still more preferably a phenyl group which may have a substituent or a naphthyl group which may have a substituent, and particularly preferably a phenyl group which may have a substituent.

In Formula (d03-2), the substituent that Rd⁰²² may have is preferably an iodine atom, an alkoxy group, a fluorine atom, a hydroxyl group, —(CH₂)ₚ—OH, a carboxy group, or —C(═O)—O-Rd^{Al} and more preferably an iodine atom, —(CH₂)ₚ—OH, a carboxy group, or —C(═O)—O-Rd^{Al}. p represents an integer of 1 to 3, preferably 1 or 2, and more preferably 1. Rd^{Al} represents a linear or branched aliphatic hydrocarbon group having 1 to 3 carbon atoms and preferably a methyl group.

In Formula (d03-2), at least one of Rd⁰²¹ or Rd⁰²² has at least one iodine atom as a substituent. The number of iodine atoms contained in the component (D032) is not particularly limited, but is preferably in a range of 1 to 6, more preferably in a range of 1 to 3, and still more preferably 1 or 2 from the viewpoints of the sensitivity and the solubility.

In Formula (d03-2), Ld⁰²¹ and Ld⁰²² each independently represent a single bond or a divalent linking group. Examples of the divalent linking group as Ld⁰²¹ and Ld⁰²² include the same groups as those for Ld⁰¹ in Formula (d03).

In Formula (d03-2), as the divalent linking group represented by Ld⁰²¹ and Ld⁰²², —C(═O)—O—, —O—C(═O)—, —O—, —C(═O)—, a linear or branched aliphatic hydrocarbon group, or a combination thereof is preferable. As the linear or branched aliphatic hydrocarbon group, a linear or branched alkylene group having 1 to 6 carbon atoms or a linear or branched alkenylene group having 2 to 6 carbon atoms is preferable.

In Formula (d03-2), Ld⁰²¹ represents more preferably a single bond, —C(═O)—O—, —O—C(═O)—, —O—, or —C(═O)— and still more preferably a single bond or —O—C(═O)—.

In Formula (d03-2), Ld⁰²² represents more preferably —C(═O)—O—, —O—C(═O)—, —O—, or —C(═O)— and preferably —C(═O)—O— or —O—.

It is preferable that the component (D032) is a compound represented by General Formula (d03-2-1) (hereinafter, also referred to as “component (D0321)”) or a compound represented by General Formula (d03-2-2) (hereinafter, also referred to as “component (D0322)”).

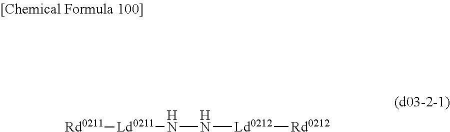

[In the formula, Rd⁰²¹¹ and Rd⁰²¹² each independently represent a monovalent organic group, and Ld⁰²¹¹ and Ld⁰²¹² each independently represent a divalent linking group. Here, at least one of Rd⁰²¹¹ or Rd⁰²¹² has at least one iodine atom as a substituent.]

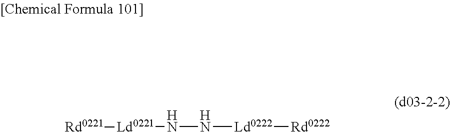

[In the formula, Rd⁰²²¹ represents a monovalent organic group, Rd⁰²²² represents a monovalent organic group having —(CH₂)ₚ—OH or a carboxy group as a substituent, p represents an integer of 1 to 3, and Ld⁰²²¹ and Ld⁰²²² each independently represent a single bond or a divalent linking group. Here, at least one of Rd⁰²²¹ or Rd⁰²²² has at least one iodine atom as a substituent.]

In Formula (d03-2-1), Rd⁰²¹¹ and Rd⁰²¹² each independently represent a monovalent organic group. Examples of Rd⁰²¹¹ and Rd⁰²¹² include the same groups as those for Rd⁰¹ in Formula (d03).

In Formula (d03-2-1), Rd⁰²¹¹ preferably represents a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent.

In Formula (d03-2-1), the substituent contained in the cyclic group as Rd⁰²¹¹ is preferably an iodine atom, an alkoxy group, a fluorine atom, a hydroxyl group, —(CH₂)ₚ—OH, a carboxy group, Rd^{Ar}-S—, or —C(═O)—O-Rd^{Al} and more preferably an iodine atom. p represents an integer of 1 to 3, preferably 1 or 2, and more preferably 1. Rd^{Al} represents a linear or branched aliphatic hydrocarbon group having 1 to 3 carbon atoms and preferably a methyl group.

In Formula (d03-2-1), Rd⁰²¹² preferably represents a cyclic group which may have a substituent or a linear or branched aliphatic hydrocarbon group, more preferably an aromatic hydrocarbon group which may have a substituent or a linear or branched aliphatic hydrocarbon group, still more preferably a phenyl group which may have a substituent or a naphthyl group which may have a substituent, and particularly preferably a phenyl group which may have a substituent.

In Formula (d03-2-1), the substituent which may be contained in Rd⁰²¹² is preferably an iodine atom, an alkoxy group, a fluorine atom, a hydroxyl group, —(CH₂)ₚ—OH, a carboxy group, or —C(═O)—O-Rd^{Al} and more preferably an iodine atom, —(CH₂)ₚ—OH, a carboxy group, or —C(═O)—O-Rd^{Al}. p represents an integer of 1 to 3, preferably 1 or 2, and more preferably 1. Rd^{Al} represents a linear or branched aliphatic hydrocarbon group having 1 to 3 carbon atoms and preferably a methyl group.

In Formula (d03-2-1), at least one of Rd⁰²¹¹ or Rd⁰²¹² has at least one iodine atom as a substituent. The number of iodine atoms contained in the component (D0321) is not particularly limited, but is preferably in a range of 1 to 6, more preferably in a range of 1 to 3, and still more preferably 1 or 2 from the viewpoint of the solubility.

In Formula (d03-2-1), Ld⁰²¹¹ and Ld⁰²¹² each independently represent a single bond or a divalent linking group. Examples of the divalent linking group as Ld⁰²¹¹ and Ld⁰²¹² include the same groups as those for Ld⁰¹ in Formula (d03).

In Formula (d03-2-1), as the divalent linking group represented by Ld⁰²¹¹ and Ld⁰²¹², —C(═O)—O—, —O—C(═O)—, —O—, —C(═O)—, a linear or branched aliphatic hydrocarbon group, or a combination thereof is preferable. As the linear or branched aliphatic hydrocarbon group, a linear or branched alkylene group having 1 to 6 carbon atoms or a linear or branched alkenylene group having 2 to 6 carbon atoms is preferable.

In Formula (d03-2-1), Ld⁰²¹¹ represents more preferably —C(═O)—O—, —O—C(═O)—, —O—, or —C(═O)— and still more preferably —O—C(═O)—.

In Formula (d03-2-1), Ld⁰²¹² represents more preferably —C(═O)—O—, —O—C(═O)—, —O—, or —C(═O)— and preferably —C(═O)—O— or —O—.

In Formula (d03-2-2), Rd⁰²²¹ represents a monovalent organic group. Examples of Rd⁰²²¹ and Rd⁰²²² include the same groups as those for Rd⁰¹ in Formula (d03).

In Formula (d03-2-2), Rd⁰²²¹ preferably represents a cyclic group which may have a substituent, more preferably an aromatic hydrocarbon group which may have a substituent, still more preferably a phenyl group which may have a substituent or a naphthyl group which may have a substituent, and particularly preferably a phenyl group which may have a substituent.

In Formula (d03-2-2), the substituent contained in the cyclic group as Rd⁰²²¹ is preferably an iodine atom, an alkoxy group, a fluorine atom, a hydroxyl group, —(CH₂)ₚ—OH, a carboxy group, Rd^{Ar}-S—, or —C(═O)—O-Rd^{Al} and more preferably an iodine atom. p represents an integer of 1 to 3, preferably 1 or 2, and more preferably 1. Rd^{Al} represents a linear or branched aliphatic hydrocarbon group having 1 to 3 carbon atoms and preferably a methyl group.

In Formula (d03-2-2), Rd⁰²²² represents a monovalent organic group having —(CH₂)ₚ—OH or a carboxy group as a substituent. p represents an integer of 1 to 3, preferably 1 or 2, and more preferably 1.

In Formula (d03-2-2), examples of the monovalent organic group as Rd⁰²²² include the same groups as those for Rd⁰¹ in Formula (d03).

In Formula (d03-2-2), the monovalent organic group as Rd⁰²²² is preferably a cyclic group which may have a substituent or a linear or branched aliphatic hydrocarbon group, more preferably an aromatic hydrocarbon group which may have a substituent or a linear or branched aliphatic hydrocarbon group, still more preferably a phenyl group which may have a substituent or a naphthyl group which may have a substituent, and particularly preferably a phenyl group which may have a substituent.

In Formula (d03-2-2), Rd⁰²² may have a substituent other than —(CH₂)ₚ—OH or a carboxy group as a substituent. As the substituent, an iodine atom, an alkoxy group, or a fluorine atom is preferable, and an iodine atom or a fluorine atom is preferable.

In Formula (d03-2-2), at least one of Rd⁰²²¹ or Rd⁰²²² has at least one iodine atom as a substituent. The number of iodine atoms contained in the component (D0322) is not particularly limited, but is preferably in a range of 1 to 6, more preferably in a range of 1 to 3, and still more preferably 1 or 2 from the viewpoints of high sensitivity and the solubility in the developing solution.

In Formula (d03-2-1), Ld⁰²¹¹ and Ld⁰²¹² each independently represent a single bond or a divalent linking group. Examples of the divalent linking group as Ld⁰²¹¹ and Ld⁰²¹² include the same groups as those for Ld⁰¹ in Formula (d03).

In Formula (d03-2-2), as the divalent linking group represented by Ld⁰²²¹ and Ld⁰²²², —C(═O)—O—, —O—C(═O)—, —O—, —C(═O)—, a linear or branched aliphatic hydrocarbon group, or a combination thereof is preferable. As the linear or branched aliphatic hydrocarbon group, a linear or branched alkylene group having 1 to 6 carbon atoms or a linear or branched alkenylene group having 2 to 6 carbon atoms is preferable.

In Formula (d03-2-2), Ld⁰²²¹ represents more preferably a single bond, —C(═O)—O—, —O—C(═O)—, —O—, or —C(═O)— and more preferably a single bond or —O—C(═O)—.

In Formula (d03-2-2), Ld⁰²²² represents more preferably —C(═O)—O—, —O—C(═O)—, —O—, or —C(═O)— and preferably —C(═O)—O— or —O—.

Specific examples of the component (D032) are shown below, but the present invention is not limited thereto.

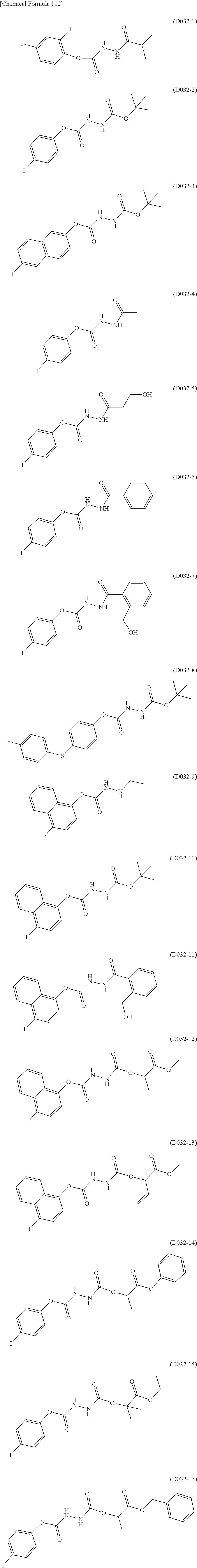

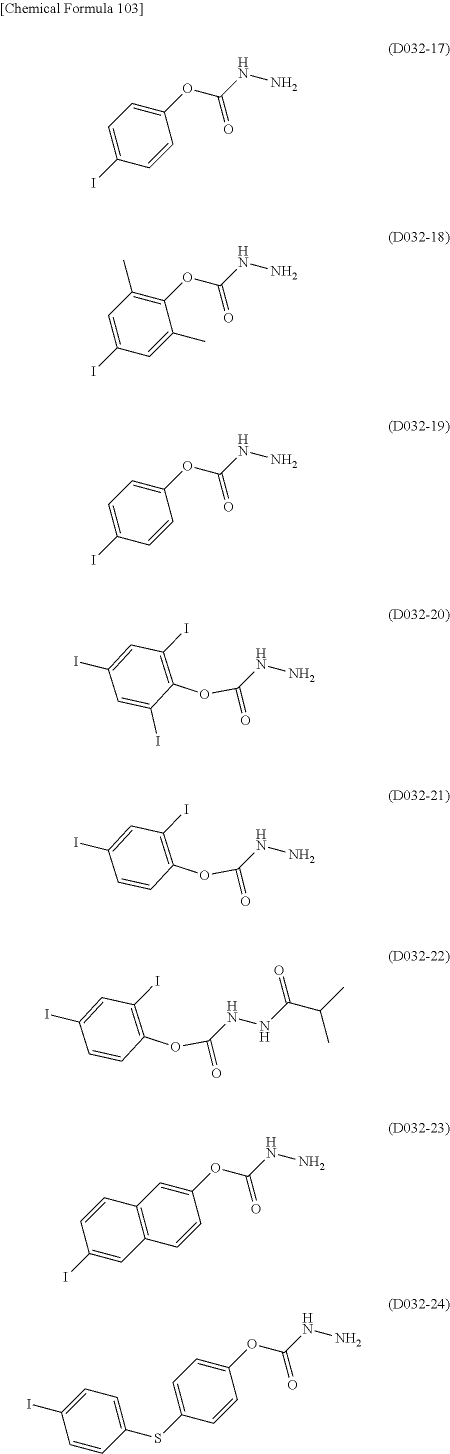

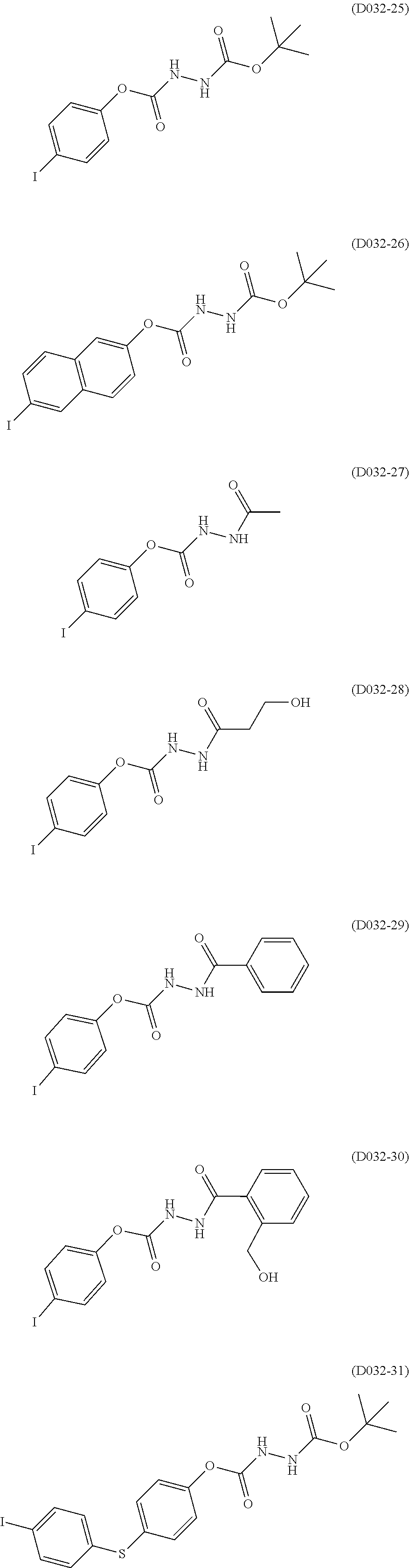

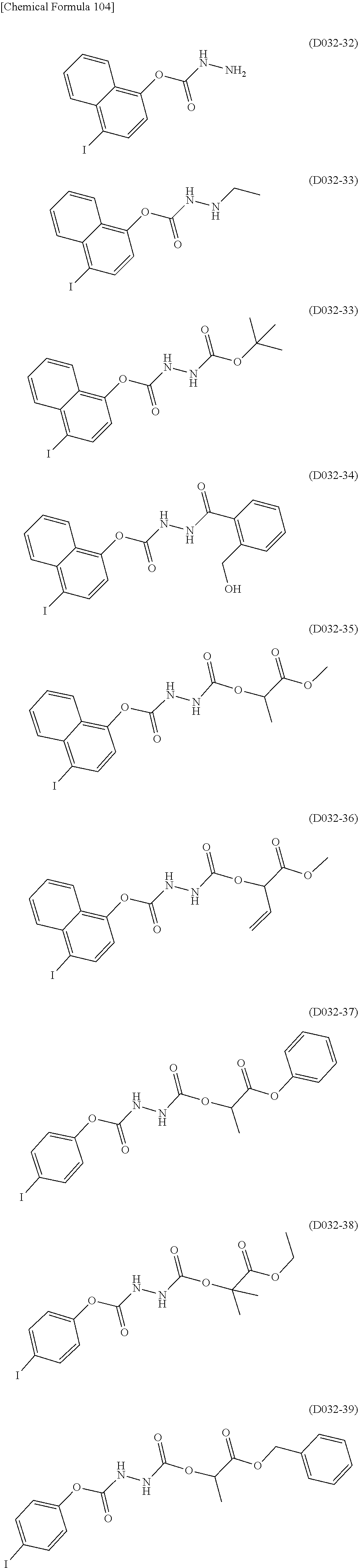

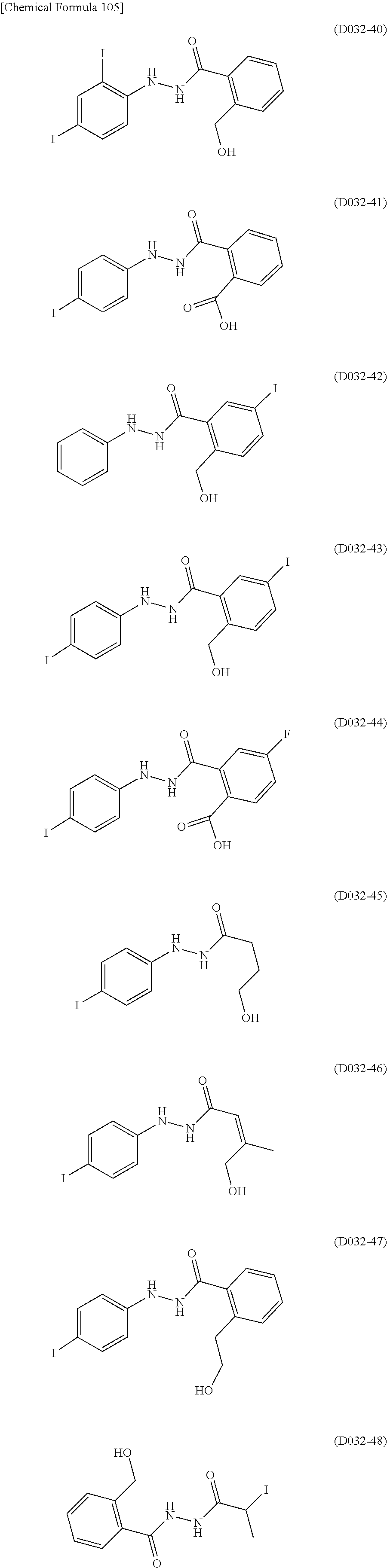

The component (D03) may be used alone or a combination of two or more kinds thereof may be used. The component (D03) may be the component (D031), the component (D032), or a combination of the component (D031) and the component (D032).

The amount of the component (D03) in the resist composition is preferably in a range of 0.5 to 20 parts by mass, more preferably in a range of 1 to 15 parts by mass, and still more preferably in a range of 3 to 10 parts by mass with respect to 100 parts by mass of the component (A).

In a case where the amount of the component (D03) is greater than or equal to the lower limits of the above-described preferable ranges, the lithography characteristics such as CDU and the like are likely to be enhanced. In a case where the amount of the component (D03) is less than or equal to the upper limits of the above-described preferable ranges, the sensitivity is likely to be satisfactorily maintained.

The amount of the component (D0) in the resist composition is preferably in a range of 0.5 to 20 parts by mass, more preferably in a range of 1 to 15 parts by mass, and still more preferably in a range of 3 to 10 parts by mass with respect to 100 parts by mass of the component (A).

In a case where the amount of the component (D0) is greater than or equal to the lower limits of the above-described preferable ranges, the lithography characteristics such as CDU and the like are likely to be enhanced. In a case where the amount of the component (D0) is less than or equal to the upper limits of the above-described preferable ranges, the sensitivity is likely to be satisfactorily maintained.

### <<Other Components (D)>>

In the resist composition of the present embodiment, the component (D) may include other base components in addition to the component (D0). Examples of the other base components include a photodecomposable base (D1) (hereinafter, referred to as “component (D1)”) that is decomposed upon light exposure and loses the acid diffusion controllability and a nitrogen-containing organic compound (D2) that does not correspond to the component (D1) (excluding components corresponding to the component (D0)) (hereinafter, referred to as “component (D2)”).

The component (D1) and the component (D2) may be contained in the form of a compound, in the form in which the component (D1) and the component (D2) are incorporated into the component (A1) as the constitutional unit (a6) described above, or in both forms. The compound described as the component (D1) below may be used as the acid generator component (component (B)) depending on the combination with other compounds.

### ⋅In Regard to Component (D1)

The component (D1) is not particularly limited as long as the component is decomposed upon light exposure and loses an acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not act as a quencher at the exposed portion of the resist film, but act as a quencher at the unexposed portion of the resist film.

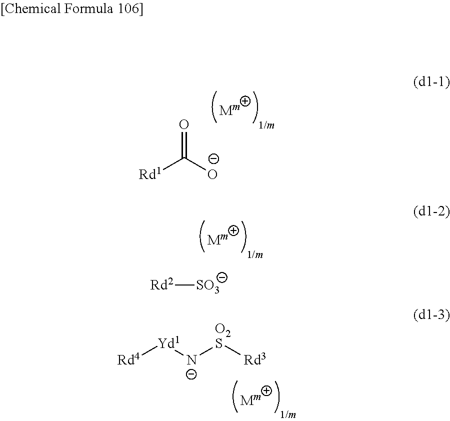

[In the formulae, Rd¹ to Rd⁴ represent a cyclic group which may have a substituent, a chain-like alkyl group which may have a substituent, or a chain-like alkenyl group which may have a substituent. Here, the carbon atom adjacent to the S atom as Rd² in Formula (d1-2) has no fluorine atom bonded thereto. Yd¹ represents a single bond or a divalent linking group. m represents an integer of 1 or greater, and each M^{m+} independently represents an m-valent organic cation.]

{Component (d1-1)}

### ⋅⋅Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that the group as Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where an ether bond or an ester bond is contained as the substituent, the substituent may be bonded via an alkylene group, and a linking group represented by any of Formulae (L-a1-1) to (L-a1-5) is preferable as the substituent in this case. Further, in a case where the aromatic hydrocarbon group, the aliphatic cyclic group, or the chain-like alkyl group as Rd¹ contains a linking group represented by any of General Formulae (L-a1-1) to (L-a1-7) as a substituent, V′¹⁰¹ in General Formula (L-a1-1) to (L-a1-7) is bonded to the carbon atom constituting the aromatic hydrocarbon group, the aliphatic cyclic group, or the chain-like alkyl group as Rd¹ in Formula (d3-1), in General Formulae (y-a1-1) to (L-a1-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclo[5.2.1.0^{2,6}]decane, or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

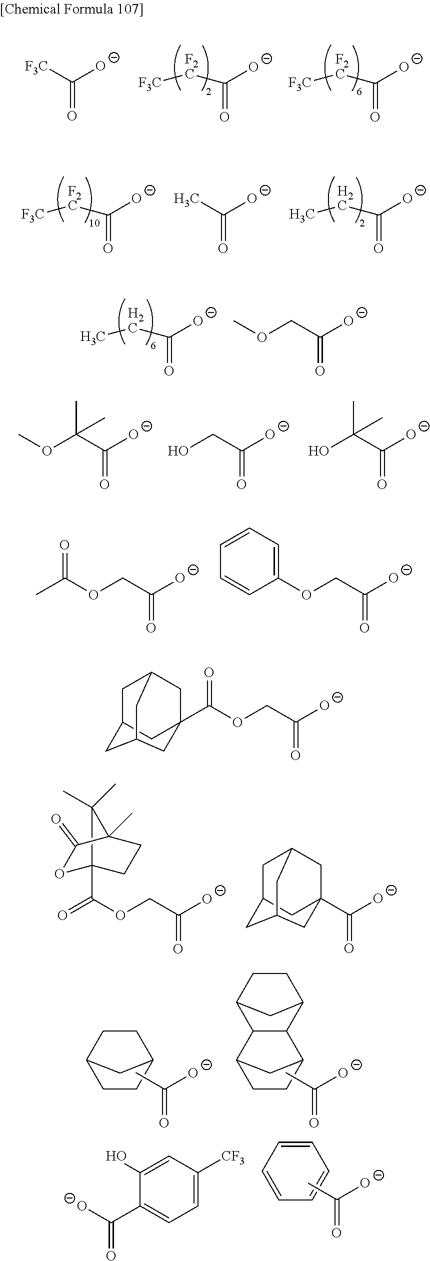

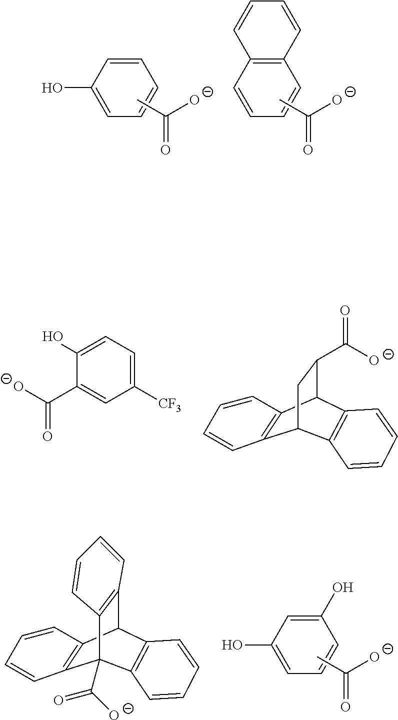

### ⋅Cation Moiety

In Formula (d1-1), M^{m+} represents an m-valent organic cation.

Examples of the organic cation as M^{m+} include the same cations as those for the cations each represented by General Formulae (ca-1) to (ca-3). Among these, a cation represented by General Formula (ca-1) is more preferable, and cations each represented by Formulae (ca-1-1) to (ca-1-75) are still more preferable.

The component (d1-1) may be used alone or a combination of two or more kinds thereof may be used.

{Component (d1-2)}

### ⋅⋅Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, the carbon atom adjacent to the S atom in Rd² has no fluorine atom bonded thereto (the carbon atom is not substituted with a fluorine atom). As a result, the anion of the component (d1-2) becomes an appropriately weak acid anion, thereby improving the quenching ability of the component (D).

Rd² preferably represents a chain-like alkyl group which may have a substituent or an aliphatic cyclic group which may have a substituent, and more preferably an aliphatic cyclic group which may have a substituent.

The chain-like alkyl group preferably has 1 to 10 carbon atoms and more preferably 3 to 10 carbon atoms.

As the aliphatic cyclic group, a group (which may have a substituent) in which one or more hydrogen atoms have been removed from adamantane, norbornane, isobornane, tricyclo[5.2.1.0^{2,6}]decane, or tetracyclododecane; and a group in which one or more hydrogen atoms have been removed from camphor is more preferable.

The hydrocarbon group as Rd² may have a substituent, and examples of the substituent include the same substituents as those that the hydrocarbon group (such as an aromatic hydrocarbon group, an aliphatic cyclic group, or a chain-like alkyl group) as Rd¹ in Formula (d1-1) may have.

Specific preferred examples of the anion moiety in the component (d1-2) are described below.

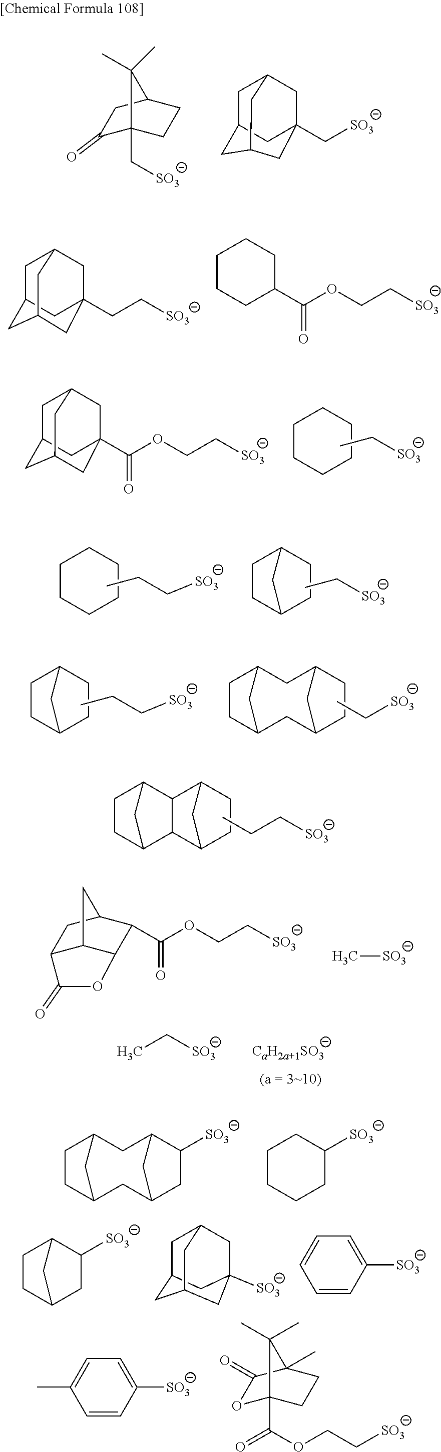

### ⋅⋅Cation Moiety

In Formula (d1-2), M^{m+} represents an m-valent organic cation and has the same definition as that for M^{m+} in Formula (d1-1).

The component (d1-2) may be used alone or a combination of two or more kinds thereof may be used.

{Component (d1-3)}

### ⋅⋅Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclo[5.2.1.0^{2,6}]decane, or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are improved. Further, in a case where Rd⁴ represents an aromatic group, the resist composition has excellent light absorption efficiency in lithography using EUV or the like as an exposure light source, and thus the sensitivity and lithography characteristics are improved.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. These divalent linking groups are the same as those for the divalent hydrocarbon group which may have a substituent and the divalent linking group having a heteroatom described in the section of the divalent linking group as Ya²¹ in Formula (a2-1).

It is preferable that Yd¹ represents a carbonyl group, an ester bond, an amide bond, an alkylene group, or a combination of these. As the alkylene group, a linear or branched alkylene group is more preferable, and a methylene group or an ethylene group is still more preferable.

Specific preferred examples of the anion moiety in the component (d1-3) are described below.

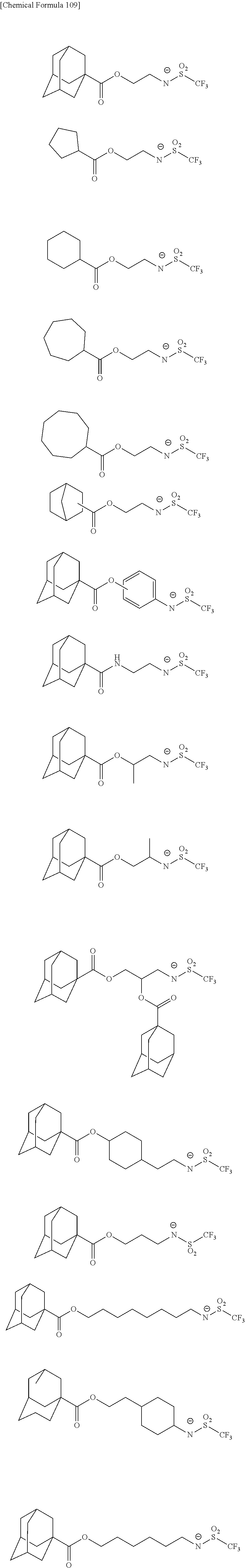

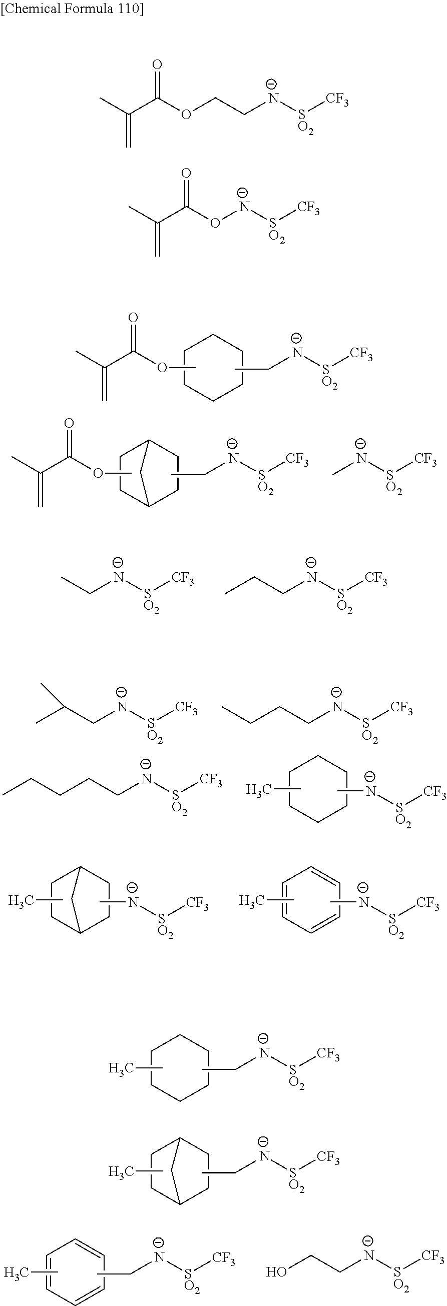

### ⋅⋅Cation Moiety

In Formula (d1-3), M^{m+} represents an m-valent organic cation and has the same definition as that for M^{m+} in Formula (d1-1).

The component (d1-3) may be used alone or a combination of two or more kinds thereof may be used.

As the component (D1), only one of the above-described components (d1-1) to (d1-3) or a combination of two or more kinds thereof may be used.

In a case where the resist composition contains the component (D1), the amount of the component (D1) in the resist composition is preferably in a range of 0.5 to 15 parts by mass, more preferably in a range of 1 to 10 parts by mass, and still more preferably in a range of 2 to 8 parts by mass with respect to 100 parts by mass of the component (A).

It is preferable that the component (D1) contains the component (d1-1).

The amount of the component (d1-1) in the total component (D1) is preferably 50% by mass or more, preferably 70% by mass or more, and still more preferably 90% by mass or more, and the component (D1) may consist of only a compound for the component (d1-1).

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

The compound of the component (D1) has been described as an example of the base component (the component (D)) that traps an acid generated upon light exposure, but the compound of the component (D1) may be used as the component (B) depending on the degree of acidity of the acid generated upon light exposure.

### ⋅In Regard to Component (D2)

The component (D) may contain a nitrogen-containing organic compound component (hereinafter, referred to as “component (D2)”) which does not correspond to the above-described component (D0) or component (D1).

The component (D2) is not particularly limited as long as the component acts as an acid diffusion control agent and does not correspond to the component (D0) or the component (D1), and any known compound may be used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of these aliphatic amines include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Among these, triethanolamine triacetate is preferable.

As the component (D2), an aromatic amine may be used.

Examples of the aromatic amine include 4-dimethylaminopyridine, pyrrole, indole, pyrazole, imidazole, and derivatives thereof, tribenzylamine, 2,6-diisopropylaniline, N-tert-butoxycarbonylpyrrolidine, 2,6-di-tert-butylpyridine, and 2,6-di-tert-butylpyridine.

Among the examples, the component (D2) is preferably an alkylamine and more preferably a trialkylamine having 5 to 10 carbon atoms.

The component (D2) may be used alone or a combination of two or more kinds thereof may be used.

In a case where the resist composition contains the component (D2), the amount of the component (D2) in the resist composition is preferably in a range of 0.01 to 5 parts by mass, more preferably in a range of 0.1 to 5 parts by mass, and still more preferably in a range of 0.5 to 5 parts by mass with respect to 100 parts by mass of the component (A).

In a case where the amount of the component (D2) is set to be greater than or equal to the lower limits or the above-described preferable ranges, excellent lithography characteristics and an excellent resist pattern shape are easily obtained. On the contrary, in a case where the content is set to be less than or equal to the upper limits of the above-described ranges, the sensitivity can be satisfactorily maintained and the throughput is also excellent.

As the component (D), only the component (D0) may be used, the component (D0) and the component (D1) may be used in combination, the component (D0) and the component (D2) may be used in combination, or the component (D0), the component (D1), and the component (D2) may be used in combination. The component (D) may not include the component (D1), and may not include the component (D2).

The proportion of the component (D0) in the entire component (D) is, for example, preferably in a range of 20% to 100% by mass, more preferably in a range of 30% to 100% by mass, and still more preferably in a range of 40% to 100% by mass.

The amount of the component (D) in the resist composition according to the present embodiment is preferably in a range of 0.5 to 20 parts by mass, more preferably in a range of 1 to 15 parts by mass, and still more preferably in a range of 3 to 10 parts by mass with respect to 100 parts by mass of the component (A).

In a case where the amount of the component (D) is greater than or equal to the lower limits of the above-described preferable ranges, particularly satisfactory lithography characteristics and a satisfactory resist pattern shape are likely to be obtained. On the contrary, in a case where the content is set to be less than or equal to the upper limits of the above-described ranges, the sensitivity can be satisfactorily maintained and the throughput is also excellent.

### <Other Components>

The resist composition according to the present embodiment may further contain other components in addition to the component (A) and the component (D) described above. Examples of the other components include a component (B), a component (E), a component (F), and a component(S), which are described below.

### <<Acid Generator Component (B)>>

The resist composition according to the present embodiment may further contain an acid generator component (B) which generates an acid upon light exposure.

The component (B) is not particularly limited, and those which have been suggested so far as an acid generator for a chemically amplified resist composition in the related art can be used.

Examples of the acid generator include various acid generators, for example, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, iminosulfonate-based acid generators, and disulfone-based acid generators. The component (B) may be contained in the form of a compound, in the form in which the component (B) is incorporated into the component (A1) as the constitutional unit (a5) described above, or in both forms.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

### <<Acid Generator Component (B)>>

The resist composition according to the present embodiment may further contain an acid generator component (B) which generates an acid upon light exposure.

The component (B) is not particularly limited, and those which have been suggested so far as an acid generator for a chemically amplified resist composition in the related art can be used.

Examples of the acid generator include various acid generators, for example, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, iminosulfonate-based acid generators, and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

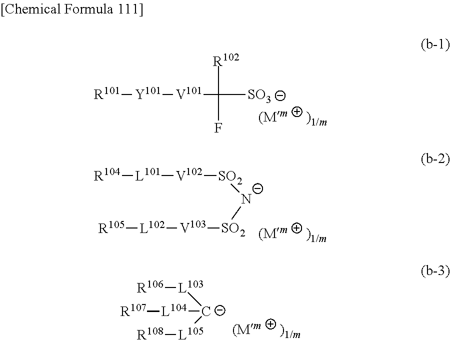

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M′^{m+} represents an m-valent onium cation.]

### {Anion Moiety}

⋅Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. In addition, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group obtained by removing one hydrogen atom from the aromatic ring (an aryl group such as a phenyl group or a naphthyl group) and a group in which one hydrogen atom of the aromatic ring is substituted with an alkylene group (for example, a benzyl group, a phenethyl group, or a 1-naphthylmethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The number of carbon atoms in the monocycloalkane is preferably 3 to 6, and specifically, cyclopentane and cyclohexane are exemplary examples. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16). In the formulae, * represents a bonding site with respect to Y¹⁰¹ in Formula (b-1).

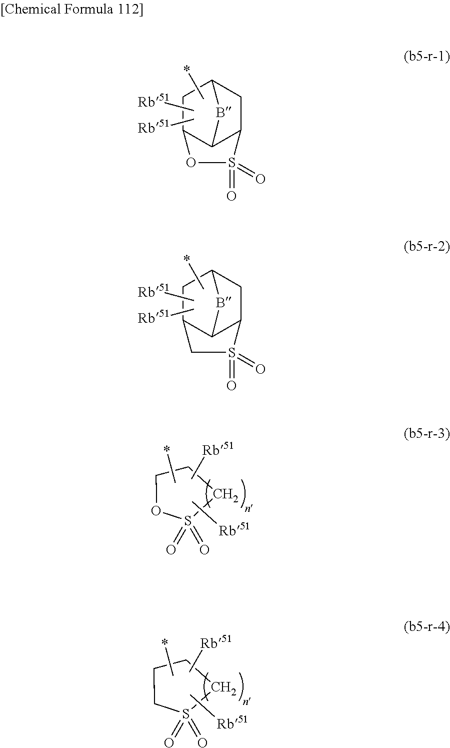

[In the formulae, Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or a —SO₂-containing cyclic group; B″ represents an oxygen atom, a sulfur atom, or an alkylene group having 1 to 5 carbon atoms, which may have an oxygen atom or a sulfur atom; and n′ represents an integer of 0 to 2. * represents a bonding site.]

In General Formulae (b5-r-1) and (b5-r-2), B″ represents an alkylene group having 1 to 5 carbon atoms which may have an oxygen atom or a sulfur atom, an oxygen atom, or a sulfur atom.

B″ preferably represents an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′51's each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, and among the examples, it is preferable that Rb′⁵¹'s each independently represent a hydrogen atom or a cyano group.

Specific examples of the groups each represented by General Formulae (b5-r-1) to (b5-r-4) are shown below. In the formulae shown below, “Ac” represents an acetyl group.

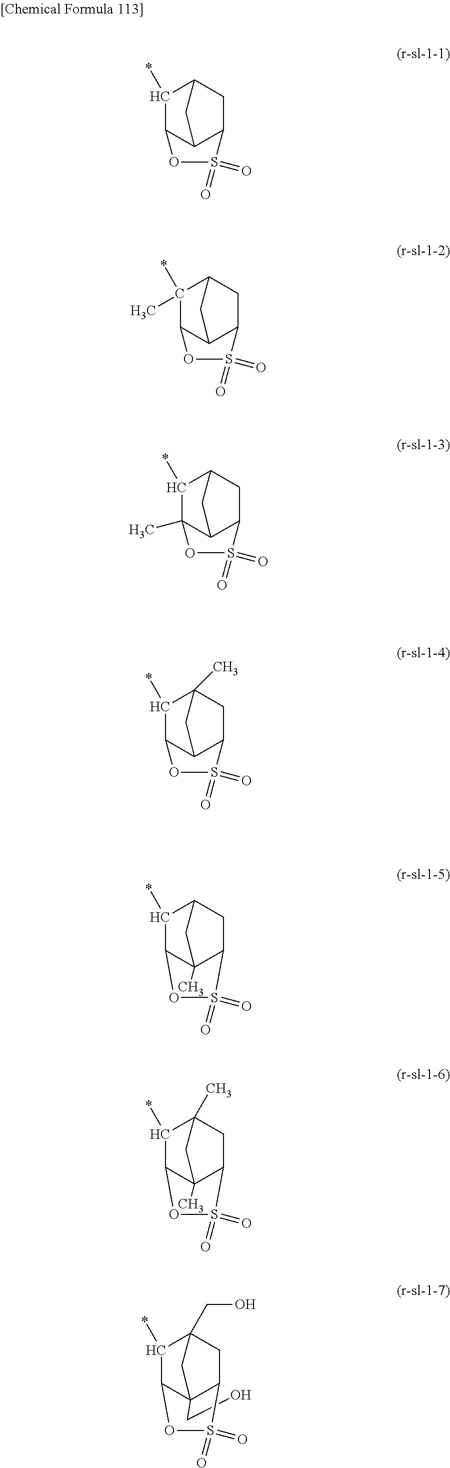

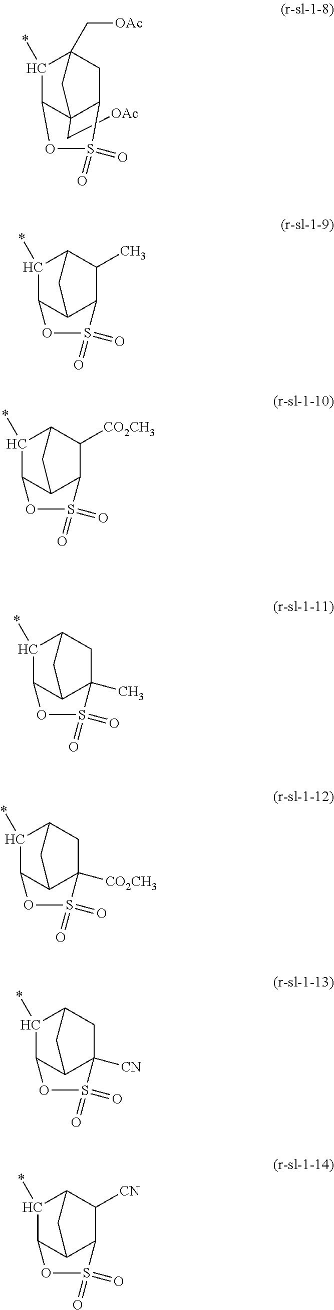

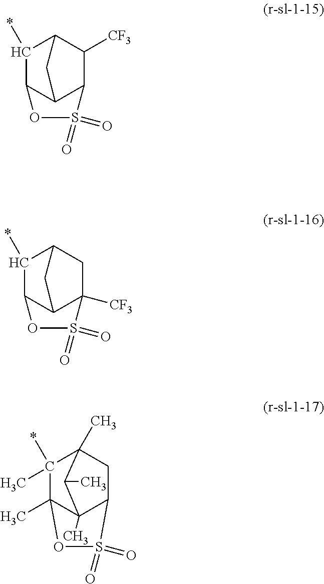

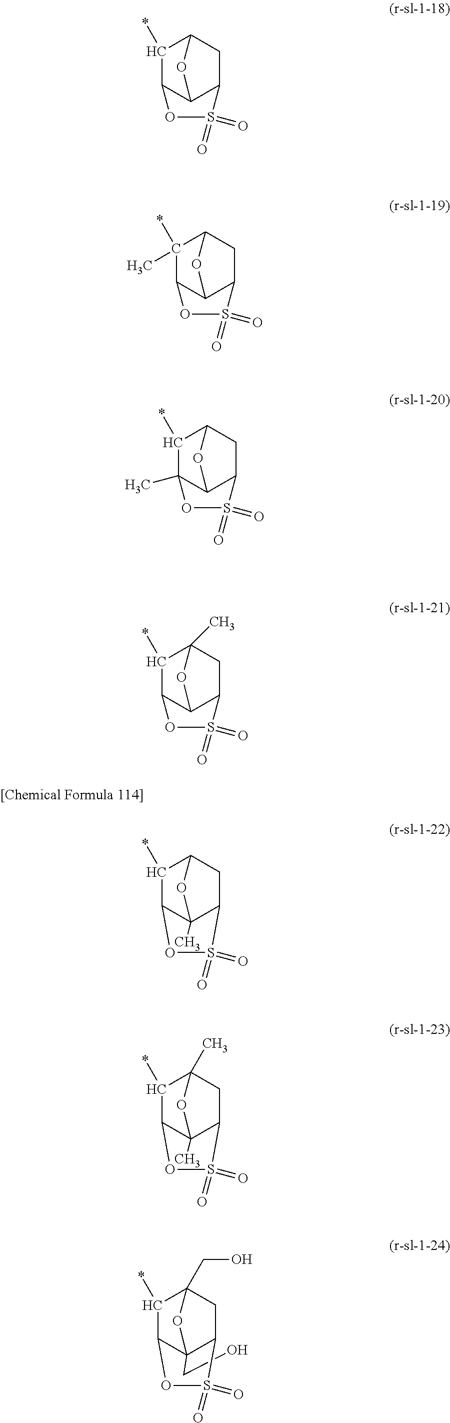

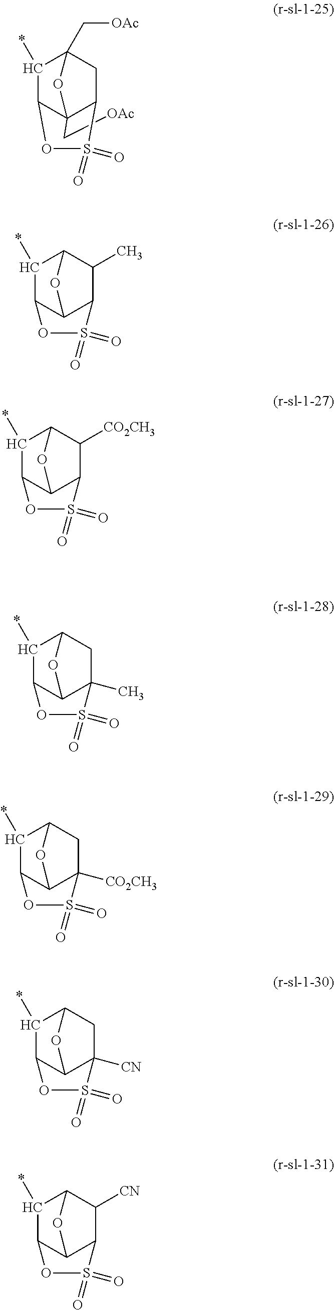

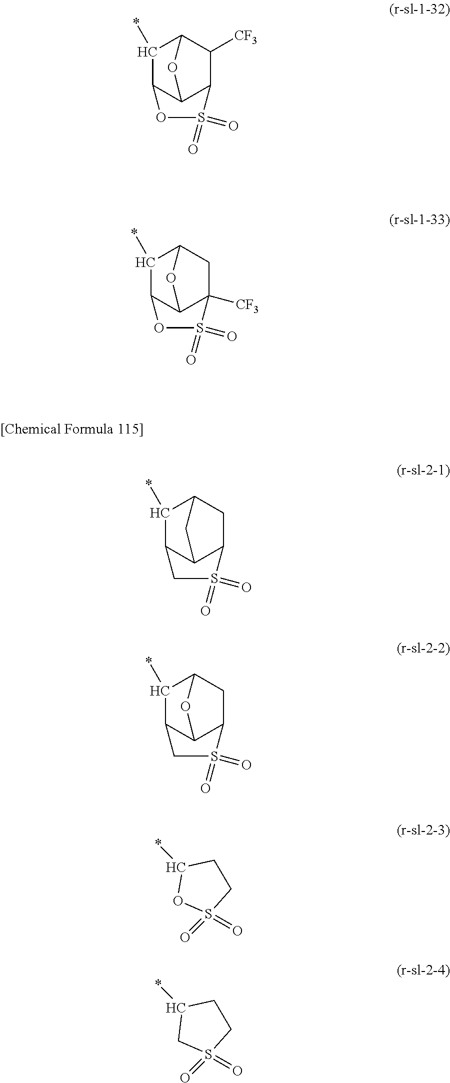

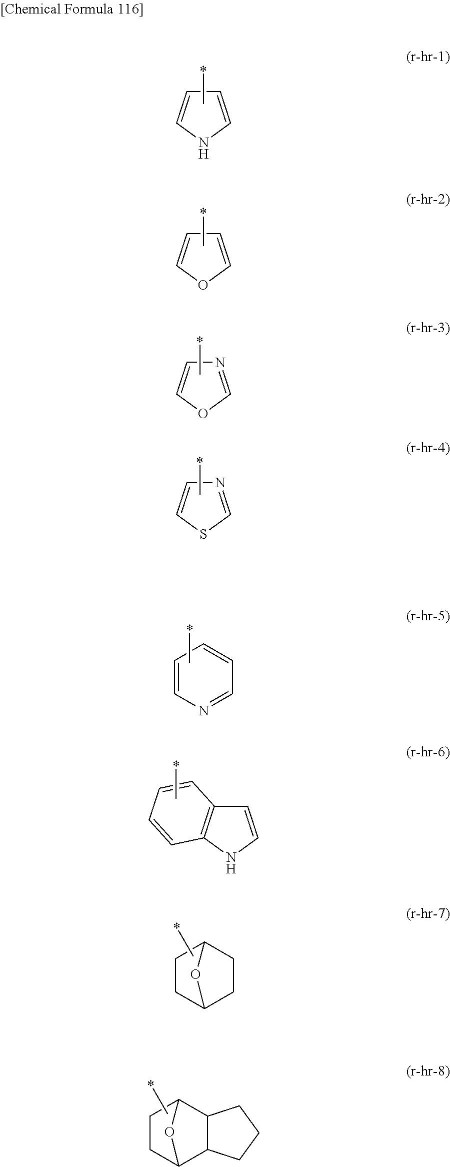

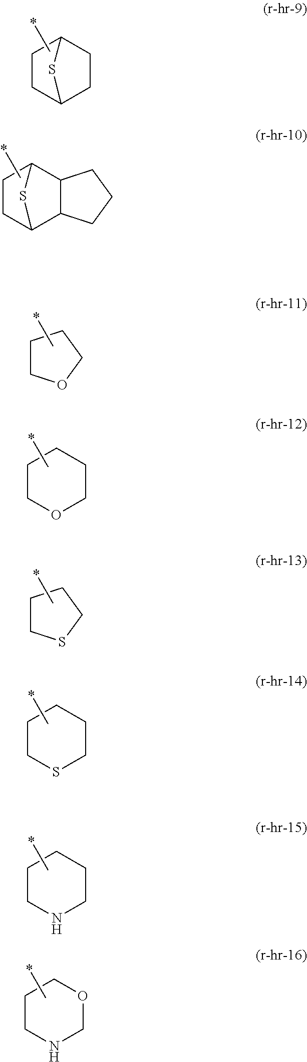

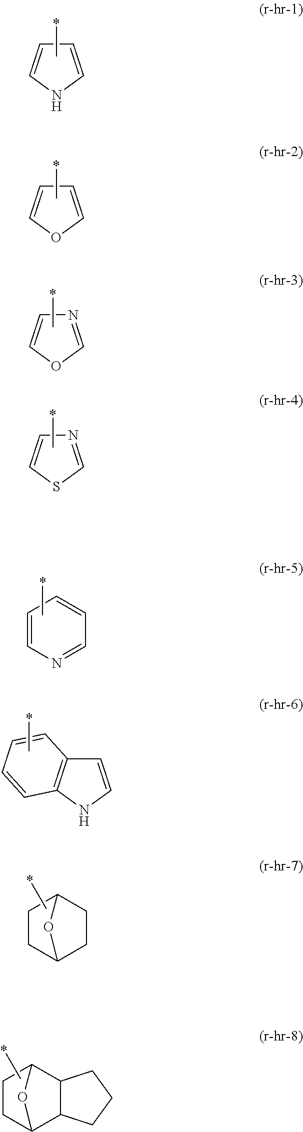

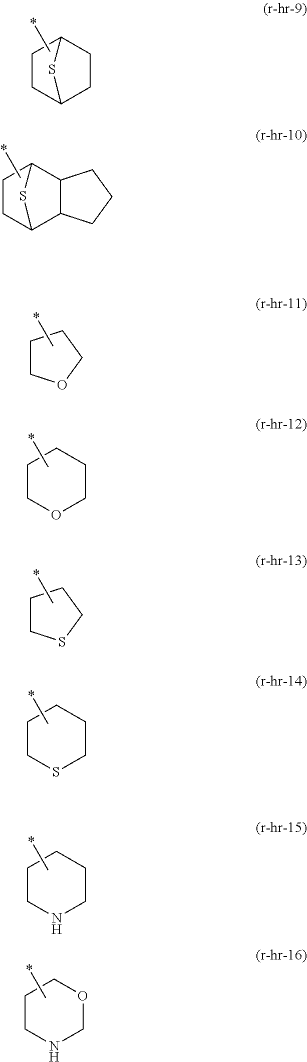

Examples of the substituent for the cyclic group as R¹⁰¹ include an alkyl group, an alkoxy group, a halogen atom, a halogenated alkyl group, a hydroxyl group, a carbonyl group, and a nitro group.

An alkyl group having 1 to 5 carbon atoms is preferable as the alkyl group serving as a substituent.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

A fluorine atom, a bromine atom, or an iodine atom is preferable as the halogen atom serving as a substituent.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a condensed cyclic group containing a condensed ring in which an aliphatic hydrocarbon ring and an aromatic ring are condensed. Examples of the condensed ring include those obtained by condensing one or more aromatic rings with a polycycloalkane having a crosslinked ring polycyclic skeleton. Specific examples of the crosslinked ring polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as R¹⁰¹ include those represented by Formulae (r-br-1) and (r-br-2). In the formulae, * represents a bonding site with respect to Y¹⁰¹ in Formula (b-1).

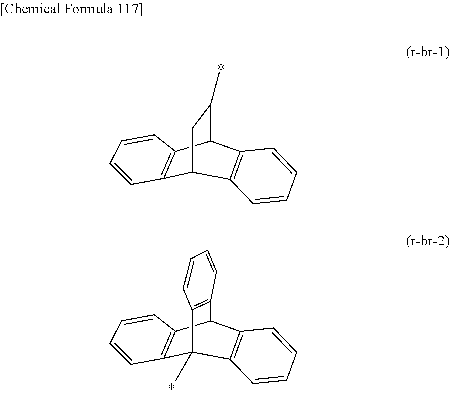

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include the same groups as those for the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane; a group in which one hydrogen atom has been removed from a polycycloalkane such as adamantane, norbornane, isobornane, tricyclo[5.2.1.0^{2,6}]decane, or tetracyclododecane; a lactone-containing cyclic group represented by any of General Formulae (a2-r-1) to (a2-r-7); a —SO₂-containing cyclic group represented by any of General Formulae (b5-r-1) to (b5-r-4); and a heterocyclic group represented by any of Formulae (r-hr-7) to (r-hr-16).

Chain-Like Alkyl Group which May have Substituent:

The chain-like alkyl group as R¹⁰¹ may be linear or branched.

The number of carbon atoms in the linear alkyl group is preferably 1 to 20, more preferably 1 to 15, and most preferably 1 to 10.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the examples, R¹⁰¹ preferably represents a cyclic group which may have a substituent and more preferably a cyclic hydrocarbon group which may have a substituent.

More specifically, as the cyclic hydrocarbon group, a group in which one or more hydrogen atoms have been removed from a phenyl group, a naphthyl group, or a polycycloalkane; a lactone-containing cyclic group represented by any of General Formulae (a2-r-1) to (a2-r-7); or a —SO₂-containing cyclic group represented by any of General Formulae (b5-r-1) to (b5-r-4) is preferable, a group in which one or more hydrogen atoms have been removed from a polycycloalkane is more preferable, and an adamantyl group is still more preferable.

In Formula (b-1), Y¹⁰¹ represents a single bond or a divalent linking group having an oxygen atom.

In a case where Y¹⁰¹ represents a divalent linking group having an oxygen atom, Y¹⁰¹ may have an atom other than the oxygen atom. Examples of atoms other than an oxygen atom include a carbon atom, a hydrogen atom, a sulfur atom, and a nitrogen atom.

Examples of the divalent linking group having an oxygen atom include the linking groups each represented by General Formulae (L-a1-1) to (L-a1-7). Further, in General Formulae (y-a1-1) to (L-a1-7), V′101 in General Formulae (L-a1-1) to (L-a1-7) is bonded to R¹⁰¹ in Formula (b-1).

Y¹⁰¹ preferably represents a divalent linking group having an ester bond or a divalent linking group having an ether bond and more preferably a linking group represented by any of Formulae (L-a1-1) to (L-a1-5).

In Formula (b-1), V¹⁰¹ represents a single bond, an alkylene group, or a fluorinated alkylene group. It is preferable that the alkylene group and the fluorinated alkylene group as V¹⁰¹ have 1 to 4 carbon atoms. Among these, it is preferable that V¹⁰¹ represents a single bond or a linear fluorinated alkylene group having 1 to 4 carbon atoms.

In Formula (b-1), R¹⁰² represents a fluorine atom or a fluorinated alkyl group having 1 to 5 carbon atoms. R¹⁰² preferably represents a fluorine atom or a perfluoroalkyl group having 1 to 5 carbon atoms and more preferably a fluorine atom.

In a case where Y¹⁰¹ represents a single bond, specific example of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

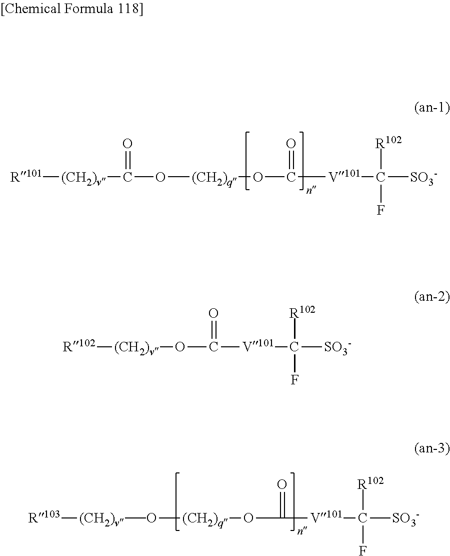

[In the formulae, R″¹⁰¹ represents an aliphatic cyclic group which may have a substituent, a monovalent heterocyclic group represented by any of Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (b5-r-1) to (b5-r-4). R″103 represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″101, R″102, and R″103 which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

The aromatic cyclic group which may have a substituent as R″¹⁰¹ and R″¹⁰³ is preferably the group described as the example of the aromatic hydrocarbon group for the cyclic hydrocarbon group as R¹⁰¹ in Formula (b-1). Examples of the substituent include the same substituents as those which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

The alkylene group and the fluorinated alkylene group as V″¹⁰¹ have preferably 1 to 3 carbon atoms and more preferably 1 or 2 carbon atoms. Specific examples of V″¹⁰¹ include —CH₂—, —(CH₂)₂—, —CFH—, —CH₂CFH—, and —CH(CF₃)—.

As the anion moiety represented by Formula (b-1), an anion moiety represented by Formula (an-1) is preferable. Among these, R″¹⁰¹ in Formula (an-1) preferably represents an aromatic cyclic group which may have a substituent and more preferably a phenyl group which may have a substituent. Examples of the substituent include a hydroxy group, an alkyl group, and a halogen atom. As the halogen atom, a bromine atom or an iodine atom is preferable, and an iodine atom is more preferable.

⋅Anions in Component (b-2)

In Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms because the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible because the acid strength increases and the transparency to high energy light with a wavelength of 250 nm or less or electron beams is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V¹⁰¹ in Formula (b-1).

In Formula (b-2), L¹⁰¹ and L¹⁰² each independently represent a single bond or an oxygen atom.

⋅Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include those for R¹⁰¹ in Formula (b-1).

In Formula (b-3), L¹⁰³ to L¹⁰⁵ each independently represent a single bond, —CO—, or —SO₂—.

Among the examples, as the anion moiety of the component (B), an anion in the component (b-1) is preferable.

### {Cation Moiety}

In Formulae (b-1), (b-2), and (b-3), M′^{m+} represents an m-valent onium cation. Among them, a sulfonium cation and an iodonium cation are preferable.

m represents an integer of 1 or greater.

Preferred examples of the cation moiety ((M′^{m+})_{1/m}) include organic cations each represented by General Formulae (ca-1) to (ca-3). As the cation moiety, a cation represented by General Formula (ca-1) is more preferable, and a cation represented by any of Formulae (ca-1-1) to (ca-1-75) is still more preferable.

It is preferable that M′^{m+} represents an m-valent organic cation having a fluorine atom. M″^{m+} preferably represents a cation represented by Formula (ca-1f) and more preferably a cation represented by Formula (ca-1-72).

In the resist composition according to the present embodiment, the component (B) may be used alone or a combination of two or more kinds thereof may be used.

In a case where the resist composition of the present embodiment contains the component (B), the amount of the component (B) in the resist composition is preferably less than 40 parts by mass, more preferably in a range of 1 to 35 parts by mass, and still more preferably in a range of 3 to 30 parts by mass with respect to 100 parts by mass of the component (A).

In a case where the amount of the component (B) is set to be in the above-described preferable range, pattern formation can be satisfactorily performed. Further, it is preferable that each component of the resist composition is dissolved in an organic solvent from the viewpoint that a uniform solution is easily obtained and the storage stability of the resist composition is enhanced.

In a case where the component (A) does not have the constitutional unit (a5), it is preferable that the resist composition of the present embodiment contains the component (B). The resist composition of the present embodiment may use the component (A) having the constitutional unit (a5) and the component (B) in combination, or may contain only one of the component (A) having the constitutional unit (a5) or the component (B).

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

In the resist composition of the present embodiment, the component (E) may be used alone or a combination of two or more kinds thereof may be used.

In a case where the resist composition contains the component (E), the amount of the component (E) is preferably in a range of 0.01 to 5 parts by mass and more preferably in a range of 0.05 to 3 parts by mass with respect to 100 parts by mass of the component (A). In a case where the content thereof is in the above-described ranges, the lithography characteristics are further improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used. Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

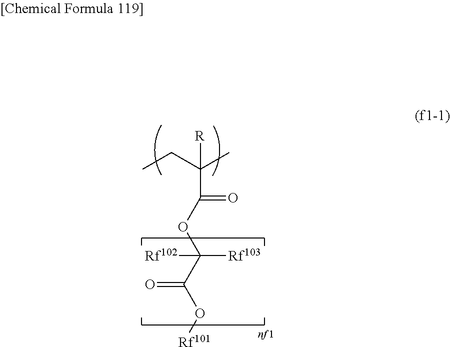

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms for Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or greater of the hydrogen atoms in the hydrocarbon group are fluorinated, more preferably 50% or greater thereof are fluorinated, and still more preferably 60% or greater thereof are fluorinated from the viewpoint of increasing the hydrophobicity of the resist film during immersion exposure.

Among examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is set to be less than or equal to the upper limits of the above-described ranges, the resist composition exhibits a satisfactory solubility in a solvent for a resist enough to be used as a resist. Meanwhile, in a case where the weight-average molecular weight thereof is set to be greater than or equal to the lower limits of the above-described ranges, water repellency of the resist film is satisfactory.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or a combination of two or more kinds thereof may be used.

In a case where the resist composition contains the component (F), the amount of the component (F) is preferably in a range of 0.5 to 10 parts by mass and more preferably in a range of 1 to 10 parts by mass with respect to 100 parts by mass of the component (A).

### <<Organic Solvent Component(S)>>

The resist composition according to the present embodiment may be produced by dissolving the resist materials in an organic solvent component (hereinafter, referred to as a “component(S)”).

In the resist composition of the present embodiment, the component(S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component(S). The blending ratio (mass ratio) may be appropriately determined in consideration of the compatibility between PGMEA and the polar solvent.

A mixed solvent of at least one selected from PGMEA and EL, and γ-butyrolactone is also preferable as the(S) component. In this case, as the mixing ratio, the mass ratio between the former and the latter is preferably in a range of 70:30 to 95:5.

The amount of the component(S) to be used is not particularly limited and is appropriately set to have a concentration which enables coating a substrate or the like depending on the thickness of the coating film. The component(S) is typically used in an amount such that the solid content concentration of the resist composition is set to be in a range of 0.1% to 20% by mass and preferably in a range of 0.2% to 15% by mass.

After the resist material is dissolved in the component(S), impurities may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition of the present embodiment described above contains the compound (D0). In this manner, high sensitivity can be achieved, and the lithography characteristics such as CDU and the like can be improved.

The reason why the above-described effects are exhibited is assumed as follows.

The compound (D0) acts as an acid diffusion control agent component. The compound (D0) is oxidatively decomposed upon light exposure and disappears from the resist film in the exposed portion. Since the decomposition of the compound (D0) due to the exposed portion does not consume electrons, the electrons are used only for the decomposition of the component (B) or the constitutional unit (a5). Therefore, it is assumed that the sensitivity is improved and the lithography characteristics such as CDU are improved.

In addition, at least one iodine atom (1) is bonded to the compound (D03). The iodine atom has a large absorption of EUV having a wavelength of 13.5 nm. Therefore, it is assumed that in a case where the compound (D03) is used as the compound (D0), the sensitivity is likely to be further improved and the lithography characteristics such as CDU are likely to be further improved.

### (Method for Forming Resist Pattern)

A method for forming a resist pattern according to a second aspect of the present invention is a method including a step of forming a resist film on a support using the resist composition according to the first aspect of the present invention described above, a step of exposing the resist film to light, and a step of developing the resist film exposed to light to form a resist pattern.

According to the embodiment of the method for forming a resist pattern, a method for forming a resist pattern, which is performed in the following manner is an exemplary example.

First, a support is coated with the resist composition of the present embodiment using a spinner or the like, and a bake (post applied bake (PAB)) treatment is performed under a temperature condition of 80° C. to 150° C. for 40 to 120 seconds and preferably 60 to 90 seconds to form a resist film.

Next, the selective exposure is performed on the resist film by, for example, light exposure through a mask (mask pattern) having a predetermined pattern formed thereon using an exposure apparatus such as an electron beam lithography apparatus or an ArF exposure apparatus, or direct irradiation with an electron beam for drawing without using a mask pattern, and a bake (post exposure bake (PEB)) treatment is carried out, for example, under a temperature condition of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post-bake) may be conducted after the developing treatment.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

The wavelength to be used for light exposure is not particularly limited and the exposure can be conducted using radiation such as an ArF excimer laser, a KrF excimer laser, an F2 excimer laser, extreme ultraviolet (EUV) rays, vacuum ultraviolet rays (VUV), electron beams (EB), X-rays, and soft X-rays.

The method of exposing the resist film to light may be general exposure (dry exposure) conducted in air or an inert gas such as nitrogen, or liquid immersion exposure (liquid immersion lithography).

The liquid immersion exposure is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

As the liquid immersion medium, a solvent having a refractive index greater than the refractive index of air but less than the refractive index of the resist film to be exposed is preferable, and examples thereof include water, a fluorine-based inert liquid, a silicon-based solvent, and a hydrocarbon-based solvent,

As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used.

These organic solvents may be used alone or a combination of two or more kinds thereof may be used. Further, an organic solvent other than the above-described solvents and water may be mixed and used.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

According to the method for forming a resist pattern of the present embodiment described above, since the resist composition described above is used, a resist pattern that can achieve high sensitivity and has excellent lithography characteristics such as CDU can be formed.

It is preferable that various materials that are used in the resist composition according to the above-described embodiment and the method for forming a pattern according to the above-described embodiment (for example, a resist solvent, a developing solution, a rinse solution, a composition for forming an antireflection film, and a composition for forming a top coat) do not contain impurities such as a metal, a metal salt containing halogen, an acid, an alkali, and a component having a sulfur atom or phosphorus atom. Here, examples of the impurities containing metal atoms include Na, K, Ca, Fe, Cu, Mn, Mg, Al, Cr, Ni, Zn, Ag, Sn, Pb, Li, and salts thereof. The amount of the impurities contained in these materials is preferably 200 ppb or less, more preferably 1 ppb or less, still more preferably 100 parts per trillion (ppt) or less, particularly preferably 10 ppt or less, and most preferably substantially zero (less than or equal to the detection limit of the measuring device).

### (Compound)

The compound according to a third aspect is a compound represented by General Formula (dc01) (hereinafter, also referred to as “compound (Dc01)”). The compound (Dc01) is the same as the component (D01) of the first aspect except that Rd⁰¹ in Formula (d01) has at least two benzene rings which may have a substituent, and is limited to a monovalent organic group having at least one heteroatom.

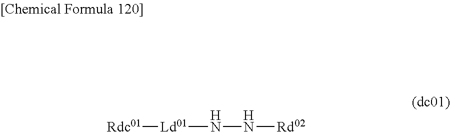

[In the formula, Rdc⁰¹ represents a monovalent organic group having at least two benzene rings which may have a substituent and having at least one heteroatom, Rd⁰² represents a monovalent organic group or a hydrogen atom, and Ld⁰¹ represents a single bond or a divalent linking group.]

It is preferable that the compound (Dc01) is a compound represented by General Formula (dc01-1) or (dc01-2).

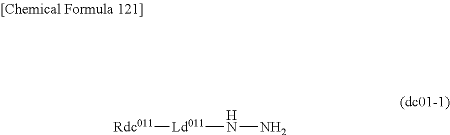

[In the formula, Rdc⁰¹¹ represents a monovalent organic group having at least two benzene rings which may have a substituent and having at least one heteroatom, and La⁰¹¹ represents a single bond or a divalent linking group.]

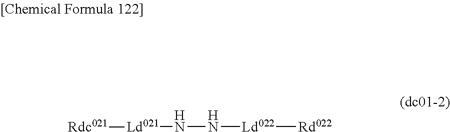

[In the formula, Rcd⁰²¹ represents a monovalent organic group having at least two benzene rings which may have a substituent and having at least one heteroatom, Rd⁰²² represents a monovalent organic group, and Ld⁰²¹ and Ld⁰²² each independently represent a single bond or a divalent linking group.]

It is preferable that Rdc⁰¹ in Formula (dc01), Rdc⁰¹¹ in Formula (dc01-1), and Rcd⁰²¹ in Formula (dc01-2) represent a group represented by General Formula (dc01-r-1) or (dc01-r-2).

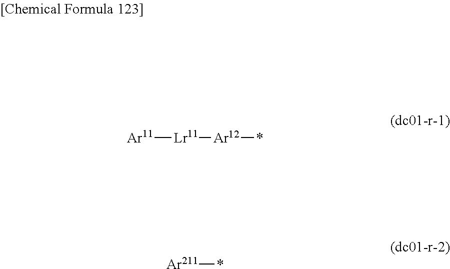

[In the formulae, Ar¹¹ represents a monovalent aromatic hydrocarbon group having at least one benzene ring which may have a substituent; Ar¹² represents a divalent aromatic hydrocarbon group having at least one benzene ring which may have a substituent; and Lr¹¹ represents —O— or —S—, where Ar¹¹ and Ar¹² may be bonded to each other to form a condensed ring together with Lr¹¹. Ar²¹¹ represents an aromatic hydrocarbon group having at least two benzene rings which may have a substituent and having at least one heteroatom. * represents a bonding site with respect to Ld⁰²¹.]

### <Method for Producing Compound (Dc01)>

A method for producing the compound (Dc01) is not particularly limited, and the compound (Dc01) can be produced by appropriately combining known methods.

A compound according to a fourth aspect is a compound represented by General Formula (d02-3) (hereinafter, also referred to as “compound (D023)”).

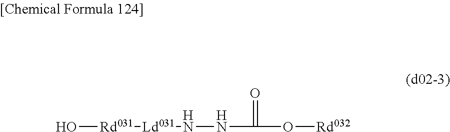

[In the formula, Rd⁰³¹ represents a divalent organic group, Rd⁰³² represents a monovalent organic group, and Ld⁰³¹ represents a single bond or a divalent linking group.]

Rd⁰³¹, Rd⁰³², and Ld⁰³¹ in General Formula (d02-3) each have the same definition as described above. It is preferable that the compound represented by General Formula (d02-3) is a compound represented by General Formula (d02-3-1).

### <Method for Producing Compound (D023)>

A method for producing the compound (D023) is not particularly limited, and a known method can be used. Examples of the method for producing the compound (D023) include a method of using the following reaction (I), reaction (II), or reaction (III).

### <<Reaction (I)>>

For example, the compound (D023) can be produced by the following reaction (I).

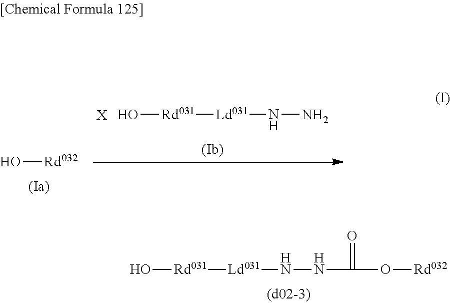

[In the formula, Rd⁰³¹, Rd⁰³², and Ld⁰³¹ each have the same definition as that for Rd⁰³¹ Rd⁰³², and Ld⁰³¹ in Formula (d02-3). X represents an active esterification agent.]

In the reaction (I), a compound represented by General Formula (d02-3) is obtained by reacting the active esterification agent (X) with a compound (la) and reacting the active esterification agent (X) with a compound (Ib).

The temperature conditions for the active esterification reaction in the reaction (I) are not particularly limited, and are, for example, about −10° C. to 120° C., preferably in a range of 0° C. to 100° C., more preferably in a range of 10° C. to 70° C., and still more preferably in a range of 10° C. to 50° C. The temperature conditions of the esterification reaction following the active esterification reaction are not particularly limited, and are, for example, about −10° C. to 120° C., preferably 0° C. to 100° C., more preferably 10° C. to 70° C., and still more preferably 10° C. to 50° C.

The reaction time of the active esterification reaction in the reaction (I) is not particularly limited, and is, for example, about 0.5 to 24 hours, preferably in a range of 1 to 12 hours, and more preferably in a range of 1 to 6 hours. The temperature conditions for the esterification reaction following the active esterification reaction are not particularly limited, and are, for example, about 1 to 72 hours and preferably in a range of 1 to 24 hours.

Examples of the active esterification agent used in the reaction (I) include 4-nitrophenyl chloroformate.

Examples of the reaction solvent used in the reaction (I) include dichloromethane, dichloroethane, chloroform, tetrahydrofuran, N,N-dimethylformamide, acetonitrile, propionitrile, N,N′-dimethylacetamide, and dimethyl sulfoxide.

A basic catalyst may be used in the reaction (I).

Specific examples of the basic catalyst include tertiary amines such as trimethylamine, triethylamine, and tributylamine, aromatic amines such as pyridine, dimethylaminopyridine (DMAP), and pyrrolidinopyridine, diazabicyclononene (DBN), and diazabicycloundecene (DBU).

### <<Reaction (II)>>

In a case where Ld⁰³¹ in Formula (d02-3) represents —C(═O)—, the compound may be produced by the following reaction (II).

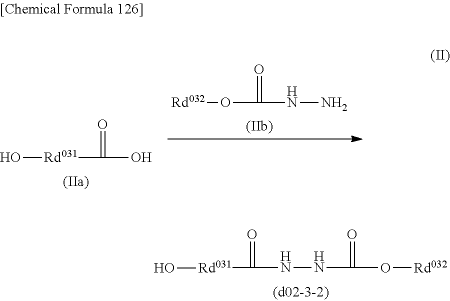

[In the formula, Rd⁰³¹ and Rd⁰³² each has the same definition as that for Rd⁰³¹ and Rd⁰³² in Formula (d02-3).]

In the reaction (II), a compound represented by General Formula (d02-3-2) is obtained by reacting a compound (IIa) with a compound (IIb).

The temperature conditions for the reaction (II) are not particularly limited, and are, for example, about-10° C. to 120° C., preferably in a range of 0° C. to 100° C., and more preferably in a range of 10° C. to 70° C.

The reaction time of the reaction (II) is not particularly limited, and is, for example, about 1 to 72 hours and preferably in a range of 1 to 24 hours.

Examples of the reaction solvent used in the above-described reaction (ii) include dichloromethane, dichloroethane, chloroform, tetrahydrofuran, N,N-dimethylformamide, acetonitrile, propionitrile, N,N′-dimethylacetamide, and dimethyl sulfoxide.

The condensation reaction in the reaction (II) may be carried out in the presence of a condensing agent.

Specific examples of the condensing agent include N,N′-dicyclohexylcarbodiimide, N,N′-diisopropylcarbodiimide (DIC), 1-ethyl-3-(3-dimethylaminopropyl) carbodiimide hydrochloride, and carbonyldiimidazole (CDI).

A basic catalyst may be used in the reaction (II).

Specific examples of the basic catalyst include tertiary amines such as trimethylamine, triethylamine, and tributylamine, aromatic amines such as pyridine, dimethylaminopyridine (DMAP), and pyrrolidinopyridine, diazabicyclononene (DBN), and diazabicycloundecene (DBU).

### <<Reaction (III)>>

In a case where -Ld⁰³¹-Rd⁰³¹-OH in Formula (d02-3) represents a 2-(hydroxymethyl)phenylcarbonyl group, the compound may be produced by the following reaction (III).

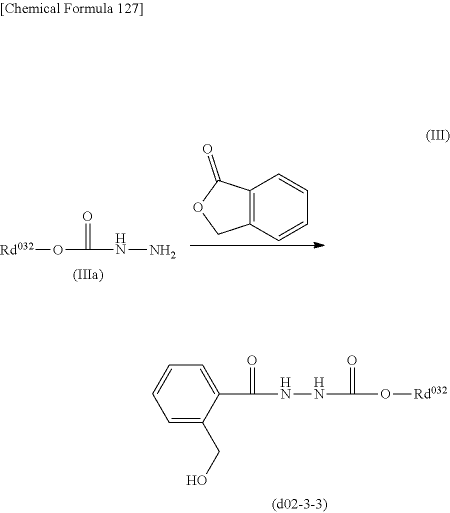

[In the formula, Rd⁰³² has the same definition as that for Rd⁰³² in Formula (d02-3).]

In the reaction (III), a compound represented by General Formula (d02-3-3) is obtained by reacting a compound (IIIa) with phthalide.

The temperature conditions for the reaction (III) are not particularly limited, and are, for example, about −10° C. to 120° C., preferably in a range of 0° C. to 100° C., and more preferably in a range of 10° C. to 70° C.

The reaction time of the reaction (I) is not particularly limited, and is, for example, about 1 to 72 hours and preferably in a range of 1 to 24 hours.

Examples of the reaction solvent used in the reaction (II) include dichloromethane, dichloroethane, chloroform, 4-methyltetrahydropyran, tetrahydrofuran, N,N-dimethylformamide, acetonitrile, propionitrile, N,N′-dimethylacetamide, and dimethyl sulfoxide.

The condensation reaction in the reaction (III) may be carried out in the presence of a condensing agent.

Specific examples of the condensing agent include N,N′-dicyclohexylcarbodiimide, N,N′-diisopropylcarbodiimide (DIC), 1-ethyl-3-(3-dimethylaminopropyl) carbodiimide hydrochloride, and carbonyldiimidazole (CDI).

A basic catalyst may be used in the reaction (III).

Specific examples of the basic catalyst include tertiary amines such as trimethylamine, triethylamine, and tributylamine, aromatic amines such as pyridine, dimethylaminopyridine (DMAP), and pyrrolidinopyridine, diazabicyclononene (DBN), and diazabicycloundecene (DBU).

After the completion of the reaction, the compound (D023) in the reaction solution may be isolated and purified.

Known methods of the related art can be used for the isolation and the purification, and for example, any one or a combination of two or more of concentrations, solvent extraction, distillation, crystallization, recrystallization, chromatography, and the like can be used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

A compound according to a fifth aspect is a compound represented by General Formula (d03-1-1) (hereinafter, also referred to as “compound (D0311)”).

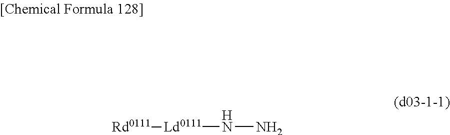

[In the formula, Rd⁰¹¹¹ represents a monovalent organic group having at least one iodine atom as a substituent, and Ld⁰¹¹¹ represents a divalent linking group.]

Rd⁰¹¹¹ and Ld⁰¹¹¹ in General Formula (d03-1-1) each have the same definition as described above.

A compound according to a sixth aspect is a compound represented by General Formula (d03-2-1) (hereinafter, also referred to as “compound (D0321)”).

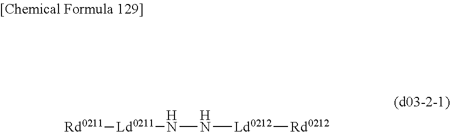

[In the formula, Rd⁰²¹¹ and Rd⁰²¹² each independently represent a monovalent organic group, and Ld⁰²¹¹ and Ld⁰²¹² each independently represent a divalent linking group. Here, at least one of Rd⁰²¹¹ or Rd⁰²¹² has at least one iodine atom as a substituent.]

Rd⁰²¹¹, Rd⁰²¹², Ld⁰²¹¹, and Ld⁰²¹² in General Formula (d03-2-1) each have the same definition as described above.

A compound according to a seventh aspect is a compound represented by General Formula (d03-2-2) (hereinafter, also referred to as “compound (D0322)”).

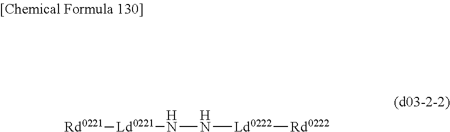

[In the formula, Rd⁰²²¹ represents a monovalent organic group, Rd⁰²²² represents a monovalent organic group having —(CH₂)ₚ—OH or a carboxy group as a substituent, p represents an integer of 1 to 3, and Ld⁰²²¹ and Ld⁰²²² each independently represent a single bond or a divalent linking group. Here, at least one of Rd⁰²²¹ or Rd⁰²²² has at least one iodine atom as a substituent.]

Rd⁰²¹¹, Rd⁰²¹², Ld⁰²¹¹, and Ld⁰²¹² in General Formula (d03-2-2) each have the same definition as described above.

### (Method for Producing Compound (D0311), (D0321), and (D0322))

A method for producing the compound (D0311), the compound (D0321), and the compound (D0322) according to the fifth to seventh aspects is not particularly limited, and the compounds can be produced by appropriately combining known methods.

The compound represented by Formula (D031-1), the compound represented by Formula (D031-4), the compound represented by Formula (D031-8), the compound represented by Formula (D032-2), and the like can be produced, for example, by reacting a phenol derivative having an iodine atom and a desired substituent with hydrazine, as described in [Synthesis Example 3-1: synthesis of compound (D03-1)] below.

The compound represented by Formula (D032-28), the compound represented by Formula (D032-30), the compound represented by Formula (D032-34), the compound represented by Formula (D032-40), and the like can be produced, for example, by a ring-opening reaction in which a reaction product of a phenol derivative having an iodine atom and a desired substituent and hydrazine reacts with a compound having a lactone ring, as described in [Synthesis Example 3-3: synthesis of compound (D03-3)] below.

The compound represented by Formula (D032-29) can be produced, for example, by reacting a reaction product of a phenol derivative having an iodine atom and a desired substituent with hydrazine, with an aromatic carboxylic acid halide, as described in [Synthesis Example 3-6: synthesis of compound (D03-6)] below.

The compound represented by Formula (D032-41) and the like can be produced, for example, by reacting a reaction product of a phenol derivative having an iodine atom and a desired substituent with hydrazine, with an aromatic carboxylic acid anhydride, as described in [Synthesis Example 3-12: synthesis of compound (D03-12)] below.

As described in [Synthesis Example 3-7: synthesis of compound (D03-7)] below, the compound represented by Formula (D031-7) and the like can be produced, for example, by reacting an aromatic thiol compound having an iodine atom with phenol or a phenol derivative to obtain a thioether compound which is an intermediate by the same method as in [Synthesis Example 3-1: synthesis of compound (D03-1)] below using the obtained thioether compound.

The compound and the like represented by Formula (D032-12) can be produced, for example, by reacting a reaction product of a phenol derivative having an iodine atom and a desired substituent with hydrazine, with lactic acid alkyl ester, as described in [Synthesis Example 3-9: synthesis of compound (D03-9)] below.

The compound and the like represented by Formulae (D032-42) to (D032-47) can be produced, for example, by a ring-opening reaction in which an aromatic hydrazine compound reacts with a compound having a lactone ring, as described in [Synthesis Example 3-13: synthesis of compound (D03-13)] below.

The compound and the like represented by Formula (D032-48) can be produced, for example, by reacting a hydrazide compound with a carboxylic acid halide, as described in [Synthesis Example 3-19: synthesis of compound (D03-19)] below.

The compound according to the present embodiment is useful as an acid diffusion control agent used in the resist composition.

### (Acid Diffusion Control Agent)

An acid diffusion control agent according to an eighth aspect of the present invention contains a compound represented by General Formula (d0) (compound (D0)).

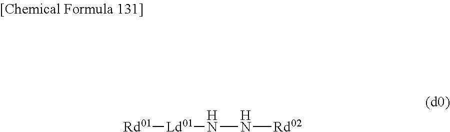

[In the formula, Rd⁰¹ represents a monovalent organic group, Rd⁰² represents a monovalent organic group or a hydrogen atom, and Ld⁰¹ represents a single bond or a divalent linking group.]

Rd⁰¹, Rd⁰², and Ld⁰¹ in General Formula (d0) each have the same definition as described above. The compound represented by General Formula (d0) may be a compound represented by General Formula (d0-1) or a compound represented by General Formula (d0-2).

The acid diffusion control agent according to the present embodiment may contain a compound represented by General Formula (d01) (compound (D01)) as the compound (D0).

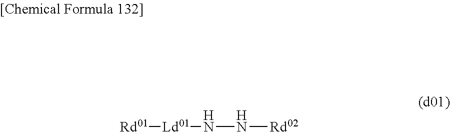

[In the formula, Rd⁰¹ represents a monovalent organic group having at least two benzene rings which may have a substituent; Rd⁰² represents a monovalent organic group or a hydrogen atom; and Ld⁰¹ represents a single bond or a divalent linking group.]

Rd⁰¹, Rd⁰², and Ld⁰¹ in General Formula (d01) each have the same definition as described above. The compound represented by General Formula (d01) may be a compound represented by General Formula (d01-1) or a compound represented by General Formula (d01-2).

The acid diffusion control agent according to the present embodiment may contain a compound represented by General Formula (d0) (compound (D02)) as the compound (D0).

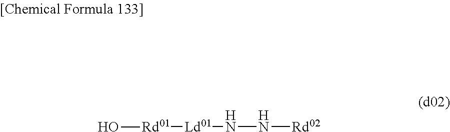

[In the formula, Rd⁰¹ represents a monovalent organic group, Rd⁰² represents a monovalent organic group or a hydrogen atom, and Ld⁰¹ represents a single bond or a divalent linking group.]

Rd⁰¹, Rd⁰², and Ld⁰¹ in General Formula (d02) each have the same definition as described above. The compound represented by General Formula (d02) may be a compound represented by General Formula (d02-1) or a compound represented by General Formula (d02-2). The compound represented by General Formula (d02-2) may be a compound represented by General Formula (d02-3) or a compound represented by General Formula (d02-3-1).

The acid diffusion control agent according to the present embodiment may contain a compound represented by General Formula (d03) (compound (D03)) as the compound (D0).

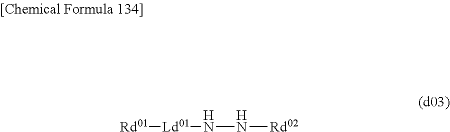

[In the formula, Rd⁰¹ represents a monovalent organic group, Rd⁰² represents a monovalent organic group or a hydrogen atom, and Ld⁰¹ represents a single bond or a divalent linking group. Here, at least one of Rd⁰¹ or Rd⁰² has at least one iodine atom as a substituent.]

Rd⁰¹, Rd⁰², and Ld⁰¹ in General Formula (d03) each have the same definition as described above. The compound represented by General Formula (d03) may be a compound represented by General Formula (d03-1) or a compound represented by General Formula (d03-2).

The acid diffusion control agent of the present embodiment can be used for producing the resist composition according to the first aspect.

### EXAMPLES

Hereinafter, the present invention will be described in more detail based on examples, but the present invention is not limited to these examples.

### First Embodiment

### <Preparation of Resist Composition>

### Examples 1 to 17 and Comparative Examples 1 and 2

Each of the components listed in Tables 1 and 2 was mixed and dissolved to prepare a resist composition of each example.

TABLE 1

| | Component | Component | | | Component |
|---|---|---|---|---|---|
| | (A) | (B) | Component (D) | | (S) |
| Example 1 | (A1)-1 | (B1)-1 | (D0)-1 | — | (S)-1 |
| | [100] | [30] | [10] | | [8000] |
| Example 2 | (A1)-1 | (B1)-1 | (D0)-2 | — | (S)-1 |
| | [100] | [30] | [10] | | [8000] |
| Example 3 | (A1)-1 | (B1)-1 | (D0)-3 | — | (S)-1 |
| | [100] | [30] | [10] | | [8000] |
| Example 4 | (A1)-1 | (B1)-1 | (D0)-4 | — | (S)-1 |
| | [100] | [30] | [10] | | [8000] |
| Example 5 | (A1)-1 | (B1)-1 | (D0)-5 | — | (S)-1 |
| | [100] | [30] | [10] | | [8000] |
| Example 6 | (A1)-1 | (B1)-1 | (D0)-6 | — | (S)-1 |
| | [100] | [30] | [10] | | [8000] |
| Example 7 | (A1)-1 | (B1)-1 | (D0)-7 | — | (S)-1 |
| | [100] | [30] | [10] | | [8000] |
| Example 8 | (A1)-1 | (B1)-1 | (D0)-8 | — | (S)-1 |
| | [100] | [30] | [10] | | [8000] |
| Example 9 | (A1)-1 | (B1)-1 | (D0)-9 | — | (S)-1 |
| | [100] | [30] | [10] | | [8000] |
| Example 10 | (A1)-1 | (B1)-1 | (D0)-10 | — | (S)-1 |
| | [100] | [30] | [10] | | [8000] |
| Example 11 | (A1)-1 | (B1)-1 | (D0)-11 | — | (S)-1 |
| | [100] | [30] | [10] | | [8000] |
| Example 12 | (A1)-2 | (B1)-1 | (D0)-4 | — | (S)-1 |
| | [100] | [30] | [10] | | [8000] |
| Example 13 | (A1)-3 | (B1)-1 | (D0)-4 | — | (S)-1 |
| | [100] | [15] | [10] | | [8000] |
| Example 14 | (A1)-3 | — | (D0)-4 | — | (S)-1 |
| | [100] | | [10] | | [8000] |
| Example 15 | (A1)-4 | (B1)-1 | (D0)-4 | — | (S)-1 |
| | [100] | [30] | [10] | | [8000] |
| Example 16 | (A1)-1 | (B1)-1 | (D0)-4 | (D1)-1 | (S)-1 |
| | [100] | [30] | [4] | [6] | [8000] |
| Example 17 | (A1)-1 | (B1)-2 | (D0)-4 | — | (S)-1 |
| | [100] | [30] | [10] | | [8000] |

TABLE 2

| | Component | Component | | | Component |
|---|---|---|---|---|---|
| | (A) | (B) | Component (D) | | (S) |
| Comparative | (A1)-1 | (B1)-1 | — | (D1)-2 | (S)-1 |
| Example 1 | [100] | [30] | | [10] | [8000] |
| Comparative | (A1)-1 | (B1)-1 | — | (D1)-1 | (S)-1 |
| Example 2 | [100] | [30] | | [10] | [8000] |

In Tables 1 and 2, each abbreviation has the following meaning. The numerical values in the brackets are blending amounts (parts by mass).

(A1)-1: polymer compound represented by Formula (A1-1). The weight-average molecular weight (Mw) determined by GPC measurement in terms of standard polystyrene was 7400, and the polydispersity (Mw/Mn) thereof was 1.67. The copolymer compositional ratio (l/m) (the proportion (molar ratio) of each constitutional unit in the structural formula) determined by ¹³C-NMR was 50/50.

(A1)-2: polymer compound represented by Formula (A1-2). The weight-average molecular weight (Mw) determined by the GPC measurement in terms of standard polystyrene was 7500, and the polydispersity (Mw/Mn) thereof was 1.63. The copolymer compositional ratio (l/m) (the proportion (molar ratio) of each constitutional unit in the structural formula) determined by ¹³C-NMR was 50/50.

(A1)-3: polymer compound represented by Formula (A1-3). The weight-average molecular weight (Mw) determined by the GPC measurement in terms of standard polystyrene was 8700, and the polydispersity (Mw/Mn) thereof was 1.72. The copolymer compositional ratio (l/m/n/o) (the proportion (molar ratio) of each constitutional unit in the structural formula) determined by ¹³C-NMR was 30/20/35/15.

(A1)-4: polymer compound represented by Formula (A1-4). The weight-average molecular weight (Mw) determined by the GPC measurement in terms of standard polystyrene was 7200, and the polydispersity (Mw/Mn) thereof was 1.66. The copolymer compositional ratio (l/m/n) (the proportion (molar ratio) of each constitutional unit in the structural formula) determined by ¹³C-NMR was 50/25/25.

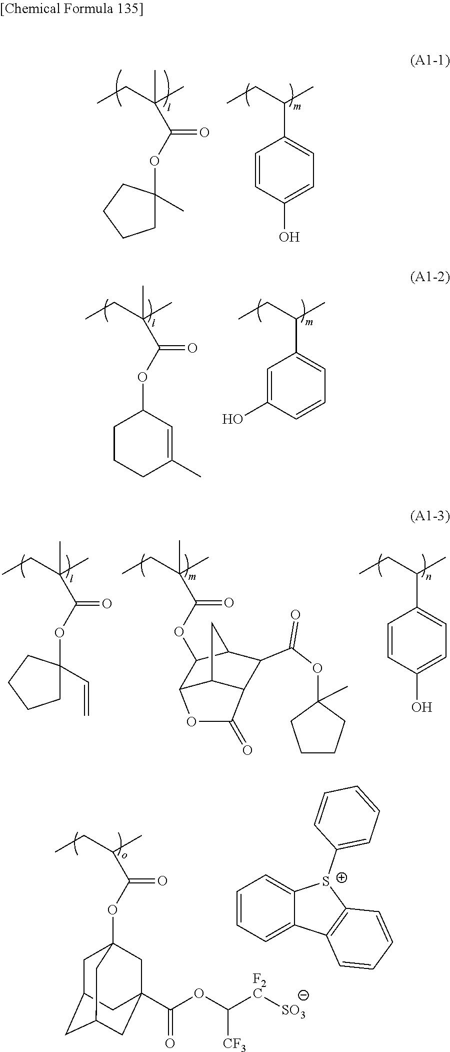

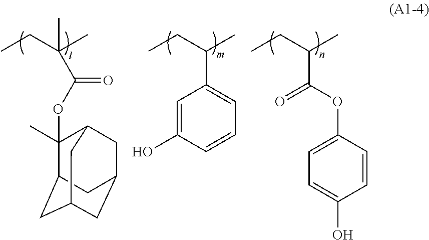

(B1)-1: acid generator consisting of compound (B1-1) shown below.

(B1)-2: acid generator consisting of compound (B1-2) shown below.

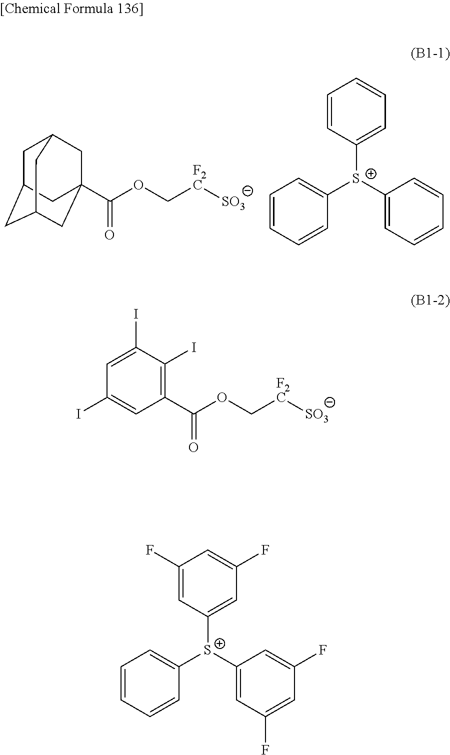

(D0)-1: acid diffusion control agent formed of compound (D0-1) shown below.

(D0)-2: acid diffusion control agent formed of compound (D0-2) shown below.

(D0)-3: acid diffusion control agent formed of compound (D0-3) shown below.

(D0)-4: acid diffusion control agent formed of compound (D0-4) shown below.

(D0)-5: acid diffusion control agent formed of compound (D0-5) shown below.

(D0)-6: acid diffusion control agent formed of compound (D0-6) shown below.

(D0)-7: acid diffusion control agent formed of compound (D0-7) shown below.

(D0)-8: acid diffusion control agent formed of compound (D0-8) shown below.

(D0)-9: acid diffusion control agent formed of compound (D0-9) shown below.

(D0)-10: acid diffusion control agent formed of compound (D0-10) shown below.

(D0)-11: acid diffusion control agent formed of compound (D0-11) shown below.

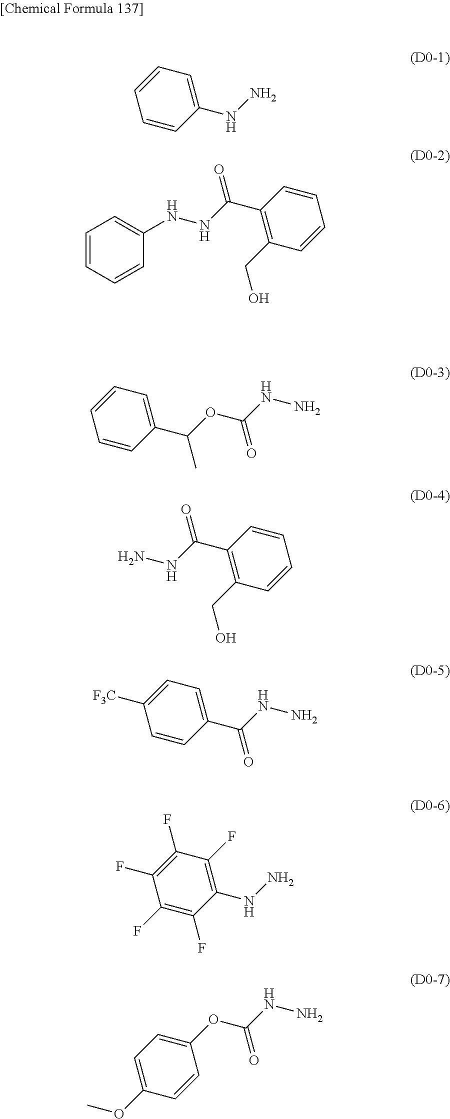

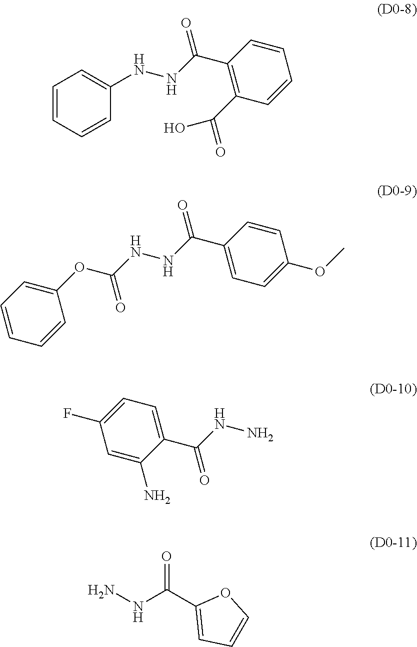 [SMILES: O=C(O)c1ccccc1C(=O)NNc1ccccc1 ; COc1ccc(C(=O)NNC(=O)Oc2ccccc2)cc1 ; NNC(=O)c1ccc(F)cc1N ; NNC(=O)c1ccco1]

(D1)-1: acid diffusion control agent consisting of compound (D1-1) shown below.

(D1)-2: acid diffusion control agent consisting of compound (D1-2) shown below.

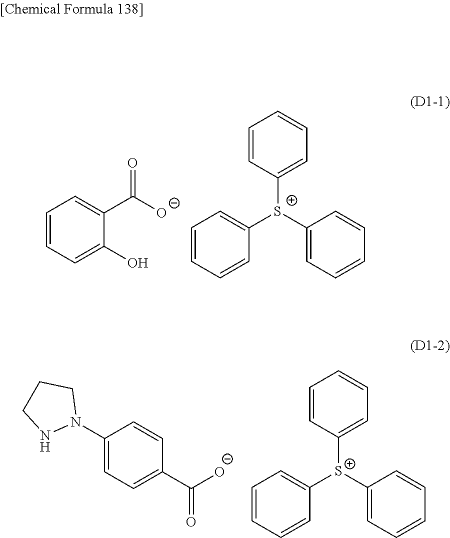 [SMILES: O=C([O-])c1ccccc1O ; c1ccc([S+](c2ccccc2)c2ccccc2)cc1 ; O=C([O-])c1ccc(N2CCCN2)cc1 ; c1ccc([S+](c2ccccc2)c2ccccc2)cc1]

(S)-1: mixed solvent of propylene glycol monomethyl ether acetate/propylene glycol monomethyl ether=60/40 (mass ratio).

### <Resist Pattern Formation>

Step (i): An 8-inch silicon substrate which had been subjected to a hexamethyldisilazane (HMDS) treatment was coated with the resist composition of each example using a spinner, and subjected to a pre-bake (PAB) treatment on a hot plate at a temperature of 110° C. for 60 seconds so that the 8-inch silicon substrate was dried, thereby forming a resist film having a film thickness of 50 nm.

Step (ii): Next, using an electron beam lithography apparatus JEOL JBX-9300FS (manufactured by JEOL Ltd.), the resist film was subjected to drawing (light exposure) of a hole pattern having a target size of a pitch width of 46 nm and a hole width of 23 nm at an acceleration voltage of 100 kV. Thereafter, a post exposure bake (PEB) treatment was performed thereon at 90° C. for 60 seconds.

Step (iii): Next, alkali development was performed for 60 seconds using a 2.38 mass % tetramethylammonium hydroxide (TMAH) aqueous solution “NMD-3” (trade name, manufactured by Tokyo Ohka Kogyo Co., Ltd.) at 23° C. Thereafter, water rinsing was carried out with pure water for 15 seconds. As a result, a hole pattern having a pitch width of 46 nm and a hole width of 23 nm was formed.

### [Evaluation of Optimum Exposure Amount (Eop)]

According to <Resist pattern formation> described above, an optimum exposure amount Eop (μC/cm²) in which a pattern having the target size was formed was determined. The results are listed in the columns of “Eop (μC/cm²)” in Tables 3 and 4.

### [Evaluation of Critical Dimension Uniformity (CDU) of Pattern Dimensions]

The CDU (dimensional variation 3σ) of the hole pattern formed in <Resist pattern formation> described above was determined. Here, “3σ” denotes the triple value (3σ) (unit: nm) of the standard deviation (σ) calculated from measurement results determined by measuring the dimensions of 50 holes with a scanning electron microscope (trade name: S-9380, manufactured by Hitachi High-Tech Corporation, acceleration voltage of 800 V). The results are listed in the columns of “CDU (nm)” in Tables 3 and 4.

The lower the value of 30 determined as described above is, the higher the critical dimension (CD) uniformity of the plurality of holes formed in the resist film is.

TABLE 3

| | Eop (μC/cm²) | CDU (nm) |
|---|---|---|
| Example 1 | 88 | 3.7 |
| Example 2 | 80 | 2.9 |
| Example 3 | 86 | 3.5 |
| Example 4 | 84 | 3.5 |
| Example 5 | 84 | 3.4 |
| Example 6 | 84 | 3.5 |
| Example 7 | 81 | 3.2 |
| Example 8 | 80 | 2.9 |
| Example 9 | 86 | 3.5 |
| Example 10 | 87 | 3.5 |
| Example 11 | 86 | 3.4 |
| Example 12 | 83 | 2.8 |
| Example 13 | 85 | 2.8 |
| Example 14 | 89 | 2.8 |
| Example 15 | 82 | 2.7 |
| Example 16 | 86 | 3.1 |
| Example 17 | 80 | 2.9 |

TABLE 4

| | Eop | CDU |
|---|---|---|
| | (μC/cm²) | (nm) |
| Comparative | 98 | 4.1 |
| Example 1 | | |
| Comparative | 92 | 4.0 |
| Example 2 | | |

As listed in Tables 3 and 4, the resist compositions of the examples had satisfactory sensitivity and satisfactory CDU as compared with those of the resist compositions of the comparative examples.

### Second Embodiment

### <Synthesis of Compounds>

### Synthesis Example 1-1: Synthesis of Compound (D01-1)

4-Phenoxyphenol (10 g) was added to substitute the inside of the system with argon. Dichloromethane (150 g) and p-nitrophenyl chloroformate (11 g) were added thereto, the reaction container was cooled to 0° C., and triethylamine (6 g) was added dropwise thereto. After the mixture was stirred at room temperature for 1 hour, a hydrazine monohydrate (4 g) was added dropwise thereto, and the mixture was stirred at room temperature for 3 hours. After the mixture was stirred, distilled water (50 g) was added to the reaction solution to stop the reaction, and the organic layer was extracted and washed with a 10% sodium carbonate aqueous solution and pure water. The obtained organic solvent was distilled off, and the resultant was purified by silica gel column chromatography, thereby obtaining a compound (D01-1) (11 g, yield=83%).

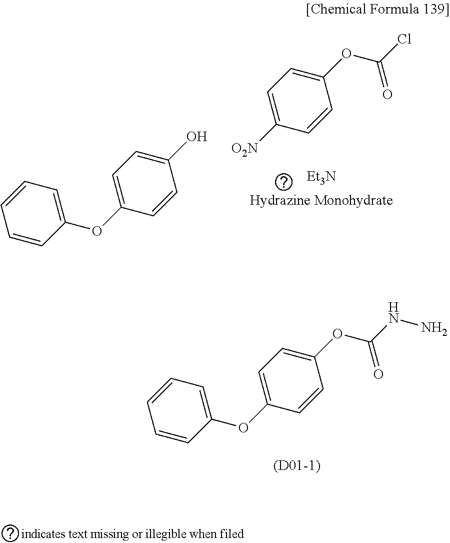 [SMILES: "Et3N" ; Oc1ccc(Oc2ccccc2)cc1 ; O=C(Cl)Oc1ccc([N+](=O)[O-])cc1 ; NNC(=O)Oc1ccc(Oc2ccccc2)cc1]

The ¹H NMR measurement results of the compound (D01-1) are shown below.

¹H NMR (400 MHz, DMSO-d6) δ (ppm)=8.89 (br, N—H, 1H), 7.5-7.0 (m, C═CH, C═CH, C═CH, C═CH, C═CH, C═CH, C═CH, C═CH, C═CH, C═CH, 9H), 4.30 (brs, NH₂, 2H)

### Synthesis Example 1-2: Synthesis of Compound (D01-2)

A compound (D01-2) (10 g, yield=69%) was obtained by the same method as in Synthesis Example 1-1 except that 4-phenoxyphenol was changed to 4-[(4-methylphenyl)thio]phenol at the equimolar amount.

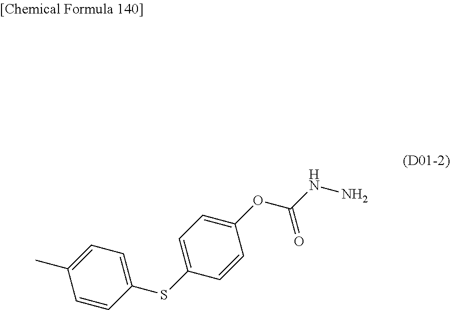 [SMILES: Cc1ccc(Sc2ccc(OC(=O)NN)cc2)cc1]

The ¹H NMR measurement results of the compound (D01-2) are shown below.

¹H NMR (400 MHz, DMSO-d6) δ (ppm)=8.99 (br, N—H, 1H), 7.6-7.0 (m, C═CH, C═CH, C═CH, C═CH, C═CH, C═CH, C═CH, C═CH, C═CH, 8H), 4.23 (brs, NH₂, 2H), 2.22 (s, CH₃, 3H)

### Synthesis Example 1-3: Synthesis of Compound (D01-3)

A compound (D01-4) (11 g, yield=77%) was obtained by the same method as in Synthesis Example 1-1 except that 4-phenoxyphenol was changed to 4-[(4-fluorophenyl)thio]phenol at the equimolar amount.

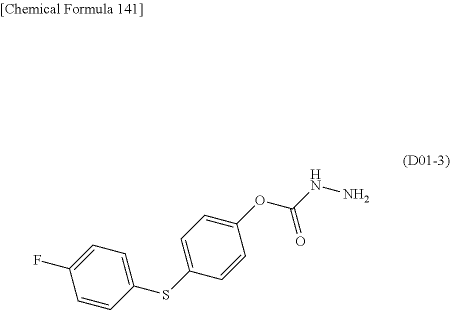 [SMILES: NNC(=O)Oc1ccc(Sc2ccc(F)cc2)cc1]

The ¹H NMR measurement results of the compound (D01-3) are shown below.

¹H NMR (400 MHz, DMSO-d6) δ (ppm)=8.85 (br, N—H, 1H), 7.6-6.8 (m, C═CH, C═CH, C═CH, C═CH, C═CH, C═CH, C═CH, C═CH, C═CH, 8H), 4.30 (brs, NH₂, 2H)

### Synthesis Example 1-4: Synthesis of Compound (D01-4)

A compound (D01-4) (10 g, yield=70%) was obtained by the same method as in Synthesis Example 1-1 except that 4-phenoxyphenol was changed to 4-[(4-methoxyphenoxy)]phenol at the equimolar amount.

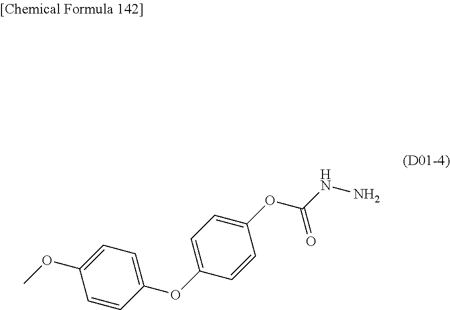 [SMILES: COc1ccc(Oc2ccc(OC(=O)NN)cc2)cc1]

The ¹H NMR measurement results of the compound (D01-4) are shown below.

¹H NMR (400 MHz, DMSO-d6) δ (ppm)=8.90 (br, N—H, 1H), 7.6-7.0 (m, C═CH, C═CH, C═CH, C═CH, C═CH, C═CH, C═CH, C═CH, C═CH, 8H), 4.29 (brs, NH₂, 2H), 3.81 (s, CH₃, 3H)

### Synthesis Example 1-5: Synthesis of Compound (D01-5)

4-Phenylthiophenol (10 g) was added to substitute the inside of the system with argon. Dichloromethane (100 g) and p-nitrophenyl chloroformate (11 g) were added thereto, the reaction container was cooled to 0° C., and triethylamine (6 g) was added dropwise thereto. After the mixture was stirred at room temperature for 1 hour, t-butyl carbazate (5 g) was added thereto, and the mixture was stirred at room temperature for 24 hours. After the mixture was stirred, distilled water (50 g) was added to the reaction solution to stop the reaction, and the organic layer was extracted and washed with a 10% sodium carbonate aqueous solution and pure water. The obtained organic solvent was distilled off, and the resultant was purified by silica gel column chromatography, thereby obtaining a compound (D01-5) (12 g, yield=67%).

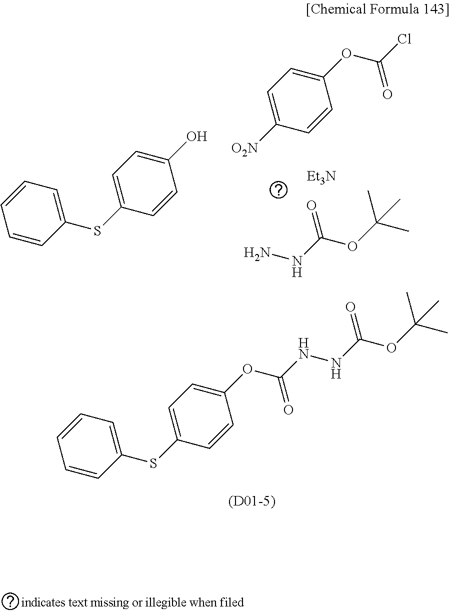

The ¹H NMR measurement results of the compound (D01-5) are shown below.

¹H NMR (400 MHz, DMSO-d6) δ (ppm)=10.21 (br, NH, NH, 2H), 7.6-7.0 (m, C═CH, C═CH, C═CH, C═CH, C═CH, C═CH, C═CH, C═CH, C═CH, C═CH, 9H), 1.48 (s, CH₃, CH₃, CH₃, 9H)

### Synthesis Example 1-6: Synthesis of Compound (D01-6)

A compound (D01-6) (9 g, yield=73%) was obtained by the same method as in Synthesis Example 1-1 except that 4-phenoxyphenol was changed to 4-methoxy-1-naphthol at the equimolar amount.

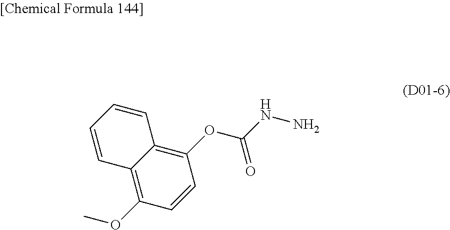 [SMILES: COc1ccc(OC(=O)NN)c2ccccc12]

The ¹H NMR measurement results of the compound (D01-6) are shown below.

¹H NMR (400 MHz, DMSO-d6) δ (ppm)=8.45 (d, C═CH, 1H), 7.80 (d, C═CH, 1H), 7.50 (m, C═CH, C═CH, 2H), 7.30 (d, C═CH, 1H), 6.80 (d, C═CH, 1H), 5.65 (brs, NH₂, 2H), 3.99 (s, CH₃, 3H)

### Synthesis Example 1-7: Synthesis of Compound (D01-7)

A mixture of a compound (D01-7-pre) (intermediate) was obtained by the same method as in Synthesis Example 1-1 except that 4-phenoxyphenol was changed to 2-hydroxydibenzofuran at the equimolar amount. The obtained compound (D01-7-pre) was dissolved in 4-methyltetrahydropyran (MTHP) (100 g) to substitute the inside of the system with argon, the container was cooled to 0° C., and p-nitrophenyl chloroformate (11 g) and triethylamine (6 g) were added thereto. The reaction container was returned to room temperature, the mixture was stirred for 2 hours, ethyl 2-hydroxybutyrate (9 g) was added thereto, and the mixture was stirred for 24 hours. The reaction solution was washed with 0.5% hydrochloric acid and pure water, the organic solvent was distilled off, and the resultant was purified by silica gel column chromatography, thereby obtaining a compound (D01-7) (10 g, yield=49%).

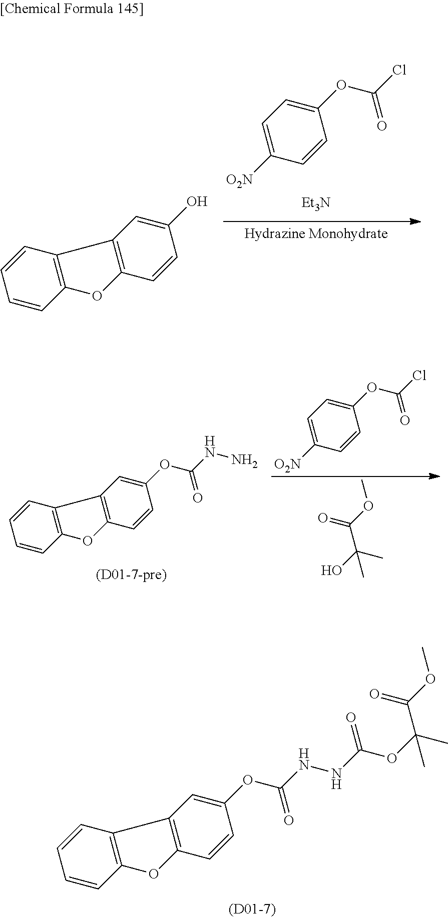

The ¹H NMR measurement results of the compound (D01-7) are shown below.

¹H NMR (400 MHz, DMSO-d6) δ (ppm)=11.21 (br, NH, NH, 2H), 8.0-7.0 (m, C═CH, C═CH, C═CH, C═CH, C═CH, C═CH, C═CH, 7H), 3.70 (s, CH₃, 3H), 1.46 (s, CH₃, CH₃, 6H)

### Synthesis Example 1-8: Synthesis of Compound (D01-8)

[4-(Phenoxy)phenyl]hydrazine (20 g) was added to substitute the inside of the system with argon. 4-Methyltetrahydropyran (MTHP) (200 g) and phthalide (26 g) were added thereto, and the mixture was stirred at room temperature for 24 hours. The reaction solution was washed twice with pure water, the organic solvent was distilled off to obtain a white solid, and the white solid was purified by silica gel column chromatography, thereby obtaining a compound (D01-8) (24 g, yield=72%).

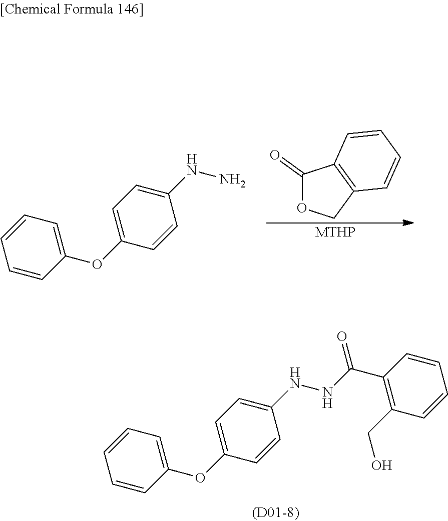

The ¹H NMR measurement results of the compound (D01-8) are shown below.

¹H NMR (400 MHz, DMSO-d6) δ (ppm)=9.10 (br, N—H, 1H), 8.67 (br, N—H, 1H), 8.0-7.0 (m, C═CH, C═CH, C═CH, C═CH, C═CH, C═CH, C═CH, C═CH, C═CH, 9H), 6.90-6.50 (m, C═CH, C═CH, C═CH, C═CH, 4H), 4.40 (brs, OH, 1H), 4.30 (s, —CH₂, 2H)

### Synthesis Example 1-9: Synthesis of Compound (D01-9)

A compound (D01-9) (25 g, yield=71%) was obtained by the same method as in Synthesis Example 1-8 except that [4-(phenoxy)phenyl]hydrazine was changed to [4-(phenylthio)phenyl]hydrazine at the equimolar amount.

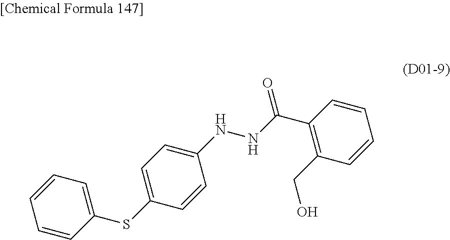 [SMILES: O=C(NNc1ccc(Sc2ccccc2)cc1)c1ccccc1CO]

The ¹H NMR measurement results of the compound (D01-9) are shown below.

¹H NMR (400 MHz, DMSO-d6) δ (ppm)=9.08 (br, N—H, 1H), 8.10 (br, N—H, 1H), 8.0-7.0 (m, C═CH, C═CH, C═CH, C═CH, C═CH, C═CH, C═CH, C═CH, C═CH, 9H), 6.90-6.50 (m, C═CH, C═CH, C═CH, C═CH, 4H), 4.60 (brs, OH, 1H), 4.25 (s, —CH₂, 2H)

### Synthesis Example 1-10: Synthesis of Compound (D01-10)

The compound (D01-6) (20 g) was added to substitute the inside of the system with argon. Dimethylformamide (DMF) (100 g) and Boc₂O (26 g) were added thereto, and the mixture was heated at 80° C. and stirred for 7 hours. Thereafter, the reaction solution was cooled to room temperature, MTHP (100 g) was added thereto, the organic layer extracted by washing the mixture three times with pure water was distilled off to obtain a white solid, and the white solid was purified by silica gel column chromatography, thereby obtaining a compound (D01-10) (21 g, yield=73%).

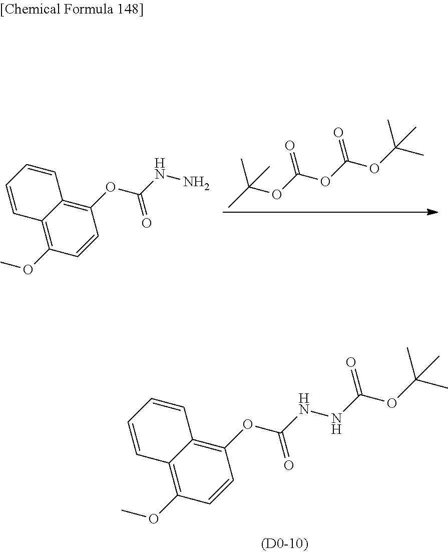 [SMILES: COc1ccc(OC(=O)NN)c2ccccc12 ; CC(C)(C)OC(=O)OC(=O)OC(C)(C)C ; COc1ccc(OC(=O)NNC(=O)OC(C)(C)C)c2ccccc12]

The ¹H NMR measurement results of the compound (D01-10) are shown below.

¹H NMR (400 MHz, DMSO-d6) δ (ppm)=8.50-7.50 (m, C═CH, C═CH, 2H), 6.01 (m, C═CH, C═CH, 1H), 5.65 (brs, NH₂, 2H), 3.99 (s, CH₃, 3H), 1.45 (s, CH₃, CH₃, CH₃, 9H)

### Synthesis Example 1-11: Synthesis of Compound (D01-11)

A compound (D01-11) (27 g, yield=58%) was obtained by the same method as in Synthesis Example 1-9 except that phthalide was changed to 4-bromophthalide at the equimolar amount.

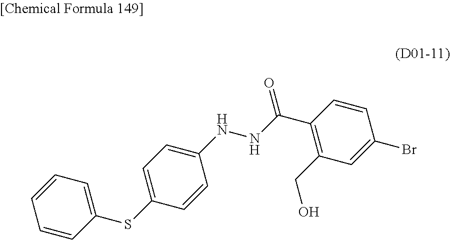 [SMILES: O=C(NNc1ccc(Sc2ccccc2)cc1)c1ccc(Br)cc1CO]

The ¹H NMR measurement results of the compound (D01-11) are shown below.

¹H NMR (400 MHz, DMSO-d6) δ (ppm)=9.11 (br, N—H, 1H), 8.10 (br, N—H, 1H), 8.0-7.0 (m, C═CH, C═CH, C═CH, C═CH, C═CH, C═CH, C═CH, C═CH, 8H), 6.90-6.50 (m, C═CH, C═CH, C═CH, C═CH, 4H), 4.42 (brs, OH, 1H), 4.40 (s, —CH₂, 2H)

### Synthesis Example 1-12: Synthesis of Compound (D01-12)

A compound (D01-12) (22 g, yield=66%) was obtained by the same method as in Synthesis Example 1-9 except that [4-(phenylthio)phenyl]hydrazine was changed to [4-(4-methoxyphenylthio)phenyl]hydrazine at the equimolar amount, and phthalide was changed to isocrotanolactone at the equimolar amount.

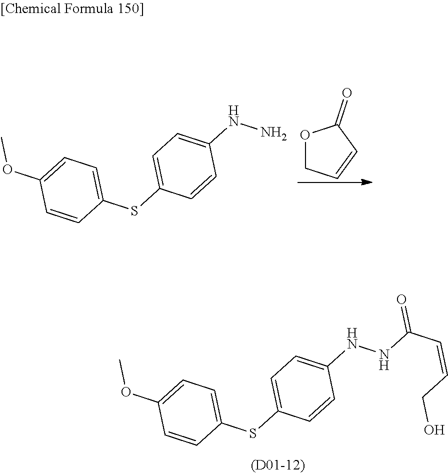

The ¹H NMR measurement results of the compound (D01-12) are shown below.

¹H NMR (400 MHz, DMSO-d6) δ (ppm)=9.11 (br, N—H, 1H), 7.90 (br, N—H, 1H), 8.5-7.5 (m, C═CH, C═CH, C═CH, C═CH, C═CH, C═CH, 6H), 7.40-6.40 (m, C═CH, C═CH, 2H), 4.89 (brs, OH, 1H), 4.20 (s, —CH₂, 2H), 3.51 (s, CH₃, 3H)

### <Preparation of Resist Composition>

### Examples 1-1 to 1-19 and Comparative Examples 1-1 to 1-4

Each of the components listed in Tables 5 and 6 was mixed and dissolved to 10 prepare a resist composition of each example.

TABLE 5

| | Component | Component | Component | | Component |
|---|---|---|---|---|---|
| | (A) | (B) | (D) | | (S) |
| Example 1-1 | (A1)-1 | (B1)-1 | (D01)-1 | — | (S)-1 |
| | [100] | [30] | [10] | | [8000] |
| Example 1-2 | (A1)-1 | (B1)-1 | (D01)-2 | — | (S)-1 |
| | [100] | [30] | [10] | | [8000] |
| Example 1-3 | (A1)-1 | (B1)-1 | (D01)-3 | — | (S)-1 |
| | [100] | [30] | [10] | | [8000] |
| Example 1-4 | (A1)-1 | (B1)-1 | (D01)-4 | — | (S)-1 |
| | [100] | [30] | [10] | | [8000] |
| Example 1-5 | (A1)-1 | (B1)-1 | (D01)-5 | — | (S)-1 |
| | [100] | [30] | [10] | | [8000] |
| Example 1-6 | (A1)-1 | (B1)-1 | (D01)-6 | — | (S)-1 |
| | [100] | [30] | [10] | | [8000] |
| Example 1-7 | (A1)-1 | (B1)-1 | (D01)-7 | — | (S)-1 |
| | [100] | [30] | [10] | | [8000] |
| Example 1-8 | (A1)-1 | (B1)-1 | (D01)-8 | — | (S)-1 |
| | [100] | [30] | [10] | | [8000] |
| Example 1-9 | (A1)-1 | (B1)-1 | (D01)-9 | — | (S)-1 |
| | [100] | [30] | [10] | | [8000] |
| Example 1-10 | (A1)-1 | (B1)-1 | (D01)-10 | — | (S)-1 |
| | [100] | [30] | [10] | | [8000] |
| Example 1-11 | (A1)-1 | (B1)-1 | (D01)-11 | — | (S)-1 |
| | [100] | [30] | [10] | | [8000] |
| Example 1-12 | (A1)-1 | (B1)-1 | (D01)-12 | — | (S)-1 |
| | [100] | [30] | [10] | | [8000] |
| Example 1-13 | (A1)-1 | (B1)-1 | (D01)-13 | — | (S)-1 |
| | [100] | [30] | [10] | | [8000] |
| Example 1-14 | (A1)-2 | (B1)-1 | (D01)-13 | — | (S)-1 |
| | [100] | [30] | [10] | | [8000] |
| Example 1-15 | (A1)-3 | (B1)-1 | (D01)-13 | — | (S)-1 |
| | [100] | [15] | [10] | | [8000] |
| Example 1-16 | (A1)-3 | — | (D01)-13 | — | (S)-1 |
| | [100] | | [10] | | [8000] |
| Example 1-17 | (A1)-4 | (B1)-1 | (D01)-13 | — | (S)-1 |
| | [100] | [30] | [10] | | [8000] |
| Example 1-18 | (A1)-1 | (B1)-1 | (D01)-13 | (D1)-1 | (S)-1 |
| | [100] | [30] | [4] | [6] | [8000] |
| Example 1-19 | (A1)-1 | (B1)-2 | (D01)-13 | — | (S)-1 |
| | [100] | [30] | [10] | | [8000] |

TABLE 6

| | Component | Component | Component | Component |
|---|---|---|---|---|
| | (A) | (B) | (D) | (S) |
| Comparative | (A1)-1 | (B1)-1 | (D2)-1 | (S)-1 |
| Example 1-1 | [100] | [30] | [10] | [8000] |
| Comparative | (A1)-1 | (B1)-1 | (D2)-2 | (S)-1 |
| Example 1-2 | [100] | [30] | [10] | [8000] |
| Comparative | (A1)-1 | (B1)-1 | (D1)-2 | (S)-1 |
| Example 1-3 | [100] | [30] | [10] | [8000] |
| Comparative | (A1)-1 | (B1)-1 | (D1)-1 | (S)-1 |
| Example 1-4 | [100] | [30] | [10] | [8000] |

In Tables 5 and 6, each abbreviation has the following meaning. The numerical values in the brackets are blending amounts (parts by mass).

(A1)-1: polymer compound represented by Formula (A1-1).

(A1)-2: polymer compound represented by Formula (A1-2).

(A1)-3: polymer compound represented by Formula (A1-3).

(A1)-4: polymer compound represented by Formula (A1-4).

(B1)-1: acid generator formed of compound (B1-1) shown above.

(B1)-2: acid generator formed of compound (B1-2) shown above.

(D01)-1: acid diffusion control agent formed of compound (D01-1) shown below

(D01)-2: acid diffusion control agent formed of compound (D01-2) shown below

(D01)-3: acid diffusion control agent formed of compound (D01-3) shown below

(D01)-4: acid diffusion control agent formed of compound (D01-4) shown below

(D01)-5: acid diffusion control agent formed of compound (D01-5) shown below

(D01)-6: acid diffusion control agent formed of compound (D01-6) shown below

(D01)-7: acid diffusion control agent formed of compound (D01-7) shown below

(D01)-8: acid diffusion control agent formed of compound (D01-8) shown below

(D01)-9: acid diffusion control agent formed of compound (D01-9) shown below

(D01)-10: acid diffusion control agent formed of compound (D01-10) shown below

(D01)-11: acid diffusion control agent formed of compound (D01-11) shown below (D01)-12: acid diffusion control agent formed of compound (D01-12) shown below

(D01)-13: acid diffusion control agent formed of compound (D01-13) shown below

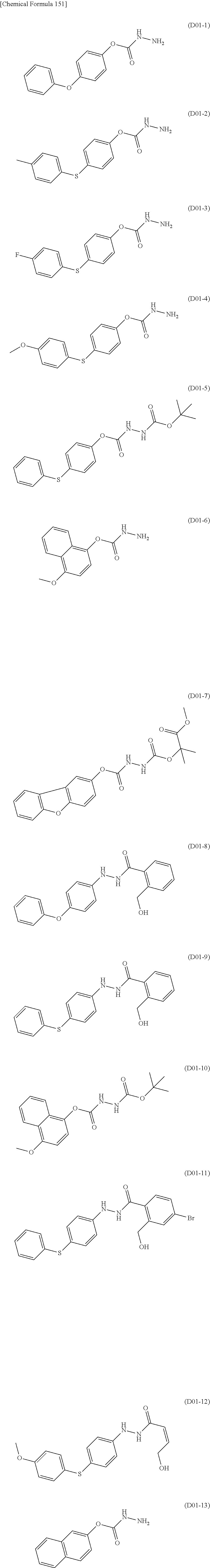

(D1)-1: acid diffusion control agent formed of compound (D1-1) shown above.

(D1)-2: acid diffusion control agent formed of compound (D1-2) shown above.

(D2)-1: acid diffusion control agent formed of compound (D2-1) shown below.

(D2)-2: acid diffusion control agent formed of compound (D2-2) shown below.

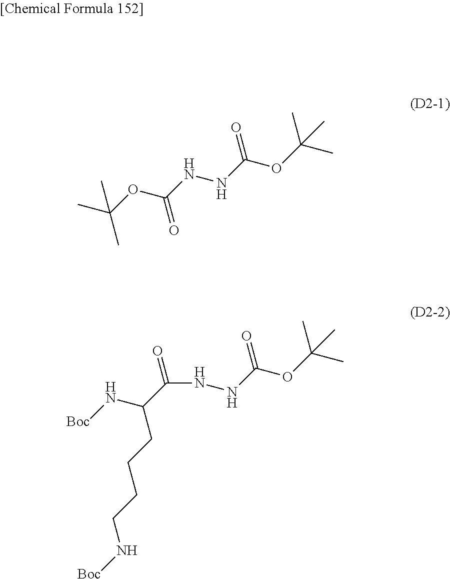 [SMILES: CC(C)(C)OC(=O)NNC(=O)OC(C)(C)C ; CC(C)(C)OC(=O)NCCCCC(NC(=O)OC(C)(C)C)C(=O)NNC(=O)OC(C)(C)C]

(S)-1: mixed solvent of propylene glycol monomethyl ether acetate/propylene glycol monomethyl ether=60/40 (mass ratio).

### <Resist Pattern Formation>

Step (i): An 8-inch silicon substrate which had been subjected to a hexamethyldisilazane (HMDS) treatment was coated with the resist composition of each example using a spinner, and subjected to a pre-bake (PAB) treatment on a hot plate at a temperature of 110° C. for 60 seconds so that the 8-inch silicon substrate was dried, thereby forming a resist film having a film thickness of 50 nm.

Step (ii): Next, using an electron beam lithography apparatus JEOL JBX-9300FS (manufactured by JEOL Ltd.), the resist film was subjected to drawing (light exposure) of a hole pattern having a target size of a pitch width of 46 nm and a hole width of 23 nm at an acceleration voltage of 100 kV. Thereafter, a post exposure bake (PEB) treatment was performed thereon at 90° C. for 60 seconds.

Step (iii): Next, alkali development was performed for 60 seconds using a 2.38 mass % tetramethylammonium hydroxide (TMAH) aqueous solution “NMD-3” (trade name, manufactured by Tokyo Ohka Kogyo Co., Ltd.) at 23° C. Thereafter, water rinsing was carried out with pure water for 15 seconds. As a result, a hole pattern having a pitch width of 46 nm and a hole width of 23 nm was formed.

### [Evaluation of Optimum Exposure Amount (Eop)]

According to <Resist pattern formation> described above, an optimum exposure amount Eop (μC/cm²) in which a pattern having the target size was formed was determined. The results thereof are listed in the columns of “Eop (μC/cm²)” in Tables 7 and 8.

### [Evaluation of Critical Dimension Uniformity (CDU) of Pattern Dimensions]

The CDU (dimensional variation 3σ) of the hole pattern formed in <Resist pattern formation> described above was determined. Here, “3σ” denotes the triple value (3σ) (unit: nm) of the standard deviation (σ) calculated from measurement results determined by measuring the dimensions of 50 holes with a scanning electron microscope (trade name: S-9380, manufactured by Hitachi High-Tech Corporation, acceleration voltage of 800 V). The results thereof are listed in the columns of “CDU (nm)” in Tables 7 and 8.

The lower the value of 36 determined as described above is, the higher the critical dimension (CD) uniformity of the plurality of holes formed in the resist film is.

TABLE 7

| | Eop | CDU |
|---|---|---|
| | (μC/cm²) | (nm) |
| Example 1-1 | 85 | 3.6 |
| Example 1-2 | 81 | 3.2 |
| Example 1-3 | 86 | 3.7 |
| Example 1-4 | 82 | 3.3 |
| Example 1-5 | 83 | 3.5 |
| Example 1-6 | 81 | 3.4 |
| Example 1-7 | 82 | 3.5 |
| Example 1-8 | 79 | 3.2 |
| Example 1-9 | 79 | 3.1 |
| Example 1-10 | 78 | 3.1 |
| Example 1-11 | 80 | 3.2 |
| Example 1-12 | 80 | 3.2 |
| Example 1-13 | 82 | 3.3 |
| Example 1-14 | 82 | 3.1 |
| Example 1-15 | 81 | 3.1 |
| Example 1-16 | 80 | 3.2 |
| Example 1-17 | 81 | 3.1 |
| Example 1-18 | 87 | 3.8 |
| Example 1-19 | 82 | 3.2 |

TABLE 8

| | Eop | CDU |
|---|---|---|
| | (μC/cm²) | (nm) |
| Comparative | 96 | 4.5 |
| Example 1-1 | | |
| Comparative | 110 | 4.1 |
| Example 1-2 | | |
| Comparative | 98 | 4.1 |
| Example 1-3 | | |
| Comparative | 92 | 4.0 |
| Example 1-4 | | |

As listed in Tables 7 and 8, the resist compositions of the examples had satisfactory sensitivity and satisfactory CDU as compared with the resist compositions of the comparative examples.

### Third Embodiment

### <Synthesis of Compounds>

### Synthesis Example 2-1

Methyl carbazate (10 g) was added to a 500 mL eggplant flask to substitute the inside of the system with argon. 4-Methyltetrahydropyran (MTHP) (100 g) and phthalide (20 g) were added thereto, and the mixture was heated to 50° C. and stirred for 24 hours. The reaction solution was washed twice with pure water, and the organic solvent was distilled off to obtain a white solid, and the white solid was purified by silica gel column chromatography, thereby obtaining a compound (D02-1) (10 g, yield=40%).

The ¹H NMR measurement results of the compound (D02-1) are shown below.

¹H NMR (400 MHz, DMSO-d6) δ (ppm)=11.27 (br, N—H, 1H), 10.22 (br, N—H, 1H), 8.0-7.1 (m, C═CH, C═CH, C═CH, C═CH, 4H), 5.25 (brs, OH, 1H), 4.61 (s, CH₂, 2H), 3.88 (s, CH₃, 3H)

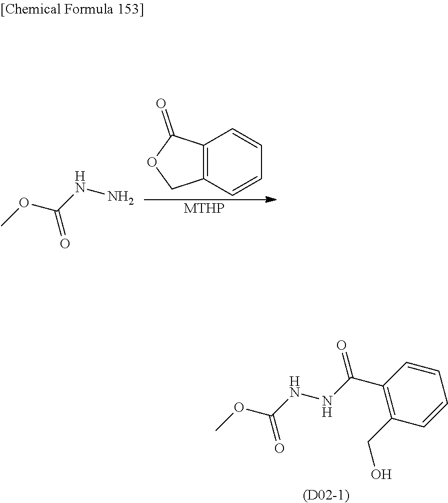

### Synthesis Example 2-2

A compound (D02-2) (10 g, yield=40%) was obtained by the same method as in Synthesis Example 2-1 except that methyl carbazate was changed to t-butyl carbazate.

The ¹H NMR measurement results of the compound (D02-2) are shown below.

¹H NMR (400 MHz, DMSO-d6) δ (ppm)=11.3 (br, N—H, 1H), 10.46 (br, N—H, 1H), 8.0-7.1 (m, C═CH, C═CH, C═CH, C═CH, 4H), 5.25 (brs, OH, 1H), 4.60 (s, CH₂, 2H), 1.53 (s, CH₃, CH₃, CH₃, 9H)

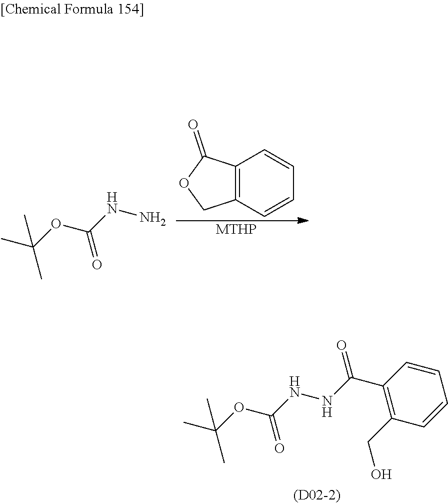

### Synthesis Example 2-3

4-Methoxy-1-naphthol (10 g) and dichloromethane were added to a 300 mL eggplant flask to substitute the inside of the system with argon. Triethylamine (TEA) (3 g) and 4-nitrophenyl chloroformate (11 g) were added thereto, the mixture was stirred at 25° C. for 1 hour, a hydrazine monohydrate (2 g) was added thereto, and the mixture was stirred for 30 minutes. Phthalide (12 g) was added to the solution, the mixture was stirred at room temperature for 24 hours, the reaction solution was washed twice with pure water, and the organic solvent was distilled off. The obtained white solid was purified by silica gel column chromatography, thereby obtaining a compound (D02-3) (9 g, yield=45%).

The ¹H NMR measurement results of the compound (D02-3) are shown below.

¹H NMR (400 MHz, DMSO-d6) δ (ppm)=11.3 (br, N—H, 1H), 10.66 (br, N—H, 1H), 8.5-7.0 (m, C═CH, C═CH, C═CH, C═CH, C═CH, C═CH, C═CH, C═CH, 8H), 5.9-5.8 (m, C═CH, C═CH, 2H), 5.31 (brs, OH, 1H), 4.60 (s, CH₂, 2H), 3.79 (s, CH₃, 3H)

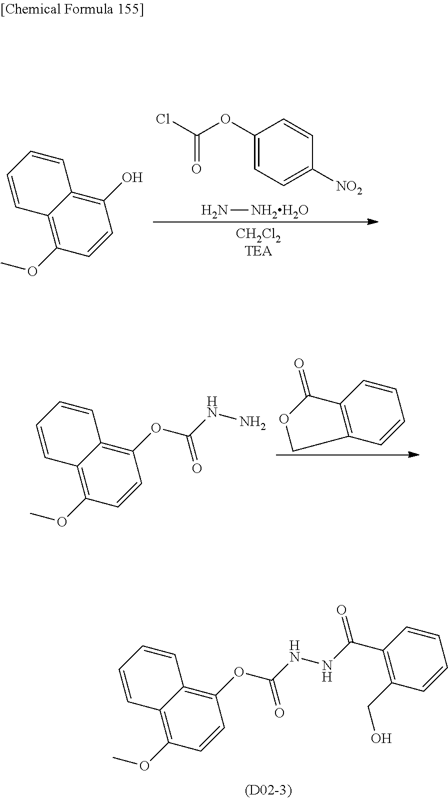

### Synthesis Example 2-4

3-(Hydroxymethyl)benzoic acid (10 g) and phenyl carbazate (8 g) were added to a 500 mL eggplant flask and dissolved in dichloromethane (DCM) (200 g) to substitute the inside of the system with argon. Next, N,N′-diisopropylcarbodiimide (DIC) (9 g) was added dropwise to the solution, and the solution was stirred at 25° C. for 24 hours. The reaction solution was washed twice with pure water, and the solid obtained by distilling off the organic solvent was purified by silica gel column chromatography, thereby obtaining a compound (D02-4) (9.4 g, yield=50%).

The ¹H NMR measurement results of the compound (D02-4) are shown below.

¹H NMR (400 MHz, DMSO-d6) δ (ppm)=11.3 (br, N—H, 1H), 10.62 (br, N—H, 1H), 8.0-7.0 (m, C═CH, C═CH, C═CH, C═CH, C═CH, C═CH, C═CH, C═CH, C═CH, 9H), 5.32 (brs, OH, 1H), 4.63 (s, CH₂, 2H)

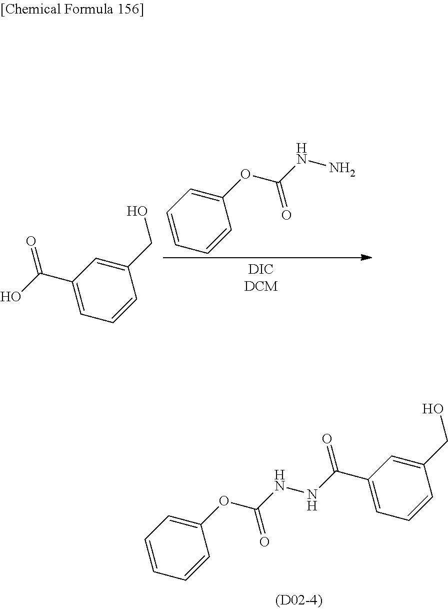

### Synthesis Example 2-5

Methyl lactate (10 g) was added to a 500 mL eggplant flask and dissolved in DCM (100 g) to substitute the inside of the system with argon. Next, 4-nitrophenyl chloroformate (6 g) and triethylamine (TEA) (7 g) were added to the solution, and the solution was stirred at 25° C. for 2 hours. 2-Hydroxymethylbenzohydrazide (18 g) was added to the reaction solution, and the reaction solution was stirred at room temperature for 24 hours. The reaction solution was washed twice with pure water, and the organic solvent was distilled off to obtain a solid, and the solid was purified by silica gel column chromatography, thereby obtaining a compound (D02-5) (18.6 g, yield=63%).

The ¹H NMR measurement results of the compound (D02-5) are shown below.

¹H NMR (400 MHz, DMSO-d6) δ (ppm)=11.24 (br, N—H, 1H), 10.80 (br, N—H, 1H), 8.0-7.1 (m, C═CH, C═CH, C═CH, C═CH, 4H), 5.40 (q, CH, 1H), 5.25 (brs, OH, 1H), 4.61 (s, CH₂, 2H), 3.80 (s, CH₃, 3H), 1.55 (d, CH₃, 3H)

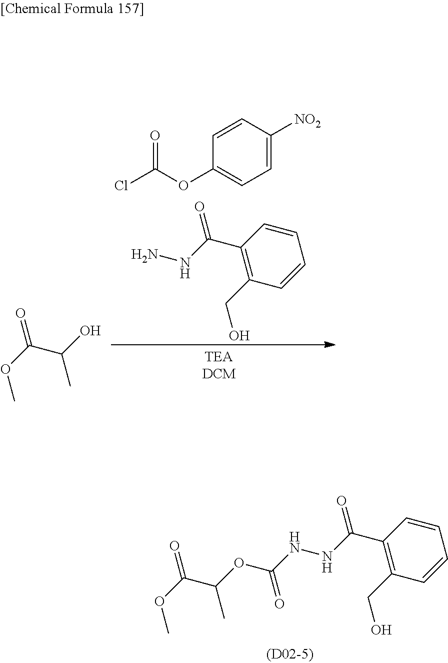

### Synthesis Example 2-6

A compound (D02-6) (17 g, yield=43%) was obtained by the same method as in Synthesis Example 2-5 except that methyl lactate was changed to 4-[(4-methylphenyl)thio]phenol.

The ¹H NMR measurement results of the compound (D02-6) are shown below.

¹H NMR (400 MHz, DMSO-d6) δ (ppm)=11.3 (br, N—H, 1H), 10.66 (br, N—H, 1H), 8.0-6.80 (m, C═CH, C═CH, C═CH, C═CH, C═CH, C═CH, C═CH, C═CH, C═CH, C═CH, C═CH, C═CH, 12H), 5.31 (brs, OH, 1H), 4.60 (s, CH₂, 2H), 3.21 (s, CH₃, 3H)

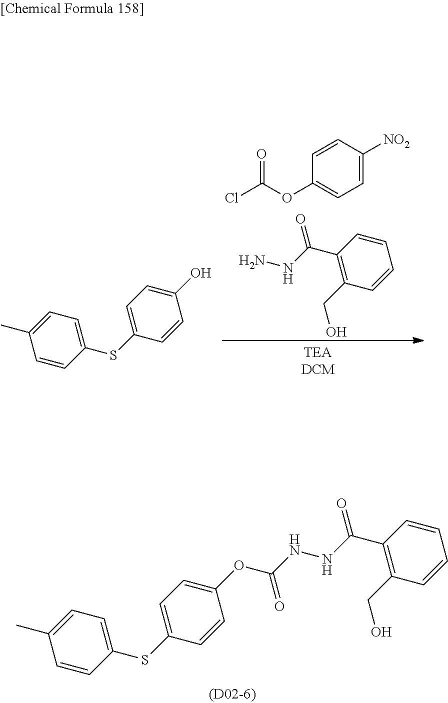

### Synthesis Example 2-7

A compound (D02-7) (20 g, yield=53%) was obtained by the same method as in Synthesis Example 2-5 except that methyl lactate was changed to 4-phenoxyphenol.

The ¹H NMR measurement results of the compound (D02-7) are shown below.

¹H NMR (400 MHz, DMSO-d6) δ (ppm)=11.27 (br, N—H, 1H), 10.49 (br, N—H, 1H), 8.0-6.80 (m, C═CH, C═CH, C═CH, C═CH, C═CH, C═CH, C═CH, C═CH, C═CH, C═CH, C═CH, C═CH, C═CH, 13H), 5.30 (brs, OH, 1H), 4.61 (s, CH₂, 2H)

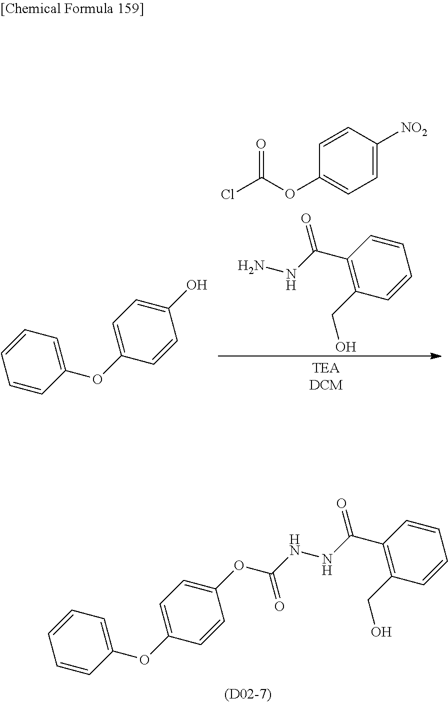

### <Preparation of Resist Composition>

### Examples 2-1 to 2-13 and Comparative Examples 2-1 to 2-4

Each of the components listed in Tables 9 and 10 was mixed and dissolved to prepare a resist composition of each example.

TABLE 9

| | Component | Component | Component | | Component |
|---|---|---|---|---|---|
| | (A) | (B) | (D) | | (S) |
| Example | (A1)-1 | (B1)-1 | (D02)-1 | — | (S)-1 |
| 2-1 | [100] | [30] | [10] | | [8000] |
| Example | (A1)-1 | (B1)-1 | (D02)-2 | — | (S)-1 |
| 2-2 | [100] | [30] | [10] | | [8000] |
| Example | (A1)-1 | (B1)-1 | (D02)-3 | — | (S)-1 |
| 2-3 | [100] | [30] | [10] | | [8000] |
| Example | (A1)-1 | (B1)-1 | (D02)-4 | — | (S)-1 |
| 2-4 | [100] | [30] | [10] | | [8000] |
| Example | (A1)-1 | (B1)-1 | (D02)-5 | — | (S)-1 |
| 2-5 | [100] | [30] | [10] | | [8000] |
| Example | (A1)-1 | (B1)-1 | (D02)-6 | — | (S)-1 |
| 2-6 | [100] | [30] | [10] | | [8000] |
| Example | (A1)-1 | (B1)-1 | (D02)-7 | — | (S)-1 |
| 2-7 | [100] | [30] | [10] | | [8000] |
| Example | (A1)-1 | (B1)-1 | (D02)-8 | — | (S)-1 |
| 2-8 | [100] | [30] | [10] | | [8000] |
| Example | (A1)-2 | (B1)-1 | (D02)-1 | — | (S)-1 |
| 2-9 | [100] | [30] | [10] | | [8000] |
| Example | (A1)-3 | (B1)-1 | (D02)-1 | — | (S)-1 |
| 2-10 | [100] | [5] | [10] | | [8000] |
| Example | (A1)-3 | — | (D02)-1 | — | (S)-1 |
| 2-11 | [100] | | [10] | | [8000] |
| Example | (A1)-4 | (B1)-1 | (D02)-1 | — | (S)-1 |
| 2-12 | [100] | [30] | [10] | | [8000] |
| Example | (A1)-1 | (B1)-1 | (D02)-1 | (D1)-1 | (S)-1 |
| 2-13 | [100] | [30] | [4] | [6] | [8000] |
| Example | (A1)-1 | (B1)-2 | (D02)-1 | — | (S)-1 |
| 2-14 | [100] | [30] | [10] | | [8000] |

TABLE 10

| | Component | Component | Component | | Component |
|---|---|---|---|---|---|
| | (A) | (B) | (D) | | (S) |
| Comparative | (A1)-1 | (B1)-1 | (D2)-1 | | (S)-1 |
| Example 2-1 | [100] | [30] | [10] | | [8000] |
| Comparative | (A1)-1 | (B1)-1 | (D2)-2 | | (S)-1 |
| Example 2-2 | [100] | [30] | [10] | | [8000] |
| Comparative | (A1)-1 | (B1)-1 | — | (D1)-1 | (S)-1 |
| Example 2-3 | [100] | [30] | | [10] | [8000] |
| Comparative | (A1)-1 | (B1)-1 | — | (D1)-2 | (S)-1 |
| Example 2-4 | [100] | [30] | | [10] | [8000] |

In Tables 9 and 10, each abbreviation has the following meaning. The numerical values in the brackets are blending amounts (parts by mass).

(A1)-1: polymer compound represented by Formula (A1-1).

(A1)-2: polymer compound represented by Formula (A1-2).

(A1)-3: polymer compound represented by Formula (A1-3).

(A1)-4: polymer compound represented by Formula (A1-4).

(B1)-1: acid generator formed of compound (B1-1) shown above.

(B1)-2: acid generator formed of compound (B1-2) shown above.

(D02)-1: acid diffusion control agent formed of compound (D02-1) shown above.

(D02)-2: acid diffusion control agent formed of compound (D02-2) shown above.

(D02)-3: acid diffusion control agent formed of compound (D02-3) shown above.

(D02)-4: acid diffusion control agent formed of compound (D02-4) shown above.

(D02)-5: acid diffusion control agent formed of compound (D02-5) shown above.

(D02)-6: acid diffusion control agent formed of compound (D02-6) shown above.

(D02)-7: acid diffusion control agent formed of compound (D02-7) shown above.

(D02)-8: acid diffusion control agent formed of compound (D02-8) shown below.

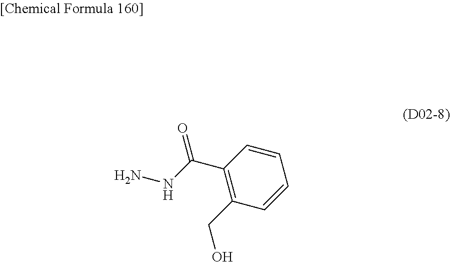 [SMILES: NNC(=O)c1ccccc1CO]

(D1)-1: acid diffusion control agent formed of compound (D1-1) shown above.

(D1)-2: acid diffusion control agent formed of compound (D1-2) shown above.

(D2)-1: acid diffusion control agent formed of compound (D2-1) shown above

(D2)-2: acid diffusion control agent formed of compound (D2-2) shown above

(S)-1: mixed solvent of propylene glycol monomethyl ether acetate/propylene glycol monomethyl ether=60/40 (mass ratio).

### <Resist Pattern Formation>

Step (i): An 8-inch silicon substrate which had been subjected to a hexamethyldisilazane (HMDS) treatment was coated with the resist composition of each example using a spinner, and subjected to a pre-bake (PAB) treatment on a hot plate at a temperature of 110° C. for 60 seconds so that the 8-inch silicon substrate was dried, thereby forming a resist film having a film thickness of 50 nm.

Step (ii): Next, using an electron beam lithography apparatus JEOL JBX-9300FS (manufactured by JEOL Ltd.), the resist film was subjected to drawing (light exposure) of a hole pattern having a target size of a pitch width of 46 nm and a hole width of 23 nm at an acceleration voltage of 100 kV. Thereafter, a post exposure bake (PEB) treatment was performed thereon at 90° C. for 60 seconds.

Step (iii): Next, alkali development was performed for 60 seconds using a 2.38 mass % tetramethylammonium hydroxide (TMAH) aqueous solution “NMD-3” (trade name, manufactured by Tokyo Ohka Kogyo Co., Ltd.) at 23° C. Thereafter, water rinsing was carried out with pure water for 15 seconds. As a result, a hole pattern having a pitch width of 46 nm and a hole width of 23 nm was formed.

### [Evaluation of Optimum Exposure Amount (Eop)]

According to <Resist pattern formation> described above, an optimum exposure amount Eop (μC/cm²) in which a pattern having the target size was formed was determined. The results thereof are listed in the columns of “Eop (μC/cm²)” in Tables 11 and 12.

[Evaluation of critical dimension uniformity (CDU) of pattern dimensions] The CDU (dimensional variation 3σ) of the hole pattern formed in <Resist pattern formation> described above was determined. Here, “3σ” denotes the triple value (3σ) (unit: nm) of the standard deviation (σ) calculated from measurement results determined by measuring the dimensions of 50 holes with a scanning electron microscope (trade name: S-9380, manufactured by Hitachi High-Tech Corporation, acceleration voltage of 800 V). The results thereof are listed in the columns of “CDU (nm)” in Tables 11 and 12.

The lower the value of 3σ determined as described above is, the higher the critical dimension (CD) uniformity of the plurality of holes formed in the resist film is.

TABLE 11

| | Eop | CDU |
|---|---|---|
| | (μC/cm²) | (nm) |
| Example 2-1 | 82 | 3.3 |
| Example 2-2 | 81 | 3.3 |
| Example 2-3 | 77 | 2.9 |
| Example 2-4 | 84 | 3.5 |
| Example 2-5 | 83 | 3.3 |
| Example 2-6 | 79 | 3.1 |
| Example 2-7 | 81 | 3.2 |
| Example 2-8 | 85 | 3.1 |
| Example 2-9 | 82 | 3.1 |
| Example 2-10 | 81 | 3.1 |
| Example 2-11 | 80 | 3.2 |
| Example 2-12 | 81 | 3.1 |
| Example 2-13 | 87 | 3.8 |
| Example 2-14 | 82 | 3.2 |

TABLE 12

| | Eop | CDU |
|---|---|---|
| | (μC/cm²) | (nm) |
| Comparative | 96 | 4.5 |
| Example 2-1 | | |
| Comparative | 110 | 4.1 |
| Example 2-2 | | |
| Comparative | 98 | 4.1 |
| Example 2-3 | | |
| Comparative | 92 | 4.0 |
| Example 2-4 | | |

As listed in Tables 11 and 12, the resist compositions of the examples had satisfactory sensitivity and satisfactory CDU as compared with the resist compositions of the comparative examples.

### Fourth Embodiment

### <Synthesis of Compounds>

### Synthesis Example 3-1: Synthesis of Compound (D03-1))

4-Iodophenol (10 g) was added to substitute the inside of the system with argon. Dichloromethane (150 g) and p-nitrophenyl chloroformate (10 g) were added thereto, the reaction container was cooled to 0° C., and triethylamine (6 g) was added dropwise thereto. After the mixture was stirred at room temperature for 1 hour, a hydrazine monohydrate (2 g) was added dropwise thereto, and the mixture was stirred at room temperature for 3 hours. After the mixture was stirred, distilled water (50 g) was added to the reaction solution to stop the reaction, and the organic layer was extracted and washed with a 10% sodium carbonate aqueous solution and pure water. The obtained organic solvent was distilled off, and the resultant was purified by silica gel column chromatography, thereby obtaining a compound (D03-1) (10 g, yield=81%).

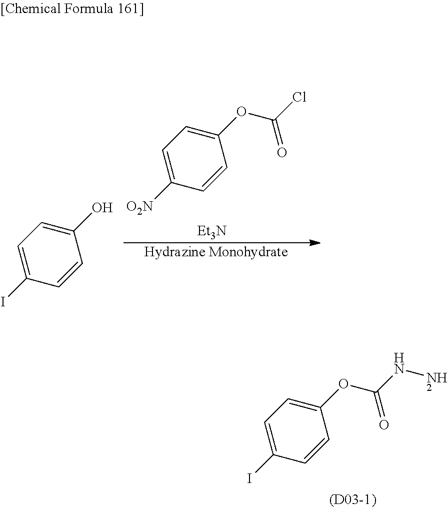 [SMILES: "Et3N" ; O=C(Cl)Oc1ccc([N+](=O)[O-])cc1 ; Oc1ccc(I)cc1 ; "Hydrazine Monohydrate" ; NNC(=O)Oc1ccc(I)cc1 ; "(D03-1)"]

The ¹H NMR measurement results of the compound (D03-1) are shown below.

¹H NMR (400 MHz, DMSO-d6) δ (ppm)=8.87 (br, N—H, 1H), 7.77 (d, C═CH, C═CH, 2H), 6.89 (d, C═CH, C═CH, 2H), 4.80 (br, NH₂, 2H)

### Synthesis Example 3-2: Synthesis of Compound (D03-2)

A compound (D03-2) (19.4 g, yield=80%) was obtained by the same method as in Synthesis Example 3-1 except that 4-iodophenol was changed to 2,4,6-triiodophenol at the equimolar amount.

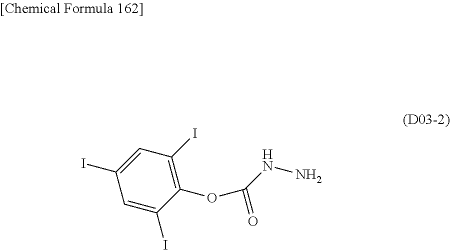 [SMILES: NNC(=O)Oc1c(I)cc(I)cc1I]

The ¹H NMR measurement results of the compound (D03-2) are shown below.

¹H NMR (400 MHz, DMSO-d6) δ (ppm)=8.88 (br, N—H, 1H), 7.98 (s, C═CH, C═CH, 2H), 4.82 (br, NH₂, 2H)

### Synthesis Example 3-3: Synthesis of Compound (D03-3)

A compound (D03-1) (10 g) was added to substitute the inside of the system with argon. Tetrahydrofuran (THF) (150 g) and γ-butyrolactone (9 g) were added thereto, the reaction container was heated to 50° C., and the mixture was stirred for 6 hours. After the mixture was stirred, the reaction solution was cooled to room temperature, and the organic layer was extracted and washed with pure water. The obtained organic solvent was distilled off, and the resultant was purified by silica gel column chromatography, thereby obtaining a compound (D03-3) (11 g, yield=79%).

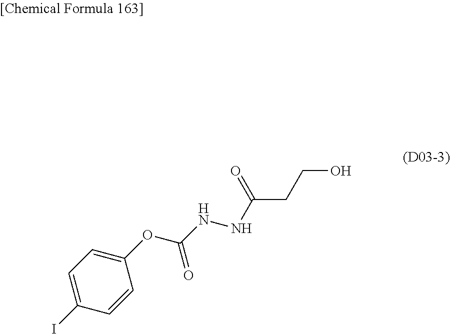 [SMILES: O=C(CCO)NNC(=O)Oc1ccc(I)cc1]

The ¹H NMR measurement results of the compound (D03-3) are shown below.

¹H NMR (400 MHz, DMSO-d6) δ (ppm)=10.27 (br, N—H, 1H), 9.98 (br, N—H, 1H), 7.74 (d, C═CH, C═CH, 2H), 6.90 (d, C═CH, C═CH, 2H), 6.34 (br, O—H, 1H), 3.81 (m, —CH₂—, 2H), 2.21 (t, —CH₂—, 2H)

### Synthesis Example 3-4: Synthesis of Compound (D03-4)

A compound (D03-4) (12.3 g, yield=82%) was obtained by the same method as in Synthesis Example 3-3 except that γ-butyrolactone was changed to phthalide at the equimolar amount.

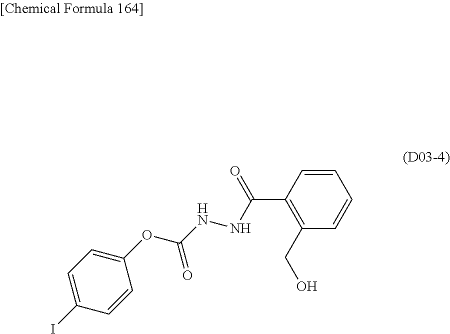 [SMILES: O=C(NNC(=O)c1ccccc1CO)Oc1ccc(I)cc1]

The ¹H NMR measurement results of the compound (D03-4) are shown below.

¹H NMR (400 MHz, DMSO-d6) δ (ppm)=11.27 (br, N—H, 1H), 10.28 (br, N—H, 1H), 8.90-6.90 (m, C═CH, C═CH, C═CH, C═CH, C═CH, C═CH, C═CH, C═CH, 8H), 6.90 (d, C═CH, C═CH, 2H), 5.64 (br, O—H, 1H), 4.76 (s, —CH₂—, 2H)

### Synthesis Example 3-5: Synthesis of Compound (D03-5)

A compound (D03-5) (13.1 g, 76%) was obtained by the same method as in Synthesis Example 3-1 except that the hydrazine monohydrate was changed to t-butyl carbazate at the equimolar amount.

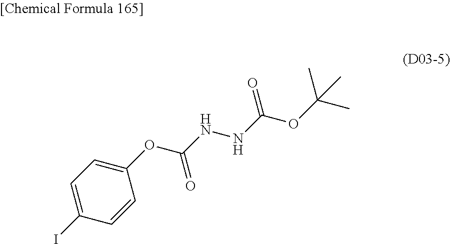 [SMILES: CC(C)(C)OC(=O)NNC(=O)Oc1ccc(I)cc1]

The ¹H NMR measurement results of the compound (D03-5) are shown below.

¹H NMR (400 MHz, DMSO-d6) δ (ppm)=11.01 (br, N—H, N—H, 2H), 7.76 (d, C═CH, C═CH, 2H), 6.92 (d, C═CH, C═CH, 2H), 1.45 (t, —CH₃, CH₃, CH₃, 9H)

### Synthesis Example 3-6: Synthesis of Compound (D03-6)

A compound (D03-1) (10 g) was added to substitute the inside of the system with argon. Dichloromethane (150 g) and benzoyl chloride (6 g) were added thereto, triethylamine (3 g) was slowly added dropwise thereto, and the mixture was stirred at room temperature for 1 hour. After the mixture was stirred, the reaction solution was filtered, and the filtrate was washed three times with TBME. The obtained white solid was purified by silica gel column chromatography, thereby obtaining a compound (D03-6) (10 g, yield=73%).

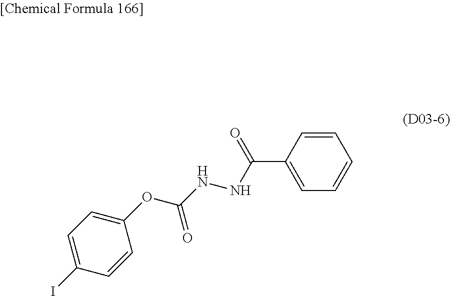 [SMILES: O=C(NNC(=O)c1ccccc1)Oc1ccc(I)cc1]

The ¹H NMR measurement results of the compound (D03-6) are shown below.

¹H NMR (400 MHz, DMSO-d6) δ (ppm)=11.27 (br, N—H, 1H), 10.27 (br, N—H, 1H), 8.90-5.92 (m, C═CH, C═CH, C═CH, C═CH, C═CH, C═CH, C═CH, C═CH, C═CH, 9H)

### Synthesis Example 3-7: Synthesis of Compound (D03-7)

4-Iodobenzene-1-thiol (20 g) was added to substitute the inside of the system with argon. Dichloromethane (DCM) (200 g) and N-chlorosuccinimide (12 g) were added thereto, and triethylamine (5 g) was added dropwise thereto. After the mixture was stirred at room temperature for 30 minutes, the reaction solution was washed with 1% hydrochloric acid and water, and the organic solvent was distilled off. The obtained white solid was dissolved in dichloromethane (200 g) to substitute the inside of the system with argon, phenol (9 g) and iron trichloride (0.5 g) were added thereto, and the mixture was stirred at room temperature for 1 hour. The reaction solution was washed with 1% hydrochloric acid and water, and the organic solvent was distilled off, thereby obtaining a mixture (23 g) of a compound (D03-7-pre) (intermediate). A compound (D03-7) (18 g, yield=55%) was obtained by the same method as in Synthesis Example 3-1 using the obtained mixture.

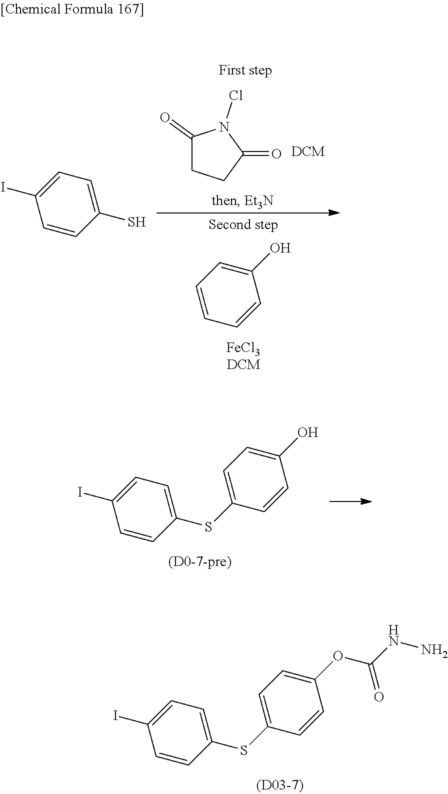 [SMILES: Sc1ccc(I)cc1 ; O=C1CCC(=O)N1Cl ; Oc1ccccc1 ; Oc1ccc(Sc2ccc(I)cc2)cc1 ; NNC(=O)Oc1ccc(Sc2ccc(I)cc2)cc1]

The ¹H NMR measurement results of the compound (D03-7) are shown below.

¹H NMR (400 MHz, DMSO-d6) δ (ppm)=8.77 (br, N—H, 1H), 8.70-5.92 (m, C═CH, C═CH, C═CH, C═CH, C═CH, C═CH, C═CH, C═CH, 8H), 4.39 (br, N—H, 1H)

### Synthesis Example 3-8: Synthesis of Compound (D03-8)

A compound (D03-8) (9.0 g, yield=75%) was obtained by the same method as in Synthesis Example 3-1 except that 4-iodophenol was changed to 4-iodonaphthalen-1-ol at the equimolar amount.

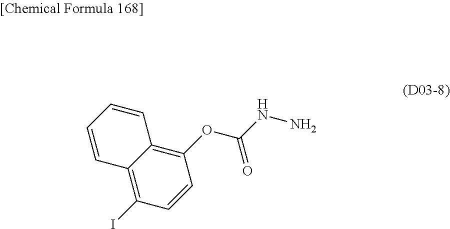 [SMILES: NNC(=O)Oc1ccc(I)c2ccccc12]

The ¹H NMR measurement results of the compound (D03-8) are shown below.

¹H NMR (400 MHz, DMSO-d6) δ (ppm)=8.57 (br, N—H, 1H), 8.70-7.50 (m, C═CH, C═CH, C═CH, C═CH, C═CH, 5H), 5.88 (d, C═CH, 2H), 4.40 (br, N—H, 1H)

### Synthesis Example 3-9: Synthesis of Compound (D03-9)

Methyl lactate (10 g) was added to substitute the inside of the system with argon. Dichloromethane (150 g) and p-nitrophenyl chloroformate (22 g) were added thereto, the reaction container was cooled to 0° C., and triethylamine (15 g) was added dropwise thereto. After the mixture was stirred at room temperature for 1 hour, the compound (D03-8) (32 g) was added thereto, and the mixture was stirred at room temperature for 24 hours. After the mixture was stirred, distilled water (50 g) was added to the reaction solution to stop the reaction, and the organic layer was extracted and washed with a 10% sodium carbonate aqueous solution and pure water. The obtained organic solvent was distilled off, and the resultant was purified by silica gel column chromatography, thereby obtaining a compound (D03-9) (21 g, yield=50%).

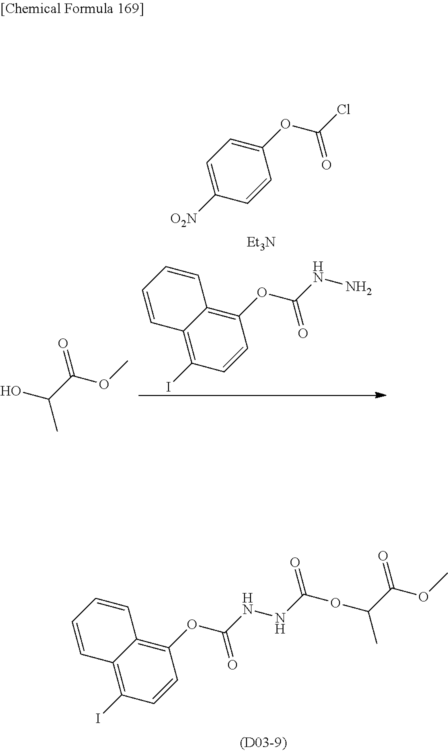

The ¹H NMR measurement results of the compound (D03-9) are shown below.

¹H NMR (400 MHz, DMSO-d6) δ (ppm)=11.22 (br, N—H, N—H, 2H), 8.70-7.50 (m, C═CH, C═CH, C═CH, C═CH, C═CH, 5H), 5.82 (d, C═CH, 2H), 5.61 (s, C—H, 1H), 3.70 (s, CH₃, 3H), 1.52 (d, CH₃, 3H)

### Synthesis Example 3-10: Synthesis of Compound (D03-10)

A compound (D03-10) (10.5 g, 74%) was obtained by the same method as in Synthesis Example 3-3 except that the compound (D03-1) was changed to the compound (D03-8) at the equimolar amount and γ-butyrolactone was changed to phthalide at the equimolar amount.

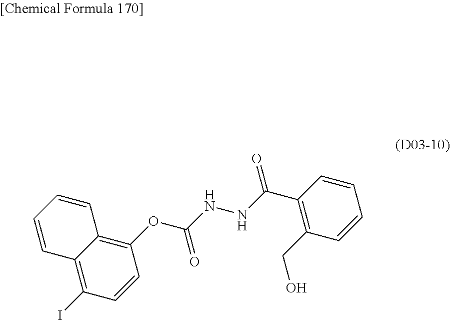 [SMILES: O=C(NNC(=O)c1ccccc1CO)Oc1ccc(I)c2ccccc12]

The ¹H NMR measurement results of the compound (D03-10) are shown below.

¹H NMR (400 MHz, DMSO-d6) δ (ppm)=11.22 (br, N—H, 1H), 10.88 (br, N—H, 1H), 8.30-7.10 (m, C═CH, C═CH, C═CH, C═CH, C═CH, C═CH, C═CH, C═CH, C═CH, 9H), 5.81 (d, C═CH, 2H), 5.20 (br, O—H, 1H), 4.67 (s, —CH₂—, 2H)

### Synthesis Example 3-11: Synthesis of Compound (D03-11)

A compound (D03-11) (11.1 g, 79%) was obtained by the same method as in Synthesis Example 3-3 except that the compound (D03-1) was changed to (2,4-diiodophenyl) hydrazine at the equimolar amount and γ-butyrolactone was changed to phthalide at the equimolar amount.

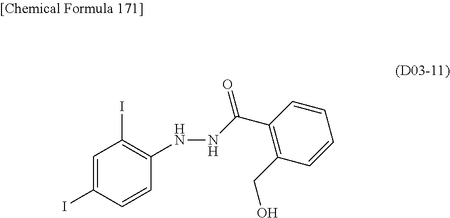 [SMILES: O=C(NNc1ccc(I)cc1I)c1ccccc1CO]

The ¹H NMR measurement results of the compound (D03-11) are shown below.

¹H NMR (400 MHz, DMSO-d6) δ (ppm)=11.5 (br, N—H, 1H) 9.01 (br, —NH, 1H), 7.94 (s, C═CH, 1H), 7.90-7.10 (m, C═CH, C═CH, C═CH, C═CH, C═CH, 5H), 6.42 (d, C═CH, 1H), 5.33 (br, —OH, 1H), 4.61 (s, CH₂, 2H)

### Synthesis Example 3-12: Synthesis of Compound (D03-12)

(4-Iodophenyl) hydrazine (10 g) was added to substitute the inside of the system with argon. 4-Methyltetrahydrofuran (MTHP) (150 g) and phthalic anhydride (9 g) were added thereto, and the mixture was heated to 80° C. and stirred for 8 hours. After the mixture was stirred, the reaction solution was cooled to 0° C., and the precipitate was filtered, and the filtrate was washed three times with TBME. The obtained white solid was purified by silica gel column chromatography, thereby obtaining a compound (D03-12) (11 g, yield=68%).

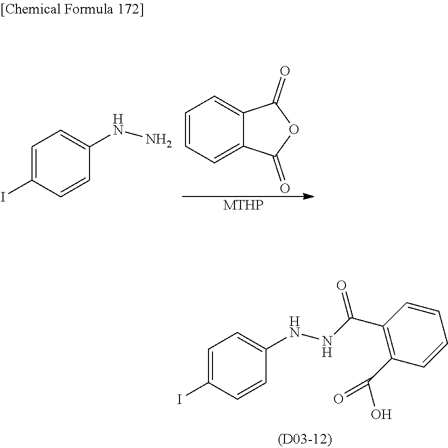

The ¹H NMR measurement results of the compound (D03-12) are shown below.

¹H NMR (400 MHz, DMSO-d6) δ (ppm)=13.89 (br, COOH, 1H), 11.61 (br, N—H, 1H) 9.57 (br, —NH, 1H), 8.50-7.10 (m, C═CH, C═CH, C═CH, C═CH, C═CH, C═H, 6H), 6.67 (d, C═CH, C═CH, 2H)

### Synthesis Example 3-13: Synthesis of Compound (D03-13)

Phenylhydrazine (10 g) was added to substitute the inside of the system with argon. THF (150 g) and 6-iodophthalide (25 g) were added thereto, the reaction container was heated to 50° C., and the mixture was stirred for 6 hours. After the mixture was stirred, the reaction solution was cooled to room temperature, and the organic layer was extracted and washed with pure water. The obtained organic solvent was distilled off, and the resultant was purified by silica gel column chromatography, thereby obtaining a compound (D03-13) (30 g, yield=88%).

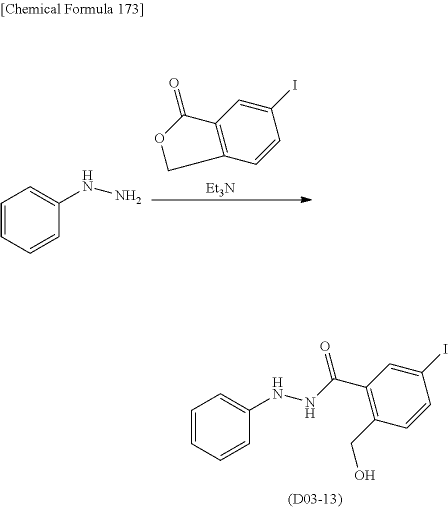 [SMILES: "Et3N" ; O=C1OCc2ccc(I)cc21 ; NNc1ccccc1 ; O=C(NNc1ccccc1)c1cc(I)ccc1CO]

The ¹H NMR measurement results of the compound (D03-13) are shown below.

¹H NMR (400 MHz, DMSO-d6) δ (ppm)=11.5 (br, N—H, 1H) 9.41 (br, —NH, 1H), 8.33 (s, C═CH, 1H), 8.20-6.90 (m, C═CH, C═CH, C═CH, C═CH, C═CH, C═CH, C═CH, 7H), 5.50 (br, —OH, 1H), 4.61 (s, CH₂, 2H)

### Synthesis Example 3-14: Synthesis of Compound (D03-14)

A compound (D03-14) (32 g, yield=71%) was obtained by the same method as in Synthesis Example 3-13 except that phenylhydrazine was changed to (4-iodophenyl) hydrazine at the equimolar amount.

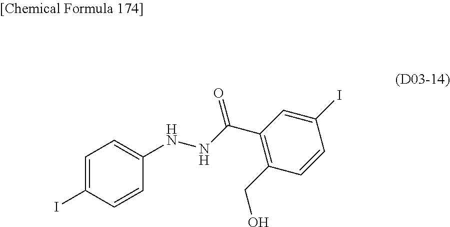 [SMILES: O=C(NNc1ccc(I)cc1)c1cc(I)ccc1CO]

The ¹H NMR measurement results of the compound (D03-14) are shown below.

¹H NMR (400 MHz, DMSO-d6) δ (ppm)=11.5 (br, N—H, 1H) 9.55 (br, —NH, 1H), 8.33 (s, C═CH, 1H), 8.20-6.50 (m, C═CH, C═CH, C═CH, C═CH, C═CH, C═CH, 6H), 5.50 (br, —OH, 1H), 4.70 (s, CH₂, 2H)

### Synthesis Example 3-15: Synthesis of Compound (D03-15)

A compound (D03-15) (26 g, yield=78%) was obtained by the same method as in Synthesis Example 3-14 except that 6-iodophthalide was changed to 4-fluorophthalic acid at the equimolar amount.

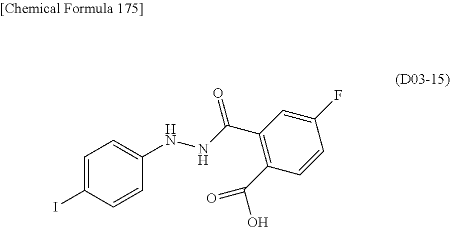 [SMILES: O=C(O)c1ccc(F)cc1C(=O)NNc1ccc(I)cc1]

The ¹H NMR measurement results of the compound (D03-15) are shown below.

¹H NMR (400 MHz, DMSO-d6) δ (ppm)=13.89 (br, COOH, 1H), 11.61 (br, N—H, 1H) 9.50 (br, —NH, 1H), 8.33 (d, C═CH, 1H), 7.80-7.20 (m, C═CH, C═CH, C═CH, C═CH, 4H), 6.67 (d, C═CH, C═CH, 2H)

### Synthesis Example 3-16: Synthesis of Compound (D03-16)

A compound (D03-16) (21 g, yield=72%) was obtained by the same method as in Synthesis Example 3-14 except that 6-iodophthalide was changed to phthalide at the equimolar amount.

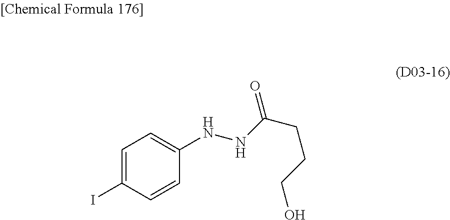 [SMILES: O=C(CCCO)NNc1ccc(I)cc1]

The ¹H NMR measurement results of the compound (D03-16) are shown below.

¹H NMR (400 MHz, DMSO-d6) δ (ppm)=11.53 (br, N—H, 1H) 9.5 (br, —NH, 1H), 7.53 (d, C═CH, C═CH, 2H), 6.65 (d, C═CH, C═CH, 2H), 4.90 (br, —OH, 1H), 3.44 (t, CH₂, 2H), 2.30-1.90, (m, CH₂, CH₂, 4H)

### Synthesis Example 3-17: Synthesis of Compound (D03-17)

A compound (D03-17) (22 g, yield=74%) was obtained by the same method as in Synthesis Example 3-14 except that 6-iodophthalide was changed to 4-methyl-2 (5H)-furanone at the equimolar amount.

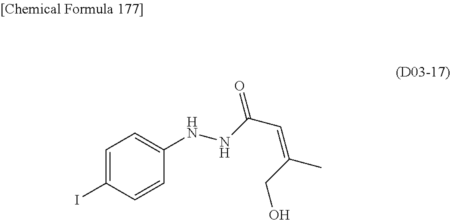 [SMILES: C/C(=C/C(=O)NNc1ccc(I)cc1)CO]

The ¹H NMR measurement results of the compound (D03-17) are shown below.

¹H NMR (400 MHz, DMSO-d6) δ (ppm)=11.53 (br, N—H, 1H) 9.18 (br, —NH, 1H), 7.43 (d, C═CH, C═CH, 2H), 6.69 (d, C═CH, C═CH, 2H), 6.30 (s, C═CH, 1H), 5.45 (br, —OH, 1H), 4.20 (d, CH₂, 2H), 2.90, (s, CH₃, 3H)

### Synthesis Example 3-18: Synthesis of Compound (D03-18)

A compound (D03-18) (28 g, yield=81%) was obtained by the same method as in Synthesis Example 3-14 except that 6-iodophthalide was changed to 1-isochromanone at the equimolar amount.

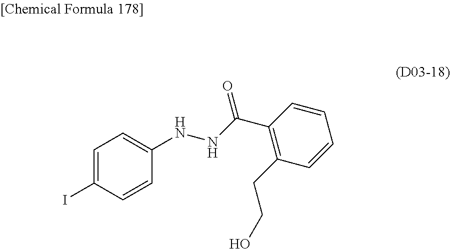 [SMILES: O=C(NNc1ccc(I)cc1)c1ccccc1CCO]

The ¹H NMR measurement results of the compound (D03-18) are shown below.

¹H NMR (400 MHz, DMSO-d6) δ (ppm)=11.52 (br, N—H, 1H) 9.51 (br, —NH, 1H), 8.20-7.0 (m, C═CH, C═CH, C═CH, C═CH, C═CH, C═CH, 6H), 6.59 (m, C═CH, C═CH, 2H), 4.21 (br, —OH, 1H), 3.58 (d, CH₂, 3H), 2.76 (d, CH₂, 2H)

### Synthesis Example 3-19: Synthesis of Compound (D03-19)

2-Hydroxymethylbenzoylhydrazide (10 g) was added to substitute the inside of the system with argon. THF (150 g) and triethylamine (5 g) were added, the container was cooled to 0° C., and 4-iodopropionyl chloride (13 g) was added thereto. The mixture was heated to room temperature and stirred for 2 hours, water was added thereto to stop the reaction, and the organic layer was extracted and washed with pure water. The obtained organic solvent was distilled off, and the resultant was purified by silica gel column chromatography, thereby obtaining a compound (D03-19) (10 g, yield=50%).

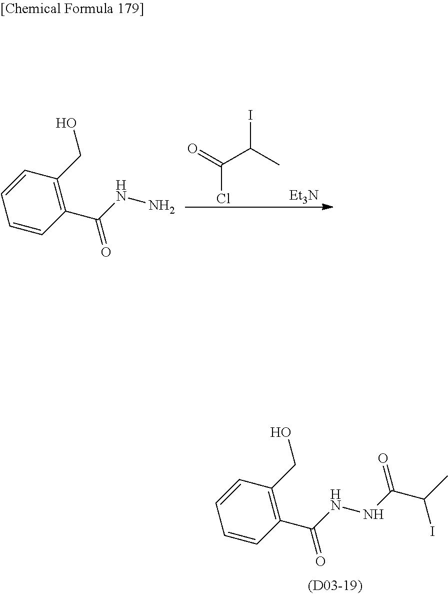

The ¹H NMR measurement results of the compound (D03-19) are shown below.

¹H NMR (400 MHz, DMSO-d6) δ (ppm)=10.5 (br, —NH, —NH, 1H), 8.0-7.0 (br, C═CH, C═CH, C═CH, C═CH, 4H), 5.40 (br, —OH, 1H), 5.01 (m, CH, 1H), 4.67, (d, CH₂, 2H), 2.20 (d, CH₃, 3H)

### <Preparation of Resist Composition>

### Examples 3-1 to 3-25 and Comparative Examples 3-1 to 3-4

Each of the components listed in Tables 13 to 15 was mixed and dissolved to prepare a resist composition of each example.

TABLE 13

| | Component | Component | Component | | Component |
|---|---|---|---|---|---|
| | (A) | (B) | (D) | | (S) |
| Example | (A1)-1 | (B1)-1 | (D03)-1 | — | (S)-1 |
| 3-1 | [100] | [30] | [10] | | [8000] |
| Example | (A1)-1 | (B1)-1 | (D03)-2 | — | (S)-1 |
| 3-2 | [100] | [30] | [10] | | [8000] |
| Example | (A1)-1 | (B1)-1 | (D03)-3 | — | (S)-1 |
| 3-3 | [100] | [30] | [10] | | [8000] |
| Example | (A1)-1 | (B1)-1 | (D03)-4 | — | (S)-1 |
| 3-4 | [100] | [30] | [10] | | [8000] |
| Example | (A1)-1 | (B1)-1 | (D03)-5 | — | (S)-1 |
| 3-5 | [100] | [30] | [10] | | [8000] |
| Example | (A1)-1 | (B1)-1 | (D03)-6 | — | (S)-1 |
| 3-6 | [100] | [30] | [10] | | [8000] |
| Example | (A1)-1 | (B1)-1 | (D03)-7 | — | (S)-1 |
| 3-7 | [100] | [30] | [10] | | [8000] |
| Example | (A1)-1 | (B1)-1 | (D03)-8 | — | (S)-1 |
| 3-8 | [100] | [30] | [10] | | [8000] |
| Example | (A1)-1 | (B1)-1 | (D03)-9 | — | (S)-1 |
| 3-9 | [100] | [30] | [10] | | [8000] |
| Example | (A1)-1 | (B1)-1 | (D03)-10 | — | (S)-1 |
| 3-10 | [100] | [30] | [10] | | [8000] |
| Example | (A1)-1 | (B1)-1 | (D03)-11 | — | (S)-1 |
| 3-11 | [100] | [30] | [10] | | [8000] |
| Example | (A1)-1 | (B1)-1 | (D03)-12 | — | (S)-1 |
| 3-12 | [100] | [30] | [10] | | [8000] |
| Example | (A1)-1 | (B1)-1 | (D03)-13 | — | (S)-1 |
| 3-13 | [100] | [30] | [10] | | [8000] |
| Example | (A1)-1 | (B1)-1 | (D03)-14 | — | (S)-1 |
| 3-14 | [100] | [30] | [10] | | [8000] |
| Example | (A1)-1 | (B1)-1 | (D03)-15 | — | (S)-1 |
| 3-15 | [100] | [30] | [10] | | [8000] |

TABLE 14

| | Component | Component | Component | | Component |
|---|---|---|---|---|---|
| | (A) | (B) | (D) | | (S) |
| Example | (A1)-1 | (B1)-1 | (D03)-16 | — | (S)-1 |
| 3-16 | [100] | [30] | [10] | | [8000] |
| Example | (A1)-1 | (B1)-1 | (D03)-17 | — | (S)-1 |
| 3-17 | [100] | [30] | [10] | | [8000] |
| Example | (A1)-1 | (B1)-1 | (D03)-18 | — | (S)-1 |
| 3-18 | [100] | [30] | [10] | | [8000] |
| Example | (A1)-1 | (B1)-1 | (D03)-19 | — | (S)-1 |
| 3-19 | [100] | [30] | [10] | | [8000] |
| Example | (A1)-2 | (B1)-1 | (D03)-4 | — | (S)-1 |
| 3-20 | [100] | [30] | [10] | | [8000] |
| Example | (A1)-3 | (B1)-1 | (D03)-4 | — | (S)-1 |
| 3-21 | [100] | [15] | [10] | | [8000] |
| Example | (A1)-3 | — | (D03)-4 | — | (S)-1 |
| 3-22 | [100] | | [10] | | [8000] |
| Example | (A1)-4 | (B1)-1 | (D03)-4 | — | (S)-1 |
| 3-23 | [100] | [30] | [10] | | [8000] |
| Example | (A1)-1 | (B1)-1 | (D03)-4 | (D1)-1 | (S)-1 |
| 3-24 | [100] | [30] | [4] | [6] | [8000] |
| Example | (A1)-1 | (B1)-2 | (D03)-4 | — | (S)-1 |
| 3-25 | [100] | [30] | [10] | | [8000] |

TABLE 15

| | Component | Component | Component | Component |
|---|---|---|---|---|
| | (A) | (B) | (D) | (S) |
| Comparative | (A1)-1 | (B1)-1 | (D2)-1 | (S)-1 |
| Example 3-1 | [100] | [30] | [10] | [8000] |
| Comparative | (A1)-1 | (B1)-1 | (D2)-2 | (S)-1 |
| Example 3-2 | [100] | [30] | [10] | [8000] |
| Comparative | (A1)-1 | (B1)-1 | (D1)-1 | (S)-1 |
| Example 3-3 | [100] | [30] | [10] | [8000] |
| Comparative | (A1)-1 | (B1)-1 | (D1)-2 | (S)-1 |
| Example 3-4 | [100] | [30] | [10] | [8000] |

In Tables 13 to 15, each abbreviation has the following meaning. The numerical values in the brackets are blending amounts (parts by mass).

(A1)-1: polymer compound represented by Formula (A1-1).

(A1)-2: polymer compound represented by Formula (A1-2).

(A1)-3: polymer compound represented by Formula (A1-3).

(A1)-4: polymer compound represented by Formula (A1-4).

(B1)-1: acid generator formed of compound (B1-1) shown above.

(B1)-2: acid generator formed of compound (B1-2) shown above.

(D03)-1: acid diffusion control agent formed of compound (D03-1) shown below.

(D03)-2: acid diffusion control agent formed of compound (D03-2) shown below.

(D03)-3: acid diffusion control agent formed of compound (D03-3) shown below.

(D03)-4: acid diffusion control agent formed of compound (D03-4) shown below.

(D03)-5: acid diffusion control agent formed of compound (D03-5) shown below.

(D03)-6: acid diffusion control agent formed of compound (D03-6) shown below.

(D03)-7: acid diffusion control agent formed of compound (D03-7) shown below.

(D03)-8: acid diffusion control agent formed of compound (D03-8) shown below.

(D03)-9: acid diffusion control agent formed of compound (D03-9) shown below.

(D03)-10: acid diffusion control agent formed of compound (D03-10) shown below.

(D03)-11: acid diffusion control agent formed of compound (D03-11) shown below.

(D03)-12: acid diffusion control agent formed of compound (D03-12) shown below.

(D03)-13: acid diffusion control agent formed of compound (D03-13) shown below.

(D03)-14: acid diffusion control agent formed of compound (D03-14) shown below.

(D03)-15: acid diffusion control agent formed of compound (D03-15) shown below.

(D03)-16: acid diffusion control agent formed of compound (D03-16) shown below.

(D03)-17: acid diffusion control agent formed of compound (D03-17) shown below.

(D03)-18: acid diffusion control agent formed of compound (D03-18) shown below.

(D03)-19: acid diffusion control agent formed of compound (D03-19) shown below.

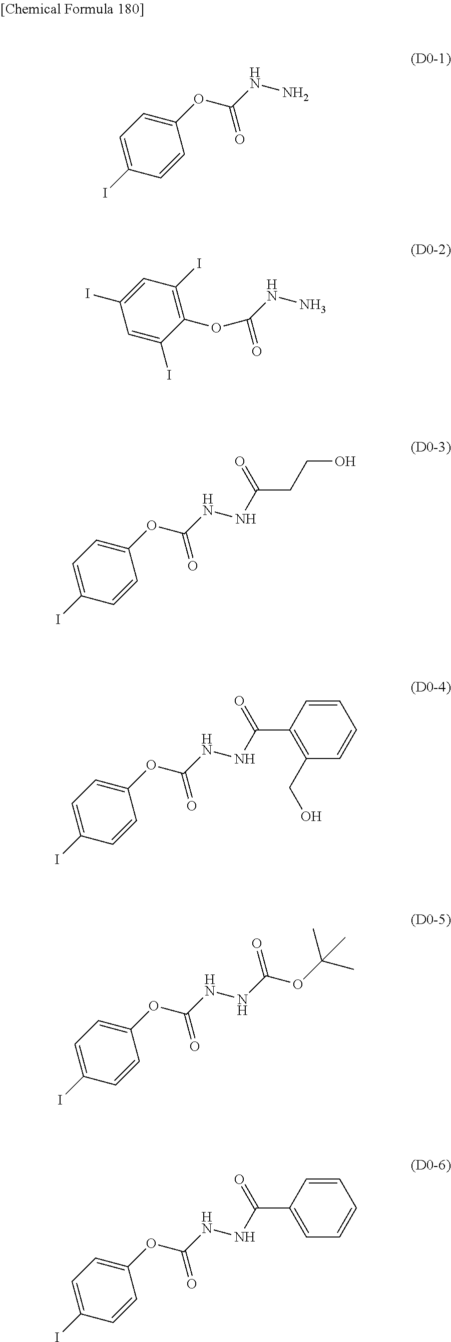 [SMILES: NNC(=O)Oc1ccc(I)cc1 ; NNC(=O)Oc1c(I)cc(I)cc1I ; O=C(CCO)NNC(=O)Oc1ccc(I)cc1 ; O=C(NNC(=O)c1ccccc1CO)Oc1ccc(I)cc1 ; CC(C)(C)OC(=O)NNC(=O)Oc1ccc(I)cc1 ; O=C(NNC(=O)c1ccccc1)Oc1ccc(I)cc1]

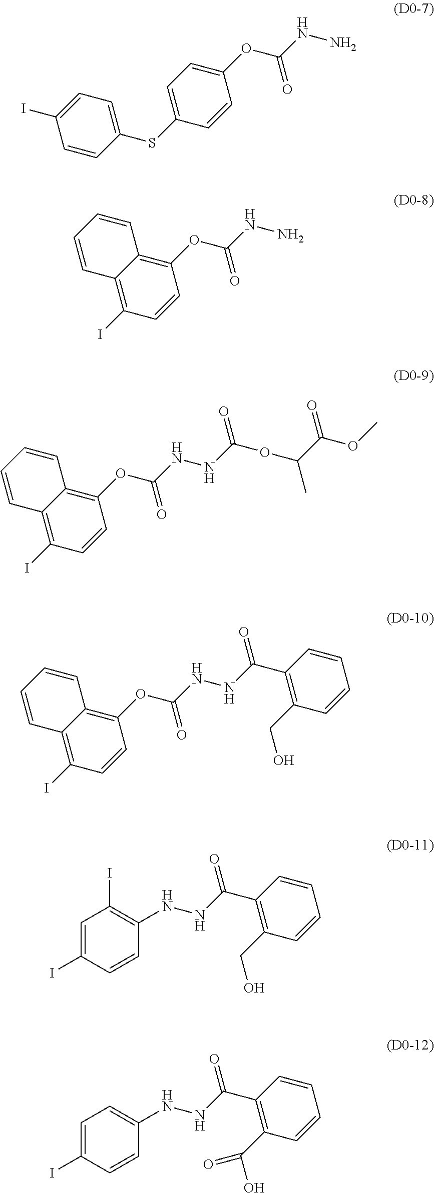 [SMILES: NNC(=O)Oc1ccc(Sc2ccc(I)cc2)cc1 ; NNC(=O)Oc1ccc(I)c2ccccc12 ; COC(=O)C(C)OC(=O)NNC(=O)Oc1ccc(I)c2ccccc12 ; O=C(NNC(=O)c1ccccc1CO)Oc1ccc(I)c2ccccc12 ; O=C(NNc1ccc(I)cc1I)c1ccccc1CO ; O=C(O)c1ccccc1C(=O)NNc1ccc(I)cc1]

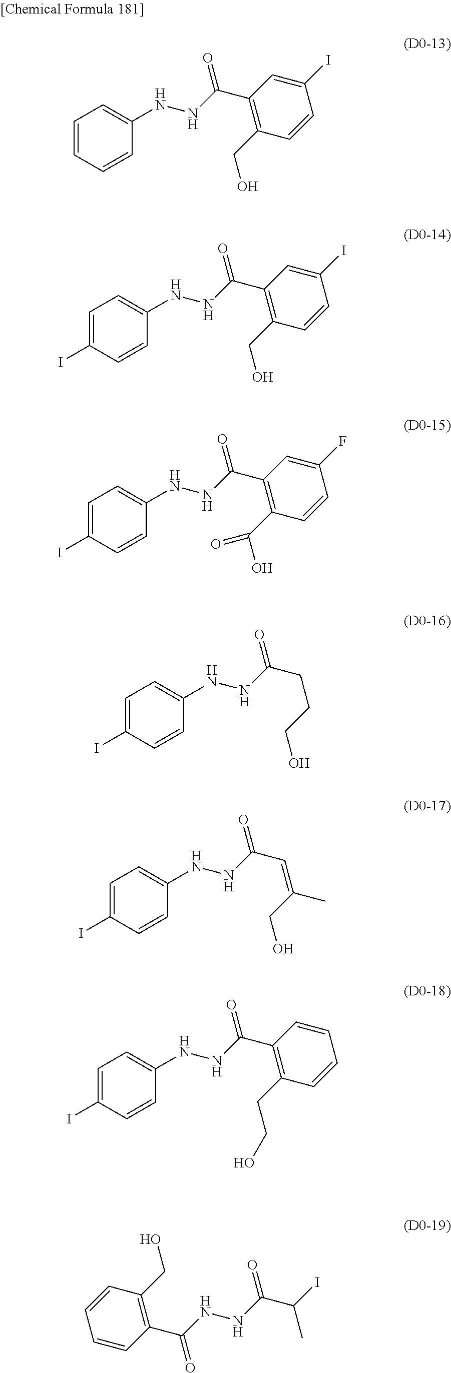

(D1)-1: acid diffusion control agent formed of compound (D1-1) shown above.

(D1)-2: acid diffusion control agent formed of compound (D1-2) shown above.

(D2)-1: acid diffusion control agent formed of compound (D2-1) shown above.

(D2)-2: acid diffusion control agent formed of compound (D2-2) shown above.

(S)-1: mixed solvent of propylene glycol monomethyl ether acetate/propylene glycol monomethyl ether=60/40 (mass ratio).

### <Resist Pattern Formation>

Step (i): An 8-inch silicon substrate which had been subjected to a hexamethyldisilazane (HMDS) treatment was coated with the resist composition of each example using a spinner, and subjected to a pre-bake (PAB) treatment on a hot plate at a temperature of 110° C. for 60 seconds so that the 8-inch silicon substrate was dried, thereby forming a resist film having a film thickness of 50 nm.

Step (ii): Next, using an electron beam lithography apparatus JEOL JBX-9300FS (manufactured by JEOL Ltd.), the resist film was subjected to drawing (light exposure) of a hole pattern having a target size of a pitch width of 46 nm and a hole width of 23 nm at an acceleration voltage of 100 kV. Thereafter, a post exposure bake (PEB) treatment was performed thereon at 90° C. for 60 seconds.

Step (iii): Next, alkali development was performed for 60 seconds using a 2.38 mass % tetramethylammonium hydroxide (TMAH) aqueous solution “NMD-3” (trade name, manufactured by Tokyo Ohka Kogyo Co., Ltd.) at 23° C. Thereafter, water rinsing was carried out with pure water for 15 seconds. As a result, a hole pattern having a pitch width of 46 nm and a hole width of 23 nm was formed.

### [Evaluation of Optimum Exposure Amount (Eop)]

According to <Resist pattern formation> described above, an optimum exposure amount Eop (μC/cm²) in which a pattern having the target size was formed was determined. The results thereof are listed in the columns of “EoP (μC/cm²)” in Tables 16 to 18.

### [Evaluation of Critical Dimension Uniformity (CDU) of Pattern Dimensions]

The CDU (dimensional variation 3σ) of the hole pattern formed in <Resist pattern formation> described above was determined. Here, “3σ” denotes the triple value (3σ) (unit: nm) of the standard deviation (σ) calculated from measurement results determined by measuring the dimensions of 50 holes with a scanning electron microscope (trade name: S-9380, manufactured by Hitachi High-Tech Corporation, acceleration voltage of 800 V). The results thereof are listed in the columns of “CDU (nm)” in Tables 16 to 18.

The lower the value of 30 determined as described above is, the higher the critical dimension (CD) uniformity of the plurality of holes formed in the resist film is.

TABLE 16

| | Eop | CDU |
|---|---|---|
| | (μC/cm²) | (nm) |
| Example 3-1 | 86 | 3.4 |
| Example 3-2 | 84 | 3.4 |
| Example 3-3 | 85 | 3.3 |
| Example 3-4 | 80 | 3.2 |
| Example 3-5 | 85 | 3.4 |
| Example 3-6 | 86 | 3.4 |
| Example 3-7 | 81 | 3.2 |
| Example 3-8 | 80 | 3.2 |
| Example 3-9 | 85 | 3.3 |
| Example 3-10 | 76 | 3.1 |
| Example 3-11 | 80 | 3.3 |
| Example 3-12 | 81 | 3.2 |
| Example 3-13 | 75 | 3.2 |
| Example 3-14 | 75 | 3.2 |
| Example 3-15 | 78 | 3.4 |

TABLE 17

| | Eop | CDU |
|---|---|---|
| | (μC/cm²) | (nm) |
| Example 3-16 | 81 | 3.3 |
| Example 3-17 | 82 | 3.2 |
| Example 3-18 | 83 | 3.2 |
| Example 3-19 | 81 | 3.1 |
| Example 3-20 | 80 | 3.1 |
| Example 3-21 | 81 | 3.1 |
| Example 3-22 | 87 | 3.0 |
| Example 3-23 | 82 | 3.0 |
| Example 3-24 | 87 | 3.2 |
| Example 3-25 | 81 | 3.2 |

TABLE 18

| | Eop | CDU |
|---|---|---|
| | (μC/cm²) | (nm) |
| Comparative | 96 | 4.5 |
| Example 3-1 | | |
| Comparative | 110 | 4.1 |
| Example 3-2 | | |
| Comparative | 98 | 4.1 |
| Example 3-3 | | |
| Comparative | 92 | 4.0 |
| Example 3-4 | | |

As listed in Tables 16 to 18, the resist compositions of the examples had satisfactory sensitivity and satisfactory CDU as compared with the resist compositions of the comparative examples.

While preferred embodiments of the present invention have been described and illustrated above, it should be understood that these are exemplary of the present invention and are not to be considered as limiting. Additions, omissions, substitutions, and other modifications can be made without departing from the scope of the invention. Accordingly, the present invention is not limited by the description above but by the scope of the appended claims.

What is claimed is:

1. A resist composition which generates an acid upon light exposure and whose solubility in a developing solution is changed by an action of the acid, the resist composition comprising:
a resin component (A1) whose solubility in a developing solution is changed by the action of the acid; and
a compound (D0) represented by General Formula (d0), 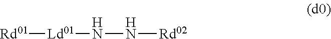
wherein Rd⁰¹ represents a monovalent organic group, Rd⁰² represents a monovalent organic group or a hydrogen atom, and Ld⁰¹ represents a single bond or a divalent linking group.

2. (canceled)

3. (canceled)

4. (canceled)

5. The resist composition according to claim 1, wherein the compound (D0) is a compound (D01) represented by General Formula (d01), 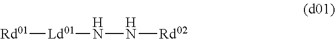
wherein Rd⁰¹ represents a monovalent organic group having at least two benzene rings which may have a substituent, Rd⁰² represents a monovalent organic group or a hydrogen atom, and Ld⁰¹ represents a single bond or a divalent linking group.

6. (canceled)

7. (canceled)

8. (canceled)

9. (canceled)

10. (canceled)

11. The resist composition according to claim 1, wherein the compound (D0) is a compound (D02) represented by General Formula (d02), 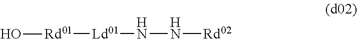
wherein Rd⁰¹ represents a divalent organic group, Rd⁰² represents a monovalent organic group or a hydrogen atom, and Ld⁰¹ represents a single bond or a divalent linking group.

12. (canceled)

13. (canceled)

14. The resist composition according to claim 1, wherein the compound (D0) is a compound (D03) represented by General Formula (d03), 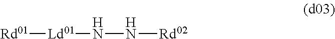
wherein Rd⁰¹ represents a monovalent organic group, Rd⁰² represents a monovalent organic group or a hydrogen atom, and Ld⁰¹ represents a single bond or a divalent linking group, where at least one of Rd⁰¹ or Rd⁰² has at least one iodine atom as a substituent.

15. (canceled)

16. (canceled)

17. (canceled)

18. A method for forming a resist pattern, comprising:
forming a resist film on a support using the resist composition according to claim 1;
exposing the resist film to light; and
developing the resist film exposed to light to form a resist pattern.

19. (canceled)

20. A compound which is represented by General Formula (dc01), 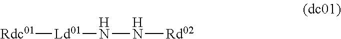
wherein Rdc⁰¹ represents a monovalent organic group having at least two benzene rings which may have a substituent and having at least one heteroatom, Rd⁰² represents a monovalent organic group or a hydrogen atom, and Ld⁰¹ represents a single bond or a divalent linking group.

21. (canceled)

22. (canceled)

23. (canceled)

24. (canceled)

25. (canceled)

26. A compound which is represented by General Formula (d02-3), 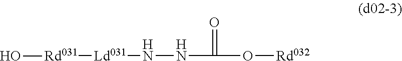
wherein Rd⁰³¹ represents a divalent organic group, Rd⁰³² represents a monovalent organic group, and Ld⁰³¹ represents a single bond or a divalent linking group.

27. A compound which is represented by General Formula (d03-1-1), 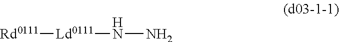
wherein Rd⁰¹¹¹ represents a monovalent organic group having at least one iodine atom as a substituent, and Ld⁰¹¹¹ represents a divalent linking group.

28. A compound which is represented by General Formula (d03-2-1), 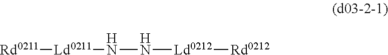
wherein Rd⁰²¹¹ and Rd⁰²¹² each independently represents a monovalent organic group, and Ld⁰²¹¹ and Ld⁰²¹² each independently represents a divalent linking group, wherein at least one of Rd⁰²¹¹ or Rd⁰²¹² has at least one iodine atom as a substituent.

29. A compound which is represented by General Formula (d03-2-2), 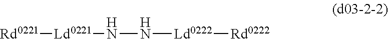
wherein Rd⁰²²¹ represents a monovalent organic group, Rd⁰²²² represents a monovalent organic group having —(CH₂)ₚ—OH or a carboxy group as a substituent, p represents an integer of 1 to 3, and Ld⁰²²¹ and Ld⁰²²² each independently represents a single bond or a divalent linking group, where at least one of Rd⁰²²¹ or Rd⁰²²² has at least one iodine atom as a substituent.

30. An acid diffusion control agent comprising:
a compound (D0) represented by General Formula (d0), 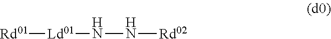
wherein Rd⁰¹ represents a monovalent organic group, Rd⁰² represents a monovalent organic group or a hydrogen atom, and Ld⁰¹ represents a single bond or a divalent linking group.

31. (canceled)

32. (canceled)

33. (canceled)

34. The acid diffusion control agent according to claim 30,
wherein the compound (D0) is a compound (D02) represented by General Formula (d02), 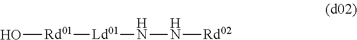
wherein Rd⁰¹ represents a monovalent organic group, Rd⁰² represents a monovalent organic group or a hydrogen atom, and Ld⁰¹ represents a single bond or a divalent linking group.

35. (canceled)

36. (canceled)

37. The acid diffusion control agent according to claim 30,
wherein the compound (D0) is a compound (D03) represented by General Formula (d03), 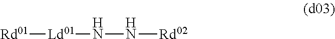
wherein Rd⁰¹ represents a monovalent organic group, Rd⁰² represents a monovalent organic group or a hydrogen atom, and Ld⁰¹ represents a single bond or a divalent linking group, where at least one of Rd⁰¹ or Rd⁰² has at least one iodine atom as a substituent.

38. (canceled)

39. (canceled)

40. (canceled)

41. The acid diffusion control agent according to claim 30, wherein the compound (D0) is the compound according to claim 20.
